SEQUENCE LISTING (76 sequences)

SEQ ID NO: 1 - protein, 1014 aa, Homo sapiens
MAESSDKLYRVEYAKSGRASCKKCSESIPKDSLRMAIMVQSPMFDGKVPHWYHFSCFWKVGHSIRHPDVEVDGFSELRWDDQQKVKKTAEAGGVTGKGQDGIGSKAEKTLGDFAAEYAKSNRSTCKGCMEKIEKGQVRLSKKMVDPEKPQLGMIDRWYHPGCFVKNREELGFRPEYSASQLKGFSLLATEDKEALKKQLPGVKSEGKRKGDEVDGVDEVAKKKSKKEKDKDSKLEKALKAQNDLIWNIKDELKKVCSTNDLKELLIFNKQQVPSGESAILDRVADGMVFGALLPCEECSGQLVFKSDAYYCTGDVTAWTKCMVKTQTPNRKEWVTPKEFREISYLKKLKVKKQDRIFPPETSASVAATPPPSTASAPAAVNSSASADKPLSNMKILTLGKLSRNKDEVKAMIEKLGGKLTGTANKASLCISTKKEVEKMNKKMEEVKEANIRVVSEDFLQDVSASTKSLQELFLAHILSPWGAEVKAEPVEVVAPRGKSGAALSKKSKGQVKEEGINKSEKRMKLTLKGGAAVDPDSGLEHSAHVLEKGGKVFSATLGLVDIVKGTNSYYKLQLLEDDKENRYWIFRSWGRVGTVIGSNKLEQMPSKEDAIEHFMKLYEEKTGNAWHSKNFTKYPKKFYPLEIDYGQDEEAVKKLTVNPGTKSKLPKPVQDLIKMIFDVESMKKAMVEYEIDLQKMPLGKLSKRQIQAAYSILSEVQQAVSQGSSDSQILDLSNRFYTLIPHDFGMKKPPLLNNADSVQAKVEMLDNLLDIEVAYSLLRGGSDDSSKDPIDVNYEKLKTDIKVVDRDSEEAEIIRKYVKNTHATTHNAYDLEVIDIFKIEREGECQRYKPFKQLHNRRLLWHGSRTTNFAGILSQGLRIAPPEAPVTGYMFGKGIYFADMVSKSANYCHTSQGDPIGLILLGEVALGNMYELKHASHISKLPKGKHSVKGLGKTTPDPSANISLDGVDVPLGTGISSGVNDTSLLYNEYIVYDIAQVNLKYLLKLKFNFKTSLW

SEQ ID NO: 2 - DNA, 3978 bp, Homo sapiens
  source 1..3978: mol_type = other DNA
agcaatctatcagggaacggcggtggccggtgcggcgtgttcggtggcggctctggccgctcaggcgcctgcggctgggtgagcgcacgcgaggcggcgaggcggcagcgtgtttctaggtcgtggcgtcgggcttccggagctttggcggcagctaggggaggatggcggagtcttcggataagctctatcgagtcgagtacgccaagagcgggcgcgcctcttgcaagaaatgcagcgagagcatccccaaggactcgctccggatggccatcatggtgcagtcgcccatgtttgatggaaaagtcccacactggtaccacttctcctgcttctggaaggtgggccactccatccggcaccctgacgttgaggtggatgggttctctgagcttcggtgggatgaccagcagaaagtcaagaagacagcggaagctggaggagtgacaggcaaaggccaggatggaattggtagcaaggcagagaagactctgggtgactttgcagcagagtatgccaagtccaacagaagtacgtgcaaggggtgtatggagaagatagaaaagggccaggtgcgcctgtccaagaagatggtggacccggagaagccacagctaggcatgattgaccgctggtaccatccaggctgctttgtcaagaacagggaggagctgggtttccggcccgagtacagtgcgagtcagctcaagggcttcagcctccttgctacagaggataaagaagccctgaagaagcagctcccaggagtcaagagtgaaggaaagagaaaaggcgatgaggtggatggagtggatgaagtggcgaagaagaaatctaaaaaagaaaaagacaaggatagtaagcttgaaaaagccctaaaggctcagaacgacctgatctggaacatcaaggacgagctaaagaaagtgtgttcaactaatgacctgaaggagctactcatcttcaacaagcagcaagtgccttctggggagtcggcgatcttggaccgagtagctgatggcatggtgttcggtgccctccttccctgcgaggaatgctcgggtcagctggtcttcaagagcgatgcctattactgcactggggacgtcactgcctggaccaagtgtatggtcaagacacagacacccaaccggaaggagtgggtaaccccaaaggaattccgagaaatctcttacctcaagaaattgaaggttaaaaaacaggaccgtatattccccccagaaaccagcgcctccgtggcggccacgcctccgccctccacagcctcggctcctgctgctgtgaactcctctgcttcagcagataagccattatccaacatgaagatcctgactctcgggaagctgtcccggaacaaggatgaagtgaaggccatgattgagaaactcggggggaagttgacggggacggccaacaaggcttccctgtgcatcagcaccaaaaaggaggtggaaaagatgaataagaagatggaggaagtaaaggaagccaacatccgagttgtgtctgaggacttcctccaggacgtctccgcctccaccaagagccttcaggagttgttcttagcgcacatcttgtccccttggggggcagaggtgaaggcagagcctgttgaagttgtggccccaagagggaagtcaggggctgcgctctccaaaaaaagcaagggccaggtcaaggaggaaggtatcaacaaatctgaaaagagaatgaaattaactcttaaaggaggagcagctgtggatcctgattctggactggaacactctgcgcatgtcctggagaaaggtgggaaggtcttcagtgccacccttggcctggtggacatcgttaaaggaaccaactcctactacaagctgcagcttctggaggacgacaaggaaaacaggtattggatattcaggtcctggggccgtgtgggtacggtgatcggtagcaacaaactggaacagatgccgtccaaggaggatgccattgagcacttcatgaaattatatgaagaaaaaaccgggaacgcttggcactccaaaaatttcacgaagtatcccaaaaagttctaccccctggagattgactatggccaggatgaagaggcagtgaagaagctgacagtaaatcctggcaccaagtccaagctccccaagccagttcaggacctcatcaagatgatctttgatgtggaaagtatgaagaaagccatggtggagtatgagatcgaccttcagaagatgcccttggggaagctgagcaaaaggcagatccaggccgcatactccatcctcagtgaggtccagcaggcggtgtctcagggcagcagcgactctcagatcctggatctctcaaatcgcttttacaccctgatcccccacgactttgggatgaagaagcctccgctcctgaacaatgcagacagtgtgcaggccaaggtggaaatgcttgacaacctgctggacatcgaggtggcctacagtctgctcaggggagggtctgatgatagcagcaaggatcccatcgatgtcaactatgagaagctcaaaactgacattaaggtggttgacagagattctgaagaagccgagatcatcaggaagtatgttaagaacactcatgcaaccacacacaatgcgtatgacttggaagtcatcgatatctttaagatagagcgtgaaggcgaatgccagcgttacaagccctttaagcagcttcataaccgaagattgctgtggcacgggtccaggaccaccaactttgctgggatcctgtcccagggtcttcggatagccccgcctgaagcgcccgtgacaggctacatgtttggtaaagggatctatttcgctgacatggtctccaagagtgccaactactgccatacgtctcagggagacccaataggcttaatcctgttgggagaagttgcccttggaaacatgtatgaactgaagcacgcttcacatatcagcaagttacccaagggcaagcacagtgtcaaaggtttgggcaaaactacccctgatccttcagctaacattagtctggatggtgtagacgttcctcttgggaccgggatttcatctggtgtgaatgacacctctctactatataacgagtacattgtctatgatattgctcaggtaaatctgaagtatctgctgaaactgaaattcaattttaagacctccctgtggtaattgggagaggtagccgagtcacacccggtggctctggtatgaattcacccgaagcgcttctgcaccaactcacctggccgctaagttgctgatgggtagtacctgtactaaaccacctcagaaaggattttacagaaacgtgttaaaggttttctctaacttctcaagtcccttgttttgtgttgtgtctgtggggaggggttgttttggggttgtttttgttttttcttgccaggtagataaaactgacatagagaaaaggctggagagagattctgttgcatagactagtcctatggaaaaaaccaagcttcgttagaatgtctgccttactggtttccccagggaaggaaaaatacacttccacccttttttctaagtgttcgtctttagttttgattttggaaagatgttaagcatttatttttagttaaaaataaaaactaatttcatactatttagattttcttttttatcttgcacttattgtcccctttttagttttttttgtttgcctcttgtggtgaggggtgtgggaagaccaaaggaaggaacgctaacaatttctcatacttagaaacaaaaagagctttccttctccaggaatactgaacatgggagctcttgaaatatgtagtattaaaagttgcatttgaaattcttgactttcttatgggcacttttgtcttccaaattaaaactctaccacaaatatacttacccaagggctaatagtaatactcgattaaaaatgcagatgccttctcta

SEQ ID NO: 3 - protein, 578 aa, Homo sapiens
MRQQDAPKPTPAACRCSGLARRVLTIAFALLILGLMTWAYAAGVPLASDRYGLLAFGLYGAFLSAHLVAQSLFAYLEHRRVAAAARGPLDAATARSVALTISAYQEDPAYLRQCLASARALLYPRARLRVLMVVDGNRAEDLYMVDMFREVFADEDPATYVWDGNYHQPWEPAAAGAVGAGAYREVEAEDPGRLAVEALVRTRRCVCVAQRWGGKREVMYTAFKALGDSVDYVQVCDSDTRLDPMALLELVRVLDEDPRVGAVGGDVRILNPLDSWVSFLSSLRYWVAFNVERACQSYFHCVSCISGPLGLYRNNLLQQFLEAWYNQKFLGTHCTFGDDRHLTNRMLSMGYATKYTSRSRCYSETPSSFLRWLSQQTRWSKSYFREWLYNALWWHRHHAWMTYEAVVSGLFPFFVAATVLRLFYAGRPWALLWVLLCVQGVALAKAAFAAWLRGCLRMVLLSLYAPLYMCGLLPAKFLALVTMNQSGWGTSGRRKLAANYVPLLPLALWALLLLGGLVRSVAHEARADWSGPSRAAEAYHLAAGAGAYVGYWVAMLTLYWVGVRRLCRRRTGGYRVQV

SEQ ID NO: 4 - DNA, 2107 bp, Homo sapiens
  source 1..2107: mol_type = other DNA
acagagaggctgggagagcaagccggagagaagagagagcccggccagacccactgcgatgagacaggacgcgcccaagcccactcctgcagcctgccgctgctccggcctggcccggagggtgctgaccatcgccttcgccctgctcatcctgggcctcatgacctgggcctacgccgccggggtgccgctggcctccgatcgctacggcctcctggccttcggcctctacggggccttcctttcagcgcacctggtggcgcagagcctcttcgcgtacctggagcaccggcgggtggcggcggcggcgcgggggccgctggatgcagccaccgcgcgcagtgtggcgctgaccatctccgcctaccaggaggaccccgcgtacctgcgccagtgcctggcgtccgcccgcgccctgctgtacccgcgcgcgcggctgcgcgtcctcatggtggtggatggcaaccgcgccgaggacctctacatggtcgacatgttccgcgaggtcttcgctgacgaggaccccgccacgtacgtgtgggacggcaactaccaccagccctgggaacccgcggcggcgggcgcggtgggcgccggagcctatcgggaggtggaggcggaggatcctgggcggctggcagtggaggcgctggtgaggactcgcaggtgcgtgtgcgtggcgcagcgctggggcggcaagcgcgaggtcatgtacacagccttcaaggcgctcggagattcggtggactacgtgcaggtctgtgactcggacacaaggttggaccccatggcactgctggagctcgtgcgggtactggacgaggacccccgggtaggggctgttggtggggacgtgcggatccttaaccctctggactcctgggtcagcttcctaagcagcctgcgatactgggtagccttcaatgtggagcgggcttgtcagagctacttccactgtgtatcctgcatcagcggtcctctaggcctatataggaataacctcttgcagcagtttcttgaggcctggtacaaccagaagttcctgggtacccactgtacttttggggatgaccggcacctcaccaaccgcatgctcagcatgggttatgctaccaagtacacctccaggtcccgctgctactcagagacgccctcgtccttcctgcggtggctgagccagcagacacgctggtccaagtcgtacttccgtgagtggctgtacaacgcgctctggtggcaccggcaccatgcgtggatgacctacgaggcggtggtctccggcctgttccccttcttcgtggcggccactgtgctgcgtctgttctacgcgggccgcccttgggcgctgctgtgggtgctgctgtgcgtgcagggcgtggcactggccaaggcggccttcgcggcctggctgcggggctgcctgcgcatggtgcttctgtcgctctacgcgcccctctacatgtgtggcctcctgcctgccaagttcctggcgctagtcaccatgaaccagagtggctggggcacctcgggccggcggaagctggccgctaactacgtccctctgctgcccctggcgctctgggcgctgctgctgcttgggggcctggtccgcagcgtagcacacgaggccagggccgactggagcggcccttcccgcgcagccgaggcctaccacttggccgcgggggccggcgcctacgtgggctactgggtggccatgttgacgctgtactgggtgggcgtgcggaggctttgccggcggcggaccgggggctaccgcgtccaggtgtgagtccagccacgcggatgccgcctcaagggtcttcaggggaggccagaggagagctgctgggccccgagccacgaacttgctgggtggttctctgggcctcagtttccctcctctgcaaaacgagggggtcagcccaagattcttcagtctggactatattgggactgggacttctgggtctccagggagggtatttattggtcagggtgtgggatctgaggagtggagggaaagggtcctgctttctcctcgttcttatttaatctccatttctactgtgtgatcaggatgtaataaagaattttatttattttc

SEQ ID NO: 5 - protein, 552 aa, Homo sapiens
MHCERFLCILRIIGTTLFGVSLLLGITAAYIVGYQFIQTDNYYFSFGLYGAFLASHLIIQSLFAFLEHRKMKKSLETPIKLNKTVALCIAAYQEDPDYLRKCLQSVKRLTYPGIKVVMVIDGNSEDDLYMMDIFSEVMGRDKSATYIWKNNFHEKGPGETDESHKESSQHVTQLVLSNKSICIMQKWGGKREVMYTAFRALGRSVDYVQVCDSDTMLDPASSVEMVKVLEEDPMVGGVGGDVQILNKYDSWISFLSSVRYWMAFNIERACQSYFGCVQCISGPLGMYRNSLLHEFVEDWYNQEFMGNQCSFGDDRHLTNRVLSLGYATKYTARSKCLTETPIEYLRWLNQQTRWSKSYFREWLYNAMWFHKHHLWMTYEAIITGFFPFFLIATVIQLFYRGKIWNILLFLLTVQLVGLIKSSFASCLRGNIVMVFMSLYSVLYMSSLLPAKMFAIATINKAGWGTSGRKTIVVNFIGLIPVSVWFTILLGGVIFTIYKESKRPFSESKQTVLIVGTLLYACYWVMLLTLYVVLINKCGRRKKGQQYDMVLDV

SEQ ID NO: 6 - DNA, 4240 bp, Homo sapiens
  source 1..4240: mol_type = other DNA
agacccccttaagttggaggaggcagaagggcaacaacggcggggaaggagaagtcaagacgtctggaaagaattacccagtcctggcttcgagcagcccattgaaccagagacttgaaacagccccagccaaagacttttctcccaattctgcgcttcctgggttctgctgagtcttccacaggctttttttttttttttttttttttttaagacgaaaaagagattttctgttatcgggggcagaaagactgaagcccaaaaaaaaaaaaaaaaaaaaaagaaaagaaaagaaaaaagaaaagttaatttatttttaaagcataatttttttaagaattagactgaagtgcaacggaaacataaagagaatattagtgaaattattttttaaagtggggaagaatcaaacatttaagactcccctatcctttttaaatgttgtttttaaatttcttattttttttggccggtcgtctcaaattcatctgatctcttattacctcaattttggaaactgcccgccaccgaccctccgggaccacacagacaggctgaggacgactttatgaccaagagctgaacaagatgcattgtgagaggtttctatgtatcctgagaataattggaaccacactctttggagtctctctcctccttggaatcacagctgcttatattgttggctaccagtttatccaaacggataattactatttctcttttggactgtatggtgcctttttggcatcacacctcatcatccaaagcctgtttgcctttttggagcaccgaaaaatgaaaaaatccctagaaacccccataaagttgaacaaaacagttgccctttgcatcgctgcctatcaagaagatccagactacttaaggaaatgtttgcaatctgtgaaaaggctaacctaccctgggattaaagttgtcatggtcatagatgggaactcagaagatgacctttacatgatggacatcttcagtgaagtcatgggcagagacaaatcagccacttatatctggaagaacaacttccacgaaaagggtcccggtgagacagatgagtcacataaagaaagctcgcaacacgtaacgcaattggtcttgtccaacaaaagtatctgcatcatgcaaaaatggggtggaaaaagagaagtcatgtacacagccttcagagcactgggacgaagtgtggattatgtacaggtttgtgattcagacactatgcttgacccagcctcatctgtggagatggtaaaagttttagaagaagatcccatggttggaggtgttgggggagatgtccagattttaaacaagtacgattcctggatctcattcctcagcagtgtaagatattggatggcttttaatatagaaagggcctgtcagtcttattttgggtgtgttcagtgcattagtggacctctgggaatgtacagaaactccttgttgcatgagtttgtggaagattggtacaatcaagaatttatgggcaaccaatgtagctttggtgatgacaggcatctcacgaaccgggtgctgagcctgggctatgcaacaaaatacacagctcgatctaagtgccttactgaaacacctatagaatatctcagatggctaaaccagcagacccgttggagcaagtcctacttccgagaatggctgtacaatgcaatgtggtttcacaaacatcacttgtggatgacctacgaagcgattatcactggattctttcctttctttctcattgccacagtaatccagctcttctaccggggtaaaatttggaacattctcctcttcttgttaactgtccagctagtaggtctcataaaatcatcttttgccagctgccttagaggaaatatcgtcatggtcttcatgtctctctactcagtgttatacatgtcgagtttacttcccgccaagatgtttgcaattgcaacaataaacaaagctgggtggggcacatcaggaaggaaaaccattgttgttaatttcataggactcattccagtatcagtttggtttacaatcctcctgggtggtgtgattttcaccatttataaggagtctaaaaggccattttcagaatccaaacagacagttctaattgttggaacgttgctctatgcatgctattgggtcatgcttttgacgctgtatgtagttctcatcaataagtgtggcaggcggaagaagggacaacaatatgacatggtgcttgatgtatgatcttccatgttttgacgtttgcagtcacacacaacaccttagttcctctaggggctgtacagtattgtggcatcagataatgccaccaaaggagacatatcactgctgctgggacttgaacaaagacatttatatgggtttattttcattctgccaaagtaaaacaatacatcaacaagaagaaactcagatttaacctgttatttctatgaaaatgggatgaattctttgtttatgcactttttccttactgtgcatccgcctgaaagtgttttgccctatatacctcactagccatgctttatgtgggttatcatggaagaaaaggattttggaaactcaaggaaaagttctttcaacctatacaacctaacttatggactgttttgatagatgataattttttttttttaggaaggattttctttttaactttaccaaatgaaatgccaaaggaagttttaaaggccgttggctgtgctgtattttgatataattgtactgtgtttttaaattttgtatgccaatcttaaagacaaattttgcatattctctattttacttttctgccaaaataaacctgttcttccttttttaaaataaaataagttcttaaaaaatttatacttaaaaaatcctgcccaaaatgtgaagcttggttgactgatgttcatgatagaaagaataaaatgtttctctctctctaccttttaaaattgaatagtttatttctgtgaaagaagtatttaaactttcaatattttaactttttgtttttatttcttttagaaaaggccaatatacctatcacactttggaagtaaaaatacacactttcgtgtgtacctaaaaaaaaaatcgttgaaaatcaaggccaaaggtagtgcaattttttcattaagatttaaaaaaaagggaatgatagtctttgaaagaaaacagtaggcatccagcactggacaaaacatgggtatcaaagatgaataatctttggagattctggcagtgttttcccagaacaagtcaagtggaaagtggagaaattatctgtataattttggacacatacaatgcagtttatcaaaggttttgttctgtggcctgaatttactgggtcctacctacacattgaacatgttttgcctgtcttttttttttttttcaacttgccagttcactttacatgttagtataatgtttacacgggtgagttggataaattataaaacatataaattcaaaattggcagatagaatcaccaagtatctatcctcttttactttcaaatgaggaattttgtttttctgaattacacagatcatcacttcctatttcctgttctggacctgtataaaaatgtctacacagtagaagtgacatcaaggtttaataagtatatcaatgattggcacatataaaaattgttgaaccacatactctgaacttggctaatttagttactgcaaggcctccattatccagttttattttttacacgattgaccttgccttgtagctggtgctgtgtagacctgtgttgaaaacacaatcggaatatatgaataattgaataaacagcattatggtgaggcagagacacatggagaagtgttaaaaaaaaaatgggcttcctgcctttctgcctcttttttatgcagtcatctatgttacatctatcctgcctaagaaaaagctgcacatcctaccttcagagtacaaaaaggtacatctgagactcaagactctcactgattggagagcttgtggaaaacaaaacacaccatgccaataaatgagatgaaaacttgagtttgcctttttaactatttatgttctaagttaagctttgataacattcaaatgtcaaattctctcattcttataaaaagttgaattaattgcctgtatttattttagcaattattcaatgtatttccagtataggatgtatagtataattaattttttgtaaataaaatatttttgataaga

SEQ ID NO: 7 - protein, 509 aa, Homo sapiens
MGVLKFKHIFFRSFVKSSGVSQIVFTFLLIPCCLTLNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLSATMFIVSILFLIISSVASL

SEQ ID NO: 8 - DNA, 1683 bp, Homo sapiens
  source 1..1683: mol_type = other DNA
attccattccctttcatctgtgctcatactttgcatcagatattgggtaaaccaaagtgtgtaggaagaaataaatgttttcatagtcattactctttacaatgggagtgctaaaattcaagcacatctttttcagaagctttgttaaatcaagtggagtatcccagatagttttcaccttccttctgattccatgttgcttgactctgaatttcagagcacctcctgttattccaaatgtgcctttcctctgggcctggaatgccccaagtgaattttgtcttggaaaatttgatgagccactagatatgagcctcttctctttcataggaagcccccgaataaacgccaccgggcaaggagttacaatattttatgttgatagacttggctactatccttacatagattcaatcacaggagtaactgtgaatggaggaatcccccagaagatttccttacaagaccatctggacaaagctaagaaagacattacattttatatgccagtagacaatttgggaatggctgttattgactgggaagaatggagacccacttgggcaagaaactggaaacctaaagatgtttacaagaataggtctattgaattggttcagcaacaaaatgtacaacttagtctcacagaggccactgagaaagcaaaacaagaatttgaaaaggcagggaaggatttcctggtagagactataaaattgggaaaattacttcggccaaatcacttgtggggttattatctttttccggattgttacaaccatcactataagaaacccggttacaatggaagttgcttcaatgtagaaataaaaagaaatgatgatctcagctggttgtggaatgaaagcactgctctttacccatccatttatttgaacactcagcagtctcctgtagctgctacactctatgtgcgcaatcgagttcgggaagccatcagagtttccaaaatacctgatgcaaaaagtccacttccggtttttgcatatacccgcatagtttttactgatcaagttttgaaattcctttctcaagatgaacttgtgtatacatttggcgaaactgttgctctgggtgcttctggaattgtaatatggggaaccctcagtataatgcgaagtatgaaatcttgcttgctcctagacaattacatggagactatactgaatccttacataatcaacgtcacactagcagccaaaatgtgtagccaagtgctttgccaggagcaaggagtgtgtataaggaaaaactggaattcaagtgactatcttcacctcaacccagataattttgctattcaacttgagaaaggtggaaagttcacagtacgtggaaaaccgacacttgaagacctggagcaattttctgaaaaattttattgcagctgttatagcaccttgagttgtaaggagaaagctgatgtaaaagacactgatgctgttgatgtgtgtattgctgatggtgtctgtatagatgcttttctaaaacctcccatggagacagaagaacctcaaattttctacaatgcttcaccctccacactatctgccacaatgttcattgttagtattttgtttcttatcatttcttctgtagcgagtttgtaattgcgcaggttagctgaaatgaacaatatgtccatcttaaagtgtgcttccc

SEQ ID NO: 9 - protein, 1050 aa, Homo sapiens
MEPGRGGTETVGKFEFSRKDLIGHGAFAVVFKGRHREKHDLEVAVKCINKKNLAKSQTLLGKEIKILKELKHENIVALYDFQEMANSVYLVMEYCNGGDLADYLHAMRTLSEDTIRLFLQQIAGAMRLLHSKGIIHRDLKPQNILLSNPAGRRANPNSIRVKIADFGFARYLQSNMMAATLCGSPMYMAPEVIMSQHYDGKADLWSIGTIVYQCLTGKAPFQASSPQDLRLFYEKNKTLVPTIPRETSAPLRQLLLALLQRNHKDRMDFDEFFHHPFLDASPSVRKSPPVPVPSYPSSGSGSSSSSSSTSHLASPPSLGEMQQLQKTLASPADTAGFLHSSRDSGGSKDSSCDTDDFVMVPAQFPGDLVAEAPSAKPPPDSLMCSGSSLVASAGLESHGRTPSPSPPCSSSPSPSGRAGPFSSSRCGASVPIPVPTQVQNYQRIERNLQSPTQFQTPRSSAIRRSGSTSPLGFARASPSPPAHAEHGGVLARKMSLGGGRPYTPSPQVGTIPERPGWSGTPSPQGAEMRGGRSPRPGSSAPEHSPRTSGLGCRLHSAPNLSDLHVVRPKLPKPPTDPLGAVFSPPQASPPQPSHGLQSCRNLRGSPKLPDFLQRNPLPPILGSPTKAVPSFDFPKTPSSQNLLALLARQGVVMTPPRNRTLPDLSEVGPFHGQPLGPGLRPGEDPKGPFGRSFSTSRLTDLLLKAAFGTQAPDPGSTESLQEKPMEIAPSAGFGGSLHPGARAGGTSSPSPVVFTVGSPPSGSTPPQGPRTRMFSAGPTGSASSSARHLVPGPCSEAPAPELPAPGHGCSFADPITANLEGAVTFEAPDLPEETLMEQEHTEILRGLRFTLLFVQHVLEIAALKGSASEAAGGPEYQLQESVVADQISLLSREWGFAEQLVLYLKVAELLSSGLQSAIDQIRAGKLCLSSTVKQVVRRLNELYKASVVSCQGLSLRLQRFFLDKQRLLDRIHSITAERLIFSHAVQMVQSAALDEMFQHREGCVPRYHKALLLLEGLQHMLSDQADIENVTKCKLCIERRLSALLTGICA

SEQ ID NO: 10 - DNA, 5228 bp, Homo sapiens
  source 1..5228: mol_type = other DNA
ggatccggattcggattagcagcccgggaagagtgccgtggcacaggcgccggagggagcgcgaccctcggaccccgcctggcccgcggggctgggacccggccccggcctgcccgatggggcgcgcggccccggagatgcgccctcgcccggccccgcgcccccggccccgcgcccccggcccgcccgccccggcccgcgcctccgcctgagtcccccgcgccttggcccgccaccccccgccccgcgcccccggcccgcctgcgccatggagcccggccgcggcggcacagagaccgtgggcaagttcgagttctcccgcaaggacctgatcggccacggcgccttcgcggtggtcttcaagggccgccaccgcgagaagcacgatttggaggtcgccgtcaagtgcattaacaagaagaacctcgccaagtctcagacgctgctggggaaggaaatcaaaatcctgaaggaactgaaacatgaaaacatcgtggccctgtacgacttccaggaaatggctaattctgtctacctggttatggagtactgcaacggtggggacctggccgactacctgcacgccatgcgcacgctgagcgaggacaccatcaggctcttcctgcagcagatcgcgggcgccatgcggcttctgcacagcaaaggcatcatccaccgcgacctgaaaccgcagaacatcctgctgtccaaccccgccggccgccgcgccaaccccaacagcatccgcgtcaagatcgctgacttcggcttcgcgcggtacctccagagcaacatgatggcggccacactctgcggctcccccatgtacatggcccccgaggtcatcatgtcccagcactacgacgggaaggcggacctgtggagcatcggcaccatcgtctaccagtgcctgacggggaaggcgcccttccaggccagcagcccccaggacctgcgcctgttctacgagaagaacaagacgttggtccccaccatcccccgggagacctcggccccgctgcggcagctgctcctggccctactgcaacgcaaccacaaggaccgcatggacttcgatgagttttttcatcaccctttcctcgatgccagcccctcggtcaggaaatccccacccgtgcctgtgccctcgtacccaagctcggggtccggcagcagctccagcagcagctccacctcccacctggcctccccgccgtccctgggcgagatgcagcagctgcagaagaccctggcctccccggctgacaccgctggcttcctgcacagctcccgggactctggtggcagcaaggactcttcctgtgacacagacgacttcgtcatggtccccgcgcagtttccaggtgacctggtggctgaggcgcccagtgccaaacccccgccagacagcctgatgtgcagtgggagctcactggtggcctctgcgggcttggagagccacggccggaccccatctccatccccaccctgcagcagctcccccagtccctcaggccgggctggcccgttctccagcagcaggtgcggcgcctctgtccccatcccagtccccacgcaggtgcagaactaccagcgcattgagcgaaacctgcagtcacccacccagttccaaacacctcggtcctctgccatccgcaggtcaggcagcaccagccccctgggctttgcaagggccagcccctcgccccctgcccacgctgagcatggaggcgtcctggccaggaagatgtctctgggtggaggccggccctacacgccatctcctcaagttggaaccatccctgagcggccaggctggagcgggacgccctccccacagggagctgagatgcggggtggcaggtcccctcgtccaggctcctctgcacccgagcactctccccgcacttccgggctgggctgccgcctgcacagcgcccccaacctgtctgacttgcacgtcgtccgccccaagctgcccaaaccccccacggaccccctgggagctgtgttcagcccaccacaggccagccctccccagccgtcccacggcctgcagtcctgccggaacctgcggggctcacccaagctgcccgacttcctgcagcgaaaccccctgccccccatcctgggctcccccaccaaggctgtgccctcctttgacttcccgaagacccccagctcccagaacctgctggccctcctagcccggcagggcgtggtgatgacgccccctcgaaaccggacgctgcccgacctctcggaggtgggacccttccatggtcagccgttgggccctggcctgcggccaggcgaggaccccaagggcccctttggccggtctttcagcaccagccgcctcactgacctgctccttaaggcggcgtttgggacacaagccccggacccgggcagcacggagagcctgcaggagaagcccatggagatcgcaccctcagctggctttggagggagcctgcacccaggagcccgtgctgggggcaccagcagcccctccccggtggtcttcaccgtgggctctcccccgagcgggagcacgcccccccagggcccccgcaccaggatgttctcagcgggccccactggctctgccagctcttctgcccgccacctggtgcctgggccctgcagcgaggccccagcccctgagctccctgctccaggacacggctgcagctttgccgaccccattgctgcgaacctggagggggctgtgaccttcgaggcccccgacctccctgaggagaccctcatggagcaagagcacacggagatcctgcgtggcctgcgcttcacgctgctgttcgtgcagcacgtcctggagatcgcagccctgaagggcagcgccagtgaggcggcggggggccctgagtaccagctgcaggagagtgtggtggccgaccagatcagcctgctgagccgagaatggggcttcgcggaacagctggtgctgtacctgaaggtggccgagctactgtcctccggcctgcaaagtgccatcgaccagatccgggccggcaagctctgcctgtcgtccactgtgaagcaggtggtgcgcaggctgaatgagctgtacaaggccagcgtggtgtcctgccagggcctgagcctgcggctgcagcgcttcttcctggacaagcagcggctcctggaccgcattcacagcatcactgccgagaggctcatcttcagccacgctgtgcagatggtgcagtcggctgccctggacgagatgttccagcaccgtgagggctgcgtcccacgctaccacaaggccctgctgctcctggaggggctgcagcacatgctctcggaccaggccgacatcgagaacgtcaccaagtgcaagctgtgcattgagcggagactctcggcgctgctgactggcatctgtgcctgacctttctggcctggctgggccccccgtcctgccgagccctgcagagtgggctctgtgtgctggctggactcctcgggacaagcccatggcgctgatcgctggtgctgagccctgccctgggccccacggacagtcagcctgccggcctccctgcagctcacggggcagaaccagcacatctggagccacacagcttggggggtgtctcccatcttttacaggtggggatcacagaatttctgcccctccagctgcctggctcagcaggcgtgggtgccaccaccctctagccccagggcagccccggaggacaggcaagggcctgagaccactgccgactcaaagccaaagcgagctcctgcttagggcaggtcagcaggcactgtgcccaggaagagcctgcggcctcggcgtcccccagtctccaggagcctctccctccgagatacccacccagctttgtcaatcacccaagcactttatgcatatagagacagaacctggacctcaccagggactgctgggcagcgattcctggcagtggcctggtgtttgtacatacacatatgcagacacatgccagggccccccaagcccgagcaccggaccacgttgctgcccaggtctggacctcagcgggagaactggctccggggggagtggggccctgcgctagaggcagaggcagttctttgttcaagcgttcctctggggaccggcagcagaggcaccgtgttctctcagccctggatacgtcttgtaatctttcacactttattcctaaaacgtgtcttatttttatgcagctcattttttctttaaaggagaaaacttgtaggtgtttaagaattggttttgggagggcgaggactgggccaggttagaggcagatggcacaggggcgtgtggcgggcgggtgaggctgctttgcacacctgtgttggtggctgtcccctgccgcccctccctgtggcagcagcaggacaggtgtgtgcccagcaccctccctacctgggcctggaagcagatgaggggaatacttcatgcaaagaaaaaagtaacatgtgcaaaagctccccgtccagctttgacagtcagttttgatgtcagctcctcggcagggtaggcctgatgacagccctgtccctccctgcctccgccttgcccaaggccacggagggcgtctgcagagaggcctgccttccggattccaggcgggcatgccctgcaaaccccgcctgggcctcccttggtctgcccagccctcggttagccctgcctgaatcagtagatacttgaacgagtccccagtctgcgggaggcagtggtggggccatggacccatgcggggggttccagggtcacacgccacataacagacaaaaatacacacacgtgtgtttttctttgcaatacttgaaatattgccactgtgcttggacttagaagaagaaaatccccgtgacttcttcctcatcaccttgatggctttattctcaccttgtggggcatgtttgaatttattgcttcatggccgactggaatcctgagtcctgggaagctggcactgcggggatcttgcccggtgtcctggtcctcttgcttccgtcgcggccgcatgtgcgtgtgtccaagcaggtcctgggcgcctcaactgctgcccctggttgaatgttctcttgatagtgctggaccctttgtctattttaaagcgaattttgtgtgatttcctgccctttgcgttatattgtataataccaacgtaaggaaataaacctttggaattgttgaaaaaaaaaaaaaaaaa

SEQ ID NO: 11 - protein, 440 aa, Homo sapiens
MASLTVKAYLLGKEDAAREIRRFSFCCSPEPEAEAEAAAGPGPCERLLSRVAALFPALRPGGFQAHYRDEDGDLVAFSSDEELTMAMSYVKDDIFRIYIKEKKECRRDHRPPCAQEAPRNMVHPNVICDGCNGPVVGTRYKCSVCPDYDLCSVCEGKGLHRGHTKLAFPSPFGHLSEGFSHSRWLRKVKHGHFGWPGWEMGPPGNWSPRPPRAGEARPGPTAESASGPSEDPSVNFLKNVGESVAAALSPLGIEVDIDVEHGGKRSRLTPVSPESSSTEEKSSSQPSSCCSDPSKPGGNVEGATQSLAEQMRKIALESEGRPEEQMESDNCSGGDDDWTHLSSKEVDPSTGELQSLQMPESEGPSSLDPSQEGPTGLKEAALYPHLPPEADPRLIESLSQMLSMGFSDEGGWLTRLLQTKNYDIGAALDTIQYSKHPPPL

SEQ ID NO: 12 - DNA, 1995 bp, Homo sapiens
  source 1..1995: mol_type = other DNA
gaattccctcgccgctcgctatggcgtcgctcaccgtgaaggcctaccttctgggcaaggaggacgcggcgcgcgagattcgccgcttcagcttctgctgcagccccgagcctgaggcggaagccgaggctgcggcgggtccgggaccctgcgagcggctgctgagccgggtggccgccctgttccccgcgctgcggcctggcggcttccaggcgcactaccgcgatgaggacggggacttggttgccttttccagtgacgaggaattgacaatggccatgtcctacgtgaaggatgacatcttccgaatctacattaaagagaaaaaagagtgccggcgggaccaccgcccaccgtgtgctcaggaggcgccccgcaacatggtgcaccccaatgtgatctgcgatggctgcaatgggcctgtggtaggaacccgctacaagtgcagcgtctgcccagactacgacttgtgtagcgtctgcgagggaaagggcttgcaccgggggcacaccaagctcgcattccccagccccttcgggcacctgtctgagggcttctcgcacagccgctggctccggaaggtgaaacacggacacttcgggtggccaggatgggaaatgggtccaccaggaaactggagcccacgtcctcctcgtgcaggggaggcccgccctggccccacggcagaatcagcttctggtccatcggaggatccgagtgtgaatttcctgaagaacgttggggagagtgtggcagctgcccttagccctctgggcattgaagttgatatcgatgtggagcacggagggaaaagaagccgcctgacccccgtctctccagagagttccagcacagaggagaagagcagctcacagccaagcagctgctgctctgaccccagcaagccgggtgggaatgttgagggcgccacgcagtctctggcggagcagatgaggaagatcgccttggagtccgagggggcccctgaggaacagatggagtcggataactgttcaggaggagatgatgactggacccatctgtcttcaaaagaagtggacccgtctacaggtgaactccagtccctacagatgccagaatccgaagggccaagctctctggacccctcccaggagggacccacagggctgaaggaagctgccttgtacccacatctcccgccagaggctgacccgcggctgattgagtccctctcccagatgctgtccatgggcttctctgatgaaggcggctggctcaccaggctcctgcagaccaagaactatgacatcggagcggctctggacaccatccagtattcaaagcatcccccgccgttgtgaccacttttgcccacctcttctgcgtgcccctcttctgtctcatagttgtgttaagcttgcgtagaattggcaggtctctgtacgggccagtttctctgccttcttccaggatcaggggttagggtgcaagaagccatttagggcagcaaaacaagtgacatgaagggagggtccctgtgtgtgtgtgtgctgatgtttcctgggtgccctggctccttgcagcagggctgggcctgcgagacccaaggctcactgcagcgcgctcctgacccctccctgcaggggctacgttagcagcccagcacatagcttgcctaatggctttcactttctcttttgttttaaatgactcataggtccctgacatttagttgattattttctgctacagacctggtacactctgattttagataaagtaagcctaggtgttgtcagcaggcaggctggggaggccagtgttgtgggcctcctgctgggactgagaaggcccacgaaggcgtccgcaatgttggtttcactgagagctgcctcctggtctcttcaccactgtagttctctcatttccaaaccatcagctgcttttaaaataagatctctttgtagccatcctgttaaatttgtaaacaatctaattaaatggcatgcgcag

SEQ ID NO: 13-19 - skipped (7 entries)

SEQ ID NO: 20 - DNA, 19 bp, synthetic construct
  source 1..19: mol_type = other DNA
gtaacccgttgaaccccat

SEQ ID NO: 21 - DNA, 20 bp, synthetic construct
  source 1..20: mol_type = other DNA
ccatccaatcggtagtagcg

SEQ ID NO: 22 - DNA, 18 bp, synthetic construct
  source 1..18: mol_type = other DNA
agcgtctgcgagggaaag

SEQ ID NO: 23 - DNA, 19 bp, synthetic construct
  source 1..19: mol_type = other DNA
acccgaagtgtccgtgttt

SEQ ID NO: 24 - DNA, 20 bp, synthetic construct
  source 1..20: mol_type = other DNA
tctgccttgctaccaattcc

SEQ ID NO: 25 - DNA, 20 bp, synthetic construct
  source 1..20: mol_type = other DNA
gatgggttctctgagcttcg

SEQ ID NO: 26 - DNA, 20 bp, synthetic construct
  source 1..20: mol_type = other DNA
caacacggctccagaagact

SEQ ID NO: 27 - DNA, 22 bp, synthetic construct
  source 1..22: mol_type = other DNA
gccttcacagattcatcttgct

SEQ ID NO: 28 - DNA, 20 bp, synthetic construct
  source 1..20: mol_type = other DNA
tgctgatgacctcacatggt

SEQ ID NO: 29 - DNA, 20 bp, synthetic construct
  source 1..20: mol_type = other DNA
ccattgagcatcacatggac

SEQ ID NO: 30 - DNA, 20 bp, synthetic construct
  source 1..20: mol_type = other DNA
tgttcctctgtactctggcc

SEQ ID NO: 31 - DNA, 20 bp, synthetic construct
  source 1..20: mol_type = other DNA
gcagtttagtagcaagcccc

SEQ ID NO: 32 - DNA, 20 bp, synthetic construct
  source 1..20: mol_type = other DNA
ctcgctcatcatgctgaacc

SEQ ID NO: 33 - DNA, 20 bp, synthetic construct
  source 1..20: mol_type = other DNA
acacaggaagacagggctac

SEQ ID NO: 34 - DNA, 20 bp, synthetic construct
  source 1..20: mol_type = other DNA
cgatagaacacggcatcatc

SEQ ID NO: 35 - DNA, 20 bp, synthetic construct
  source 1..20: mol_type = other DNA
catcaggcagttcgtagctc

SEQ ID NO: 36 - DNA, 22 bp, synthetic construct
  source 1..22: mol_type = other DNA
cgtggagctgtaccagaaatac

SEQ ID NO: 37 - DNA, 20 bp, synthetic construct
  source 1..20: mol_type = other DNA
cacaactccggtgacatcaa

SEQ ID NO: 38 - DNA, 23 bp, synthetic construct
  source 1..23: mol_type = other DNA
gactcctgggtcagcttcctaag

SEQ ID NO: 39 - DNA, 24 bp, synthetic construct
  source 1..24: mol_type = other DNA
aaactgctgcaagaggttattcct

SEQ ID NO: 40 - DNA, 22 bp, synthetic construct
  source 1..22: mol_type = other DNA
agcaccttctcgtgcatcatgc

SEQ ID NO: 41 - DNA, 22 bp, synthetic construct
  source 1..22: mol_type = other DNA
tcctccaggactcgaagcatct

SEQ ID NO: 42 - DNA, 21 bp, synthetic construct
  source 1..21: mol_type = other DNA
tcaggggcttcttcttctttg

SEQ ID NO: 43 - DNA, 20 bp, synthetic construct
  source 1..20: mol_type = other DNA
tctgaggccatcattgaaca

SEQ ID NO: 44 - DNA, 22 bp, synthetic construct
  source 1..22: mol_type = other DNA
tcttccctggagacctgagaac

SEQ ID NO: 45 - DNA, 22 bp, synthetic construct
  source 1..22: mol_type = other DNA
gacaccaaactggatgacgatg

SEQ ID NO: 46 - DNA, 21 bp, synthetic construct
  source 1..21: mol_type = other DNA
tgctggcctaatagagtggca

SEQ ID NO: 47 - DNA, 20 bp, synthetic construct
  source 1..20: mol_type = other DNA
ctcagcgccatggaaaatgt

SEQ ID NO: 48 - DNA, 21 bp, synthetic construct
  source 1..21: mol_type = other DNA
ccatctgtcactacttcatgc

SEQ ID NO: 49 - DNA, 18 bp, synthetic construct
  source 1..18: mol_type = other DNA
aagtcctcctgttccagc

SEQ ID NO: 50 - DNA, 18 bp, synthetic construct
  source 1..18: mol_type = other DNA
gctggacagaagagatgc

SEQ ID NO: 51 - DNA, 21 bp, synthetic construct
  source 1..21: mol_type = other DNA
gtggatgtctcctatgttacc

SEQ ID NO: 52 - DNA, 20 bp, synthetic construct
  source 1..20: mol_type = other DNA
gcgtgtaaaagaagccgact

SEQ ID NO: 53 - DNA, 20 bp, synthetic construct
  source 1..20: mol_type = other DNA
ttcttctcccaggcagtcag

SEQ ID NO: 54 - DNA, 18 bp, synthetic construct
  source 1..18: mol_type = other DNA
cccagaacatcgatgagc

SEQ ID NO: 55 - DNA, 19 bp, synthetic construct
  source 1..19: mol_type = other DNA
gccacaactccacaagagg

SEQ ID NO: 56 - DNA, 19 bp, synthetic construct
  source 1..19: mol_type = other DNA
agggacaaactggcagagc

SEQ ID NO: 57 - DNA, 18 bp, synthetic construct
  source 1..18: mol_type = other DNA
ttagccacggtgcagagc

SEQ ID NO: 58 - DNA, 18 bp, synthetic construct
  source 1..18: mol_type = other DNA
gcagagcagacaccatcc

SEQ ID NO: 59 - DNA, 20 bp, synthetic construct
  source 1..20: mol_type = other DNA
gttcacgatgtaaagcttcg

SEQ ID NO: 60 - DNA, 20 bp, synthetic construct
  source 1..20: mol_type = other DNA
atccccctattacgacagcg

SEQ ID NO: 61 - DNA, 19 bp, synthetic construct
  source 1..19: mol_type = other DNA
ggcctcccggaggtaaaca

SEQ ID NO: 62 - DNA, 43 bp, synthetic construct
  source 1..43: mol_type = other DNA
cggtcatgggacgctctgtcactgccggccagaccacctgacc

SEQ ID NO: 63 - DNA, 43 bp, synthetic construct
  source 1..43: mol_type = other DNA
ggtcaggtggtctggccggcagtgacagagcgtcccatgaccg

SEQ ID NO: 64 - DNA, 42 bp, synthetic construct
  source 1..42: mol_type = other DNA
caggcgcctgggctgctctgtcacgcttggccagcacctgtc

SEQ ID NO: 65 - DNA, 42 bp, synthetic construct
  source 1..42: mol_type = other DNA
gacaggtgctggccaagcgtgacagagcagcccaggcgcctg

SEQ ID NO: 66 - DNA, 41 bp, synthetic construct
  source 1..41: mol_type = other DNA
cccaactgaggatattgctctgtcatggccaggcccaagcc

SEQ ID NO: 67 - DNA, 41 bp, synthetic construct
  source 1..41: mol_type = other DNA
ggcttgggcctggccatgacagagcaatatcctcagttggg

SEQ ID NO: 68 - RNA, 20 nt, Homo sapiens
  source 1..20: mol_type = other RNA
gggacttttccatcaaacat

SEQ ID NO: 69 - RNA, 20 nt, Homo sapiens
  source 1..20: mol_type = other RNA
atgggacgctgactcactgc

SEQ ID NO: 70 - DNA, 133 bp, synthetic construct
  source 1..133: mol_type = other DNA
tccccagcccagcctccaggtaagagtcactgagatgggtggcagcaggggccggggatccccctattacgacagcggtcatgggacgcctgccggccagaccacctgacctccgcggcgggaggagagggcc

SEQ ID NO: 71 - protein, 233 aa, Homo sapiens
MSTESMIRDVELAEEALPKKTGGPQGSRRCLFLSLFSFLIVAGATTLFCLLHFGVIGPQREEFPRDLSLISPLAQAVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 72 - DNA, 1678 bp, Homo sapiens
  source 1..1678: mol_type = other DNA
agcagacgctccctcagcaaggacagcagaggaccagctaagagggagagaagcaactacagaccccccctgaaaacaaccctcagacgccacatcccctgacaagctgccaggcaggttctcttcctctcacatactgacccacggctccaccctctctcccctggaaaggacaccatgagcactgaaagcatgatccgggacgtggagctggccgaggaggcgctccccaagaagacaggggggccccagggctccaggcggtgcttgttcctcagcctcttctccttcctgatcgtggcaggcgccaccacgctcttctgcctgctgcactttggagtgatcggcccccagagggaagagttccccagggacctctctctaatcagccctctggcccaggcagtcagatcatcttctcgaaccccgagtgacaagcctgtagcccatgttgtagcaaaccctcaagctgaggggcagctccagtggctgaaccgccgggccaatgccctcctggccaatggcgtggagctgagagataaccagctggtggtgccatcagagggcctgtacctcatctactcccaggtcctcttcaagggccaaggctgcccctccacccatgtgctcctcacccacaccatcagccgcatcgccgtctcctaccagaccaaggtcaacctcctctctgccatcaagagcccctgccagagggagaccccagagggggctgaggccaagccctggtatgagcccatctatctgggaggggtcttccagctggagaagggtgaccgactcagcgctgagatcaatcggcccgactatctcgactttgccgagtctgggcaggtctactttgggatcattgccctgtgaggaggacgaacatccaaccttcccaaacgcctcccctgccccaatccctttattaccccctccttcagacaccctcaacctcttctggctcaaaaagagaattgggggcttagggtcggaacccaagcttagaactttaagcaacaagaccaccacttcgaaacctgggattcaggaatgtgtggcctgcacagtgaagtgctggcaaccactaagaattcaaactggggcctccagaactcactggggcctacagctttgatccctgacatctggaatctggagaccagggagcctttggttctggccagaatgctgcaggacttgagaagacctcacctagaaattgacacaagtggaccttaggccttcctctctccagatgtttccagacttccttgagacacggagcccagccctccccatggagccagctccctctatttatgtttgcacttgtgattatttattatttatttattatttatttatttacagatgaatgtatttatttgggagaccggggtatcctgggggacccaatgtaggagctgccttggctcagacatgttttccgtgaaaacggagctgaacaataggctgttcccatgtagccccctggcctctgtgccttcttttgattatgttttttaaaatatttatctgattaagttgtctaaacaatgctgatttggtgaccaactgtcactcattgctgagcctctgctccccaggggagttgtgtctgtaatcgccctactattcagtggcgagaaataaagtttgcttagaaaagaaa

SEQ ID NO: 73 - protein, 212 aa, Homo sapiens
MNSFSTSAFGPVAFSLGLLLVLPAAFPAPVPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 74 - DNA, 1127 bp, Homo sapiens
  source 1..1127: mol_type = other DNA
attctgccctcgagcccaccgggaacgaaagagaagctctatctcccctccaggagcccagctatgaactccttctccacaagcgccttcggtccagttgccttctccctggggctgctcctggtgttgcctgctgccttccctgccccagtacccccaggagaagattccaaagatgtagccgccccacacagacagccactcacctcttcagaacgaattgacaaacaaattcggtacatcctcgacggcatctcagccctgagaaaggagacatgtaacaagagtaacatgtgtgaaagcagcaaagaggcactggcagaaaacaacctgaaccttccaaagatggctgaaaaagatggatgcttccaatctggattcaatgaggagacttgcctggtgaaaatcatcactggtcttttggagtttgaggtatacctagagtacctccagaacagatttgagagtagtgaggaacaagccagagctgtgcagatgagtacaaaagtcctgatccagttcctgcagaaaaaggcaaagaatctagatgcaataaccacccctgacccaaccacaaatgccagcctgctgacgaagctgcaggcacagaaccagtggctgcaggacatgacaactcatctcattctgcgcagctttaaggagttcctgcagtccagcctgagggctcttcggcaaatgtagcatgggcacctcagattgttgttgttaatgggcattccttcttctggtcagaaacctgtccactgggcacagaacttatgttgttctctatggagaactaaaagtatgagcgttaggacactattttaattatttttaatttattaatatttaaatatgtgaagctgagttaatttatgtaagtcatatttatatttttaagaagtaccacttgaaacattttatgtattagttttgaaataataatggaaagtggctatgcagtttgaatatcctttgtttcagagccagatcatttcttggaaagtgtaggcttacctcaaataaatggctaacttatacatatttttaaagaaatatttatattgtatttatataatgtataaatggtttttataccaataaatggcattttaaaaaattca

SEQ ID NO: 75 - protein, 1094 aa, Homo sapiens
MQYLNIKEDCNAMAFCAKMRSSKKTEVNLEAPEPGVEVIFYLSDREPLRLGSGEYTAEELCIRAAQACRISPLCHNLFALYDENTKLWYAPNRTITVDDKMSLRLHYRMRFYFTNWHGTNDGHDIENECLGMAVLAISHYAMMKKMQLPELPKDISYKRYIPETLNKSIRQRNLLTRMRINNVFKDFLKEFNNKTICDSSVSTHDLKVKYLATLETLTKHYGAEIFETSMLLISSENEMNWFHSNDGGNVLYYEVMVTGNLGIQWRHKPNVVSVEKEKNKLKRKKLENKHKKDEEKNKIREEWNNFSYFPEITHIVIKESVVSINKQDNKKMELKLSSHEEALSFVSLVDGYFRLTADAHHYLCTDVAPPLIVHNIQNGCHGPICTEYAINKLRQEGSEEGMYVLRWSCTDFDNILMTVTCFEKSEQVQGAQKQFKNFQIEVQKGRYSLHGSDRSFPSLGDLMSHLKKQILRTDNISFMLKRCCQPKPREISNLLVATKKAQEWQPVYPMSQLSFDRILKKDLVQGEHLGRGTRTHIYSGTLMDYKDDEGTSEEKKIKVILKVLDPSHRDISLAFFEAASMMRQVSHKHIVYLYGVCVRDVENIMVEEFVEGGPLDLFMHRKSDVLTTPWKFKVAKQLASALSYLEDKDLVHGNVCTKNLLLAREGIDSECGPFIKLSDPGIPITVLSRQECIERIPWIAPECVEDSKNLSVAADKWSFGTTLWEICYNGEIPLKDKTLIEKERFYESRCRPVTPSCKELADLMTRCMNYDPNQRPFFRAIMRDINKLEEQNPDIVSEKKPATEVDPTHFEKRFLKRIRDLGEGHFGKVELCRYDPEGDNTGEQVAVKSLKPESGGNHIADLKKEIEILRNLYHENIVKYKGICTEDGGNGIKLIMEFLPSGSLKEYLPKNKNKINLKQQLKYAVQICKGMDYLGSRQYVHRDLAARNVLVESEHQVKIGDFGLTKAIETDKEYYTVKDDRDSPVFWYAPECLMQSKFYIASDVWSFGVTLHELLTYCDSDSSPMALFLKMIGPTHGQMTVTRLVNTLKEGKRLPCPPNCPDEVYQLMRKCWEFQPSNRTSFQNLIEGFEALLK

SEQ ID NO: 76 - DNA, 5092 bp, Homo sapiens
  source 1..5092: mol_type = other DNA
gcgtcgctgagcgcaggccgcggcggccgcggagtatcctggagctgcagacagtgcgggcctgcgcccagtcccggctgtcctcgccgcgacccctcctcagccctgggcgcgcgcacgctggggccccgcggggctggccgcctagcgagcctgccggtcgaccccagccagcgcagcgacggggcgctgcctggcccaggcgcacacggaagtgcgcttctctgaagtagctttggaaagtagagaagaaaatccagtttgcttcttggagaacactggacagctgaataaatgcagtatctaaatataaaagaggactgcaatgccatggctttctgtgctaaaatgaggagctccaagaagactgaggtgaacctggaggcccctgagccaggggtggaagtgatcttctatctgtcggacagggagcccctccggctgggcagtggagagtacacagcagaggaactgtgcatcagggctgcacaggcatgccgtatctctcctctttgtcacaacctctttgccctgtatgacgagaacaccaagctctggtatgctccaaatcgcaccatcaccgttgatgacaagatgtccctccggctccactaccggatgaggttctatttcaccaattggcatggaaccaacgacaatgagcagtcagtgtggcgtcattctccaaagaagcagaaaaatggctacgagaaaaaaaagattccagatgcaacccctctccttgatgccagctcactggagtatctgtttgctcagggacagtatgatttggtgaaatgcctggctcctattcgagaccccaagaccgagcaggatggacatgatattgagaacgagtgtctagggatggctgtcctggccatctcacactatgccatgatgaagaagatgcagttgccagaactgcccaaggacatcagctacaagcgatatattccagaaacattgaataagtccatcagacagaggaaccttctcaccaggatgcggataaataatgttttcaaggatttcctaaaggaatttaacaacaagaccatttgtgacagcagcgtgtccacgcatgacctgaaggtgaaatacttggctaccttggaaactttgacaaaacattacggtgctgaaatatttgagacttccatgttactgatttcatcagaaaatgagatgaattggtttcattcgaatgacggtggaaacgttctctactacgaagtgatggtgactgggaatcttggaatccagtggaggcataaaccaaatgttgtttctgttgaaaaggaaaaaaataaactgaagcggaaaaaactggaaaataaacacaagaaggatgaggagaaaaacaagatccgggaagagtggaacaatttttcttacttccctgaaatcactcacattgtaataaaggagtctgtggtcagcattaacaagcaggacaacaagaaaatggaactgaagctctcttcccacgaggaggccttgtcctttgtgtccctggtagatggctacttccggctcacagcagatgcccatcattacctctgcaccgacgtggcccccccgttgatcgtccacaacatacagaatggctgtcatggtccaatctgtacagaatacgccatcaataaattgcggcaagaaggaagcgaggaggggatgtacgtgctgaggtggagctgcaccgactttgacaacatcctcatgaccgtcacctgctttgagaagtctgagcaggtgcagggtgcccagaagcagttcaagaactttcagatcgaggtgcagaagggccgctacagtctgcacggttcggaccgcagcttccccagcttgggagacctcatgagccacctcaagaagcagatcctgcgcacggataacatcagcttcatgctaaaacgctgctgccagcccaagccccgagaaatctccaacctgctggtggctactaagaaagcccaggagtggcagcccgtctaccccatgagccagctgagtttcgatcggatcctcaagaaggatctggtgcagggcgagcaccttgggagaggcacgagaacacacatctattctgggaccctgatggattacaaggatgacgaaggaacttctgaagagaagaagataaaagtgatcctcaaagtcttagaccccagccacagggatatttccctggccttcttcgaggcagccagcatgatgagacaggtctcccacaaacacatcgtgtacctctatggcgtctgtgtccgcgacgtggagaatatcatggtggaagagtttgtggaagggggtcctctggatctcttcatgcaccggaaaagcgatgtccttaccacaccatggaaattcaaagttgccaaacagctggccagtgccctgagctacttggaggataaagacctggtccatggaaatgtgtgtactaaaaacctcctcctggcccgtgagggcatcgacagtgagtgtggcccattcatcaagctcagtgaccccggcatccccattacggtgctgtctaggcaagaatgcattgaacgaatcccatggattgctcctgagtgtgttgaggactccaagaacctgagtgtggctgctgacaagtggagctttggaaccacgctctgggaaatctgctacaatggcgagatccccttgaaagacaagacgctgattgagaaagagagattctatgaaagccggtgcaggccagtgacaccatcatgtaaggagctggctgacctcatgacccgctgcatgaactatgaccccaatcagaggcctttcttccgagccatcatgagagacattaataagcttgaagagcagaatccagatattgtttcagaaaaaaaaccagcaactgaagtggaccccacacattttgaaaagcgcttcctaaagaggatccgtgacttgggagagggccactttgggaaggttgagctctgcaggtatgaccccgaaggggacaatacaggggagcaggtggctgttaaatctctgaagcctgagagtggaggtaaccacatagctgatctgaaaaaggaaatcgagatcttaaggaacctctatcatgagaacattgtgaagtacaaaggaatctgcacagaagacggaggaaatggtattaagctcatcatggaatttctgccttcgggaagccttaaggaatatcttccaaagaataagaacaaaataaacctcaaacagcagctaaaatatgccgttcagatttgtaaggggatggactatttgggttctcggcaatacgttcaccgggacttggcagcaagaaatgtccttgttgagagtgaacaccaagtgaaaattggagacttcggtttaaccaaagcaattgaaaccgataaggagtattacaccgtcaaggatgaccgggacagccctgtgttttggtatgctccagaatgtttaatgcaatctaaattttatattgcctctgacgtctggtcttttggagtcactctgcatgagctgctgacttactgtgattcagattctagtcccatggctttgttcctgaaaatgataggcccaacccatggccagatgacagtcacaagacttgtgaatacgttaaaagaaggaaaacgcctgccgtgcccacctaactgtccagatgaggtttatcaacttatgaggaaatgctgggaattccaaccatccaatcggacaagctttcagaaccttattgaaggatttgaagcacttttaaaataagaagcatgaataacatttaaattccacagattatcaagtccttctcctgcaacaaatgcccaagtcattttttaaaaatttctaatgaaagaagtttgtgttctgtccaaaaagtcactgaactcatacttcagtacatatacatgtataaggcacactgtagtgcttaatatgtgtaaggacttcctctttaaatttggtaccagtaacttagtgacacataatgacaaccaaaatatttgaaagcacttaagcactcctccttgtggaaagaatataccaccatttcatctggctagttcaccatcacaactgcattaccaaaaggggatttttgaaaacgaggagttgaccaaaataatatctgaagatgattgcttttccctgctgccagctgatctgaaatgttttgctggcacattaatcatagataaagaaagattgatggacttagccctcaaatttcagtatctatacagtactagaccatgcattcttaaaatattagataccaggtagtatatattgtttctgtacaaaaatgactgtattctctcaccagtaggacttaaactttgtttctccagtggcttagctcctgttcctttgggtgatcactagcacccatttttgagaaagctggttctacatggggggatagctgtggaatagataatttgctgcatgttaattctcaagaactaagcctgtgccagtgctttcctaagcagtatacctttaatcagaactcattcccagaacctggatgctattacacatgcttttaagaaacgtcaatgtatatccttttataactctaccactttggggcaagctattccagcactggttttgaatgctgtatgcaaccagtctgaataccacatacgctgcactgttcttagagggtttccatacttaccaccgatctacaagggttgatccctgtttttaccatcaatcatcaccctgtggtgcaacacttgaaagacccggctagaggcactatggacttcaggatccactagacagttttcagtttgcttggaggtagctgggtaatcaaaaatgtttagtcattgattcaatgtgaacgattacggtctttatgaccaagagtctgaaaatctttttgttatgctgtttagtattcgtttgatattgttacttttcacctgttgagcccaaattcaggattggttcagtggcagcaatgaagttgccatttaaatttgttcatagcctacatcaccaaggtctctgtgtcaaacctgtggccactctatatgcactttgtttactctttatacaaataaatatactaaagacttta

(19) United States
(12) Patent Application Publication
(10) Pub. No.: US 2025/0073234 A1
(43) Pub. Date: Mar. 6, 2025

(54) COMBINATION THERAPIES INCLUDING PARP1 INHIBITORS

(71) Applicant: Cornell University, Ithaca, NY (US)

(72) Inventors: Jorge Moscat-Guillen et al., New York, NY (US); Maria T. Diaz-Meco Conde, New York, NY (US); Juan F. Linares Rodriguez, New York, NY (US); Tania Cid Diaz, New York, NY (US)

(21) Appl. No.: 18/726,342

(22) PCT Filed: Jan. 4, 2023
(86) PCT No.: PCT/US2023/060098

§ 371 Date: Jul. 2, 2024

Related U.S. Application Data
(60) Provisional application No. 63/296,266, filed on Jan. 4, 2022.

(51) Int. Cl. A61K 31/502; A61K 31/4418; A61K 31/5377; A61K 31/538; A61K 38/47; A61K 45/06; A61P 35/00; C07K 16/24

(52) U.S. Cl. CPC A61K 31/502; A61K 31/4418; A61K 31/5377; A61K 31/538; A61K 38/47; A61K 45/06; A61P 35/00; C07K 16/248

(57) ABSTRACT

Described herein are methods and compositions that include use of one or more poly (ADP-ribose) polymerase 1 (PARP1) inhibitors and one or more inhibitors of activated stromal/activated cancer-associated fibroblasts. Such methods and compositions are useful for treating cancer. Further disclosed are PARP1 inhibitors, and inhibitors of activated stromal/activated cancer-associated fibroblasts used for the methods.

### CROSS-REFERENCE TO RELATED APPLICATIONS

This application claims the benefit of the filing date of U.S. application No. 63/296,266, filed on Jan. 4, 2022, the disclosure of which is incorporated by reference herein.

### GOVERNMENT FUNDING

This invention was made with government support under R01CA218254 awarded by the National Institutes of Health. The government has certain rights in the invention.

### INCORPORATION BY REFERENCE OF SEQUENCE LISTING

A Sequence Listing is provided herewith as an xml file, “2298710.xml” created on Jan. 3, 2023, 2022 and having a size of 95,728 bytes. The content of the xml file is incorporated by reference herein in its entirety.

### BACKGROUND

The tumor microenvironment (TME), including stromal fibroblasts, can be a driver of tumorigenesis in many types of tumors. Activation of tumor stromal fibroblasts to generate cancer-associated fibroblasts (CAFs) can embolden epithelial cancer cells to progress to more malignant stages and affect disease outcomes (Barron and Rowley. 2012; Cunha et al., 2002; Rowley, 1998; Tuxhorn et al., 2001). Cancer-associated fibroblasts impact the tumor epithelium by modulating many functions through the secretion of stromal growth factors and inflammatory mediators, by reprogramming their metabolism to provide nutrients and survival factors, and by remodeling the extracellular matrix (Hanahan and Weinberg. 2011; Kasashima et al., 2020; Sahai et al . . . 2020).

However, the master regulators of the acquisition of the CAF phenotype, as well as the molecular mechanisms whereby the tumor epithelium drives this process, are currently unknown.

### SUMMARY

As described herein, tumor-secreted lactate activates cancer associated fibroblasts (CAF) through downregulation of p62, a master regulator of CAF. Lactate reduces the NAD+/NADH ratio, which impairs PARP1 activity and inhibits p62. NAD+ supplementation can revert stromal activation. PARP1 inhibitors (PARP1i) such as Olaparib, mimic lactate and activate CAF in vitro and in vivo. Olaparib-induced stroma is rich in hyaluronan. Co-treatment of Olaparib and PEGPH20, a clinical grade hyaluronidase reverts stroma activation and enhances Olaparib anti-tumor activity.

Combinatorial therapies are described herein that can improve the efficacy of currently available cancer treatments. For example, compositions and methods that include use of PARP1 inhibitors can be combined with drugs that target the stroma to enhance PARP1 inhibitor efficacy or drugs that block or inhibit TNFalpha, IL-6 or Janus kinase (JAK).

The PARP1 inhibitors can include olaparib (Lynparza), niraparib (Zejula), rucaparib (Rubraca), talazoparib (Talzenna), veliparib (ABT-888), or a combination thereof.

Examples of agents that can be used to treat or inhibit activated stromal/activated cancer-associated fibroblasts include but are not limited to hyaluronan (HA) synthase inhibitors, hyaluronan build-up inhibitors (e.g., PEGylated Recombinant Human Hyaluronidase, PEGPH20), autophagy inhibitors (e.g., ULK inhibitors such as SBI-0202965), fibroblast activation protein alpha (FAPa) inhibitors, GW4064 (farnesoid X receptor (FXR) agonists, Pirfenidone (PDF) combined with doxorubicin, SMO-inhibitors (e.g., vismodegib, sonidegib), amphiregulin inhibitors, CXCL12 antagonists (e.g., an E5 antagonistic peptide), DDR2 inhibitors (e.g., WRG-28, which is a small molecule inhibitor of DDR2), or a combination thereof.

Examples of agents that can inhibit JAK include but are not limited to lesataurtinib, baricitinib, tofacitinib, upadacitinib, filgonitib, delgocitinib, deucravacitinib, LS104, ON044580,NVP-BBT594, or NVP-CHZ868, or a combination thereof.

Examples of agents that can inhibit IL6 include but are not limited to tocilizumab, sarilumab, siltuximab, olokizumab, elsilimomab, clazakizumab, sirukumab, levimumab, CPSI-2364, ARGX-109, FE301 or FM101, or any combination thereof.

Examples of agents that can inhibit TNFalpha include but are not limited to adalimumab, certolizumab pegol, etanercept, golimumab, or infliximab, or a combination thereof.

### BRIEF DESCRIPTION OF THE FIGURES

FIGS. 1A-1L. PCa cells secrete a soluble factor that reduces p62 expression in stromal cells. FIG. 1A shows qPCR of Sqstm1, Sdf-1 and Tgfβ levels in mPSC-GFP cells mixed with TRAMPC2 cells and cultured for 7 days and western blot. FIGS. 1B and 1C show immunoblot analysis for the indicated proteins (FIG. 1B) and qPCR of SQSTM1 (FIG. 1C) in WPMY-1 cells co-cultured with PCa cells during 48 h (n=3). FIGS. 1D and 1E provide immunoblot analysis for the indicated proteins (FIG. 1D) and qPCR of SQSTM1 (FIG. 1E) in WPMY-1 cells incubated 48 hours with conditioned media (CM) from PrEC, RWEP1, PC3, DU145, TRAMPC2 and LNCAP cells (n=3). FIG. 1F illustrates SQSTM1 promoter-driven luciferase in WPMY-1 incubated 48 hours with conditioned media from PrEC, RWEP1, PC3, DU145, TRAMPC2 and LNCAP cells (n=3). FIGS. 1G and 1H show immunoblot analysis for the indicated proteins (FIG. 1G) and qPCR of SQSTM1 levels (FIG. 1H) in WPMY-1 cells incubated 48 h with fractionated CM from PC3 cells (n=3). FIG. 1I shows SQSTM1 promoter-driven luciferase in WPMY-1 cells incubated 48 hours with fractionated CM from PC3 cells (n=3). FIGS. 1J and 1K show immunoblot analysis for the indicated proteins (FIG. 1J) and qPCR of SQSTM1 levels (FIG. 1K) in WPMY-1 cells incubated 48 hours with normal or boiled CM from PC3 cells (n=3). FIG. 1L) SQSTM1 promoter-driven luciferase in WPMY-1 cells incubated 48 hours with normal or boiled CM from PC3 cells (n=3). Results are shown as mean±SEM *p<0.05, **p<0.01, ***p<0.001, ****p<0.0001.

FIGS. 2A-2L. Lactate is sufficient to downregulate p62. FIG. 2A shows lactate secretion was determined by YSI2950 analyzer from PCa cells for 72 hours. Results are normalized to total protein content (n=3). FIGS. 2B and 2C show immunoblot analysis for the indicated proteins (FIG. 2B) and qPCR of SQSTM1 and MCT1 levels (FIG. 2C) in WPMY-1 cells, transduced with the indicated siRNAs, incubated with CM from PC3 cells for 48 hours (n=3). FIGS. 2D and 2E show immunoblot analysis for the indicated proteins (FIG. 2D) and qPCR of SQSTM1 and MCT4 levels (FIG. 2E) in WPMY-1 cells incubated with CM from PC3 cells, transduced with the indicated siRNAs, for 48 hours (n=3). FIGS. 2F and 2G show immunoblot analysis for the indicated proteins (FIG. 2F) and qPCR of SQSTM1 levels (FIG. 2G) in WPMY-1 cells, treated with AZD3965, and incubated with CM from PC3 cells for 48 hours (n=3). FIGS. 2H and 21 show immunoblot analysis for the indicated proteins (FIG. 2H) and qPCR of SQSTM1 levels (FIG. 2I) in WPMY-1 cells incubated with Lactate (24 mM) at different times (n=3). FIGS. 2J and 2K provide immunoblot analysis for the indicated proteins (FIG. 2J) and qPCR of SQSTM1 levels (FIG. 2K) in WPMY-1 cells incubated with different dose of Lactate for 48 hours (n=3). FIG. 2L provides immunoblot analysis for the indicated proteins in WPMY-1 cells incubated with Lactate 24 mM (pH 6.5) or DMEM at different pH for 48 hours (n=3). Results are shown as mean±SEM *p<0.05, **p<0.01, ***p<0.001, ****p<0.0001.

FIGS. 3A-3M. API elements control p62 downregulation by lactate. FIG. 3A illustrates four clusters (C1, C2, C3 and C4) heatmap of ATAC-seq peaks, +/−2 Kb from TSS in WPMY-1 cells treated or not with lactate for 48 hours. FIG. 3B illustrates HOMER discovery analysis of TFs enriched promoter regions (1-2 Kb) closed in WPMY-1 cells treated with Lactate (Control vs Lactate). FIG. 3C shows genome browser view at SQSTM1 promoter. Chromatin accessibility (ATAC-seq) and RNA-seq profiles are shown in WPMY-1 cells treated or not with Lactate. FIG. 3D illustrates AP1-driven luciferase in WPMY-1 cells treated with lactate for 48 hours (n=3). FIG. 3E shows SQSTM1 promoter-driven luciferase in WPMY-1 cells, transduced with the indicated plasmids, and treated with lactate for 48 hours (n=3). FIG. 3F shows immunoblot analysis in sgC and sgAP-1A WPMY-1 cells for the indicated proteins. FIGS. 3G and 3H show immunoblot analysis for the indicated proteins (FIG. 3G) and qPCR of SQSTM1 levels (FIG. 3H) in sgC and sgAP-1A WPMY-1cells incubated with lactate (n=3). FIG. 3I illustrates ChIP-PCR analysis of SQSTM1 promoter (AP-1A) occupancy of c-JUN, c-FOS, FOSB or JUNB in WPMY-1 cells treated or not with lactate (24 mM) for 48 hours (n=3). FIGS. 3J and 3K show immunoblot analysis for the indicated proteins (FIG. 3J) and qPCR of SQSTM1 levels (FIG. 3K) in WPMY-1 cells incubated with lactate and transduced with the indicated siRNAs (n=3). FIGS. 3L and 3M show immunoblot analysis for the indicated proteins (FIG. 3L) and qPCR of SQSTM1 levels in WPMY-1 cells incubated with lactate and treated with 10 μM of SP600125 (n=3). Results are shown as mean±SEM *p<0.05, **p<0.01, ***p<0.001, ****p<0.0001.

FIGS. 4A-4G. Changes in NAD+/NADH levels by lactate are critical for p62 downregulation. FIGS. 4A and 4B show immunoblot analysis for the indicated proteins (FIG. 4E) and qPCR of SQSTM1 levels (FIG. 4F) in WPMY-1 incubated with Lactate or Pyruvate for 48 hours (n=3). FIG. 4C illustrates intracellular NAD levels in WPMY-1 cells treated or not with lactate (n=3). FIGS. 4D and 4E show immunoblot analysis for the indicated proteins and qPCR of SQSTM1 levels in WPMY-1 cells incubated with Lactate and NAD (150 μM) for 48 hours (n=3). FIGSs. 4F and 4G show immunoblot analysis for the indicated proteins and qPCR of SQSTM1 levels in WPMY-1 cells incubated with Lactate or NR (200 μM) for 48 hours (n=3). Results are shown as mean±SEM **p<0.01, ***p<0.001, ****p<0.0001.

FIGS. 5A-5T. PARP-1 is critical for the control of p62 levels in stromal cells. FIG. 5A shows immunoblot analysis for the indicated proteins in WPMY-1 cells transduced with the indicated siRNAs. FIGS. 5B and 5C show immunoblot analysis for the indicated proteins (B) and qPCR of SQSTM1 levels (FIG. 5C) in WPMY-1 cells transduced with the indicated siRNAs. FIGS. 5D and 5E show immunoblot analysis for the indicated proteins and qPCR of PARP-1 (FIG. 5D) and SQSTM1 (FIG. 5E) levels in WPMY-1 cells transduced with the indicated siRNAs. FIGS. 5F and 5G show immunoblot analysis for the indicated proteins (FIG. 5F) and qPCR of SQSTM1 levels (FIG. 5G) in WPMY-1 cells incubated with PJ34 for 24 hours (n=3). FIGS. 5H and 5I show immunoblot analysis for the indicated proteins (FIG. 5H) and qPCR of SQSTM1 levels (FIG. 5I) in WPMY-1 cells incubated with Olaparib for 48 hours (n=3). FIGS. 5J and 5K illustrate ChIP-PCR analysis of SQSTM1 promoter (AP-1A) occupancy of c-FOS (FIG. 5J) or c-JUN (FIG. 5K) in WPMY-1 cells treated or not with Olaparib (20 μM) for 48 hours (n=3). FIG. 5L shows immunoblot analysis for the indicated proteins and PARylated protein levels in WPMY-1 cells incubated with lactate for the indicated times. FIGS. 5M and 5N illustrate PARylation of c-JUN (FIG. 5M) or c-FOS (FIG. 5N) in WPMY-1 treated or not with lactate for 48 h (n=2). FIGS. 5O and 5P illustrate PARylation of c-JUN (FIG. 5O) or c-FOS (FIG. 5P) in WPMY-1 treated or not with lactate and NAD for 48 hours (n=2). FIGS. 5Q and 5R illustrate ChIP-PCR analysis of SQSTM1 promoter (AP-1A) occupancy of c-FOS (FIG. 5Q) or c-JUN (FIG. 5R) in WPMY-1 cells treated or not with lactate and NAD for 48 hours (n=3). FIGS. 5S and 5T illustrate ChIP-PCR analysis of SQSTM1 promoter (AP-1A) occupancy of c-FOS (FIG. 5S) or c-JUN (FIG. 5T) in WPMY-1 cells treated or not with lactate and NR for 48 hours (n=3). Results are shown as mean±SEM *p<0.05, **p<0.01, ***p<0.001, ****p<0.0001.

FIGS. 6A-6M. API control of the SQSTM1 promoter in stromal cells is critical for PCa tumor growth and invasion. FIG. 6A shows qPCR analysis of mRNA levels of the indicated genes in WPMY-1 cells incubated with Lactate (24 mM) for 48 hours (n=3). FIG. 6B shows qPCR analysis of mRNA levels of the indicated genes in WPMY-1 cells treated or not with lactate and NAD for 48 hours (n=3). FIG. 6C illustrates GSEA analysis of “LACTATE UP” and “LACTATE DOWN” gene signatures in CAFs vs. NAFs gene set GSE34312. FIG. 6D illustrates qPCR analysis of mRNA levels of indicated genes in sgC and sgAP-1A WPMY-1 cells (n=3). FIG. 6E-6G show experimental design (FIG. 6E), Migration (FIG. 6F), and Invasion (FIG. 6G) of PC3 cells co-cultured with sgC or sgAP-1A WPMY-1 cells for 16 h. Representative images and quantification (n=3). FIGS. 6H and 6I show experimental design (FIG. 6H) and H&E staining (FIG. 6I) of organotypic gels combining PC3 cells with sgC and sgAP-1A WPMY-1 cells. Quantification of PC3 cells invasion and proliferation of experiments shown in (FIG. 6I). FIG. 6J-6M illustrate subcutaneous xenograft co-implantation in castrated NSG mice (male, 7 weeks old) of PC3 PCa cells with sgC or sgAP-1A WPMY-1 cells (PC3+sgC WPMY-1 n=8; PC3+sgAP-1A WPMY-1 n=8). Experimental design (FIG. 6J), Tumor weight (FIG. 6K), gross images (FIG. 6L), IHC for Masson Trichrome, HA and aSMA (FIG. 6M), scale bars 50 μm. Results are shown as mean±SEM *p<0.05, **p<0.01, ***p<0.001, ****p<0.0001.

FIGS. 7A-7T. Olaparib treatment mimics lactate loss of p62 and promotes stromal activation. FIG. 7A illustrates enriched pathways obtained by GSEA pre-ranked analysis using DEG in Olaparib-treated WPMY-1 cells. FIG. 7B illustrates GSEA analysis for stromal activation and CAF phenotype gene signatures in Olaparib-treated WPMY-1 cells. FIG. 7C illustrates GSEA analysis of “Olaparib_UP”, “Olaparib_DN”, “Olaparib_ADT_UP”, “Olaparib_ADT_DN” gene signatures in CAFs vs. NAFs gene set GSE34312. FIG. 7D shows qPCR analysis of mRNA levels of indicated genes in Olaparib-treated WPMY-1 cells (n=3). FIG. 7E shows four clusters (C1, C2, C3 and C4) heatmap of ATAC-seq peaks, +/−2 Kb from TSS, upon Olaparib treatment for 96 hours in basal conditions. FIG. 7F shows HOMER discovery analysis of TFs enriched in promoter regions (1-2 Kb) closed upon Olaparib treatment in WPMY-1 cells (DMSO vs Olaparib). FIG. 7G shows integrative Genomics Viewer (IGV) of ATAC-seq peaks in SQSTM1 promoter in Olaparib-treated WPMY-1 cells in basal conditions. FIGS. 7H-7J show trans-well migration and invasion assays. Experimental design (FIG. 7H), left, trans-well migration and invasion assay of PC3 PCa cultured with Olaparib pre-treated WPMY-1 cells for 20 hours, scale bar 50 μm. Right, relative number of migrating and invading cells (FIGS. 7I-7J). FIGS. 7K and 7L show click-iT EdU incorporation assay. Experimental design (FIG. 7K), left, representative images of EdU incorporation in PC3 PCa cells treated with CM from Olaparib-treated WPMY-1 cells for 24 hours. Right, percentage of Edu positive cells (n=3) (FIG. 7L), scale bar 50 μm. FIGS. 7M and 7N show survival assay of GFP-labeled PC3 PCa cells co-cultured with WPMY-1 cells or alone, treated with DMSO or Olaparib, in basal and ADT conditions. Experimental design (FIG. 7M), relative GFP positive area after days of culture (n=6) (FIG. 7N). FIGS. 7O-7T illustrate subcutaneous xenograft co-implantation in NSG mice (male, 7 weeks old) of PC3 PCa cells with WPMY-1 cells (PC3+WPMY-1 n=20). Mouse were treated twice a week with Olaparib 40 mg/Kg for 2 weeks. Experimental design (FIG. 7O), tumor weight (g) (FIG. 7P), tumor volume (mm³) (FIG. 7Q), gross images (FIG. 7R), IHC for Masson Trichrome, HA, and aSMA (FIG. 7S). Immunofluorescence (IF) for aSMA, p62, and DAPI. scale bars 50 μm (FIG. 7T). Results are shown as mean±SEM *p<0.05, **p<0.01, ***p<0.001, ****p<0.0001.

FIGS. 8A-8N. Stromal activation upon Olaparib treatment impacts prostate epithelial cells. FIGS. 8A and 8B illustrate TRAMP⁺ mice treated with Olaparib 40 mg/Kg, 5 days a week for 4 weeks. Experimental design (FIG. 8A), IHC for HA, and aSMA (FIG. 8B), scale bars 50 μm. FIGS. 8C and 8D show scRNAseq compartment identification. UMAP plot with clustering results color-coded by compartment types (FIG. 8C). Proportion cell number of the identified cell compartments (FIG. 8D). FIGS. 8E and 8F show scRNAseq fibroblast subset. UMAP plot with re-clustering of fibroblast showing the main markers of each cluster (FIG. 8E). Feature plot for the expression of indicated genes (FIG. 8F). (FIG. 8G shows qPCR analysis of mRNA levels of indicated genes in Olaparib-treated WPMY-1 cells (n=3). FIG. 8H shows IHC for SPINK1, scale bars 50 μm. FIG. 8I-8L illustrate scRNAseq epithelial subset. UMAP plot with re-clustering of epithelial cells color-coded by cell type (FIG. 8I). Violin plots of specific epithelial cell markers (FIG. 8J). Proportion cell number of the identified epithelial cell populations (FIG. 8K). Violin plots of indicated gene signatures (FIG. 8L). FIG. 8M shows IHC for pSMAD2, scale bars 50 μm. FIG. 8N illustrates Cellphone DB analysis showing ligand-receptor interactions between identified epithelial and fibroblast populations.

FIGS. 9A-9G. Stroma activation blockade reduce the tumor supporting effects of Olaparib. FIG. 9A shows IF for HABP in WPMY-cells treated with vehicle (DMSO), Olaparib 10 μM or Olaparib 10 μM+PEGPH20 2.5 μg/ml for 96-hour, scale bars 50 μm. FIGS. 9B and 9C illustrate trans-well migration assays. Experimental design (FIG. 9B), relative number of migrating PC3 PCa cultured with Olaparib 10 μM or Olaparib 10 μM+PEGPH20 2.5 μg/m pre-treated WPMY-1 cells for 20 hours, basal and ADT conditions (FIG. 9C). FIGS. 9D and 9E show Click-iT EdU incorporation assay. Experimental design (FIG. 9D), left, representative images of EdU incorporation in PC3 PCa cells treated with CM from Olaparib 10 μM, Olaparib 10 μM+PEGPH20 2.5 μg/m or Olaparib 10 μM+CM incubation with Galunisertib 10 μM WPMY-1 cells for 24 hours. Right, percentage of Edu positive cells (n=3) (FIG. 9E), scale bar 50 μm. FIGS. 9F and 9G illustrate subcutaneous xenograft co-implantation in NSG mice (male, 7 weeks old) of PC3 PCa cells with WPMY-1 cells (PC3+WPMY-1 n=20). Mouse were treated twice a week with Olaparib 40 mg/Kg or Olaparib+PEGPH20 0.0375 mg/Kg for 2 weeks. Experimental design (FIG. 9F), tumor volume (mm³) (FIG. 9G).

FIGS. 10A-10F. Lactate-driven p62 downregulation is not affected by different degradative mechanisms. FIG. 10A shows WPMY-1 cells, incubated or not with lactate for 48 hours, were incubated with 50 μg/ml of cycloheximide (CHX) and protein stability was determined by immunoblotting at indicated time points. P62 levels were normalized to actin (n=3). FIGS. 10B and 10C show immunoblot analysis for indicated proteins in WPMY-1 cells treated or not with lactate and MG132 (FIG. 10B) or Bafilomycin Al (FIG. 10C) for 48 hours. FIG. 10D shows immunoblot analysis for indicated proteins in WPMY-1 cells, treated or not with lactate for 48 hours, and transduced with the indicated siRNAs. FIGS. 10E and 10F show immunoblot analysis for indicated proteins in WPMY-1 cells, treated or not with lactate for 48 hours and a dose-response of the Calpain inhibitor (FIG. 10E) or Caspase inhibitor (FIG. 10F).

FIG. 11A-11F. Selective downregulation of p62 by PARP1 deletion. FIGS. 11A and 11B show qPCR analysis of mRNA levels of the indicated genes in WPMY-1 cells, transduced with the indicated siRNAs. FIG. 11C shows qPCR analysis of mRNA levels of the indicated genes and immunoblot analysis for indicated proteins in sgPARP-1 and sgC WPMY-1 cells. FIG. 11D-11E illustrate Olaparib induces autophagy. Immunoblot analysis of p62 (FIG. 11D) and LC3 (FIG. 11E) in WPMY-1 cells treated with Olaparib in the presence of lysosomal inhibitors (PI). FIG. 11F shows treatment with an autophagy inhibitor (ULK inhibitor, SBI-0202965) reverts the stroma activation induced by Olaparib as determined by qPCR of the CAF marker, ACTA2.

FIGS. 12A-12D. Olaparib induces fibroblast activation in different stromal cells. FIG. 12A-12D show qPCR analysis of mRNA levels of the indicated genes in (FIG. 12A) WPMY-1 cells (FIG. 12B) human normal primary fibroblast cells (FIG. 12C) mouse normal fibroblast cells (FIG. 12D) shows mouse hepatic stellate cells treated with Olaparib.

FIGS. 13A-F. PARP inhibitors induce stromal activation in different cell lines and in vivo models. FIG. 13A-13D show immunoblot analysis for P62 protein in primary prostate fibroblasts (FIG. 13A), lung primary fibroblasts (FIG. 13B), breast primary fibroblasts (FIG. 13C), and endometrial primary fibroblasts (FIG. 13D), veh-treated or Olaparib-treated, 20 μM, for 4 days. FIG. 13E-13F show GSEA analysis for the indicated stromal activation signatures comparing Talazoparib-(PARP inhibitor) vs veh-treated in mammary gland tumors (GSE125206) (FIG. 13E) and Niraparib (PARP inhibitor) vs veh-treated in human ovarian cancer PDXs (GSE163854) (FIG. 13F) showing stromal activation in PARP inhibitors treated samples.

FIGS. 14A-14F. Targeting IL6/JAK/STAT or TNFa signaling decreases stromal activation and rescue the proliferative effect conferred to PC3 prostate tumor epithelial cells. FIG. 14A-14C show GSEA analysis for the indicated signatures in Olaparib 20 μM vs DMSO-treated WPMY-1 cells (FIG. 14A), Talazoparib-(PARP inhibitor) vs saline-treated in mammary gland tumors (GSE125206, FIG. 14B) and Niraparib-(PARP inhibitor) vs veh-treated in human ovarian cancer PDXs (GSE163854, FIG. 14C), showing increased inflammatory related signatures upon PARP inhibitor treatment. FIG. 14D shows experimental design. FIG. 14E shows qPCR analysis of mRNA levels of indicated genes in WPMY-1 cells treated with DMSO, Olaparib 10 μM, Lestaurtinib (JAKi) 50 nM or Olaparib+Lestaurtinib 50 nM (n=3). FIG. 14F shows Click-iT EdU incorporation assay, left, representative images of EdU incorporation in PC3 PCa cells treated 24 h with CM from WPMY-1 treated with DMSO, Olaparib 10 μM, Olaparib 10 μM+Lestaurtinib 50 nM, Olaparib 10 μM+IL-6 capture antibody (2h), Olaparib 10 μM+TNFa neutralizing antibody 1 μg/ml (1 h), Lestaurtinib 50 nM, IL-6 capture antibody (2 h) or TNFa neutralizing antibody 1 μg/m (1 h). Right, percentage of Edu positive cells (n=3) scale bars 50 μm.

FIGS. 15A-15K. Olaparib induces stromal activation in vivo both in healthy and metastatic tissues. FIG. 15A-15C shows Olaparib induces stromal activation in tumor-invaded lymph nodes and bones in TRAMP⁺/FVB castrated mice. Experimental design (FIG. 15A). IHC for HA and «SMA in tumor-invaded lymph nodes from Olaparib- or Veh-treated mice (n=16) showing increased stromal content upon Olaparib treatment, scale bars 50 μm FIG. 15B shows IHC for HA in bone from Olaparib- or Veh-treated mice (n=3) showing increased HA deposition in the growth plate and stromal areas, scale bars 100 μm (C). FIG. 15D-15E show bone in culture (BICA) assay on femur pieces pre-treated or not with Olaparib 20 u M for 48 h cocultured with RM1-BoMe3 tumor epithelial cells for 48 h. Experimental design (FIG. 15D). Left representative images of RFP⁺ RM1-BoMe3 cells attached to the bone. Right, graph or RFP⁺ positive area showing increased tumor homing of RM1-BoMe3 in Olaparib pre-treated bones (FIG. 15E). FIG. 15F shows qPCR analysis of mRNA levels of indicated genes in isolated bone marrow cells from Olaparib-40 mg/kg or veh-treated mice (n=3). FIG. 15G shows qPCR analysis of mRNA levels of indicated genes in cultured primary bone marrow cells treated with Olaparib 10 μM or DMSO (96 h). FIG. 15H-15J illustrate caudal artery injection of RM1-BoMe3 mouse metastasis model showing that Olaparib induces HA deposition and activation of TGFβ signaling. Experimental design (FIG. 15H). IHC for HA showing increased HA deposition in Olaparib-treated liver metastasis (n=3), scale bars 100 μm (FIG. 15I). IHC for pSMAD2 showing an increased pSMAD2 staining in bone metastasis in Olaparib-treated mice (n=3), scale bars 100 μm (FIG. 15J).

FIG. 15K shows IHC for HA in pre- and post-Olaparib treatment in a prostate cancer bone metastasis showing increased HA deposition in Olaparib-treated mice, scale bars 100 μm.

### DETAILED DESCRIPTION

Mounting evidence identifies the tumor microenvironment (TME), including stromal fibroblasts, as a driver of tumorigenesis in many types of tumors, including prostate cancer (PCa) (Klemm and Joyce, 2015; Nakanishi et al., 2018; Zhang et al., 2013; Zhang et al., 2020). It is well accepted now that the activation of tumor stromal fibroblasts to a state commonly known as cancer-associated fibroblasts (CAFs) can embolden epithelial cancer cells to progress to more malignant stages and affect disease outcomes (Barron and Rowley, 2012; Cunha et al., 2002; Rowley, 1998; Tuxhorn et al., 2001). CAFs impact the tumor epithelium by modulating many functions through the secretion of stromal growth factors and inflammatory mediators, by reprogramming their metabolism to provide nutrients and survival factors, and by remodeling the extracellular matrix (Hanahan and Weinberg, 2011; Kasashima et al., 2020; Sahai et al., 2020). This fibroblast-epithelium synergistic crosstalk promotes malignancy and is believed to negatively impact cancer treatment by promoting the accumulation of “persisting” or “resistant” tumor cells, which limit therapy efficacy and promote resistance (Bluemn et al., 2017; Linares et al., 2017; Zhang et al., 2020). The renewed interest in the study of CAFs has resulted in the extensive characterization of their cell heterogeneity as well as the establishment of a complex catalogue of the myriad of secreted intermediates that likely impact tumor progression and response to therapy. However, a major gap in the field is the identification of the key master regulators of the acquisition of the CAF phenotype, as well as the molecular mechanisms whereby the tumor epithelium drives this process.

The autophagy substrate and signaling adaptor p62 (encoded by SQSTM1 gene) is a regulator of CAF biology (Huang et al., 2018; Linares et al., 2017; Valencia et al., 2014). P62 has a dual role in cancer (Duran et al., 2011; Duran et al., 2016; Duran et al., 2008; Moscat et al., 2016; Umemura et al., 2016). Thus, while it plays an important oncogenic function in the tumor epithelium, it is a suppressor of tumor progression by restraining CAF activation (Duran et al., 2016; Goruppi and Dotto, 2013; Linares et al., 2017; Reina-Campos et al., 2018; Valencia et al., 2014). It is well established that p62 is upregulated in the epithelium of many types of tumors, which invariably associates with more aggressive cancer and poorer overall survival (Moscat and Diaz-Meco, 2009; Moscat et al., 2016). However, many tumors display reduced levels of p62 in their stroma, especially in CAFs (Duran et al., 2016; Goruppi and Dotto, 2013; Linares et al., 2017; Valencia et al., 2014). p62 downregulation is a central event in the acquisition of the CAF phenotype and in creating a pro-tumorigenic microenvironment conducive to cancer (Linares et al., 2017; Valencia et al., 2014). From a mechanistic point of view, p62 inactivation in prostate stromal fibroblasts impairs their metabolic detoxification capacity and leads to the accumulation of reactive oxygen species, which in turn results in the release of pro-survival inflammatory cytokines (Valencia et al., 2014). Interestingly, under nutrient stress conditions, the downregulation of p62 in fibroblasts also promotes tumorigenesis by enabling their own survival and that of tumor epithelial cells (Linares et al., 2017). Specifically, under conditions of glutamine deprivation, a common situation in the TME of highly aggressive cancers, the loss of p62 in stromal fibroblasts reprograms their metabolism to produce and secrete asparagine to maintain protein synthesis in the tumor epithelium (Linares et al., 2017). These studies highlight the pivotal role of p62 as a tumor suppressor in the stroma by activating pro-survival pathways and the cellular adaptation to nutrient deprivation.

Therefore, whereas the genetic inactivation of p62 selectively in the tissue epithelium impairs tumorigenesis, that in the stromal fibroblasts has the opposite effect by impacting several metabolic and inflammatory pathways (Moscat et al., 2016). The fact that whole body genetic inactivation of p62 results in increased tumorigenesis, mimicking the phenotype of mice with p62 deficiency in the fibroblast stromal compartment, strongly suggests that the stromal role of p62 as a tumor suppressor is what determines the global impact of p62 in cancer (Valencia et al., 2014). This indicates that the tumor-promoting signals produced by the loss of p62 in CAFs override the requirement for p62 in the tumor epithelium, which highlights the importance of studying how p62 is lost in the tumor's fibroblast compartment. In this regard, a key pending question is to determine how tumors downregulate p62 in the stroma to promote the CAF phenotype. Identifying this mechanism is of paramount importance because, given the role of p62 in CAF activation and function, this will provide a paradigm for understanding of the master regulators of stromal activation, which will be more amenable to therapeutic intervention than trying to affect the complex downstream effectors unleashed upon stromal activation.

### PARP1

The poly (ADP-ribose) polymerase 1 (PARP1) enzyme helps cells repair themselves if they become damaged by serving as a “sensor” for DNA strand breaks. Increased PARP1 expression is sometimes observed in melanomas, breast cancer, lung cancer, and other neoplastic diseases. PARP inhibitors stop PARP from repairing cancer cells.

The human PARP1 gene (gene ID: 142) is located on chromosome 1 (NCBI NC_000001.11 (226360691 . . . 226408093, complement)). An amino acid sequence for human PARP1 is available in the Uniprot database as accession no. P09874 and shown below as SEQ ID NO:1.

```
                10 20 30 40
MAESSDKLYR VEYAKSGRAS CKKCSESIPK DSLRMAIMVQ
                50 60 70 80
SPMEDGKVPH WYHFSCFWKV GHSIRHPDVE VDGFSELRWD
                90 100 110 120
DQQKVKKTAE AGGVTGKGQD GIGSKAEKTL GDFAAEYAKS
              130 140 150 160
NRSTCKGCME KIEKGQVRLS KKMVDPEKPQ LGMIDRWYHP
              170 180 190 200
GCFVKNREEL GFRPEYSASQ LKGFSLLATE DKEALKKQLP
              210 220 230 240
GVKSEGKRKG DEVDGVDEVA KKKSKKEKDK DSKLEKALKA
              250 260 270 280
QNDLIWNIKD ELKKVCSIND LKELLIFNKQ QVPSGESAIL
              290 300 310 320
DRVADGMVFG ALLPCEECSG QLVFKSDAYY CTGDVTAWTK
              330 340 350 360
CMVKTQTPNR KEWVTPKEFR EISYLKKLKV KKQDRIFPPE
              370 380 390 400
TSASVAATPP PSTASAPAAV NSSASADKPL SNMKILTLGK
              410 420 430 440
LSRNKDEVKA MIEKLGGKLT GTANKASLCI STKKEVEKMN
              450 460 470 480
KKMEEVKEAN IRVVSEDFLQ DVSASTKSLQ ELFLAHILSP
              490 500 510 520
WGAEVKAEPV EVVAPRGKSG AALSKKSKGQ VKEEGINKSE
              530 540 550 560
KRMKLTLKGG AAVDPDSGLE HSAHVLEKGG KVFSATLGLV
              570 580 590 600
DIVKGINSYY KLQLLEDDKE NRYWIFRSWG RVGTVIGSNK
              610 620 630 640
LEQMPSKEDA IEHFMKLYEE KTGNAWHSKN FTKYPKKFYP
              650 660 670 680
LEIDYGQDEE AVKKLTVNPG TKSKLPKPVQ DLIKMIFDVE
              690 700 710 720
SMKKAMVEYE IDLQKMPLGK LSKRQIQAAY SILSEVQQAV
              730 740 750 760
SQGSSDSQIL DLSNRFYTLI PHDFGMKKPP LLNNADSVQA
              770 780 790 800
KVEMLDNLLD IEVAYSLLRG GSDDSSKDPI DVNYEKLKTD
              810 820 830 840
IKVVDRDSEE AEIIRKYVKN THATTHNAYD LEVIDIFKIE
850 860 870 880
REGECQRYKP FKQLHNRRLL WHGSRTTNFA GILSQGLRIA
              890 900 910 920
PPEAPVTGYM FGKGIYFADM VSKSANYCHT SQGDPIGLIL
              930 940 950 960
LGEVALGNMY ELKHASHISK LPKGKHSVKG LGKTTPDPSA
              970 980 990 1000
NISLDGVDVP LGTGISSGVN DTSLLYNEYI VYDIAQVNLK
            1010
YLLKLKFNFK TSLW
```

A cDNA sequence for human PARP1 is available in the NCBI database as accession no. NM_001618.4 and shown below as SEQ ID NO:2.

| | |
|---|---|
| 1 | AGCAATCTAT CAGGGAACGG CGGTGGCCGG TGCGGCGTGT |
| 41 | TCGGTGGCGG CTCTGGCCGC TCAGGCGCCT GCGGCTGGGT |
| 81 | GAGCGCACGC GAGGCGGCGA GGCGGCAGCG TGTTTCTAGG |
| 121 | TCGTGGCGTC GGGCTTCCGG AGCTTTGGCG GCAGCTAGGG |
| 161 | GAGGATGGCG GAGTCTTCGG ATAAGCTCTA TCGAGTCGAG |
| 201 | TACGCCAAGA GCGGGCGCGC CTCTTGCAAG AAATGCAGCG |
| 241 | AGAGCATCCC CAAGGACTCG CTCCGGATGG CCATCATGGT |
| 281 | GCAGTCGCCC ATGTTTGATG GAAAAGTCCC ACACTGGTAC |
| 321 | CACTTCTCCT GCTTCTGGAA GGTGGGCCAC TCCATCCGGC |
| 361 | ACCCTGACGT TGAGGTGGAT GGGTTCTCTG AGCTTCGGTG |
| 401 | GGATGACCAG CAGAAAGTCA AGAAGACAGC GGAAGCTGGA |
| 441 | GGAGTGACAG GCAAAGGCCA GGATGGAATT GGTAGCAAGG |
| 481 | CAGAGAAGAC TCTGGGTGAC TTTGCAGCAG AGTATGCCAA |
| 521 | GTCCAACAGA AGTACGTGCA AGGGGTGTAT GGAGAAGATA |
| 561 | GAAAAGGGCC AGGTGCGCCT GTCCAAGAAG ATGGTGGACC |
| 601 | CGGAGAAGCC ACAGCTAGGC ATGATTGACC GCTGGTACCA |
| 641 | TCCAGGCTGC TTTGTCAAGA ACAGGGAGGA GCTGGGTTTC |
| 681 | CGGCCCGAGT ACAGTGCGAG TCAGCTCAAG GGCTTCAGCC |
| 721 | TCCTTGCTAC AGAGGATAAA GAAGCCCTGA AGAAGCAGCT |
| 761 | CCCAGGAGTC AAGAGTGAAG GAAAGAGAAA AGGCGATGAG |
| 801 | GTGGATGGAG TGGATGAAGT GGCGAAGAAG AAATCTAAAA |
| 841 | AAGAAAAAGA CAAGGATAGT AAGCTTGAAA AAGCCCTAAA |
| 881 | GGCTCAGAAC GACCTGATCT GGAACATCAA GGACGAGCTA |
| 921 | AAGAAAGTGT GTTCAACTAA TGACCTGAAG GAGCTACTCA |
| 961 | TCTTCAACAA GCAGCAAGTG CCTTCTGGGG AGTCGGCGAT |
| 1001 | CTTGGACCGA GTAGCTGATG GCATGGTGTT CGGTGCCCTC |
| 1041 | CTTCCCTGCG AGGAATGCTC GGGTCAGCTG GTCTTCAAGA |
| 1081 | GCGATGCCTA TTACTGCACT GGGGACGTCA CTGCCTGGAC |
| 1121 | CAAGTGTATG GTCAAGACAC AGACACCCAA CCGGAAGGAG |
| 1161 | TGGGTAACCC CAAAGGAATT CCGAGAAATC TCTTACCTCA |
| 1201 | AGAAATTGAA GGTTAAAAAA CAGGACCGTA TATTCCCCCC |
| 1241 | AGAAACCAGC GCCTCCGTGG CGGCCACGCC TCCGCCCTCC |
| 1281 | ACAGCCTCGG CTCCTGCTGC TGTGAACTCC TCTGCTTCAG |
| 1321 | CAGATAAGCC ATTATCCAAC ATGAAGATCC TGACTCTCGG |
| 1361 | GAAGCTGTCC CGGAACAAGG ATGAAGTGAA GGCCATGATT |
| 1401 | GAGAAACTCG GGGGGAAGTT GACGGGGACG GCCAACAAGG |
| 1441 | CTTCCCTGTG CATCAGCACC AAAAAGGAGG TGGAAAAGAT |
| 1481 | GAATAAGAAG ATGGAGGAAG TAAAGGAAGC CAACATCCGA |
| 1521 | GTTGTGTCTG AGGACTTCCT CCAGGACGTC TCCGCCTCCA |
| 1561 | CCAAGAGCCT TCAGGAGTTG TTCTTAGCGC ACATCTTGTC |
| 1601 | CCCTTGGGGG GCAGAGGTGA AGGCAGAGCC TGTTGAAGTT |
| 1641 | GTGGCCCCAA GAGGGAAGTC AGGGGCTGCG CTCTCCAAAA |
| 1681 | AAAGCAAGGG CCAGGTCAAG GAGGAAGGTA TCAACAAATC |
| 1721 | TGAAAAGAGA ATGAAATTAA CTCTTAAAGG AGGAGCAGCT |
| 1761 | GTGGATCCTG ATTCTGGACT GGAACACTCT GCGCATGTCC |
| 1801 | TGGAGAAAGG TGGGAAGGTC TTCAGTGCCA CCCTTGGCCT |
| 1841 | GGTGGACATC GTTAAAGGAA CCAACTCCTA CTACAAGCTG |
| 1881 | CAGCTTCTGG AGGACGACAA GGAAAACAGG TATTGGATAT |
| 1921 | TCAGGTCCTG GGGCCGTGTG GGTACGGTGA TCGGTAGCAA |
| 1961 | CAAACTGGAA CAGATGCCGT CCAAGGAGGA TGCCATTGAG |
| 2001 | CACTTCATGA AATTATATGA AGAAAAAACC GGGAACGCTT |
| 2041 | GGCACTCCAA AAATTTCACG AAGTATCCCA AAAAGTTCTA |
| 2081 | CCCCCTGGAG ATTGACTATG GCCAGGATGA AGAGGCAGTG |
| 2121 | AAGAAGCTGA CAGTAAATCC TGGCACCAAG TCCAAGCTCC |
| 2161 | CCAAGCCAGT TCAGGACCTC ATCAAGATGA TCTTTGATGT |
| 2201 | GGAAAGTATG AAGAAAGCCA TGGTGGAGTA TGAGATCGAC |
| 2241 | CTTCAGAAGA TGCCCTTGGG GAAGCTGAGC AAAAGGCAGA |
| 2281 | TCCAGGCCGC ATACTCCATC CTCAGTGAGG TCCAGCAGGC |
| 2321 | GGTGTCTCAG GGCAGCAGCG ACTCTCAGAT CCTGGATCTC |
| 2361 | TCAAATCGCT TTTACACCCT GATCCCCCAC GACTTTGGGA |
| 2401 | TGAAGAAGCC TCCGCTCCTG AACAATGCAG ACAGTGTGCA |
| 2441 | GGCCAAGGTG GAAATGCTTG ACAACCTGCT GGACATCGAG |
| 2481 | GTGGCCTACA GTCTGCTCAG GGGAGGGTCT GATGATAGCA |
| 2521 | GCAAGGATCC CATCGATGTC AACTATGAGA AGCTCAAAAC |
| 2561 | TGACATTAAG GTGGTTGACA GAGATTCTGA AGAAGCCGAG |
| 2601 | ATCATCAGGA AGTATGTTAA GAACACTCAT GCAACCACAC |
| 2641 | ACAATGCGTA TGACTTGGAA GTCATCGATA TCTTTAAGAT |
| 2681 | AGAGCGTGAA GGCGAATGCC AGCGTTACAA GCCCTTTAAG |
| 2721 | CAGCTTCATA ACCGAAGATT GCTGTGGCAC GGGTCCAGGA |
| 2761 | CCACCAACTT TGCTGGGATC CTGTCCCAGG GTCTTCGGAT |
| 2801 | AGCCCCGCCT GAAGCGCCCG TGACAGGCTA CATGTTTGGT |
| 2841 | AAAGGGATCT ATTTCGCTGA CATGGTCTCC AAGAGTGCCA |
| 2881 | ACTACTGCCA TACGTCTCAG GGAGACCCAA TAGGCITAAT |
| 2921 | CCTGTTGGGA GAAGTTGCCC TTGGAAACAT GTATGAACTG |
| 2961 | AAGCACGCTT CACATATCAG CAAGTTACCC AAGGGCAAGC |
| 3001 | ACAGTGTCAA AGGTTTGGGC AAAACTACCC CTGATCCTTC |
| 3041 | AGCTAACATT AGTCTGGATG GTGTAGACGT TCCTCTTGGG |
| 3081 | ACCGGGATTT CATCTGGTGT GAATGACACC TCTCTACTAT |
| 3121 | ATAACGAGTA CATTGTCTAT GATATTGCTC AGGTAAATCT |
| 3161 | GAAGTATCTG CTGAAACTGA AATTCAATTT TAAGACCTCC |
| 3201 | CTGTGGTAAT TGGGAGAGGT AGCCGAGTCA CACCCGGTGG |
| 3241 | CTCTGGTATG AATTCACCCG AAGCGCTTCT GCACCAACTC |
| 3281 | ACCTGGCCGC TAAGTTGCTG ATGGGTAGTA CCTGTACTAA |
| 3321 | ACCACCTCAG AAAGGATTTT ACAGAAACGT GTTAAAGGTT |
| 3361 | TTCTCTAACT TCTCAAGTCC CTTGTTTTGT GTTGTGTCTG |
| 3401 | TGGGGAGGGG TTGTTTTGGG GTTGTTTTTG TTTTTTCTTG |
| 3441 | CCAGGTAGAT AAAACTGACA TAGAGAAAAG GCTGGAGAGA |
| 3481 | GATTCTGTTG CATAGACTAG TCCTATGGAA AAAACCAAGC |
| 3521 | TTCGTTAGAA TGTCTGCCTT ACTGGTTTCC CCAGGGAAGG |
| 3561 | AAAAATACAC TTCCACCCTT TTTTCTAAGT GTTCGTCTTT |
| 3601 | AGTTTTGATT TTGGAAAGAT GTTAAGCATT TATTTTTAGT |
| 3641 | TAAAAATAAA AACTAATTTC ATACTATTTA GATTTTCTTT |
| 3681 | TTTATCTTGC ACTTATTGTC CCCTTTTTAG TTTTTTTTGT |
| 3721 | TTGCCTCTTG TGGTGAGGGG TGTGGGAAGA CCAAAGGAAG |
| 3761 | GAACGCTAAC AATTTCTCAT ACTTAGAAAC AAAAAGAGCT |
| 3801 | TTCCTTCTCC AGGAATACTG AACATGGGAG CTCTTGAAAT |
| 3841 | ATGTAGTATT AAAAGTTGCA TTTGAAATTC TTGACTTTCT |
| 3881 | TATGGGCACT TTTGTCTTCC AAATTAAAAC TCTACCACAA |
| 3921 | ATATACTTAC CCAAGGGCTA ATAGTAATAC TCGATTAAAA |
| 3961 | ATGCAGATGC CTTCTCTA |

Subjects can be effectively treated by the compositions and methods described herein even though those subjects have PARP1 isoforms and variants with somewhat different PARP1 sequences than the examples of PARP1 sequences described herein. Such PARP1 isoforms and variants can have polypeptide or nucleic acid sequences with between 55-100% sequence identity to a reference sequence (e.g., a sequence described herein). For example, PARP1 isoforms and variants can have at least 55% sequence identity, e.g., at least 60%, at least 70%, at least 80%, at least 90%, at least 95%, at least 96%, at least 97% sequence, at least 98%, at least 99% identity to a reference sequence (e.g., a sequence described herein) over a specified comparison window. Optimal alignment may be ascertained or conducted using the homology alignment algorithm of Needleman and Wunsch, J. Mol. Biol. 48:443-53 (1970).

### Hyaluronan Synthase

Hyaluronan or hyaluronic acid is a high molecular weight unbranched polysaccharide synthesized within a variety of organisms, including mammals. Hyaluronan or hyaluronic acid is a constituent of the extracellular matrix. It consists of alternating glucuronic acid and N-acetylglucosamine residues that are linked by beta-1-3 and beta-1-4 glycosidic bonds. Hyaluronic acid is synthesized by membrane-bound synthase at the inner surface of the plasma membrane, and the chains are extruded via ABC-Transporter into the extracellular space. It serves a variety of functions, including space filling, lubrication of joints, and provision of a matrix through which cells can migrate.

For example, a human hyaluronan synthase 1 (HAS1) gene is on chromosome 19 (NBCI NC_000019.10 (51713112 . . . 51723991, complement), and HAS1 encodes the following human amino acid sequence (NCBI accession no. Q92839.2; SEQ ID NO:3).

| | |
|---|---|
| 1 | MRQQDAPKPT PAACRCSGLA RRVLTIAFAL LILGLMTWAY |
| 41 | AAGVPLASDR YGLLAFGLYG AFLSAHLVAQ SLFAYLEHRR |
| 81 | VAAAARGPLD AATARSVALT ISAYQEDPAY LRQCLASARA |
| 121 | LLYPRARLRV LMVVDGNRAE DLYMVDMFRE VFADEDPATY |
| 161 | VWDGNYHQPW EPAAAGAVGA GAYREVEAED PGRLAVEALV |
| 201 | RTRRCVCVAQ RWGGKREVMY TAFKALGDSV DYVQVCDSDT |
| 241 | RLDPMALLEL VRVLDEDPRV GAVGGDVRIL NPLDSWVSFL |
| 281 | SSLRYWVAFN VERACQSYFH CVSCISGPLG LYRNNLLQQF |
| 321 | LEAWYNQKFL GTHCTFGDDR HLTNRMLSMG YATKYTSRSR |
| 361 | CYSETPSSFL RWLSQQTRWS KSYFREWLYN ALWWHRHHAW |
| 401 | MTYEAVVSGL FPFFVAATVL RLFYAGRPWA LLWVLLCVQG |
| 441 | VALAKAAFAA WLRGCLRMVL LSLYAPLYMC GLLPAKFLAL |
| 481 | VTMNQSGWGT SGRRKLAANY VPLLPLALWA LLLLGGLVRS |
| 521 | VAHEARADWS GPSRAAEAYH LAAGAGAYVG YWVAMLTLYW |
| 561 | VGVRRLCRRR TGGYRVQV |

A cDNA sequence for an HAS1 polypeptide can have the following human nucleotide sequence (NCBI accession no. NM_001297436.2; SEQ ID NO:4).

| | |
|---|---|
| 1 | ACAGAGAGGC TGGGAGAGCA AGCCGGAGAG AAGAGAGAGC |
| 41 | CCGGCCAGAC CCACTGCGAT GAGACAGGAC GCGCCCAAGC |
| 81 | CCACTCCTGC AGCCTGCCGC TGCTCCGGCC TGGCCCGGAG |
| 121 | GGTGCTGACC ATCGCCTTCG CCCTGCTCAT CCTGGGCCTC |
| 161 | ATGACCTGGG CCTACGCCGC CGGGGTGCCG CTGGCCTCCG |
| 201 | ATCGCTACGG CCTCCTGGCC TTCGGCCTCT ACGGGGCCTT |
| 241 | CCTTTCAGCG CACCIGGTGG CGCAGAGCCT CTTCGCGTAC |
| 281 | CTGGAGCACC GGCGGGTGGC GGCGGCGGCG CGGGGGCCGC |
| 321 | TGGATGCAGC CACCGCGCGC AGTGTGGCGC TGACCATCTC |
| 361 | CGCCTACCAG GAGGACCCCG CGTACCTGCG CCAGTGCCTG |
| 401 | GCGTCCGCCC GCGCCCTGCT GTACCCGCGC GCGCGGCTGC |
| 441 | GCGTCCTCAT GGTGGTGGAT GGCAACCGCG CCGAGGACCT |
| 481 | CTACATGGTC GACATGTTCC GCGAGGTCTT CGCTGACGAG |
| 521 | GACCCCGCCA CGTACGTGTG GGACGGCAAC TACCACCAGC |
| 561 | CCTGGGAACC CGCGGCGGCG GGCGCGGTGG GCGCCGGAGC |
| 601 | CTATCGGGAG GTGGAGGCGG AGGATCCTGG GCGGCTGGCA |
| 641 | GTGGAGGCGC TGGTGAGGAC TCGCAGGTGC GTGTGCGTGG |
| 681 | CGCAGCGCTG GGGCGGCAAG CGCGAGGTCA TGTACACAGC |
| 721 | CTTCAAGGCG CTCGGAGATT CGGTGGACTA CGTGCAGGTC |
| 761 | TGTGACTCGG ACACAAGGTT GGACCCCATG GCACTGCTGG |
| 801 | AGCTCGTGCG GGTACTGGAC GAGGACCCCC GGGTAGGGGC |
| 841 | TGTTGGTGGG GACGTGCGGA TCCTTAACCC TCTGGACTCC |
| 881 | TGGGTCAGCT TCCTAAGCAG CCTGCGATAC TGGGTAGCCT |
| 921 | TCAATGTGGA GCGGGCTTGT CAGAGCTACT TCCACTGTGT |
| 961 | ATCCTGCATC AGCGGTCCTC TAGGCCTATA TAGGAATAAC |
| 1001 | CTCTTGCAGC AGTTTCTTGA GGCCTGGTAC AACCAGAAGT |
| 1041 | TCCTGGGTAC CCACTGTACT TTTGGGGATG ACCGGCACCT |
| 1081 | CACCAACCGC ATGCTCAGCA TGGGTTATGC TACCAAGTAC |
| 1121 | ACCTCCAGGT CCCGCTGCTA CTCAGAGACG CCCTCGTCCT |
| 1161 | TCCTGCGGTG GCTGAGCCAG CAGACACGCT GGTCCAAGTC |
| 1201 | GTACTTCCGT GAGTGGCTGT ACAACGCGCT CTGGTGGCAC |
| 1241 | CGGCACCATG CGTGGATGAC CTACGAGGCG GTGGTCTCCG |
| 1281 | GCCTGTTCCC CTTCTTCGTG GCGGCCACTG TGCTGCGTCT |
| 1321 | GTTCTACGCG GGCCGCCCTT GGGCGCTGCT GTGGGTGCTG |
| 1361 | CTGTGCGTGC AGGGCGTGGC ACTGGCCAAG GCGGCCTTCG |
| 1401 | CGGCCTGGCT GCGGGGCTGC CTGCGCATGG TGCTTCTGTC |
| 1441 | GCTCTACGCG CCCCTCTACA TGTGTGGCCT CCTGCCTGCC |
| 1481 | AAGTTCCTGG CGCTAGTCAC CATGAACCAG AGTGGCTGGG |
| 1521 | GCACCTCGGG CCGGCGGAAG CTGGCCGCTA ACTACGTCCC |
| 1561 | TCTGCTGCCC CTGGCGCTCT GGGCGCTGCT GCTGCTTGGG |
| 1601 | GGCCTGGTCC GCAGCGTAGC ACACGAGGCC AGGGCCGACT |
| 1641 | GGAGCGGCCC TTCCCGCGCA GCCGAGGCCT ACCACTTGGC |
| 1681 | CGCGGGGGCC GGCGCCTACG TGGGCTACTG GGTGGCCATG |
| 1721 | TTGACGCTGT ACTGGGTGGG CGTGCGGAGG CTTTGCCGGC |
| 1761 | GGCGGACCGG GGGCTACCGC GTCCAGGTGT GAGTCCAGCC |
| 1801 | ACGCGGATGC CGCCTCAAGG GTCTTCAGGG GAGGCCAGAG |
| 1841 | GAGAGCTGCT GGGCCCCGAG CCACGAACTT GCTGGGTGGT |
| 1881 | TCTCTGGGCC TCAGTTTCCC TCCTCTGCAA AACGAGGGGG |
| 1921 | TCAGCCCAAG ATTCTTCAGT CTGGACTATA TTGGGACTGG |
| 1961 | GACTTCTGGG TCTCCAGGGA GGGTATTTAT TGGTCAGGGT |
| 2001 | GTGGGATCTG AGGAGTGGAG GGAAAGGGTC CTGCTTTCTC |
| 2041 | CTCGTTCTTA TTTAATCTCC ATTTCTACTG TGTGATCAGG |
| 2081 | ATGTAATAAA GAATTTTATT TATTTTC |

In another example, a human hyaluronan synthase 2 (HAS2) gene is on chromosome 8 (NBCI NC_000008.11 (121612116 . . . 121641440, complement), and HAS2 encodes the following human amino acid sequence (NCBI accession no. NP_005319.1; SEQ ID NO:5).

| | |
|---|---|
| 1 | MHCERFLCIL RIIGTTLFGV SLLLGITAAY IVGYQFIQTD |
| 41 | NYYFSFGLYG AFLASHLIIQ SLFAFLEHRK MKKSLETPIK |
| 81 | LNKTVALCIA AYQEDPDYLR KCLQSVKRLT YPGIKVVMVI |
| 121 | DGNSEDDLYM MDIFSEVMGR DKSATYIWKN NFHEKGPGET |
| 161 | DESHKESSQH VTQLVLSNKS ICIMQKWGGK REVMYTAFRA |
| 201 | LGRSVDYVQV CDSDTMLDPA SSVEMVKVLE EDPMVGGVGG |
| 241 | DVQILNKYDS WISFLSSVRY WMAFNIERAC QSYFGCVQCI |
| 281 | SGPLGMYRNS LLHEFVEDWY NQEFMGNQCS FGDDRHLTNR |
| 321 | VLSLGYATKY TARSKCLTET PIEYLRWLNQ QTRWSKSYFR |
| 361 | EWLYNAMWFH KHHLWMTYEA IITGFFPFFL IATVIQLFYR |
| 401 | GKIWNILLFL LTVQLVGLIK SSFASCLRGN IVMVFMSLYS |
| 441 | VLYMSSLLPA KMFAIATINK AGWGTSGRKT IVVNFIGLIP |
| 481 | VSVWFTILLG GVIFTIYKES KRPFSESKQT VLIVGTLLYA |
| 521 | CYWVMLLTLY VVLINKCGRR KKGQQYDMVL DV |

A cDNA encoding this HAS2 amino acid sequence is shown below as SEQ ID NO:6 (NCBI accession no. NM_005328.3)

| | |
|---|---|
| 1 | AGACCCCCTT AAGTTGGAGG AGGCAGAAGG GCAACAACGG |
| 41 | CGGGGAAGGA GAAGTCAAGA CGTCTGGAAA GAATTACCCA |
| 81 | GTCCTGGCTT CGAGCAGCCC ATTGAACCAG AGACTTGAAA |
| 121 | CAGCCCCAGC CAAAGACTTT TCTCCCAATT CTGCGCTTCC |
| 161 | TGGGTTCTGC TGAGTCTTCC ACAGGCTTTT TTTTTTTTTT |
| 201 | TTTTTTTTTT TAAGACGAAA AAGAGATTTT CTGTTATCGG |
| 241 | GGGCAGAAAG ACTGAAGCCC AAAAAAAAAA AAAAAAAAAA |
| 281 | AAGAAAAGAA AAGAAAAAAG AAAAGTTAAT TTATTTTTAA |
| 321 | AGCATAATTT TTTTAAGAAT TAGACTGAAG TGCAACGGAA |
| 361 | ACATAAAGAG AATATTAGTG AAATTATTTT TTAAAGTGGG |
| 401 | GAAGAATCAA ACATTTAAGA CTCCCCTATC CTTTTTAAAT |
| 441 | GTTGTTTTTA AATTTCTTAT TTTTTTTGGC CGGTCGTCTC |
| 481 | AAATTCATCT GATCTCTTAT TACCTCAATT TTGGAAACTG |
| 521 | CCCGCCACCG ACCCTCCGGG ACCACACAGA CAGGCTGAGG |
| 561 | ACGACTTTAT GACCAAGAGC TGAACAAGAT GCATTGTGAG |
| 601 | AGGTTTCTAT GTATCCTGAG AATAATTGGA ACCACACTCT |
| 641 | TTGGAGTCTC TCTCCTCCTT GGAATCACAG CTGCTTATAT |
| 681 | TGTTGGCTAC CAGTTTATCC AAACGGATAA TTACTATTTC |
| 721 | TCTTTTGGAC TGTATGGTGC CTTTTTGGCA TCACACCTCA |
| 761 | TCATCCAAAG CCTGTTTGCC TTTTTGGAGC ACCGAAAAAT |
| 801 | GAAAAAATCC CTAGAAACCC CCATAAAGTT GAACAAAACA |
| 841 | GTTGCCCTTT GCATCGCTGC CTATCAAGAA GATCCAGACT |
| 881 | ACTTAAGGAA ATGTTTGCAA TCTGTGAAAA GGCTAACCTA |
| 921 | CCCTGGGATT AAAGTTGTCA TGGTCATAGA TGGGAACTCA |
| 961 | GAAGATGACC TTTACATGAT GGACATCTTC AGTGAAGTCA |
| 1001 | TGGGCAGAGA CAAATCAGCC ACTTATATCT GGAAGAACAA |
| 1041 | CTTCCACGAA AAGGGTCCCG GTGAGACAGA TGAGTCACAT |
| 1081 | AAAGAAAGCT CGCAACACGT AACGCAATTG GTCTTGTCCA |
| 1121 | ACAAAAGTAT CTGCATCATG CAAAAATGGG GTGGAAAAAG |
| 1161 | AGAAGTCATG TACACAGCCT TCAGAGCACT GGGACGAAGT |
| 1201 | GTGGATTATG TACAGGTTTG TGATTCAGAC ACTATGCTTG |
| 1241 | ACCCAGCCTC ATCTGTGGAG ATGGTAAAAG TTTTAGAAGA |
| 1281 | AGATCCCATG GTTGGAGGTG TTGGGGGAGA TGTCCAGATT |
| 1321 | TTAAACAAGT ACGATTCCTG GATCTCATTC CTCAGCAGTG |
| 1361 | TAAGATATTG GATGGCTTTT AATATAGAAA GGGCCTGTCA |
| 1401 | GTCTTATTTT GGGTGTGTTC AGTGCATTAG TGGACCTCTG |
| 1441 | GGAATGTACA GAAACTCCTT GTTGCATGAG TTTGTGGAAG |
| 1481 | ATTGGTACAA TCAAGAATTT ATGGGCAACC AATGTAGCTT |
| 1521 | TGGTGATGAC AGGCATCTCA CGAACCGGGT GCTGAGCCTG |
| 1561 | GGCTATGCAA CAAAATACAC AGCTCGATCT AAGTGCCTTA |
| 1601 | CTGAAACACC TATAGAATAT CTCAGATGGC TAAACCAGCA |
| 1641 | GACCCGTTGG AGCAAGTCCT ACTTCCGAGA ATGGCTGTAC |
| 1681 | AATGCAATGT GGTTTCACAA ACATCACTTG TGGATGACCT |
| 1721 | ACGAAGCGAT TATCACTGGA TTCTTTCCTT TCTTTCTCAT |
| 1761 | TGCCACAGTA ATCCAGCTCT TCTACCGGGG TAAAATTTGG |
| 1801 | AACATTCTCC TCTTCTTGTT AACTGTCCAG CTAGTAGGTC |
| 1841 | TCATAAAATC ATCTTTTGCC AGCTGCCTTA GAGGAAATAT |
| 1881 | CGTCATGGTC TTCATGTCTC TCTACTCAGT GTTATACATG |
| 1921 | TCGAGTTTAC TTCCCGCCAA GATGTTTGCA ATTGCAACAA |
| 1961 | TAAACAAAGC TGGGTGGGGC ACATCAGGAA GGAAAACCAT |
| 2001 | TGTTGTTAAT TTCATAGGAC TCATTCCAGT ATCAGTTTGG |
| 2041 | TTTACAATCC TCCTGGGTGG TGTGATTTTC ACCATTTATA |
| 2081 | AGGAGTCTAA AAGGCCATTT TCAGAATCCA AACAGACAGT |
| 2121 | TCTAATTGTT GGAACGTTGC TCTATGCATG CTATTGGGTC |
| 2161 | ATGCTTTTGA CGCTGTATGT AGTTCTCATC AATAAGTGTG |
| 2201 | GCAGGCGGAA GAAGGGACAA CAATATGACA TGGTGCTTGA |
| 2241 | TGTATGATCT TCCATGTTTT GACGTTTGCA GTCACACACA |
| 2281 | ACACCTTAGT TCCTCTAGGG GCTGTACAGT ATTGTGGCAT |
| 2321 | CAGATAATGC CACCAAAGGA GACATATCAC TGCTGCTGGG |
| 2361 | ACTTGAACAA AGACATTTAT ATGGGTTTAT TTTCATTCTG |
| 2401 | CCAAAGTAAA ACAATACATC AACAAGAAGA AACTCAGATT |
| 2441 | TAACCTGTTA TTTCTATGAA AATGGGATGA ATTCTTTGTT |
| 2481 | TATGCACTTT TTCCTTACTG TGCATCCGCC TGAAAGTGTT |
| 2521 | TTGCCCTATA TACCTCACTA GCCATGCTTT ATGTGGGTTA |
| 2561 | TCATGGAAGA AAAGGATTTT GGAAACTCAA GGAAAAGTTC |
| 2601 | TTTCAACCTA TACAACCTAA CTTATGGACT GTTTTGATAG |
| 2641 | ATGATAATTT TTTTTTTTTA GGAAGGATTT TCTTTTTAAC |
| 2681 | TTTACCAAAT GAAATGCCAA AGGAAGTTTT AAAGGCCGTT |
| 2721 | GGCTGTGCTG TATTTTGATA TAATTGTACT GTGTTTTTAA |
| 2761 | ATTTTGTATG CCAATCTTAA AGACAAATTT TGCATATTCT |
| 2801 | CTATTTTACT TTTCTGCCAA AATAAACCTG TTCTTCCTTT |
| 2841 | TTTAAAATAA AATAAGTTCT TAAAAAATTT ATACTTAAAA |
| 2881 | AATCCTGCCC AAAATGTGAA GCTTGGTTGA CTGATGTTCA |
| 2921 | TGATAGAAAG AATAAAATGT TTCTCTCTCT CTACCTTTTA |
| 2961 | AAATTGAATA GTTTATTTCT GTGAAAGAAG TATTTAAACT |
| 3001 | TTCAATATTT TAACTTTTTG TTTTTATTTC TTTTAGAAAA |
| 3041 | GGCCAATATA CCTATCACAC TTTGGAAGTA AAAATACACA |
| 3081 | CTTTCGTGTG TACCTAAAAA AAAAATCGTT GAAAATCAAG |
| 3121 | GCCAAAGGTA GTGCAATTTT TTCATTAAGA TTTAAAAAAA |
| 3161 | AGGGAATGAT AGTCTTTGAA AGAAAACAGT AGGCATCCAG |
| 3201 | CACTGGACAA AACATGGGTA TCAAAGATGA ATAATCTTTG |
| 3241 | GAGATTCTGG CAGTGTTTTC CCAGAACAAG TCAAGTGGAA |
| 3281 | AGTGGAGAAA TTATCTGTAT AATTTTGGAC ACATACAATG |
| 3321 | CAGTTTATCA AAGGTTTTGT TCTGTGGCCT GAATTTACTG |
| 3361 | GGTCCTACCT ACACATTGAA CATGTTTTGC CTGTCTTTTT |
| 3401 | TTTTTTTTTC AACTTGCCAG TTCACTTTAC ATGTTAGTAT |
| 3441 | AATGTTTACA CGGGTGAGTT GGATAAATTA TAAAACATAT |
| 3481 | AAATTCAAAA TTGGCAGATA GAATCACCAA GTATCTATCC |
| 3521 | TCTTTTACTT TCAAATGAGG AATTTTGTTT TTCTGAATTA |
| 3561 | CACAGATCAT CACTTCCTAT TTCCTGTTCT GGACCTGTAT |
| 3601 | AAAAATGTCT ACACAGTAGA AGTGACATCA AGGTTTAATA |
| 3641 | AGTATATCAA TGATTGGCAC ATATAAAAAT TGTTGAACCA |
| 3681 | CATACTCTGA ACTTGGCTAA TTTAGTTACT GCAAGGCCTC |
| 3721 | CATTATCCAG TTTTATTTTT TACACGATTG ACCTTGCCTT |
| 3761 | GTAGCTGGTG CTGTGTAGAC CTGTGTTGAA AACACAATCG |
| 3801 | GAATATATGA ATAATTGAAT AAACAGCATT ATGGTGAGGC |
| 3841 | AGAGACACAT GGAGAAGTGT TAAAAAAAAA ATGGGCTTCC |
| 3881 | TGCCTTTCTG CCTCTTTTTT ATGCAGTCAT CTATGTTACA |
| 3921 | TCTATCCTGC CTAAGAAAAA GCTGCACATC CTACCTTCAG |
| 3961 | AGTACAAAAA GGTACATCTG AGACTCAAGA CTCTCACTGA |
| 4001 | TTGGAGAGCT TGTGGAAAAC AAAACACACC ATGCCAATAA |
| 4041 | ATGAGATGAA AACTTGAGTT TGCCTTTTTA ACTATTTATG |
| 4081 | TTCTAAGTTA AGCTTTGATA ACATTCAAAT GTCAAATTCT |
| 4121 | CTCATTCTTA TAAAAAGTTG AATTAATTGC CTGTATTTAT |
| 4161 | TTTAGCAATT ATTCAATGTA TTTCCAGTAT AGGATGTATA |
| 4201 | GTATAATTAA TTTTTTGTAA ATAAAATATT TTTGATAAGA |

Subjects can be effectively treated by the compositions and methods described herein even though those subjects have HAS1 or HAS2 isoforms and variants with somewhat different sequences than the examples of HAS sequences described herein. Such HAS isoforms and variants can have polypeptide or nucleic acid sequences with between 55-100% sequence identity to a reference sequence (e.g., a sequence described herein). For example, HAS isoforms and variants can have at least 55% sequence identity, e.g., at least 60%, at least 70%, at least 80%, at least 90%, at least 95%, at least 96%, at least 97% sequence, at least 98%, at least 99% identity to a reference sequence (e.g., a sequence described herein) over a specified comparison window. Optimal alignment may be ascertained or conducted using the homology alignment algorithm of Needleman and Wunsch, J. Mol. Biol. 48:443-53 (1970).

### Hyaluronidase

Hyaluronidase is an enzyme that breaks down hyaluronic acid. Although it has been used in medical applications for over 60 years, it is thought exacerbate the spread (metastasis) of cancer. Clinical trials of PEGPH20, a clinical grade hyaluronidase, were discontinued after the investigational drug failed to meet the primary endpoint in a phase 3 trial in metastatic pancreas cancer. However, as illustrated herein, PEGPH20 reverts stroma activation and enhances Olaparib anti-tumor activity.

An example of a hyaluronidase sequence is the following hyaluronidase PH20 sequence (Uniprot P38567-1; SEQ ID NO:7).

```
                10 20 30 40
MGVLKFKHIF FRSFVKSSGV SQIVFTFLLI PCCLTLNFRA
                50 60 70 80
PPVIPNVPFL WAWNAPSEFC LGKFDEPLDM SLFSFIGSPR
                90 100 110 120
INATGQGVTI FYVDRLGYYP YIDSITGVTV NGGIPQKISL
              130 140 150 160
QDHLDKAKKD ITFYMPVDNL GMAVIDWEEW RPTWARNWKP
              170 180 190 200
KDVYKNRSIE LVQQQNVQLS LTEATEKAKQ EFEKAGKDFL
              210 220 230 240
VETIKLGKLL RPNHLWGYYL FPDCYNHHYK KPGYNGSCFN
              250 260 270 280
VEIKRNDDLS WLWNESTALY PSIYLNTQQS PVAATLYVRN
              290 300 310 320
RVREAIRVSK IPDAKSPLPV FAYTRIVFTD QVLKFLSQDE
              330 340 350 360
LVYTFGETVA LGASGIVIWG TLSIMRSMKS CLLLDNYMET
              370 380 390 400
ILNPYIINVT LAAKMCSQVL CQEQGVCIRK NWNSSDYLHL
              410 420 430 440
NPDNFAIQLE KGGKFTVRGK PTLEDLEQFS EKFYCSCYST
              450 460 470 480
LSCKEKADVK DTDAVDVCIA DGVCIDAFLK PPMETEEPQI
              490 500
FYNASPSTLS LIISSVASL
```

A cDNA sequence for such a hyaluronidase is shown below (NCBI L13781.1; SEQ ID NO: 8).

| | |
|---|---|
| 1 | ATTCCATTCC CTTTCATCTG TGCTCATACT TTGCATCAGA |
| 41 | TATTGGGTAA ACCAAAGTGT GTAGGAAGAA ATAAATGTTT |
| 81 | TCATAGTCAT TACTCTTTAC AATGGGAGTG CTAAAATTCA |
| 121 | AGCACATCTT TTTCAGAAGC TTTGTTAAAT CAAGTGGAGT |
| 161 | ATCCCAGATA GTTTTCACCT TCCTTCTGAT TCCATGTTGC |
| 201 | TTGACTCTGA ATTTCAGAGC ACCTCCTGTT ATTCCAAATG |
| 241 | TGCCTTTCCT CTGGGCCTGG AATGCCCCAA GTGAATTTTG |
| 281 | TCTTGGAAAA TTTGATGAGC CACTAGATAT GAGCCTCTTC |
| 321 | TCTTTCATAG GAAGCCCCCG AATAAACGCC ACCGGGCAAG |
| 361 | GAGTTACAAT ATTTTATGTT GATAGACTTG GCTACTATCC |
| 401 | TTACATAGAT TCAATCACAG GAGTAACTGT GAATGGAGGA |
| 441 | ATCCCCCAGA AGATTTCCTT ACAAGACCAT CTGGACAAAG |
| 481 | CTAAGAAAGA CATTACATTT TATATGCCAG TAGACAATTT |
| 521 | GGGAATGGCT GTTATTGACT GGGAAGAATG GAGACCCACT |
| 561 | TGGGCAAGAA ACTGGAAACC TAAAGATGTT TACAAGAATA |
| 601 | GGTCTATTGA ATTGGTTCAG CAACAAAATG TACAACTTAG |
| 641 | TCTCACAGAG GCCACTGAGA AAGCAAAACA AGAATTTGAA |
| 681 | AAGGCAGGGA AGGATTTCCT GGTAGAGACT ATAAAATTGG |
| 721 | GAAAATTACT TCGGCCAAAT CACTTGTGGG GTTATTATCT |
| 761 | TTTTCCGGAT TGTTACAACC ATCACTATAA GAAACCCGGT |
| 801 | TACAATGGAA GTTGCTTCAA TGTAGAAATA AAAAGAAATG |
| 841 | ATGATCTCAG CTGGTTGTGG AATGAAAGCA CTGCTCTTTA |
| 881 | CCCATCCATT TATTTGAACA CTCAGCAGTC TCCTGTAGCT |
| 921 | GCTACACTCT ATGTGCGCAA TCGAGTTCGG GAAGCCATCA |
| 961 | GAGTTTCCAA AATACCTGAT GCAAAAAGTC CACTTCCGGT |
| 1001 | TTTTGCATAT ACCCGCATAG TTTTTACTGA TCAAGTTTTG |
| 1041 | AAATTCCTTT CTCAAGATGA ACTTGTGTAT ACATTTGGCG |
| 1081 | AAACTGTTGC TCTGGGTGCT TCTGGAATTG TAATATGGGG |
| 1121 | AACCCTCAGT ATAATGCGAA GTATGAAATC TTGCTTGCTC |
| 1161 | CTAGACAATT ACATGGAGAC TATACTGAAT CCTTACATAA |
| 1201 | TCAACGTCAC ACTAGCAGCC AAAATGTGTA GCCAAGTGCT |
| 1241 | TTGCCAGGAG CAAGGAGTGT GTATAAGGAA AAACTGGAAT |
| 1281 | TCAAGTGACT ATCTTCACCT CAACCCAGAT AATTTTGCTA |
| 1321 | TTCAACTTGA GAAAGGTGGA AAGTTCACAG TACGTGGAAA |
| 1361 | ACCGACACTT GAAGACCTGG AGCAATTTTC TGAAAAATTT |
| 1401 | TATTGCAGCT GTTATAGCAC CTTGAGTTGT AAGGAGAAAG |
| 1441 | CTGATGTAAA AGACACTGAT GCTGTTGATG TGTGTATTGC |
| 1481 | TGATGGTGTC TGTATAGATG CTTTTCTAAA ACCTCCCATG |
| 1521 | GAGACAGAAG AACCTCAAAT TTTCTACAAT GCTTCACCCT |
| 1561 | CCACACTATC TGCCACAATG TTCATTGTTA GTATTTTGTT |
| 1601 | TCTTATCATT TCTTCTGTAG CGAGTTTGTA ATTGCGCAGG |
| 1641 | TTAGCTGAAA TGAACAATAT GTCCATCTTA AAGTGTGCTT |
| 1681 | CCC |

Subjects can be effectively treated by the compositions and methods described herein even though those subjects have hyaluronidase isoforms and variants with somewhat different sequences than the examples of hyaluronidase sequences described herein. Such hyaluronidase isoforms and variants can have polypeptide or nucleic acid sequences with between 55-100% sequence identity to a reference sequence (e.g., a sequence described herein). For example, hyaluronidase isoforms and variants can have at least 55% sequence identity, e.g., at least 60%, at least 70%, at least 80%, at least 90%, at least 95%, at least 96%, at least 97% sequence, at least 98%, at least 99% identity to a reference sequence (e.g., a sequence described herein) over a specified comparison window. Optimal alignment may be ascertained or conducted using the homology alignment algorithm of Needleman and Wunsch, J. Mol. Biol. 48:443-53 (1970).

### ULK

Autophagy plays a crucial role in several cellular functions, and its dysregulation is associated with both tumorigenesis and drug resistance. Autophagy is a process of metabolic degradation at the cellular level in which organelles or portions of the cytosol are sequestered by double-membrane autophagosomes and fused with lysosomes for degradation. Unc-51-like kinase 1 (ULK1) is a serine/threonine kinase that participates in the initiation of autophagy. Some studies have indicated that compounds that directly or indirectly target ULK1 could be used for tumor therapy. However, reports of the therapeutic effects of these compounds have come to conflicting conclusions.

ULK1 is a central kinase of the ULK1 complex involved in autophagy initiation, which promotes autophagosome-lysosome fusion. The human ULK1 gene is on chromosome 12 (NC_000012.12 (131894622 . . . 131923150). An example of a human ULK1 amino acid sequence is shown below (Uniprot 075385; SEQ ID NO: 9).

```
                10 20 30 40
MEPGRGGTET VFKGEGSRKD LIGHGAFAVV FKGRHREKHD
                50 60 70 80
LEVAVKCINK KNLAKSQTLL GKEIKILKEL KHENIVALYD
                90 100 110 120
FQEMANSVYL VMEYCNGGDL ADYLHAMRTL SEDTIRLFLQ
              130 140 150 160
QIAGAMRLLH SKGIIHRDLK PQNILLSNPA GRRANPNSIR
              170 180 190 200
VKIADFGFAR YLQSNMMAAT LCGSPMYMAP EVIMSQHYDG
              210 220 230 240
KADLWSIGTI VYQCLTGKAP FQASSPQDLR LFYEKNKTLV
              250 260 270 280
PTIPRETSAP LRQLLLALLQ RNHKDRMDFD EFFHHPFLDA
              290 300 310 320
SPSVRKSPPV PVPSYPSSGS GSSSSSSSTS HLASPPSLGE
              330 340 350 360
MQQLQKTLAS PADTAGFLHS SRDSGGSKDS SCDTTDFVMV
              370 380 390 400
PAQFPGDLVA EAPSAKPPPD SLMCSGSSLV ASAGLESHGR
              410 420 430 440
TPSPSPPCSS SPSPSGRAGP FSSSRCGASV PIPVPTQVQN
              450 460 470 480
YQRIERNLQS PTQFQTPRSS AIRRSGSTSP LGFARASPSP
              490 500 510 520
PAHAEHGGVL ARKMSLGGGR PYTPSPQVGT IPERPGWSGT
              500 510 520 530
ARKMSLGGGR PYTPSPQVGT IPERPGWSGT PSPQGAEMRG
              540 550 560 570
GRSPRPGSSA PEHSPRTSGL GCRLHSAPNL SDLHVVRPKL
              580 590 600 610
PKPPTDPLGA VFSPPQASPP QPSHGLQSCR NLRGSPKLPD
              620 630 640 650
FLQRNPLPPI LGSPTKAVPS FDFPKTPSSQ NLLALLARQG
              660 670 680 690
VVMTPPRNRT LPDLSEVGPF HGQPLGPGLR PGEDPKGPFG
              700 710 720 730
RSFSTSRLTD LLLKAAFGTQ APDPGSTESL QEKPMEIAPS
              740 750 760 770
AGFGGSLHPG ARAGGTSSPS SGSTPPQGPR TRMFSAGPTP
              780 790 800 810
TRMFSAGPTP SASSSARHLV PGPCSEAPAP ELPAPGHGCS
              820 830 840 850
FADPTIANLE GAVTFEAPDL PEETLMEQEH TEILRGLRFT
              860 870 880 890
LLFVQHVLEI AALKGSASEA AGGPEYQLQE SVVADQISLL
              900 910 920 930
SREWGFAEQL VLYLKVAELL SSGLQSAIDQ IRAGKLCLSS
              940 950 960 970
TVKQVVRRLN ELYKASVVSC QGLSLRLQRF FLDKQRLLDR
              980 990 1000 1010
IHSITAERLI FSHAVQMVQS AALDEMFQHR EGCVPRYHKA
            1020 1030 1040 1050
LLLLEGLQHM LSDQADIENV TKCKLCIERR LSALLTGICA
```

An example of a human ULK1 cDNA is shown below (NCBI; SEQ ID NO:10).

| | |
|---|---|
| 1 | GGATCCGGAT TCGGATTAGC AGCCCGGGAA GAGTGCCGTG |
| 41 | GCACAGGCGC CGGAGGGAGC GCGACCCTCG GACCCCGCCT |
| 81 | GGCCCGCGGG GCTGGGACCC GGCCCCGGCC TGCCCGATGG |
| 121 | GGCGCGCGGC CCCGGAGATG CGCCCTCGCC CGGCCCCGCG |
| 161 | CCCCCGGCCC CGCGCCCCCG GCCCGCCCGC CCCGGCCCGC |
| 201 | GCCTCCGCCT GAGTCCCCCG CGCCTTGGCC CGCCACCCCC |
| 241 | CGCCCCGCGC CCCCGGCCCG CCTGCGCCAT GGAGCCCGGC |
| 281 | CGCGGCGGCA CAGAGACCGT GGGCAAGTTC GAGTTCTCCC |
| 321 | GCAAGGACCT GATCGGCCAC GGCGCCTTCG CGGTGGTCTT |
| 361 | CAAGGGCCGC CACCGCGAGA AGCACGATTT GGAGGTCGCC |
| 401 | GTCAAGTGCA TTAACAAGAA GAACCTCGCC AAGTCTCAGA |
| 441 | CGCTGCTGGG GAAGGAAATC AAAATCCTGA AGGAACTGAA |
| 481 | ACATGAAAAC ATCGTGGCCC TGTACGACTT CCAGGAAATG |
| 521 | GCTAATTCTG TCTACCTGGT TATGGAGTAC TGCAACGGTG |
| 561 | GGGACCTGGC CGACTACCTG CACGCCATGC GCACGCTGAG |
| 601 | CGAGGACACC ATCAGGCTCT TCCTGCAGCA GATCGCGGGC |
| 641 | GCCATGCGGC TTCTGCACAG CAAAGGCATC ATCCACCGCG |
| 681 | ACCTGAAACC GCAGAACATC CTGCTGTCCA ACCCCGCCGG |
| 721 | CCGCCGCGCC AACCCCAACA GCATCCGCGT CAAGATCGCT |
| 761 | GACTTCGGCT TCGCGCGGTA CCTCCAGAGC AACATGATGG |
| 801 | CGGCCACACT CTGCGGCTCC CCCATGTACA TGGCCCCCGA |
| 841 | GGTCATCATG TCCCAGCACT ACGACGGGAA GGCGGACCTG |
| 881 | TGGAGCATCG GCACCATCGT CTACCAGTGC CTGACGGGGA |
| 921 | AGGCGCCCTT CCAGGCCAGC AGCCCCCAGG ACCTGCGCCT |
| 961 | GTTCTACGAG AAGAACAAGA CGTTGGTCCC CACCATCCCC |
| 1001 | CGGGAGACCT CGGCCCCGCT GCGGCAGCTG CTCCTGGCCC |
| 1041 | TACTGCAACG CAACCACAAG GACCGCATGG ACTTCGATGA |
| 1081 | GTTTTTTCAT CACCCTTTCC TCGATGCCAG CCCCTCGGTC |
| 1121 | AGGAAATCCC CACCCGTGCC TGTGCCCTCG TACCCAAGCT |
| 1161 | CGGGGTCCGG CAGCAGCTCC AGCAGCAGCT CCACCTCCCA |
| 1201 | CCTGGCCTCC CCGCCGTCCC TGGGCGAGAT GCAGCAGCTG |
| 1241 | CAGAAGACCC TGGCCTCCCC GGCTGACACC GCTGGCTTCC |
| 1281 | TGCACAGCTC CCGGGACTCT GGTGGCAGCA AGGACTCTTC |
| 1321 | CTGTGACACA GACGACTTCG TCATGGTCCC CGCGCAGTTT |
| 1361 | CCAGGTGACC TGGTGGCTGA GGCGCCCAGT GCCAAACCCC |
| 1401 | CGCCAGACAG CCTGATGTGC AGTGGGAGCT CACTGGTGGC |
| 1441 | CTCTGCGGGC TTGGAGAGCC ACGGCCGGAC CCCATCTCCA |
| 1481 | TCCCCACCCT GCAGCAGCTC CCCCAGTCCC TCAGGCCGGG |
| 1521 | CTGGCCCGTT CTCCAGCAGC AGGTGCGGCG CCTCTGTCCC |
| 1561 | CATCCCAGTC CCCACGCAGG TGCAGAACTA CCAGCGCATT |
| 1601 | GAGCGAAACC TGCAGTCACC CACCCAGTTC CAAACACCTC |
| 1641 | GGTCCTCTGC CATCCGCAGG TCAGGCAGCA CCAGCCCCCT |
| 1681 | GGGCTTTGCA AGGGCCAGCC CCTCGCCCCC TGCCCACGCT |
| 1721 | GAGCATGGAG GCGTCCTGGC CAGGAAGATG TCTCTGGGTG |
| 1761 | GAGGCCGGCC CTACACGCCA TCTCCTCAAG TTGGAACCAT |
| 1801 | CCCTGAGCGG CCAGGCTGGA GCGGGACGCC CTCCCCACAG |
| 1841 | GGAGCTGAGA TGCGGGGTGG CAGGTCCCCT CGTCCAGGCT |
| 1881 | CCTCTGCACC CGAGCACTCT CCCCGCACTT CCGGGCTGGG |
| 1921 | CTGCCGCCTG CACAGCGCCC CCAACCTGTC TGACTTGCAC |
| 1961 | GTCGTCCGCC CCAAGCTGCC CAAACCCCCC ACGGACCCCC |
| 2001 | TGGGAGCTGT GTTCAGCCCA CCACAGGCCA GCCCTCCCCA |
| 2041 | GCCGTCCCAC GGCCTGCAGT CCTGCCGGAA CCTGCGGGGC |
| 2081 | TCACCCAAGC TGCCCGACTT CCTGCAGCGA AACCCCCTGC |
| 2121 | CCCCCATCCT GGGCTCCCCC ACCAAGGCTG TGCCCTCCTT |
| 2161 | TGACTTCCCG AAGACCCCCA GCTCCCAGAA CCTGCTGGCC |
| 2201 | CTCCTAGCCC GGCAGGGCGT GGTGATGACG CCCCCTCGAA |
| 2241 | ACCGGACGCT GCCCGACCTC TCGGAGGTGG GACCCTTCCA |
| 2281 | TGGTCAGCCG TTGGGCCCTG GCCTGCGGCC AGGCGAGGAC |
| 2321 | CCCAAGGGCC CCTTTGGCCG GTCTTTCAGC ACCAGCCGCC |
| 2361 | TCACTGACCT GCTCCTTAAG GCGGCGTTTG GGACACAAGC |
| 2401 | CCCGGACCCG GGCAGCACGG AGAGCCTGCA GGAGAAGCCC |
| 2441 | ATGGAGATCG CACCCTCAGC TGGCTTTGGA GGGAGCCTGC |
| 2481 | ACCCAGGAGC CCGTGCTGGG GGCACCAGCA GCCCCTCCCC |
| 2521 | GGTGGTCTTC ACCGTGGGCT CTCCCCCGAG CGGGAGCACG |
| 2561 | CCCCCCCAGG GCCCCCGCAC CAGGATGTTC TCAGCGGGCC |
| 2601 | CCACTGGCTC TGCCAGCTCT TCTGCCCGCC ACCTGGTGCC |
| 2641 | TGGGCCCTGC AGCGAGGCCC CAGCCCCTGA GCTCCCTGCT |
| 2681 | CCAGGACACG GCTGCAGCTT TGCCGACCCC ATTGCTGCGA |
| 2721 | ACCTGGAGGG GGCTGTGACC TTCGAGGCCC CCGACCTCCC |
| 2761 | TGAGGAGACC CTCATGGAGC AAGAGCACAC GGAGATCCTG |
| 2801 | CGTGGCCTGC GCTTCACGCT GCTGTTCGTG CAGCACGTCC |
| 2841 | TGGAGATCGC AGCCCTGAAG GGCAGCGCCA GTGAGGCGGC |
| 2881 | GGGGGGCCCT GAGTACCAGC TGCAGGAGAG TGTGGTGGCC |
| 2921 | GACCAGATCA GCCTGCTGAG CCGAGAATGG GGCTTCGCGG |
| 2961 | AACAGCTGGT GCTGTACCTG AAGGTGGCCG AGCTACTGTC |
| 3001 | CTCCGGCCTG CAAAGTGCCA TCGACCAGAT CCGGGCCGGC |
| 3041 | AAGCTCTGCC TGTCGTCCAC TGTGAAGCAG GTGGTGCGCA |
| 3081 | GGCTGAATGA GCTGTACAAG GCCAGCGTGG TGTCCTGCCA |
| 3121 | GGGCCTGAGC CTGCGGCTGC AGCGCTTCTT CCTGGACAAG |
| 3161 | CAGCGGCTCC TGGACCGCAT TCACAGCATC ACTGCCGAGA |
| 3201 | GGCTCATCTT CAGCCACGCT GTGCAGATGG TGCAGTCGGC |
| 3241 | TGCCCTGGAC GAGATGTTCC AGCACCGTGA GGGCTGCGTC |
| 3281 | CCACGCTACC ACAAGGCCCT GCTGCTCCTG GAGGGGCTGC |
| 3321 | AGCACATGCT CTCGGACCAG GCCGACATCG AGAACGTCAC |
| 3361 | CAAGTGCAAG CTGTGCATTG AGCGGAGACT CTCGGCGCTG |
| 3401 | CTGACTGGCA TCTGTGCCTG ACCTTTCTGG CCTGGCTGGG |
| 3441 | CCCCCCGTCC TGCCGAGCCC TGCAGAGTGG GCTCTGTGTG |
| 3481 | CTGGCTGGAC TCCTCGGGAC AAGCCCATGG CGCTGATCGC |
| 3521 | TGGTGCTGAG CCCTGCCCTG GGCCCCACGG ACAGTCAGCC |
| 3561 | TGCCGGCCTC CCTGCAGCTC ACGGGGCAGA ACCAGCACAT |
| 3601 | CTGGAGCCAC ACAGCTTGGG GGGTGTCTCC CATCTTTTAC |
| 3641 | AGGTGGGGAT CACAGAATTT CTGCCCCTCC AGCTGCCTGG |
| 3681 | CTCAGCAGGC GTGGGTGCCA CCACCCTCTA GCCCCAGGGC |
| 3721 | AGCCCCGGAG GACAGGCAAG GGCCTGAGAC CACTGCCGAC |
| 3761 | TCAAAGCCAA AGCGAGCTCC TGCTTAGGGC AGGTCAGCAG |
| 3801 | GCACTGTGCC CAGGAAGAGC CTGCGGCCTC GGCGTCCCCC |
| 3841 | AGTCTCCAGG AGCCTCTCCC TCCGAGATAC CCACCCAGCT |
| 3881 | TTGTCAATCA CCCAAGCACT TTATGCATAT AGAGACAGAA |
| 3921 | CCTGGACCTC ACCAGGGACT GCTGGGCAGC GATTCCTGGC |
| 3961 | AGTGGCCTGG TGTTTGTACA TACACATATG CAGACACATG |
| 4001 | CCAGGGCCCC CCAAGCCCGA GCACCGGACC ACGTTGCTGC |
| 4041 | CCAGGTCTGG ACCTCAGCGG GAGAACTGGC TCCGGGGGGA |
| 4081 | GTGGGGCCCT GCGCTAGAGG CAGAGGCAGT TCTTTGTTCA |
| 4121 | AGCGTTCCTC TGGGGACCGG CAGCAGAGGC ACCGTGTTCT |
| 4161 | CTCAGCCCTG GATACGTCTT GTAATCTTTC ACACTTTATT |
| 4201 | CCTAAAACGT GTCTTATTTT TATGCAGCTC ATTTTTTCTT |
| 4241 | TAAAGGAGAA AACTTGTAGG TGTTTAAGAA TTGGTTTTGG |
| 4281 | GAGGGCGAGG ACTGGGCCAG GTTAGAGGCA GATGGCACAG |
| 4321 | GGGCGTGTGG CGGGCGGGTG AGGCTGCTTT GCACACCTGT |
| 4361 | GTTGGTGGCT GTCCCCTGCC GCCCCTCCCT GTGGCAGCAG |
| 4401 | CAGGACAGGT GTGTGCCCAG CACCCTCCCT ACCTGGGCCT |
| 4441 | GGAAGCAGAT GAGGGGAATA CTTCATGCAA AGAAAAAAGT |
| 4481 | AACATGTGCA AAAGCTCCCC GTCCAGCTTT GACAGTCAGT |
| 4521 | TTTGATGTCA GCTCCTCGGC AGGGTAGGCC TGATGACAGC |
| 4561 | CCTGTCCCTC CCTGCCTCCG CCTTGCCCAA GGCCACGGAG |
| 4601 | GGCGTCTGCA GAGAGGCCTG CCTTCCGGAT TCCAGGCGGG |
| 4641 | CATGCCCTGC AAACCCCGCC TGGGCCTCCC TTGGTCTGCC |
| 4681 | CAGCCCTCGG TTAGCCCTGC CTGAATCAGT AGATACTTGA |
| 4721 | ACGAGTCCCC AGTCTGCGGG AGGCAGTGGT GGGGCCATGG |
| 4761 | ACCCATGCGG GGGGTTCCAG GGTCACACGC CACATAACAG |
| 4801 | ACAAAAATAC ACACACGTGT GTTTTTCTTT GCAATACTTG |
| 4841 | AAATATTGCC ACTGTGCTTG GACTTAGAAG AAGAAAATCC |
| 4881 | CCGTGACTTC TTCCTCATCA CCTTGATGGC TTTATTCTCA |
| 4921 | CCTTGTGGGG CATGTTTGAA TTTATTGCTT CATGGCCGAC |
| 4961 | TGGAATCCTG AGTCCTGGGA AGCTGGCACT GCGGGGATCT |
| 5001 | TGCCCGGTGT CCTGGTCCTC TTGCTTCCGT CGCGGCCGCA |
| 5041 | TGTGCGTGTG TCCAAGCAGG TCCTGGGCGC CTCAACTGCT |
| 5081 | GCCCCTGGTT GAATGTTCTC TTGATAGTGC TGGACCCTTT |
| 5121 | GTCTATTTTA AAGCGAATTT TGTGTGATTT CCTGCCCTTT |
| 5161 | GCGTTATATT GTATAATACC AACGTAAGGA AATAAACCTT |
| 5201 | TGGAATTGTT GAAAAAAAAA AAAAAAAA |

Subjects can be effectively treated by the compositions and methods described herein even though those subjects have ULK isoforms and variants with somewhat different sequences than the examples of ULK sequences described herein. Such ULK isoforms and variants can have polypeptide or nucleic acid sequences with between 55-100% sequence identity to a reference sequence (e.g., a sequence described herein). For example, ULK isoforms and variants can have at least 55% sequence identity, e.g., at least 60%, at least 70%, at least 80%, at least 90%, at least 95%, at least 96%, at least 97% sequence, at least 98%, at least 99% identity to a reference sequence (e.g., a sequence described herein) over a specified comparison window. Optimal alignment may be ascertained or conducted using the homology alignment algorithm of Needleman and Wunsch, J. Mol. Biol. 48:443-53 (1970).

p62

The p62 protein, also referred to as sequestosome-1, is encoded by the human SQSTM1 gene, which is found on chromosome 5 (NC_000005.10 (179806393 . . . 179838078). An example of a human p62 amino acid sequence is shown below (Uniprot Q13501-1; SEQ ID NO:11).

```
                10 20 30 40
MASLTVKAYL LGKEDAAREI RRFSFCCSPE PEAEAEAAAG
                50 60 70 80
PGPCERLLSR VAALFPALRP GGFQAHYRDE DGDLVAFSSD
                90 100 110 120
EELTMAMSYV KDDIFRIYIK EKKECRRDHR PPCAQEAPRN
              130 140 150 160
MVHPNVICDG CNGPVVGTRY KCSVCPDYDL CSVCEGKGLH
              170 180 190 200
RGHTKLAFPS PFGHLSEGFS HSRWLRKVKH GHFGWPGWEM
              210 220 230 240
GPPGNWSPRP PRAGEARPGP TAESASGPSE DPSVNFLKNV
              250 260 270 280
GESVAAALSP LGIEVDIDVE HGGKRSRLTP VSPESSSTEE
              290 300 310 320
KSSSQPSSCC SDPSKPGGVN EGATQSLAEQ MRKIALESEG
              330 340 350 360
RPEEQMESDN CSGGDDDWTH LSSKEVDPST GELQSLQMPE
              370 380 390 400
SEGPSSLDPS QEGPTGLKEA ALYPHLPPEA DPRLIESLSQ
              410 420 430 440
MLSMGFSDEG GWLTRLLQTK NYDIGAALDT IQYSHKPPPL
```

A cDNA encoding a human p62 protein is shown below (SEQ ID NO:12).

| | |
|---|---|
| 1 | GAATTCCCTC GCCGCTCGCT ATGGCGTCGC TCACCGTGAA |
| 41 | GGCCTACCTT CTGGGCAAGG AGGACGCGGC GCGCGAGATT |
| 81 | CGCCGCTTCA GCTTCTGCTG CAGCCCCGAG CCTGAGGCGG |
| 121 | AAGCCGAGGC TGCGGCGGGT CCGGGACCCT GCGAGCGGCT |
| 161 | GCTGAGCCGG GTGGCCGCCC TGTTCCCCGC GCTGCGGCCT |
| 201 | GGCGGCTTCC AGGCGCACTA CCGCGATGAG GACGGGGACT |
| 241 | TGGTTGCCTT TTCCAGTGAC GAGGAATTGA CAATGGCCAT |
| 281 | GTCCTACGTG AAGGATGACA TCTTCCGAAT CTACATTAAA |
| 321 | GAGAAAAAAG AGTGCCGGCG GGACCACCGC CCACCGTGTG |
| 361 | CTCAGGAGGC GCCCCGCAAC ATGGTGCACC CCAATGTGAT |
| 401 | CTGCGATGGC TGCAATGGGC CTGTGGTAGG AACCCGCTAC |
| 441 | AAGTGCAGCG TCTGCCCAGA CTACGACTTG TGTAGCGTCT |
| 481 | GCGAGGGAAA GGGCTTGCAC CGGGGGCACA CCAAGCTCGC |
| 521 | ATTCCCCAGC CCCTTCGGGC ACCTGTCTGA GGGCTTCTCG |
| 561 | CACAGCCGCT GGCTCCGGAA GGTGAAACAC GGACACTTCG |
| 601 | GGTGGCCAGG ATGGGAAATG GGTCCACCAG GAAACTGGAG |
| 641 | CCCACGTCCT CCTCGTGCAG GGGAGGCCCG CCCTGGCCCC |
| 681 | ACGGCAGAAT CAGCTTCTGG TCCATCGGAG GATCCGAGTG |
| 721 | TGAATTTCCT GAAGAACGTT GGGGAGAGTG TGGCAGCTGC |
| 761 | CCTTAGCCCT CTGGGCATTG AAGTTGATAT CGATGTGGAG |
| 801 | CACGGAGGGA AAAGAAGCCG CCTGACCCCC GTCTCTCCAG |
| 841 | AGAGTTCCAG CACAGAGGAG AAGAGCAGCT CACAGCCAAG |
| 881 | CAGCTGCTGC TCTGACCCCA GCAAGCCGGG TGGGAATGTT |
| 921 | GAGGGCGCCA CGCAGTCTCT GGCGGAGCAG ATGAGGAAGA |
| 961 | TCGCCTTGGA GTCCGAGGGG GCCCCTGAGG AACAGATGGA |
| 1001 | GTCGGATAAC TGTTCAGGAG GAGATGATGA CTGGACCCAT |
| 1041 | CTGTCTTCAA AAGAAGTGGA CCCGTCTACA GGTGAACTCC |
| 1081 | AGTCCCTACA GATGCCAGAA TCCGAAGGGC CAAGCTCTCT |
| 1121 | GGACCCCTCC CAGGAGGGAC CCACAGGGCT GAAGGAAGCT |
| 1161 | GCCTTGTACC CACATCTCCC GCCAGAGGCT GACCCGCGGC |
| 1201 | TGATTGAGTC CCTCTCCCAG ATGCTGTCCA TGGGCTTCTC |
| 1241 | TGATGAAGGC GGCTGGCTCA CCAGGCTCCT GCAGACCAAG |
| 1281 | AACTATGACA TCGGAGCGGC TCTGGACACC ATCCAGTATT |
| 1321 | CAAAGCATCC CCCGCCGTTG TGACCACTTT TGCCCACCTC |
| 1361 | TTCTGCGTGC CCCTCTTCTG TCTCATAGTT GTGTTAAGCT |
| 1401 | TGCGTAGAAT TGGCAGGTCT CTGTACGGGC CAGTTTCTCT |
| 1441 | GCCTTCTTCC AGGATCAGGG GTTAGGGTGC AAGAAGCCAT |
| 1481 | TTAGGGCAGC AAAACAAGTG ACATGAAGGG AGGGTCCCTG |
| 1521 | TGTGTGTGTG TGCTGATGTT TCCTGGGTGC CCTGGCTCCT |
| 1561 | TGCAGCAGGG CTGGGCCTGC GAGACCCAAG GCTCACTGCA |
| 1601 | GCGCGCTCCT GACCCCTCCC TGCAGGGGCT ACGTTAGCAG |
| 1641 | CCCAGCACAT AGCTTGCCTA ATGGCTTTCA CTTTCTCTTT |
| 1681 | TGTTTTAAAT GACTCATAGG TCCCTGACAT TTAGTTGATT |
| 1721 | ATTTTCTGCT ACAGACCTGG TACACTCTGA TTTTAGATAA |
| 1761 | AGTAAGCCTA GGTGTTGTCA GCAGGCAGGC TGGGGAGGCC |
| 1801 | AGTGTTGTGG GCCTCCTGCT GGGACTGAGA AGGCCCACGA |
| 1841 | AGGCGTCCGC AATGTTGGTT TCACTGAGAG CTGCCTCCTG |
| 1881 | GTCTCTTCAC CACTGTAGTT CTCTCATTTC CAAACCATCA |
| 1921 | GCTGCTTTTA AAATAAGATC TCTTTGTAGC CATCCTGTTA |
| 1961 | AATTTGTAAA CAATCTAATT AAATGGCATG CGCAG |

Subjects can be effectively treated by the compositions and methods described herein even though those subjects have p62 isoforms and variants with somewhat different sequences than the examples of p62 sequences described herein. Such p62 isoforms and variants can have polypeptide or nucleic acid sequences with between 55-100% sequence identity to a reference sequence (e.g., a sequence described herein). For example, p62 isoforms and variants can have at least 55% sequence identity, e.g., at least 60%, at least 70%, at least 80%, at least 90%, at least 95%, at least 96%, at least 97% sequence, at least 98%, at least 99% identity to a reference sequence (e.g., a sequence described herein) over a specified comparison window. Optimal alignment may be ascertained or conducted using the homology alignment algorithm of Needleman and Wunsch, J. Mol. Biol. 48:443-53 (1970).

### TNFα

An example of a human TNFa amino acid sequence is shown below (SEQ ID NO: 71).

| | |
|---|---|
| 1 | MSTESMIRDV ELAEEALPKK TGGPQGSRRC LFLSLFSFLI VAGATTLFCL LHFGVIGPQR |
| 61 | EEFPRDLSLI SPLAQAVRSS SRTPSDKPVA HVVANPQAEG QLQWLNRRAN ALLANGVELR |
| 121 | DNQLVVPSEG LYLIYSQVLF KGQGCPSTHV LLTHTISRIA VSYQTKVNLL SAIKSPCQRE |
| 181 | TPEGAEAKPW YEPIYLGGVF QLEKGDRLSA EINRPDYLDF AESGQVYFGI IAL |

A cDNA encoding a human TNFa protein is shown below (SEQ ID NO:72).

| | |
|---|---|
| 1 | AGCAGACGCT CCCTCAGCAA GGACAGCAGA GGACCAGCTA AGAGGGAGAG AAGCAACTAC |
| 61 | AGACCCCCCC TGAAAACAAC CCTCAGACGC CACATCCCCT GACAAGCTGC CAGGCAGGTT |
| 121 | CTCTTCCTCT CACATACTGA CCCACGGCTC CACCCTCTCT CCCCTGGAAA GGACACCATG |
| 181 | AGCACTGAAA GCATGATCCG GGACGTGGAG CTGGCCGAGG AGGCGCTCCC CAAGAAGACA |
| 241 | GGGGGGCCCC AGGGCTCCAG GCGGTGCTTG TTCCTCAGCC TCTTCTCCTT CCTGATCGTG |
| 301 | GCAGGCGCCA CCACGCTCTT CTGCCTGCTG CACTTTGGAG TGATCGGCCC CCAGAGGGAA |
| 361 | GAGTTCCCCA GGGACCTCTC TCTAATCAGC CCTCTGGCCC AGGCAGTCAG ATCATCTTCT |
| 421 | CGAACCCCGA GTGACAAGCC TGTAGCCCAT GTTGTAGCAA ACCCTCAAGC TGAGGGGCAG |
| 481 | CTCCAGTGGC TGAACCGCCG GGCCAATGCC CTCCTGGCCA ATGGCGTGGA GCTGAGAGAT |
| 541 | AACCAGCTGG TGGTGCCATC AGAGGGCCTG TACCTCATCT ACTCCCAGGT CCTCTTCAAG |
| 604 | GGCCAAGGCT GCCCCTCCAC CCATGTGCTC CTCACCCACA CCATCAGCCG CATCGCCGTC |
| 661 | TCCTACCAGA CCAAGGTCAA CCTCCTCTCT GCCATCAAGA GCCCCTGCCA GAGGGAGACC |
| 721 | CCAGAGGGGG CTGAGGCCAA GCCCTGGTAT GAGCCCATCT ATCTGGGAGG GGTCTTCCAG |
| 781 | CTGGAGAAGG GTGACCGACT CAGCGCTGAG ATCAATCGGC CCGACTATCT CGACTTTGCC |
| 841 | GAGTCTGGGC AGGTCTACTT TGGGATCATT GCCCTGTGAG GAGGACGAAC ATCCAACCTT |
| 901 | CCCAAACGCC TCCCCTGCCC CAATCCCTTT ATTACCCCCT CCTTCAGACA CCCTCAACCT |
| 961 | CTTCTGGCTC AAAAAGAGAA TTGGGGGCTT AGGGTCGGAA CCCAAGCTTA GAACTTTAAG |
| 1021 | CAACAAGACC ACCACTTCGA AACCTGGGAT TCAGGAATGT GTGGCCTGCA CAGTGAAGTG |
| 1081 | CTGGCAACCA CTAAGAATTC AAACTGGGGC CTCCAGAACT CACTGGGGCC TACAGCTTTG |
| 1141 | ATCCCTGACA TCTGGAATCT GGAGACCAGG GAGCCTTTGG TTCTGGCCAG AATGCTGCAG |
| 1201 | GACTTGAGAA GACCTCACCT AGAAATTGAC ACAAGTGGAC CTTAGGCCTT CCTCTCTCCA |
| 1261 | GATGTTTCCA GACTTCCTTG AGACACGGAG CCCAGCCCTC CCCATGGAGC CAGCTCCCTC |
| 1321 | TATTTATGTT TGCACTTGTG ATTATTTATT ATTTATTTAT TATTTATTTA TTTACAGATG |
| 1381 | AATGTATTTA TTTGGGAGAC CGGGGTATCC TGGGGGACCC AATGTAGGAG CTGCCTTGGC |
| 1441 | TCAGACATGT TTTCCGTGAA AACGGAGCTG AACAATAGGC TGTTCCCATG TAGCCCCCTG |
| 1501 | GCCTCTGTGC CTTCTTTTGA TTATGTTTTT TAAAATATTT ATCTGATTAA GTTGTCTAAA |
| 1561 | CAATGCTGAT TTGGTGACCA ACTGTCACTC ATTGCTGAGC CTCTGCTCCC CAGGGGAGTT |
| 1621 | GTGTCTGTAA TCGCCCTACT ATTCAGTGGC GAGAAATAAA GTTTGCTTAG AAAAGAAA |

Subjects can be effectively treated by the compositions and methods described herein even though those subjects have TNFa isoforms and variants with somewhat different sequences than the examples of TNFa sequences described herein. Such TNFa isoforms and variants can have polypeptide or nucleic acid sequences with between 55-100% sequence identity to a reference sequence (e.g., a sequence described herein). For example, TNFa isoforms and variants can have at least 55% sequence identity, e.g., at least 60%, at least 70%, at least 80%, at least 90%, at least 95%, at least 96%, at least 97% sequence, at least 98%, at least 99% identity to a reference sequence (e.g., a sequence described herein) over a specified comparison window. Optimal alignment may be ascertained or conducted using the homology alignment algorithm of Needleman and Wunsch, J. Mol. Biol. 48:443-53 (1970).

### IL-6

An example of a human IL-6 amino acid sequence is shown below (SEQ ID NO: 73).

| | |
|---|---|
| 1 | MNSFSTSAFG PVAFSLGLLL VLPAAFPAPV PPGEDSKDVA APHRQPLTSS ERIDKQIRYI |
| 61 | LDGISALRKE TCNKSNMCES SKEALAENNL NLPKMAEKDG CFQSGFNEET CLVKIITGLL |
| 121 | EFEVYLEYLQ NRFESSEEQA RAVQMSTKVL IQFLQKKAKN LDAITTPDPT TNASLLTKLQ |
| 181 | AQNQWLQDMT THLILRSFKE FLQSSLRALR QM |

A cDNA encoding a human IL-6 protein is shown below (SEQ ID NO:74).

| | |
|---|---|
| 1 | ATTCTGCCCT CGAGCCCACC GGGAACGAAA GAGAAGCTCT ATCTCCCCTC CAGGAGCCCA |
| 61 | GCTATGAACT CCTTCTCCAC AAGCGCCTTC GGTCCAGTTG CCTTCTCCCT GGGGCTGCTC |
| 121 | CTGGTGTTGC CTGCTGCCTT CCCTGCCCCA GTACCCCCAG GAGAAGATTC CAAAGATGTA |
| 181 | GCCGCCCCAC ACAGACAGCC ACTCACCTCT TCAGAACGAA TTGACAAACA AATTCGGTAC |
| 241 | ATCCTCGACG GCATCTCAGC CCTGAGAAAG GAGACATGTA ACAAGAGTAA CATGTGTGAA |
| 301 | AGCAGCAAAG AGGCACTGGC AGAAAACAAC CTGAACCTTC CAAAGATGGC TGAAAAAGAT |
| 361 | GGATGCTTCC AATCTGGATT CAATGAGGAG ACTTGCCTGG TGAAAATCAT CACTGGTCTT |
| 421 | TTGGAGTTTG AGGTATACCT AGAGTACCTC CAGAACAGAT TTGAGAGTAG TGAGGAACAA |
| 481 | GCCAGAGCTG TGCAGATGAG TACAAAAGTC CTGATCCAGT TCCTGCAGAA AAAGGCAAAG |
| 541 | AATCTAGATG CAATAACCAC CCCTGACCCA ACCACAAATG CCAGCCTGCT GACGAAGCTG |
| 601 | CAGGCACAGA ACCAGTGGCT GCAGGACATG ACAACTCATC TCATTCTGCG CAGCTTTAAG |
| 661 | GAGTTCCTGC AGTCCAGCCT GAGGGCTCTT CGGCAAATGT AGCATGGGCA CCTCAGATTG |
| 721 | TTGTTGTTAA TGGGCATTCC TTCTTCTGGT CAGAAACCTG TCCACTGGGC ACAGAACTTA |
| 781 | TGTTGTTCTC TATGGAGAAC TAAAAGTATG AGCGTTAGGA CACTATTTTA ATTATTTTTA |
| 841 | ATTTATTAAT ATTTAAATAT GTGAAGCTGA GTTAATTTAT GTAAGTCATA TTTATATTTT |
| 901 | TAAGAAGTAC CACTTGAAAC ATTTTATGTA TTAGTTTTGA AATAATAATG GAAAGTGGCT |
| 961 | ATGCAGTTTG AATATCCTTT GTTTCAGAGC CAGATCATTT CTTGGAAAGT GTAGGCTTAC |
| 1021 | CTCAAATAAA TGGCTAACTT ATACATATTT TTAAAGAAAT ATTTATATTG TATTTATATA |
| 1081 | ATGTATAAAT GGTTTTTATA CCAATAAATG GCATTTTAAA AAATTCA |

Subjects can be effectively treated by the compositions and methods described herein even though those subjects have IL-6 isoforms and variants with somewhat different sequences than the examples of IL-6 sequences described herein. Such IL-6 isoforms and variants can have polypeptide or nucleic acid sequences with between 55-100% sequence identity to a reference sequence (e.g., a sequence described herein). For example, IL-6 isoforms and variants can have at least 55% sequence identity, e.g., at least 60%, at least 70%, at least 80%, at least 90%, at least 95%, at least 96%, at least 97% sequence, at least 98%, at least 99% identity to a reference sequence (e.g., a sequence described herein) over a specified comparison window. Optimal alignment may be ascertained or conducted using the homology alignment algorithm of Needleman and Wunsch, J. Mol. Biol. 48:443-53 (1970).

### JAK

An example of a human JAK amino acid sequence is shown below (SEQ ID NO: 75).

| | |
|---|---|
| 1 | MQYLNIKEDC NAMAFCAKMR SSKKTEVNLE APEPGVEVIF YLSDREPLRL GSGEYTAEEL |
| 61 | CIRAAQACRI SPLCHNLFAL YDENTKLWYA PNRTITVDDK MSLRLHYRMR FYFTNWHGTN |
| 181 | DGHDIENECL GMAVLAISHY AMMKKMQLPE LPKDISYKRY IPETLNKSIR QRNLLTRMRI |
| 241 | NNVFKDFLKE FNNKTICDSS VSTHDLKVKY LATLETLTKH YGAEIFETSM LLISSENEMN |
| 301 | WFHSNDGGNV LYYEVMVTGN LGIQWRHKPN VVSVEKEKNK LKRKKLENKH KKDEEKNKIR |
| 361 | EEWNNFSYFP EITHIVIKES VVSINKQDNK KMELKLSSHE EALSFVSLVD GYFRLTADAH |
| 421 | HYLCTDVAPP LIVHNIQNGC HGPICTEYAI NKLRQEGSEE GMYVLRWSCT DFDNILMTVT |
| 481 | CFEKSEQVQG AQKQFKNFQI EVQKGRYSLH GSDRSFPSLG DLMSHLKKQI LRTDNISFML |
| 541 | KRCCQPKPRE ISNLLVATKK AQEWQPVYPM SQLSFDRILK KDLVQGEHLG RGTRTHIYSG |
| 601 | TLMDYKDDEG TSEEKKIKVI LKVLDPSHRD ISLAFFEAAS MMRQVSHKHI VYLYGVCVRD |
| 661 | VENIMVEEFV EGGPLDLFMH RKSDVLTTPW KFKVAKQLAS ALSYLEDKDL VHGNVCTKNL |
| 721 | LLAREGIDSE CGPFIKLSDP GIPITVLSRQ ECIERIPWIA PECVEDSKNL SVAADKWSFG |
| 781 | TTLWEICYNG EIPLKDKTLI EKERFYESRC RPVTPSCKEL ADLMTRCMNY DPNQRPFFRA |
| 841 | IMRDINKLEE QNPDIVSEKK PATEVDPTHF EKRFLKRIRD LGEGHFGKVE LCRYDPEGDN |
| 901 | TGEQVAVKSL KPESGGNHIA DLKKEIEILR NLYHENIVKY KGICTEDGGN GIKLIMEFLP |
| 961 | SGSLKEYLPK NKNKINLKQQ LKYAVQICKG MDYLGSRQYV HRDLAARNVL VESEHQVKIG |
| 1021 | DFGLTKAIET DKEYYTVKDD RDSPVFWYAP ECLMQSKFYI ASDVWSFGVT LHELLTYCDS |
| 1081 | DSSPMALFLK MIGPTHGQMT VTRLVNTLKE GKRLPCPPNC PDEVYQLMRK CWEFQPSNRT |
| 1141 | SFQNLIEGFE ALLK |

A cDNA encoding a human JAK protein is shown below (SEQ ID NO:76).

| | |
|---|---|
| 1 | GCGTCGCTGA GCGCAGGCCG CGGCGGCCGC GGAGTATCCT GGAGCTGCAG ACAGTGCGGG |
| 61 | CCTGCGCCCA GTCCCGGCTG TCCTCGCCGC GACCCCTCCT CAGCCCTGGG CGCGCGCACG |
| 121 | CTGGGGCCCC GCGGGGCTGG CCGCCTAGCG AGCCTGCCGG TCGACCCCAG CCAGCGCAGC |
| 181 | GACGGGGCGC TGCCTGGCCC AGGCGCACAC GGAAGTGCGC TTCTCTGAAG TAGCTTTGGA |
| 241 | AAGTAGAGAA GAAAATCCAG TTTGCTTCTT GGAGAACACT GGACAGCTGA ATAAATGCAG |
| 301 | TATCTAAATA TAAAAGAGGA CTGCAATGCC ATGGCTTTCT GTGCTAAAAT GAGGAGCTCC |
| 361 | AAGAAGACTG AGGTGAACCT GGAGGCCCCT GAGCCAGGGG TGGAAGTGAT CTTCTATCTG |
| 421 | TCGGACAGGG AGCCCCTCCG GCTGGGCAGT GGAGAGTACA CAGCAGAGGA ACTGTGCATC |
| 481 | AGGGCTGCAC AGGCATGCCG TATCTCTCCT CTTTGTCACA ACCTCTTTGC CCTGTATGAC |
| 541 | GAGAACACCA AGCTCTGGTA TGCTCCAAAT CGCACCATCA CCGTTGATGA CAAGATGTCC |
| 601 | CTCCGGCTCC ACTACCGGAT GAGGTTCTAT TTCACCAATT GGCATGGAAC CAACGACAAT |
| 661 | GAGCAGTCAG TGTGGCGTCA TTCTCCAAAG AAGCAGAAAA ATGGCTACGA GAAAAAAAAG |
| 721 | ATTCCAGATG CAACCCCTCT CCTTGATGCC AGCTCACTGG AGTATCTGTT TGCTCAGGGA |
| 781 | CAGTATGATT TGGTGAAATG CCTGGCTCCT ATTCGAGACC CCAAGACCGA GCAGGATGGA |
| 841 | CATGATATTG AGAACGAGTG TCTAGGGATG GCTGTCCTGG CCATCTCACA CTATGCCATG |
| 901 | ATGAAGAAGA TGCAGTTGCC AGAACTGCCC AAGGACATCA GCTACAAGCG ATATATTCCA |
| 961 | GAAACATTGA ATAAGTCCAT CAGACAGAGG AACCTTCTCA CCAGGATGCG GATAAATAAT |
| 1021 | GTTTTCAAGG ATTTCCTAAA GGAATTTAAC AACAAGACCA TTTGTGACAG CAGCGTGTCC |
| 1081 | ACGCATGACC TGAAGGTGAA ATACTTGGCT ACCTTGGAAA CTTTGACAAA ACATTACGGT |
| 1141 | GCTGAAATAT TTGAGACTTC CATGTTACTG ATTTCATCAG AAAATGAGAT GAATTGGTTT |
| 1201 | CATTCGAATG ACGGTGGAAA CGTTCTCTAC TACGAAGTGA TGGTGACTGG GAATCTTGGA |
| 1261 | ATCCAGTGGA GGCATAAACC AAATGTTGTT TCTGTTGAAA AGGAAAAAAA TAAACTGAAG |
| 1321 | CGGAAAAAAC TGGAAAATAA ACACAAGAAG GATGAGGAGA AAAACAAGAT CCGGGAAGAG |
| 1381 | TGGAACAATT TTTCTTACTT CCCTGAAATC ACTCACATTG TAATAAAGGA GTCTGTGGTC |
| 1441 | AGCATTAACA AGCAGGACAA CAAGAAAATG GAACTGAAGC TCTCTTCCCA CGAGGAGGCC |
| 1501 | TTGTCCTTTG TGTCCCTGGT AGATGGCTAC TTCCGGCTCA CAGCAGATGC CCATCATTAC |
| 1561 | CTCTGCACCG ACGTGGCCCC CCCGTTGATC GTCCACAACA TACAGAATGG CTGTCATGGT |
| 1621 | CCAATCTGTA CAGAATACGC CATCAATAAA TTGCGGCAAG AAGGAAGCGA GGAGGGGATG |
| 1681 | TACGTGCTGA GGTGGAGCTG CACCGACTTT GACAACATCC TCATGACCGT CACCTGCTTT |
| 1741 | GAGAAGTCTG AGCAGGTGCA GGGTGCCCAG AAGCAGTTCA AGAACTTTCA GATCGAGGTG |
| 1801 | CAGAAGGGCC GCTACAGTCT GCACGGTTCG GACCGCAGCT TCCCCAGCTT GGGAGACCTC |
| 1861 | ATGAGCCACC TCAAGAAGCA GATCCTGCGC ACGGATAACA TCAGCTTCAT GCTAAAACGC |
| 1921 | TGCTGCCAGC CCAAGCCCCG AGAAATCTCC AACCTGCTGG TGGCTACTAA GAAAGCCCAG |
| 1981 | GAGTGGCAGC CCGTCTACCC CATGAGCCAG CTGAGTTTCG ATCGGATCCT CAAGAAGGAT |
| 2041 | CTGGTGCAGG GCGAGCACCT TGGGAGAGGC ACGAGAACAC ACATCTATTC TGGGACCCTG |
| 2101 | ATGGATTACA AGGATGACGA AGGAACTTCT GAAGAGAAGA AGATAAAAGT GATCCTCAAA |
| 2161 | GTCTTAGACC CCAGCCACAG GGATATTTCC CTGGCCTTCT TCGAGGCAGC CAGCATGATG |
| 2221 | AGACAGGTCT CCCACAAACA CATCGTGTAC CTCTATGGCG TCTGTGTCCG CGACGTGGAG |
| 2281 | AATATCATGG TGGAAGAGTT TGTGGAAGGG GGTCCTCTGG ATCTCTTCAT GCACCGGAAA |
| 2341 | AGCGATGTCC TTACCACACC ATGGAAATTC AAAGTTGCCA AACAGCTGGC CAGTGCCCTG |
| 2401 | AGCTACTTGG AGGATAAAGA CCTGGTCCAT GGAAATGTGT GTACTAAAAA CCTCCTCCTG |
| 2461 | GCCCGTGAGG GCATCGACAG TGAGTGTGGC CCATTCATCA AGCTCAGTGA CCCCGGCATC |
| 2521 | CCCATTACGG TGCTGTCTAG GCAAGAATGC ATTGAACGAA TCCCATGGAT TGCTCCTGAG |
| 2581 | TGTGTTGAGG ACTCCAAGAA CCTGAGTGTG GCTGCTGACA AGTGGAGCTT TGGAACCACG |
| 2641 | CTCTGGGAAA TCTGCTACAA TGGCGAGATC CCCTTGAAAG ACAAGACGCT GATTGAGAAA |
| 2701 | GAGAGATTCT ATGAAAGCCG GTGCAGGCCA GTGACACCAT CATGTAAGGA GCTGGCTGAC |
| 2761 | CTCATGACCC GCTGCATGAA CTATGACCCC AATCAGAGGC CTTTCTTCCG AGCCATCATG |
| 2821 | AGAGACATTA ATAAGCTTGA AGAGCAGAAT CCAGATATTG TTTCAGAAAA AAAACCAGCA |
| 2881 | ACTGAAGTGG ACCCCACACA TTTTGAAAAG CGCTTCCTAA AGAGGATCCG TGACTTGGGA |
| 2941 | GAGGGCCACT TTGGGAAGGT TGAGCTCTGC AGGTATGACC CCGAAGGGGA CAATACAGGG |
| 3001 | GAGCAGGTGG CTGTTAAATC TCTGAAGCCT GAGAGTGGAG GTAACCACAT AGCTGATCTG |
| 3061 | AAAAAGGAAA TCGAGATCTT AAGGAACCTC TATCATGAGA ACATTGTGAA GTACAAAGGA |
| 3121 | ATCTGCACAG AAGACGGAGG AAATGGTATT AAGCTCATCA TGGAATTTCT GCCTTCGGGA |
| 3181 | AGCCTTAAGG AATATCTTCC AAAGAATAAG AACAAAATAA ACCTCAAACA GCAGCTAAAA |
| 3241 | TATGCCGTTC AGATTTGTAA GGGGATGGAC TATTTGGGTT CTCGGCAATA CGTTCACCGG |
| 3301 | GACTTGGCAG CAAGAAATGT CCTTGTTGAG AGTGAACACC AAGTGAAAAT TGGAGACTTC |
| 3361 | GGTTTAACCA AAGCAATTGA AACCGATAAG GAGTATTACA CCGTCAAGGA TGACCGGGAC |
| 3421 | AGCCCTGTGT TTTGGTATGC TCCAGAATGT TTAATGCAAT CTAAATTTTA TATTGCCTCT |
| 3481 | GACGTCTGGT CTTTTGGAGT CACTCTGCAT GAGCTGCTGA CTTACTGTGA TTCAGATTCT |
| 3541 | AGTCCCATGG CTTTGTTCCT GAAAATGATA GGCCCAACCC ATGGCCAGAT GACAGTCACA |
| 3601 | AGACTTGTGA ATACGTTAAA AGAAGGAAAA CGCCTGCCGT GCCCACCTAA CTGTCCAGAT |
| 3661 | GAGGTTTATC AACTTATGAG GAAATGCTGG GAATTCCAAC CATCCAATCG GACAAGCTTT |
| 3721 | CAGAACCTTA TTGAAGGATT TGAAGCACTT TTAAAATAAG AAGCATGAAT AACATTTAAA |
| 3781 | TTCCACAGAT TATCAAGTCC TTCTCCTGCA ACAAATGCCC AAGTCATTTT TTAAAAATTT |
| 3841 | CTAATGAAAG AAGTTTGTGT TCTGTCCAAA AAGTCACTGA ACTCATACTT CAGTACATAT |
| 3901 | ACATGTATAA GGCACACTGT AGTGCTTAAT ATGTGTAAGG ACTTCCTCTT TAAATTTGGT |
| 3961 | ACCAGTAACT TAGTGACACA TAATGACAAC CAAAATATTT GAAAGCACTT AAGCACTCCT |
| 4021 | CCTTGTGGAA AGAATATACC ACCATTTCAT CTGGCTAGTT CACCATCACA ACTGCATTAC |
| 4081 | CAAAAGGGGA TTTTTGAAAA CGAGGAGTTG ACCAAAATAA TATCTGAAGA TGATTGCTTT |
| 4141 | TCCCTGCTGC CAGCTGATCT GAAATGTTTT GCTGGCACAT TAATCATAGA TAAAGAAAGA |
| 4201 | TTGATGGACT TAGCCCTCAA ATTTCAGTAT CTATACAGTA CTAGACCATG CATTCTTAAA |
| 4261 | ATATTAGATA CCAGGTAGTA TATATTGTTT CTGTACAAAA ATGACTGTAT TCTCTCACCA |
| 4321 | GTAGGACTTA AACTTTGTTT CTCCAGTGGC TTAGCTCCTG TTCCTTTGGG TGATCACTAG |
| 4381 | CACCCATTTT TGAGAAAGCT GGTTCTACAT GGGGGGATAG CTGTGGAATA GATAATTTGC |
| 4441 | TGCATGTTAA TTCTCAAGAA CTAAGCCTGT GCCAGTGCTT TCCTAAGCAG TATACCTTTA |
| 4501 | ATCAGAACTC ATTCCCAGAA CCTGGATGCT ATTACACATG CTTTTAAGAA ACGTCAATGT |
| 4561 | ATATCCTTTT ATAACTCTAC CACTTTGGGG CAAGCTATTC CAGCACTGGT TTTGAATGCT |
| 4621 | GTATGCAACC AGTCTGAATA CCACATACGC TGCACTGTTC TTAGAGGGTT TCCATACTTA |
| 4681 | CCACCGATCT ACAAGGGTTG ATCCCTGTTT TTACCATCAA TCATCACCCT GTGGTGCAAC |
| 4741 | ACTTGAAAGA CCCGGCTAGA GGCACTATGG ACTTCAGGAT CCACTAGACA GTTTTCAGTT |
| 4801 | TGCTTGGAGG TAGCTGGGTA ATCAAAAATG TTTAGTCATT GATTCAATGT GAACGATTAC |
| 4861 | GGTCTTTATG ACCAAGAGTC TGAAAATCTT TTTGTTATGC TGTTTAGTAT TCGTTTGATA |
| 4921 | TTGTTACTTT TCACCTGTTG AGCCCAAATT CAGGATTGGT TCAGTGGCAG CAATGAAGTT |
| 4981 | GCCATTTAAA TTTGTTCATA GCCTACATCA CCAAGGTCTC TGTGTCAAAC CTGTGGCCAC |
| 5041 | TCTATATGCA CTTTGTTTAC TCTTTATACA AATAAATATA CTAAAGACTT TA |

Subjects can be effectively treated by the compositions and methods described herein even though those subjects have JAK isoforms and variants with somewhat different sequences than the examples of JAK sequences described herein. Such JAK isoforms and variants can have polypeptide or nucleic acid sequences with between 55-100% sequence identity to a reference sequence (e.g., a sequence described herein). For example, JAK isoforms and variants can have at least 55% sequence identity, e.g., at least 60%, at least 70%, at least 80%, at least 90%, at least 95%, at least 96%, at least 97% sequence, at least 98%, at least 99% identity to a reference sequence (e.g., a sequence described herein) over a specified comparison window. Optimal alignment may be ascertained or conducted using the homology alignment algorithm of Needleman and Wunsch, J. Mol. Biol. 48:443-53 (1970).

### Inhibitory Nucleic Acids

The expression of hyaluronan synthase can be inhibited, for example by use of an inhibitory nucleic acid that specifically recognizes a nucleic acid that encodes the hyaluronan synthase.

An inhibitory nucleic acid can have at least one segment that will hybridize to a hyaluronan synthase nucleic acid, ULK1 nucleic acid, TNFa nucleic acid, IL6 nucleic acid or JAK nucleic acid under intracellular or stringent conditions. The inhibitory nucleic acid can reduce expression of a nucleic acid encoding hyaluronan synthase, ULK1, TNFα, IL6 or JAK. A nucleic acid may hybridize to a genomic DNA, a messenger RNA, or a combination thereof. An inhibitory nucleic acid may be incorporated into a plasmid vector or viral DNA. It may be single stranded or double stranded, circular or linear.

An inhibitory nucleic acid is a polymer of ribose nucleotides or deoxyribose nucleotides having more than 13 nucleotides in length. An inhibitory nucleic acid may include naturally occurring nucleotides; synthetic, modified, or pseudo-nucleotides such as phosphorothiolates; as well as nucleotides having a detectable label such as P³², biotin or digoxigenin. An inhibitory nucleic acid can reduce the expression and/or activity of a hyaluronan synthase nucleic acid, ULK1 nucleic acid, TNFα nucleic acid, IL6 nucleic acid or JAK nucleic acid. Such an inhibitory nucleic acid may be completely complementary to a segment of an endogenous hyaluronan synthase nucleic acid (e.g., a hyaluronan synthase RNA) or ULK1 nucleic acid (e.g., an ULK1 mRNA) or TNFα nucleic acid (e.g., a TNFα mRNA) or IL-6 nucleic acid (e.g., a IL-6 mRNA) or JAK nucleic acid (e.g., a JAK mRNA). Alternatively, some variability is permitted in the inhibitory nucleic acid sequences relative to hyaluronan synthase, ULK1, TNFα, IL6 or JAK. An inhibitory nucleic acid can hybridize to a hyaluronan synthase nucleic acid or ULK1 nucleic acid or a TNFα nucleic acid or a IL-6 nucleic acid or a JAK nucleic acid under intracellular conditions or under stringent hybridization conditions and is sufficiently complementary to inhibit expression of the endogenous hyaluronan synthase nucleic acid or ULK1 nucleic acid or TNFα nucleic acid or IL-6 nucleic acid or JAK nucleic acid. Intracellular conditions refer to conditions such as temperature, pH and salt concentrations typically found inside a cell, e.g., an animal or mammalian cell. One example of such an animal or mammalian cell is a stromal or epithelial cell. Another example of such an animal or mammalian cell is a cancer cell. Generally, stringent hybridization conditions are selected to be about 5° C. lower than the thermal melting point (Tm) for the specific sequence at a defined ionic strength and pH. However, stringent conditions encompass temperatures in the range of about 1° C. to about 20° C. lower than the thermal melting point of the selected sequence, depending upon the desired degree of stringency as otherwise qualified herein. Inhibitory oligonucleotides can, for example, have 2, 3, 4, or 5 or more stretches of contiguous nucleotides that are precisely complementary to a hyaluronan synthase coding sequence or ULK1 coding sequence or TNFα coding sequence or IL-6 coding sequence or JAK coding sequence, wherein each complementary stretch is separated by one or more contiguous nucleotides that are not complementary to adjacent coding sequences, can inhibit the function of a hyaluronan synthase nucleic acid or ULK1 nucleic acid or TNFα nucleic acid or IL-6 nucleic acid or JAK nucleic acid. In general, each stretch of contiguous nucleotides is at least 4, 5, 6, 7, or 8 or more nucleotides in length. Non-complementary intervening sequences may be 1, 2, 3, or 4 nucleotides in length. One skilled in the art can easily use the calculated melting point of an inhibitory nucleic acid hybridized to a sense nucleic acid to estimate the degree of mismatching that will be tolerated for inhibiting expression of a particular target nucleic acid. Inhibitory nucleic acids of the invention include, for example, a short hairpin RNA, a small interfering RNA, a ribozyme or an antisense nucleic acid molecule.

The inhibitory nucleic acid molecule may be single or double stranded (e.g., a small interfering RNA (siRNA)) and may function in an enzyme-dependent manner or by steric blocking. Inhibitory nucleic acid molecules that function in an enzyme-dependent manner include forms dependent on RNase H activity to degrade target mRNA. These include single-stranded DNA, RNA, and phosphorothioate molecules, as well as the double-stranded RNAi/siRNA system that involves target mRNA recognition through sense-antisense strand pairing followed by degradation of the target mRNA by the RNA-induced silencing complex. Steric blocking inhibitory nucleic acids, which are RNase-H independent, interfere with gene expression or other mRNA-dependent cellular processes by binding to a target mRNA and getting in the way of other processes. Steric blocking inhibitory nucleic acids include 2′-O alkyl (usually in chimeras with RNase-H dependent antisense), peptide nucleic acid (PNA), locked nucleic acid (LNA) and morpholino antisense.

Small interfering RNAs, for example, may be used to specifically reduce translation of hyaluronan synthase or ULK1 or TNFα or IL-6 or JAK nucleic acid such that translation of the encoded hyaluronan synthase, ULK1, TNFα, IL-6 or JAK is inhibited. SiRNAs mediate post-transcriptional gene silencing in a sequence-specific manner. See, for example, website at invitrogen.com/site/us/en/home/Products-and-Services/Applications/rnai.html. Once incorporated into an RNA-induced silencing complex, siRNA mediate cleavage of the homologous endogenous mRNA transcript by guiding the complex to the homologous mRNA transcript, which is then cleaved by the complex. The siRNA may be homologous and/or complementary to any region of the hyaluronan synthase transcript or to any region of an ULK1 transcript or to any region of a TNFα transcript or any region of a IL-6 transcript or any region of a JAKtranscript. The region of homology may be 30 nucleotides or less in length, e.g., less than 25 nucleotides such as about 21 to 23 nucleotides in length. SiRNA is typically double stranded and may have two-nucleotide 3′ overhangs, for example, 3′ overhanging UU dinucleotides. Methods for designing siRNAs are known to those skilled in the art. See, for example, Elbashir et al. Nature 411:494-498 (2001); Harborth et al. Antisense Nucleic Acid Drug Dev. 13:83-106 (2003).

The pSuppressorNeo vector for expressing hairpin siRNA, commercially available from IMGENEX (San Diego, California), can be used to generate siRNA for inhibiting expression of hyaluronan synthase or ULK1. The construction of the siRNA expression plasmid involves the selection of the target region of the mRNA, which can be a trial-and-error process. However, Elbashir et al. have provided guidelines that appear to work ˜80% of the time. Elbashir, S. M., et al., Analysis of gene function in somatic mammalian cells using small interfering RNAs. Methods, 2002. 26(2): p. 199-213. Accordingly, for synthesis of synthetic siRNA, a target region may be selected, e.g., 50 to 100 nucleotides downstream of the start codon. The 5′ and 3′ untranslated regions and regions close to the start codon should be avoided as these may be richer in regulatory protein binding sites. As siRNA can begin with AA, have 3′ UU overhangs for both the sense and antisense siRNA strands, and have an approximate 50% G/C content. An example of a sequence for a synthetic siRNA is 5′-AA(N19) UU, where N is any nucleotide in the mRNA sequence and the siRNA should be approximately 50% G-C content. The selected sequence(s) can be compared to others in the human genome database to minimize homology to other known coding sequences (e.g., by Blast search, for example, through the NCBI website).

SiRNAs may be chemically synthesized, created by in vitro transcription, or expressed from an siRNA expression vector or a PCR expression cassette. See, e.g., website at invitrogen.com/site/us/en/home/Products-and-Services/Applications/rnai.html. When an siRNA is expressed from an expression vector or a PCR expression cassette, the insert encoding the siRNA may be expressed as an RNA transcript that folds into an siRNA hairpin. Thus, the RNA transcript may include a sense siRNA sequence that is linked to its reverse complementary antisense siRNA sequence by a spacer sequence that forms the loop of the hairpin as well as a string of U's at the 3′ end. The loop of the hairpin may be of any appropriate lengths, for example, 3 to 30 nucleotides in length, e.g., 3 to 23 nucleotides in length, and may be of various nucleotide sequences including, AUG, CCC, UUCG, CCACC, CTCGAG, AAGCUU, CCACACC and UUCAAGAGA. SiRNAs also may be produced in vivo by cleavage of double-stranded RNA introduced directly or via a transgene or virus. Amplification by an RNA-dependent RNA polymerase may occur in some organisms.

An inhibitory nucleic acid such as a short hairpin RNA siRNA or an antisense oligonucleotide may be prepared using methods such as by expression from an expression vector or expression cassette that includes the sequence of the inhibitory nucleic acid. Alternatively, it may be prepared by chemical synthesis using naturally occurring nucleotides, modified nucleotides or any combinations thereof. In some embodiments, the inhibitory nucleic acids are made from modified nucleotides or non-phosphodiester bonds, for example, that are designed to increase biological stability of the inhibitory nucleic acid or to increase intracellular stability of the duplex formed between the inhibitory nucleic acid and the target hyaluronan synthase nucleic acid.

An inhibitory nucleic acid may be generated, for example, by expression from an expression vector encoding a complementarity sequence of the hyaluronan synthase nucleic acid, ULK1 nucleic acid, TNFα nucleic acid, IL-6 nucleic acid or JAK nucleic acid. Alternatively, it may be prepared by chemical synthesis using naturally occurring nucleotides, modified nucleotides or any mixture of combination thereof. In some embodiments, the hyaluronan synthase nucleic acids or ULK1 nucleic acids or TNFα nucleic acids or IL-6 nucleic acids or JAK nucleic acids are made from modified nucleotides or non-phosphodiester bonds, for example, that are designed to increase biological stability of the nucleic acids or to increase intracellular stability of the duplex formed between the inhibitory nucleic acids and other (e.g., endogenous) nucleic acids.

For example, the inhibitory hyaluronan synthase nucleic acids or inhibitory ULK1 nucleic acids or inhibitory TNFα nucleic acids or inhibitory IL-6 nucleic acids or inhibitory JAK nucleic acids can be peptide nucleic acids that have peptide bonds rather than phosphodiester bonds.

The inhibitory nucleic acids of the hyaluronan synthase or ULK1 or TNFα or IL-6 or JAK described herein may include modified nucleotides, as well as natural nucleotides such as combinations of ribose and deoxyribose nucleotides.

Naturally occurring nucleotides that can be employed in the hyaluronan synthase nucleic acids or ULK1 nucleic acids or TNFα nucleic acids or IL-6 nucleic acids or JAK nucleic acids can include the ribose or deoxyribose nucleotides adenosine, guanine, cytosine, thymine and uracil.

Examples of modified nucleotides that can be employed in the inhibitory hyaluronan synthase nucleic acids or inhibitory ULK1 nucleic acids or inhibitory TNFα nucleic acids or inhibitory IL-6 nucleic acids or inhibitory JAK nucleic acids include 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methythio-N6-isopentenyladeninje, uracil-5oxyacetic acid, wybutoxosine, pseudouracil, queosine, 2-thiocytosine, 5-methyl-2-thiouracil, 2-thiouracil, 4-thiouracil, 5-methyluracil, uracil-5-oxacetic acid methylester, uracil-5-oxacetic acid, 5-methyl-2-thiouracil, 3-(3-amino-3-N-2-carboxypropyl) uracil, (acp3)w, and 2,6-diaminopurine.

The inhibitory nucleic acids and may be of same length as wild type hyaluronan synthase or as any isoforms or variants of hyaluronan synthase or ULK1 or TNFα or IL-6 or JAK. The inhibitory nucleic acids of the hyaluronan synthase, ULK1, TNFα, IL-6 or JAK described herein can also be longer and include other useful sequences. In some embodiments, the inhibitory nucleic acids of the hyaluronan synthase, ULK1, TNFα, IL-6 or JAK are somewhat shorter. For example, inhibitory nucleic acids of hyaluronan synthase, ULK1, TNFα, IL-6 or JAK can include a segment that has a nucleic acid sequence that can be missing up to 5 nucleotides, or missing up to 10 nucleotides, or missing up to 20 nucleotides, or missing up to 30 nucleotides, or missing up to 50 nucleotides, or missing up to 100 nucleotides from the 5′ or 3′ end.

The inhibitory nucleic acids can be introduced via one or more vehicles such as via expression vectors (e.g., viral vectors), via virus like particles, via ribonucleoproteins (RNPs), via nanoparticles, via liposomes, or a combination thereof. The vehicles can include components or agents that can target particular cell types, facilitate cell penetration, reduce degradation, or a combination thereof.

### Antibodies

Antibodies can be used as inhibitors of hyaluronan synthase, ULK1, TNFα, IL-6 or JAK. Antibodies can be raised against various epitopes of hyaluronan synthase, ULK1, TNFα, IL-6 or JAK. Some antibodies for hyaluronan synthase, ULK1, TNFα, IL-6 or JAK may also be available commercially. However, the antibodies contemplated for treatment pursuant to the methods and compositions described herein are in one embodiment human or humanized antibodies and are highly specific for their targets.

In one aspect, the present disclosure relates to use of isolated antibodies that bind specifically to hyaluronan synthase, ULK1, TNFα, IL-6 or JAK. Such antibodies may be monoclonal antibodies. Such antibodies may also be humanized or fully human monoclonal antibodies. The antibodies can exhibit one or more desirable functional properties, such as high affinity binding to hyaluronan synthase, ULK1, TNFα, IL-6 or JAK.

Methods and compositions described herein can include antibodies that bind hyaluronan synthase, ULK1, TNFα, IL-6 or JAK, or a combination of antibodies where each antibody type can separately bind hyaluronan synthase, ULK1, TNFα, IL-6 or JAK.

The term “antibody” as referred to herein includes whole antibodies and any antigen binding fragment (i.e., “antigen-binding portion”) or single chains thereof. An “antibody” refers to a glycoprotein comprising at least two heavy (H) chains and two light (L) chains inter-connected by disulfide bonds, or an antigen binding portion thereof. Each heavy chain is comprised of a heavy chain variable region (abbreviated herein as V_{H}) and a heavy chain constant region. The heavy chain constant region is comprised of three domains, C_{H1}, C_{H2} and C_{H3}. Each light chain is comprised of a light chain variable region (abbreviated herein as V_{L}) and a light chain constant region. The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and

V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system.

The term “antigen-binding portion” of an antibody (or simply “antibody portion”), as used herein, refers to one or more fragments of an antibody that retain the ability to specifically bind to an antigen (e.g., a peptide or domain of hyaluronan synthase, ULK1, TNFα, IL-6 or JAK). It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the V_{L}, V_{H}, C_{L} and C_{H1} domains; (ii) a F(ab′)₂ fragment, a bivalent fragment comprising two Fab fragments linked by a disulfide bridge at the hinge region; (iii) a Fd fragment consisting of the V_{H} and C_{H1} domains; (iv) a Fv fragment consisting of the V_{L} and V_{H} domains of a single arm of an antibody, (v) a dAb fragment (Ward et al., (1989) Nature 341:544-546), which consists of a V_{H} domain; and (vi) an isolated complementarity determining region (CDR). Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. These antibody fragments are obtained using available techniques, and the fragments are screened for utility in the same manner as are intact antibodies.

An “isolated antibody,” as used herein, is intended to refer to an antibody that is substantially free of other antibodies having different antigenic specificities (e.g., an isolated antibody that specifically binds hyaluronan synthase, ULK1, TNFα, IL-6 or JAK is substantially free of antibodies that specifically bind antigens other than a hyaluronan synthase, ULK1, TNFα, IL-6 or JAK). An isolated antibody that specifically binds hyaluronan synthase, ULK1, TNFα, IL-6 or JAK may, however, have cross-reactivity to related antigens, such as isoforms or variants of hyaluronan synthase, ULK1, TNFα, IL-6 or JAK from various humans or from other species. Moreover, an isolated antibody may be substantially free of other cellular material and/or chemicals.

The terms “monoclonal antibody” or “monoclonal antibody composition” as used herein refer to a preparation of antibody molecules of single molecular composition. A monoclonal antibody composition displays a single binding specificity and affinity for a particular epitope.

The term “human antibody,” as used herein, is intended to include antibodies having variable regions in which both the framework and CDR regions are derived from human germline immunoglobulin sequences. Furthermore, if the antibody contains a constant region, the constant region also is derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo). However, the term “human antibody,” as used herein, is not intended to include antibodies in which CDR sequences derived from the germline of another mammalian species, such as a mouse, have been grafted onto human framework sequences.

The term “human monoclonal antibody” refers to antibodies displaying a single binding specificity which have variable regions in which both the framework and CDR regions are derived from human germline immunoglobulin sequences. In one embodiment, the human monoclonal antibodies are produced by a hybridoma which includes a B cell obtained from a transgenic nonhuman animal, e.g., a transgenic mouse, having a genome comprising a human heavy chain transgene and a light chain transgene fused to an immortalized cell.

The term “recombinant human antibody,” as used herein, includes all human antibodies that are prepared, expressed, created or isolated by recombinant means, such as (a) antibodies isolated from an animal (e.g., a mouse) that is transgenic or transchromosomal for human immunoglobulin genes or a hybridoma prepared therefrom (described further below), (b) antibodies isolated from a host cell transformed to express the human antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the V_{L} and V_{H} regions of the recombinant antibodies are sequences that, while derived from and related to human germline V_{L} and V_{H} sequences, may not naturally exist within the human antibody germline repertoire in vivo.

As used herein, “isotype” refers to the antibody class (e.g., IgM or IgG1) that is encoded by the heavy chain constant region genes.

The phrases “an antibody recognizing an antigen” and “an antibody specific for an antigen” are used interchangeably herein with the term “an antibody which binds specifically to an antigen.”

The term “human antibody derivatives” refers to any modified form of the human antibody, e.g., a conjugate of the antibody and another agent or antibody.

The term “humanized antibody” is intended to refer to antibodies in which CDR sequences derived from the germline of another mammalian species, such as a mouse, have been grafted onto human framework sequences. Additional framework region modifications may be made within the human framework sequences.

The term “chimeric antibody” is intended to refer to antibodies in which the variable region sequences are derived from one species and the constant region sequences are derived from another species, such as an antibody in which the variable region sequences are derived from a mouse antibody and the constant region sequences are derived from a human antibody.

As used herein, an antibody that “specifically binds to human hyaluronan synthase” or an antibody that “specifically binds to human ULK1” or an antibody that “specifically binds to TNFα” or an antibody that “specifically binds to IL-6” or an antibody that “specifically binds to JAK” is intended to refer to an antibody that binds to human hyaluronan synthase or ULK1 or TNFα or IL-6 or JAK with a K_{D} of 1×10⁻⁷ M or less, 5×10⁻⁸ M or less, 1×10⁻⁸ M or less, 5×10⁻⁹ M or less, or between 1×10⁻⁸ M and 1×10⁻¹⁰ M or less.

The term “K_{assoc}” or “Kₐ,” as used herein, is intended to refer to the association rate of a particular antibody-antigen interaction, whereas the term “Kdis” or “K_{d},” as used herein, is intended to refer to the dissociation rate of a particular antibody-antigen interaction. The term “K_{D},” as used herein, is intended to refer to the dissociation constant, which is obtained from the ratio of K_{d} to Kₐ (i.e., K_{d}/Kₐ) and is expressed as a molar concentration (M). K_{D} values for antibodies can be determined using methods well established in the art. One method for determining the K_{D} of an antibody is by using surface plasmon resonance, e.g., using a biosensor system such as a Biacore™ system.

The antibodies of the invention are characterized by particular functional features or properties of the antibodies. For example, the antibodies bind specifically to human hyaluronan synthase, ULK1, TNFα, IL-6 or JAK. In one embodiment, an antibody of the invention binds to hyaluronan synthase, ULK1, TNFα, IL-6 or JAK with high affinity, for example with a K_{D} of 1×10⁻⁷ M or less. The antibodies can exhibit one or more of the following characteristics:
(a) binds to human hyaluronan synthase, ULK1, TNFα, IL-6 or JAK with a K_{D} of 1×10⁻⁷ M or less;
(b) inhibits the function or activity of hyaluronan synthase, ULK1, TNFα, IL-6 or JAK;
(c) inhibits cancer (e.g., metastatic cancer); or
(d) a combination thereof.

Assays to evaluate the binding ability of the antibodies toward hyaluronan synthase, ULK1, TNFα, IL-6 or JAK can be used, including for example, ELISAs, Western blots and RIAs. The binding kinetics (e.g., binding affinity) of the antibodies also can be assessed by standard assays known in the art, such as by Biacore™. analysis.

Given that each of the subject antibodies can bind to hyaluronan synthase, ULK1, TNFα, IL-6 or JAK the V_{L} and V_{H} sequences or the respective antibodies can be “mixed and matched” to create other binding molecules that bind to hyaluronan synthase, ULK1, TNFα, IL-6 or JAK, respectively. The binding properties of such “mixed and matched” antibodies can be tested using the binding assays described above and assessed in assays described in the examples. When V_{L} and V_{H} chains are mixed and matched, a V_{H} sequence from a particular V_{H}/V_{L} pairing can be replaced with a structurally similar V_{H} sequence. Likewise, a V_{L} sequence from a particular V_{H}/V_{L} pairing may be replaced with a structurally similar V_{L} sequence.

Accordingly, in one aspect, the invention provides an isolated monoclonal antibody, or antigen binding portion thereof comprising:
(a) a heavy chain variable region comprising an amino acid sequence; and
(b) a light chain variable region comprising an amino acid sequence;
wherein the antibody specifically binds hyaluronan synthase, ULK1, TNFα, IL-6 or JAK.

In some cases, the CDR3 domain, independently from the CDR1 and/or CDR2 domain(s), alone can determine the binding specificity of an antibody for a cognate antigen and that multiple antibodies can predictably be generated having the same binding specificity based on a common CDR3 sequence. See, for example, Klimka et al., British J. of Cancer 83 (2): 252-260 (2000) (describing the production of a humanized anti-CD30 antibody using only the heavy chain variable domain CDR3 of murine anti-CD30 antibody Ki-4); Beiboer et al., J. Mol. Biol. 296:833-849 (2000) (describing recombinant epithelial glycoprotein-2 (EGP-2) antibodies using only the heavy chain CDR3 sequence of the parental murine MOC-31 anti-EGP-2 antibody); Rader et al., Proc. Natl. Acad. Sci. U.S.A. 95:8910-8915 (1998) (describing a panel of humanized anti-integrin alpha, beta3 antibodies using a heavy and light chain variable CDR3 domain. Hence, in some cases a mixed and matched antibody or a humanized antibody contains a CDR3 antigen binding domain that is specific for hyaluronan synthase, ULK1, TNFα, IL-6 or JAK.

### Expression Systems

Nucleic acid segments encoding one or more inhibitory nucleic acids, proteins (e.g., hyaluronidase), or antibodies can be inserted into or employed with any suitable expression system. Commercially useful and/or therapeutically effective quantities of one or more inhibitory nucleic acids, proteins (e.g., hyaluronidase), or antibodies can also be generated from such expression systems. In some cases, the expression systems can be used to express hyaluronidases. In some cases, the expression systems can be used to express inhibitory nucleic acids that can reduce endogenous hyaluronan synthase levels.

Recombinant expression of nucleic acids is usefully accomplished using a vector, such as a plasmid. The vector can include a promoter operably linked to nucleic acid segment encoding one or more of the hyaluronidase or an inhibitory nucleic acid for hyaluronan synthase nucleic acids, or other proteins (e.g., hyaluronidase), or antibodies.

The vector can also include other elements required for transcription and translation. As used herein, vector refers to any carrier containing exogenous DNA. Thus, vectors are agents that transport the exogenous nucleic acid into a cell without degradation and include a promoter yielding expression of the nucleic acid in the cells into which it is delivered. Vectors include but are not limited to plasmids, viral nucleic acids, viruses, phage nucleic acids, phages, cosmids, and artificial chromosomes.

A variety of prokaryotic and eukaryotic expression vectors suitable for carrying, encoding and/or expressing hyaluronidase, inhibitory nucleic acids, proteins, or antibodies can be used. A variety of prokaryotic and eukaryotic expression vectors suitable for carrying, encoding and/or expressing hyaluronidase, inhibitory nucleic acids, proteins, or antibodies can be employed. Such expression vectors include, for example, pET, pET3d, pCR2.1, pBAD, pUC, and yeast vectors. The vectors can be used, for example, in a variety of in vivo and in vitro situations.

The expression cassette, expression vector, and sequences in the cassette or vector can be heterologous. As used herein, the term “heterologous” when used in reference to an expression cassette, expression vector, regulatory sequence, promoter, or nucleic acid refers to an expression cassette, expression vector, regulatory sequence, or nucleic acid that has been manipulated in some way. For example, a heterologous promoter can be a promoter that is not naturally linked to a nucleic acid of interest, or that has been introduced into cells by cell transformation procedures. A heterologous nucleic acid or promoter also includes a nucleic acid or promoter that is native to an organism but that has been altered in some way (e.g., placed in a different chromosomal location, mutated, added in multiple copies, linked to a non-native promoter or enhancer sequence, etc.). Heterologous nucleic acids may comprise sequences that comprise cDNA forms. Heterologous coding regions can be distinguished from endogenous coding regions, for example, when the heterologous coding regions are joined to nucleotide sequences comprising regulatory elements such as promoters that are not found naturally associated with the coding region, or when the heterologous coding regions are associated with portions of a chromosome not found in nature (e.g., genes expressed in loci where the protein encoded by the coding region is not normally expressed). Similarly, heterologous promoters can be promoters that at linked to a coding region to which they are not linked in nature.

Viral vectors that can be employed include those relating to lentivirus, adenovirus, adeno-associated virus, herpes virus, vaccinia virus, polio virus, AIDS virus, neuronal trophic virus, Sindbis and other viruses. Also useful are any viral families which share the properties of these viruses which make them suitable for use as vectors. Retroviral vectors that can be employed include those described in by Verma, I. M., Retroviral vectors for gene transfer. In Microbiology-1985, American Society for Microbiology, pp. 229-232, Washington, (1985). For example, such retroviral vectors can include Murine Maloney Leukemia virus, MMLV, and other retroviruses that express desirable properties. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promoter cassette is inserted into the viral genome in place of the removed viral nucleic acid.

A variety of regulatory elements can be included in the expression cassettes and/or expression vectors, including promoters, enhancers, translational initiation sequences, transcription termination sequences and other elements. A “promoter” is generally a sequence or sequences of DNA that function when in a relatively fixed location in regard to the transcription start site. For example, the promoter can be upstream of the nucleic acid segment encoding one or more hyaluronidase, inhibitory nucleic acid, protein, or antibody.

A “promoter” contains core elements required for basic interaction of RNA polymerase and transcription factors and can contain upstream elements and response elements. “Enhancer” generally refers to a sequence of DNA that functions at no fixed distance from the transcription start site and can be either 5′ or 3′ to the transcription unit. Furthermore, enhancers can be within an intron as well as within the coding sequence itself. They are usually between 10 and 300 by in length, and they function in cis. Enhancers function to increase transcription from nearby promoters. Enhancers, like promoters, also often contain response elements that mediate the regulation of transcription. Enhancers often determine the regulation of expression.

Expression vectors used in eukaryotic host cells (yeast, fungi, insect, plant, animal, human or nucleated cells) can also contain sequences for the termination of transcription, which can affect mRNA expression. These regions are transcribed as polyadenylated segments in the untranslated portion of the mRNA encoding tissue factor protein. The 3′ untranslated regions also include transcription termination sites. The transcription unit may also contain a polyadenylation region. One benefit of this region is that it increases the likelihood that the transcribed unit will be processed and transported like mRNA. The identification and use of polyadenylation signals in expression constructs is well established. Homologous polyadenylation signals may be used in the transgene constructs.

The expression of one or more type of hyaluronidase, inhibitory nucleic acid, protein, or antibody from an expression cassette or expression vector can be controlled by any promoter capable of expression in prokaryotic cells or eukaryotic cells. Examples of prokaryotic promoters that can be used include, but are not limited to, SP6, T7, T5, tac, bla, trp, gal, lac, or maltose promoters. Examples of eukaryotic promoters that can be used include, but are not limited to, constitutive promoters, e.g., viral promoters such as CMV, SV40 and RSV promoters, as well as regulatable promoters, e.g., an inducible or repressible promoter such as the tet promoter, the hsp70 promoter and a synthetic promoter regulated by CRE. Vectors for bacterial expression include pGEX-5X-3, and for eukaryotic expression include pCIneo-CMV.

The expression cassette or vector can include nucleic acid sequence encoding a marker product. This marker product is used to determine if a vector or expression cassette encoding one or more hyaluronidase, inhibitory nucleic acid, protein, or antibody have been delivered to the cell and once delivered, is being expressed. Marker genes can include the E. coli lacZ gene which encodes β-galactosidase, and green fluorescent protein. In some embodiments the marker can be a selectable marker. When such selectable markers are successfully transferred into a host cell, the transformed host cell can survive if placed under selective pressure. There are two widely used distinct categories of selective regimes. The first category is based on a cell's metabolism and the use of a mutant cell line which lacks the ability to grow independent of a supplemented media. The second category is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin (Southern P. and Berg, P., J. Molec. Appl. Genet. 1:327 (1982)), mycophenolic acid, (Mulligan, R. C. and Berg, P. Science 209:1422 (1980)) or hygromycin, (Sugden, B. et al., Mol. Cell. Biol. 5:410-413 (1985)).

Gene transfer can be obtained using direct transfer of genetic material, in but not limited to, plasmids, viral vectors, viral nucleic acids, phage nucleic acids, phages, cosmids, and artificial chromosomes, or via transfer of genetic material in cells or carriers such as cationic liposomes. Such methods are available in the art and readily adaptable for use in the method described herein. Transfer vectors can be any nucleotide construction used to deliver genes into cells (e.g., a plasmid), or as part of a general strategy to deliver genes, e.g., as part of recombinant retrovirus or adenovirus (Ram et al. Cancer Res. 53:83-88, (1993)). Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991).

For example, the nucleic acid molecules, expression cassette and/or vectors encoding one or more hyaluronidase, inhibitory nucleic acid, protein, or antibody can be introduced to a cell by any method including, but not limited to, calcium-mediated transformation, electroporation, microinjection, lipofection, particle bombardment and the like. The cells can also be expanded in culture and then administered to a subject, e.g. a mammal such as a human. The amount or number of cells administered can vary but amounts in the range of about 106 to about 109 cells can be used. The cells are generally delivered in a physiological solution such as saline or buffered saline. The cells can also be delivered in a vehicle such as a population of liposomes, exosomes or microvesicles.

In some cases, the transgenic cell can produce exosomes or microvesicles that contain nucleic acid molecules, expression cassettes and/or vectors expressing hyaluronidases, inhibitory nucleic acids, proteins, antibodies, or a combination thereof. In some cases, the transgenic cell can produce exosomes or microvesicles that contain nucleic acid molecules that encode one or more hyaluronidases, inhibitory nucleic acids, proteins, or antibodies to particular tissues. Microvesicles can mediate the secretion of a wide variety of proteins (e.g., hyaluronidase), lipids, mRNAs, and micro RNAs, interact with neighboring cells, and can thereby transmit signals, proteins (e.g., hyaluronidase), lipids, and nucleic acids from cell to cell (see, e.g., Shen et al., J Biol Chem. 286 (16): 14383-14395 (2011); Hu et al., Frontiers in Genetics 3 (April 2012); Pegtel et al., Proc. Nat'l Acad Sci 107 (14): 6328-6333 (2010); WO/2013/084000; each of which is incorporated herein by reference in its entirety. Cells producing such microvesicles can be used to provide one or more hyaluronidase, inhibitory nucleic acid, protein, or antibody.

Transgenic vectors or cells with a heterologous expression cassette or expression vector can express one or more hyaluronidase, inhibitory nucleic acid, protein, or antibody. Any of these vectors or cells can be administered to a subject. Exosomes produced by transgenic cells can also be used to administer one or more hyaluronidase, inhibitory nucleic acid, protein, or antibody to the subject.

### Compositions

The invention also relates to compositions containing PARP1 inhibitors and activated stromal/activated cancer-associated fibroblast inhibitors or inhibitors of TNFα, IL-6 or JAK. Such inhibitors can be small molecules, antibodies, polypeptides, inhibitory nucleic acids, nucleic acids encoding inhibitory nucleic acids, nucleic acids encoding a polypeptide (e.g., within an expression cassette or expression vector), or a combination thereof. The compositions can be pharmaceutical compositions. In some embodiments, the compositions can include a pharmaceutically acceptable carrier. By “pharmaceutically acceptable” it is meant that a carrier, diluent, excipient, and/or salt is compatible with the other ingredients of the formulation, and not deleterious to the recipient thereof.

The composition can be formulated in any convenient form. In some embodiments, the therapeutic agents of the invention (e.g., small molecules, inhibitory nucleic acids, nucleic acids encoding inhibitory nucleic acids, polypeptides, nucleic acid encoding polypeptides (e.g., within an expression cassette or expression vector), a compound identified by a method described herein, or a combination thereof), are administered in a “therapeutically effective amount.” Such a therapeutically effective amount is an amount sufficient to obtain the desired physiological effect, such a reduction of at least one symptom of cancer. For example, chemotherapeutic agents can reduce cell metastasis by 5%, or 10%, or 15%, or 20%, or 25%, or 30%, or 35%, or 40%, or 45%, or 50%, or 55%, or 60%, or 65%, or % 70, or 80%, or 90%, 095%, or 97%, or 99%, or any numerical percentage between 5% and 100%. Symptoms of cancer can also include tumor cachexia, tumor-induced pain conditions, tumor-induced fatigue, tumor growth, and metastatic spread. Hence, the chemotherapeutic agents may also reduce tumor cachexia, tumor-induced pain conditions, tumor-induced fatigue, tumor growth, or a combination thereof by 5%, or 10%, or 15%, or 20%, or 25%, or 30%, or 35%, or 40%, or 45%, or 50%, or 55%, or 60%, or 65%, or % 70, or 80%, or 90%, 095%, or 97%, or 99%, or any numerical percentage between 5% and 100%.

To achieve the desired effect(s), the therapeutic agents may be administered as single or divided dosages. For example, therapeutic agents can be administered in dosages of at least about 0.01 mg/kg to about 500 to 750 mg/kg, of at least about 0.01 mg/kg to about 300 to 500 mg/kg, at least about 0.1 mg/kg to about 100 to 300 mg/kg or at least about 1 mg/kg to about 50 to 100 mg/kg of body weight, although other dosages may provide beneficial results. The amount administered will vary depending on various factors including, but not limited to, the type of small molecules, compounds, polypeptides, antibodies, nucleic acids, or combinations thereof chosen for administration, the disease, the weight, the physical condition, the health, and the age of the mammal. Such factors can be readily determined by the clinician employing animal models or other test systems that are available in the art.

Administration of the therapeutic agents in accordance with the present invention may be in a single dose, in multiple doses, in a continuous or intermittent manner, depending, for example, upon the recipient's physiological condition, whether the purpose of the administration is therapeutic or prophylactic, and other factors known to skilled practitioners. The administration of the therapeutic agents and compositions of the invention may be essentially continuous over a preselected period of time or may be in a series of spaced doses. Both local and systemic administration is contemplated.

To prepare the composition, small molecules, compounds, polypeptides, nucleic acids, expression cassettes, and other agents are synthesized or otherwise obtained, purified as necessary or desired. These small molecules, compounds, polypeptides, antibodies, nucleic acids, expression cassettes, and other agents can be suspended in a pharmaceutically acceptable carrier and/or lyophilized or otherwise stabilized. The small molecules, compounds, polypeptides, nucleic acids, expression cassettes, other agents, and combinations thereof can be adjusted to an appropriate concentration, and optionally combined with other agents. The absolute weight of a given small molecule, compound, polypeptide, antibodies, nucleic acid, and/or other agents included in a unit dose can vary widely. For example, about 0.01 to about 2 g, or about 0.1 to about 500 mg, of at least one molecule, compound, polypeptide, nucleic acid, and/or other agent, or a plurality of molecules, compounds, polypeptides, nucleic acids, and/or other agents can be administered. Alternatively, the unit dosage can vary from about 0.01 g to about 50 g, from about 0.01 g to about 35 g, from about 0.1 g to about 25 g, from about 0.5 g to about 12 g, from about 0.5 g to about 8 g, from about 0.5 g to about 4 g, or from about 0.5 g to about 2 g.

Daily doses of the chemotherapeutic agents of the invention can vary as well. Such daily doses can range, for example, from about 0.1 g/day to about 50 g/day, from about 0.1 g/day to about 25 g/day, from about 0.1 g/day to about 12 g/day, from about 0.5 g/day to about 8 g/day, from about 0.5 g/day to about 4 g/day, and from about 0.5 g/day to about 2 g/day.

It will be appreciated that the amount of therapeutic agent for use in treatment will vary not only with the particular carrier selected but also with the route of administration, the nature of the cancer condition being treated and the age and condition of the patient. Ultimately the attendant health care provider can determine proper dosage. In addition, a pharmaceutical composition can be formulated as a single unit dosage form.

Thus, one or more suitable unit dosage forms comprising the therapeutic agent(s) can be administered by a variety of routes including parenteral (including subcutaneous, intravenous, intramuscular and intraperitoneal), oral, rectal, dermal, transdermal, intrathoracic, intrapulmonary and intranasal (respiratory) routes. The therapeutic agent(s) may also be formulated for sustained release (for example, using microencapsulation, see WO 94/07529, and U.S. Pat. No. 4,962,091). The formulations may, where appropriate, be conveniently presented in discrete unit dosage forms and may be prepared by any of the methods well known to the pharmaceutical arts. Such methods may include the step of mixing the therapeutic agent with liquid carriers, solid matrices, semi-solid carriers, finely divided solid carriers or combinations thereof, and then, if necessary, introducing or shaping the product into the desired delivery system. For example, the therapeutic agent(s) can be linked to a convenient carrier such as a nanoparticle, albumin, polyalkylene glycol, or be supplied in prodrug form. The therapeutic agent(s), and combinations thereof can be combined with a carrier and/or encapsulated in a vesicle such as a liposome.

The compositions of the invention may be prepared in many forms that include aqueous solutions, suspensions, tablets, hard or soft gelatin capsules, and liposomes and other slow-release formulations, such as shaped polymeric gels. Administration of inhibitors can also involve parenteral or local administration of the in an aqueous solution or sustained release vehicle.

Thus, while the therapeutic agent(s) and/or other agents can sometimes be administered in an oral dosage form, that oral dosage form can be formulated so as to protect the small molecules, compounds, polypeptides, nucleic acids, expression cassettes, and combinations thereof from degradation or breakdown before the small molecules, compounds, antibodies, polypeptides, inhibitory nucleic acids, nucleic acids encoding such polypeptides, and combinations thereof provide therapeutic utility. For example, in some cases the small molecules, compounds, inhibitory nucleic acids, polypeptides, nucleic acids encoding such polypeptide, antibodies, and/or other agents can be formulated for release into the intestine after passing through the stomach. Such formulations are described, for example, in U.S. Pat. No. 6,306,434 and in the references contained therein.

Liquid pharmaceutical compositions may be in the form of, for example, aqueous or oily suspensions, solutions, emulsions, syrups or elixirs, dry powders for constitution with water or other suitable vehicle before use. Such liquid pharmaceutical compositions may contain conventional additives such as suspending agents, emulsifying agents, non-aqueous vehicles (which may include edible oils), or preservatives. The pharmaceutical compositions may take such forms as suspensions, solutions, or emulsions in oily or aqueous vehicles, and may contain formulatory agents such as suspending, stabilizing and/or dispersing agents. Suitable carriers include saline solution, encapsulating agents (e.g., liposomes), and other materials. The chemotherapeutic agent(s) and/or other agents can be formulated in dry form (e.g., in freeze-dried form), in the presence or absence of a carrier. If a carrier is desired, the carrier can be included in the pharmaceutical formulation, or can be separately packaged in a separate container, for addition to the inhibitor that is packaged in dry form, in suspension or in soluble concentrated form in a convenient liquid.

Therapeutic agent(s) and/or other agents can be formulated for parenteral administration (e.g., by injection, for example, bolus injection or continuous infusion) and may be presented in unit dosage form in ampoules, prefilled syringes, small volume infusion containers or multi-dose containers with an added preservative.

The compositions can also contain other ingredients such as chemotherapeutic agents, anti-viral agents, antibacterial agents, antimicrobial agents and/or preservatives. Examples of additional therapeutic agents that may be used include, but are not limited to: alkylating agents, such as nitrogen mustards, alkyl sulfonates, nitrosoureas, ethylenimines, and triazenes; antimetabolites, such as folate antagonists, purine analogues, and pyrimidine analogues; antibiotics, such as anthracyclines, bleomycins, mitomycin, dactinomycin, and plicamycin; enzymes, such as L-asparaginase; farnesyl-protein transferase inhibitors; hormonal agents, such as glucocorticoids, estrogens/antiestrogens, androgens/antiandrogens, progestins, and luteinizing hormone-releasing hormone anatagonists, octreotide acetate; microtubule-disruptor agents, such as ecteinascidins or their analogs and derivatives; microtubule-stabilizing agents such as paclitaxel (Taxol®), docetaxel (Taxotere®), and epothilones A-F or their analogs or derivatives; plant-derived products, such as vinca alkaloids, epipodophyllotoxins, taxanes; and topoisomerase inhibitors; prenyl-protein transferase inhibitors; and miscellaneous agents such as, hydroxyurea, procarbazine, mitotane, hexamethylmelamine, platinum coordination complexes such as cisplatin and carboplatin; and other agents used as anti-cancer and cytotoxic agents such as biological response modifiers, growth factors; immune modulators, and monoclonal antibodies. The compositions can also be used in conjunction with radiation therapy.

### Treatment

The one or more PARP1 inhibitors and inhibitors of activated stromal/activated cancer-associated fibroblasts, TNFα, IL-6 or JAK are useful for selectively targeting tumors or treating cancers.

“Treatment” or “treating” refers to both therapeutic treatment, and prophylactic or preventative measures. Those in need of treatment include those already with the disorder as well as those prone to have the disorder, or those in whom the disorder is to be prevented.

“Subject” for purposes of treatment refers to any animal classified as a mammal or bird, including humans, domestic and farm animals, and zoo, sports, or pet animals, such as dogs, horses, cats, cows, etc. In one embodiment, the subject is human.

As used herein, the term “cancer” includes solid animal tumors as well as hematological malignancies. The terms “tumor cell(s)” and “cancer cell(s)” are used interchangeably herein.

“Solid animal tumors” include cancers of the head and neck, lung, mesothelioma, mediastinum, lung, esophagus, stomach, pancreas, hepatobiliary system, small intestine, colon, colorectal, rectum, anus, kidney, urethra, bladder, prostate, urethra, penis, testis, gynecological organs, ovaries, breast, endocrine system, skin central nervous system; sarcomas of the soft tissue and bone; and melanoma of cutaneous and intraocular origin. In addition, a metastatic cancer at any stage of progression can be treated, such as micrometastatic tumors, megametastatic tumors, and recurrent cancers.

The term “hematological malignancies” includes adult or childhood leukemia and lymphomas, Hodgkin's disease, lymphomas of lymphocytic and cutaneous origin, acute and chronic leukemia, plasma cell neoplasm and cancers associated with AIDS.

The inventive methods and compositions can also be used to treat cancer of the breast, cancer of the lung, cancer of the adrenal cortex, cancer of the cervix, cancer of the endometrium, cancer of the esophagus, cancer of the head and neck, cancer of the liver, cancer of the pancreas, cancer of the prostate, cancer of the thymus, carcinoid tumors, chronic lymphocytic leukemia, Ewing's sarcoma, gestational trophoblastic tumors, hepatoblastoma, multiple myeloma, non-small cell lung cancer, retinoblastoma, or tumors in the ovaries. A cancer at any stage of progression can be treated or detected, such as primary, metastatic, and recurrent cancers. In some cases, metastatic cancers are treated but primary cancers are not treated. Information regarding numerous types of cancer can be found, e.g., from the American Cancer Society (cancer.org), or from, e.g., Wilson et al. (1991) Harrison's Principles of Internal Medicine, 12th Edition, McGraw-Hill, Inc.

In some embodiments, the cancer and/or tumors to be treated are prostate cancers, lung cancers, or breast cancers.

Treatment of, or treating, metastatic cancer can include the reduction in cancer cell migration or the reduction in establishment of at least one metastatic tumor. The treatment also includes alleviation or diminishment of more than one symptom of metastatic cancer such as coughing, shortness of breath, hemoptysis, lymphadenopathy, enlarged liver, nausea, jaundice, bone pain, bone fractures, headaches, seizures, systemic pain and combinations thereof. The treatment may cure the cancer, e.g., it may prevent metastatic cancer, it may substantially eliminate metastatic tumor formation and growth, and/or it may arrest or inhibit the migration of metastatic cancer cells.

Anti-cancer activity can reduce the progression of a variety of cancers (e.g., breast, lung, or prostate cancer) using methods available to one of skill in the art. Anti-cancer activity, for example, can determined by identifying the lethal dose (LD100) or the 50% effective dose (ED50) or the dose that inhibits growth at 50% (GI50) of an agent of the present invention that prevents the migration of cancer cells. In one aspect, anti-cancer activity is the amount of the agent that reduces 50%, 60%, 70%, 80%, 90%, 95%, 97%, 98%, 99% or 100% of cancer cell migration, for example, when measured by detecting expression of a cancer cell marker at sites proximal or distal from a primary tumor site, or when assessed using available methods for detecting metastases.

In another example, PARP1 inhibitors and one or more inhibitors of activated stromal/activated cancer-associated fibroblasts, TNFα, IL-6 or JAK can be administered to sensitize tumor cells to immune therapies. By administering PARP1 inhibitors and one or more inhibitors of activated stromal/activated cancer-associated fibroblasts, TNFα, IL-6 or JAK, tumor cells can become more sensitive to the immune system and to various immune therapies.

The invention will be further described by the following non-limiting examples.

### Example 1

Reduced p62 levels are associated with the induction of the cancer-associated fibroblast (CAF) phenotype, which promotes tumorigenesis in vitro and in vivo through inflammation and metabolic reprogramming. However, how p62 is downregulated in the stroma fibroblasts by tumor cells to drive CAF activation is an unresolved central issue in the field. As disclosed herein, tumor-secreted lactate downregulate p62 transcriptionally through a mechanism involving reduction of the NAD+/NADH ratio, which impaired poly(ADP-Ribose)-polymerase 1 (PARP1) activity. PARP1 inhibition blocked the poly(ADP-ribosyl)ation of the AP1 transcription factors, c-FOS and c-JUN, which is an obligate step for p62 downregulation. Importantly, restoring p62 levels in CAFs by NAD⁺ rendered CAFs less active. PARP1 inhibitors, such as Olaparib, mimicked lactate in the reduction of stromal p62 levels, as well as the subsequent stromal activation both in vitro and in vivo, which suggests that therapies utilizing Olaparib would benefit from strategies aimed at inhibiting CAF activity.

The mechanisms whereby cancer cells promote the acquisition of the pro-tumorigenic CAF phenotype was investigated. As disclosed herein, the lactate secreted by tumor cells downregulates p62 at the transcriptional level to induce stromal activation. Lactate caused a reduction in the NAD⁺/NADH ratio that impaired Poly (ADP-ribose) polymerase 1 (PARP1) activity. The results demonstrate that NAD⁺ supplementation restored p62 levels and impaired CAF activation established the functional relevance of the lactate-PARP1 axis for p62 downregulation and the generation of a reactive stroma conducive to malignancy. Furthermore, the fact that, PARP1 inhibitors used in the clinic, such as Olaparib, mimicked the effect of lactate in downregulating p62 and promoting CAF activity, reveals an unanticipated potential weakness of therapies based on PARP1 inhibitors, and suggest that reprogramming the stroma would enhance the anti-cancer effects of Olaparib.

### Experimental Model and Subject Details

### Mice

Animal handling and experimental procedures conformed to institutional guidelines and were approved by the Sanford-Burnham-Prebys Medical Discovery Institute Institutional Animal Care and Use Committee, and by the Weill Cornell Medicine Institutional Animal Care and Use Committee. For Olaparib treatment experiments, 13 week-old male TRAMP⁺ mice (C57BL/6-Tg (TRAMP) 8247Ng/J, stock No: 003135) were purchased from The Jackson Laboratory, (Bar Harbor, ME, USA). TRAMP⁺ mice were generated in a C57BL/6 background and were born and maintained under pathogen-free conditions. All genotyping was done by PCR. Age-matched mice were used for all experiments. For xenograft experiments, 7-week-old male JAX NSG mice (572NCG) were purchased from Charles River Labs (Wilmington, MA, USA). NSG mice were purchased and maintained under pathogen-free conditions. All mice were maintained on food and water ad libitum and were age-matched and co-housed for all experiments. Mice were sacrificed and prostate, tumors or other organs were collected for analysis.

### Cell Lines

WPMY-1 (sex: male), mNAF (sex: male), mHSC (sex: male), hNAF (sex: male), PC3 (sex: male), DU145 (sex: male), TRAMPC2 (sex: male), PrEC (sex: male), RWPE1 (sex: male), LNCAP (sex: male), HEK293T (sex: female) and Phoenix-GP (sex: female) cell lines were purchased from ATCC. WPMY-1, mNAF, mHSC, PC3, DU145, TRAMPC2, HEK293T and Phoenix-GP were cultured in Dulbecco's Modified Eagles Medium (DMEM, Corning). LNCAP, PrEC and RWPEI cells were cultured in Roswell Park Memorial Institute Medium (RPMI, Corning), hNAF cells were cultured in Fibroblast media (FM, ScienceCell Research Laboratories). All base mediums were supplemented with 10% fetal bovine serum (FBS), 2 mM glutamine in an atmosphere of 95% air and 5% CO2. Androgen Deprivation Therapy conditions, cells were culture in RPMI media without phenol red (GIBCO) supplemented with 10% charcoal stripped FBS (F6765, Sigma), Glutamax and 100 U/mL penicillin 100 and 100 μg/mL streptomycin. Cultures were tested weekly for mycoplasma contamination.

### Method Details

### Xenograft Experiments

For mouse xenografts using PC3 PCa cells and sgC or sgAP-1 WPMY-1 stromal cells. 7 weeks old NSG mice were surgical castrated and let androgen levels drop for 10 days. Cells were trypsinized washed two times in PBS and resuspend in DMEN. 1×10⁶ PC3 and 1×10⁶ sgC or sgAP1-A WPMY-1 for each mouse were resuspended in 100 μl DMEM: Matrigel (1:1) and injected subcutaneously into both flanks of immunocompromised NSG mice (PC3+sgC WPMY-1 n=4; PC3+sgAP-1 WPMY-1 n=4). Tumors were allowed to grow for 1 month. Mice were euthanized and tumors were collected and analyzed histologically. For Olaparib treatment in mouse xenografts. PC3 PCa cells and WPMY-1 stromal cells were trypsinized washed two times in PBS and resuspend in DMEN. 1×10⁶ PC3 and 1×10⁶ WPMY-1 for each mouse were resuspended in 100 μl DMEM: Matrigel (1:1) and injected subcutaneously into both flanks of immunocompromised NSG mice. Tumors were allowed to grow for 14 days, and mouse were randomly divided to receive vehicle, n=10, Olaparib (40 mg/kg, 2 days each week, i.p.), n=10 or Olaparib (40 mg/kg, 2 days each week, i.p.)+_PEGPH20 (0.0375 mg/Kg, 2 days each week, retroorbital injection) n=10. Tumors were measured twice a week. Mice were euthanized 14 days after the initiation of the treatment and tumors were collected and analyzed histologically. For Olaparib treatment in mice, 13 weeks-old male TRAMP⁺, n=18, were randomly distributed to receive vehicle, n=9, or Olaparib (40 mg/kg/, 5 days each week, i.p.), n=9. Mice were euthanized 1 month after the initiation of the treatment. Prostate and other organs were collected and analyzed histologically.

### Cell Culture Experiments

To simulate the amounts of lactate that accumulates in the tumor microenvironment as consequence of the Warburg effect, WPMY-1 cells were incubated in culture medium with or without lactic acidosis (24 mM of Lactic acid buffered to pH 6.7-6.8 with NaOH) for 48 hours. To avoid fluctuations in extracellular pH during the Lactate treatment, WPMY-1 cells were grown in a bicarbonate-free DMEM with 30 mM of HEPES in absence of CO2. To knock out PARP-1 in WPMY-1 cells, single-guide RNA sequences targeting PARP-1 exon2 were purchased from Synthego and transduced into WPMY-1 cells with recombinant Streptococcus pyogenes Cas9 protein (Truecut Cas9 Protein v2, Thermo), using the Neon Transfection System 1 (Invitrogen) following the manufacturer's protocol and single clones were expanded and screened by protein immunoblotting. To perform AP1-A binding site editing in the SQSTM promoter in WPMY-1 cells, single-guide RNA sequences targeting the human AP1-A binding site (Synthego) was transduced into cells with a Cas9 protein and a single-stranded donor oligonucleotide (ssODN, IDT) using Neon Electroporation System. Single clones were expanded and screened for AP1-A editing by Sanger sequencing. A point mutation to disrupt the API enhancer element in the p62 promoter was introduced by Site Directed Mutagenesis (Stratagene). Knockdown of MCT1, MCT4, FOS, PARP1, PARP2, CD38, ATG5, and SIRT1-7 genes in WPMY-1 cells were achieved by siRNA transfection using Lipofectamine RNAiMAX Transfection Reagent (Invitrogen). Transient overexpression was achieved by transfection using X-tremeGENE HP transfection reagent (Roche). Transfected cells were examined 48 hours after transfection. Establishing stably GFP or RFP expressing cells was achieved by lentivirus-mediated transduction. Lentivirus were produced in HEK293T cells using X-tremeGENE HP transfection reagent (Roche). Virus-containing supernatants were collected 48, 72 and 96 hours after transfection, filtered to eliminate cells, and supplemented with 8 μg/ml polybrene. Cells were infected with three rounds of viral supernatants and selected with puromycin (3 μg/ml). For autophagy or proteasome inhibition, WPMY-1 cells were treated for 12 hours with 100 nM bafilomycin Al or 12 hours with 20 μM of MG132, respectively, or vehicle (DMSO). For PARP1 inhibition, WPMY-1 cells were treated with PJ34 (2 days) or Olaparib (4 days) at the doses indicated in each experiment. For MCT1 inhibition, WPMY-1 cells were treated for 48 hours with 10 μM of AZD3965. For cJUN inhibition, WPMY-1 cells were treated for 48 hours with 10 μM of SP600125. For calpains and caspases inhibition, WPMY-1 cells were treated for 48 hours with 1-20 μM of Calpeptin (Tocris) or z-VAD-FMK (Tocris), respectively. For protein synthesis inhibition, WPMY-1 cells were treated with 50 μg/ml of cycloheximide (Sigma). Conditioned media (CM) was generated by collecting supernatant on day 3. CM was transferred to a 15 ml BD Falcon tube and centrifuged at 1300 rpm for 10 minutes. The supernatant was sterile filtered using a 22 mm filter (Millex-GV) with a 10 ml syringe barrel. The samples were stored at 20° C. for future experiments. Fractionation of the CM was achieved using Amicon Ultra centrifugal filters (3KUltracel, Millipore). The supernatant was centrifuged at 4000 rpm for 1 hour. The supernatant fraction that was >3 kDa remained above the filter and that which was <3 kDa passed through to the lower chamber. The >3-kDa fraction was resuspended in DMEM to the pre-filtration volume.

### Organotypic Cultures

Organotypic cultures were performed as described previously (Valencia et al., 2014). Briefly, gels were composed of one ml of a mixture of 1.75 volumes of Matrigel, 5.25 volumes of collagen type I, 1 volume of 1×DMEM, 1 volume of 10× DMEM, and 1 volume of filtered FBS. The mixture was plated onto 24-well plates coated with diluted collagen type I. Gels were allowed to equilibrate with 1 ml of 1× DMEM overnight at 37° C. 5×105 cells PCa cells and prostate stromal cells (50:50) were then seeded on top of the matrix. Gel rafts were placed onto collagen-coated nylon sheets and lifted using a sterile supporting steel mesh to set up a raised air-liquid culture. Normal medium was changed in alternate days and organotypic cultures were allowed to grow for 14 days. Afterwards, organotypic gels were harvested, fixed in 10% neutral buffered formalin, bisected, and embedded in paraffin. H&E-stained sections were analyzed with a Zeiss light microscope supplemented with Axiovision40 software. Quantification of the invasion assays was performed as described previously (Valencia et al., 2014) using ImageProPlus software.

### Migration and Invasion Assay

For co-culture migration and invasion assays, 8×10⁴ WPMY-1 cells were plated onto the lower chamber of a 24 well plate in DMEN containing 10% FBS (basal conditions) or 10% charcoal stripped FBS (ADT conditions) and vehicle (DMSO) or Olaparib 10 μM or Olaparib 10 μM+PEGPH20 2.5 μg/ml for 48 hours. 5×10⁴ PC3 were seeded in a transwell chamber (Corning Biocoat control inserts) or in a transwell invasion chamber (Corning BioCoat Matrigel Invasion Chambers), both with 8 μm membrane. PC3 were allowed to migrate or invade for 20 hours at 37° C., 5% CO₂. Cells were fixed in cold methanol and stained with crystal violet.

### Proliferation Assay

WPMY-1 cells were cultured in DMEN containing 10% FBS (basal conditions) or 10% charcoal stripped FBS (ADT conditions), then the cells were treated with vehicle (DMSO), Olaparib 10 μM, Olaparib 10 μM+PEGPH20 2.5 μg/ml for 96 hours, refreshing media and stimuli at 48 hours, conditioned media (CM) was generated by collecting supernatant on day 4. PC3 cells were seeded in a black-walled well (Thermo Fisher) and serum starved overnight. CM from WPMY-1 cells was added to PC3 for 24 h. PC3 were exposed to EdU incorporation for the last 2 hours. Proliferating cells were detected using Click-iT EdU Alexa fluor 488 (Thermo Fisher) following the manufacturer's instructions. Pictures were taken using EVOS M5000 Imaging System and EdU positive were measured using Fiji (Schindelin et al., 2012).

### Co-Culture Assays

Co-culture assays were performed as described previously (Valencia et al., 2014). Briefly, stromal cells were seeded in TC-treated 6-well plates and allowed to attach for 6 hours before PCa cells were seeded on top of Milicell Cell Culture Inserts (Millipore). Inserts were then placed in the pre-seeded 6-well plate. For co-culture experiments of PC3-GFP label with WPMY-1 cells, 500 PC3 and 1000 WPMY-1 were seeded in a 96 well plate in DMEN containing 10% FBS (basal conditions) or 10% charcoal stripped FBS (ADT conditions) and vehicle (DMSO) or Olaparib 5 μM for 10 days. Media and treatments were refresh every 48 hours. Pictures were taken using EVOS M5000 Imaging System and GFP positive were measured using Fiji (Schindelin et al., 2012).

### Isotopic Labeling

WPMY-1 cells were cultured in DMEM (Cat. #5030, Sigma) medium supplemented with 10% FBS, 4 mM glutamine, 25 mM glucose, 30 mM HEPES, 10% FBS, 100 U/ml penicillin, and 100 μg/ml streptomycin in a humidified cell culture incubator at 37° C. with no CO₂. For isotopic tracing, cells were cultured for 48 hours in growth medium, medium was then changed to growth medium containing 10 mM [3-13C]lactate (Cat. #CLM-1578, Cambridge Isotopes) or 10 mM [2-2H]lactate (Cat. #693987, Sigma). Cells were cultured in tracer media for 24 hours. All media was adjusted to pH=6.8.

### Gas Chromatography-Mass Spectrometry (GC/MS)

Metabolites were extracted using a modified Bligh and Dyer method and analyzed as previously described in detail (Linares et al., 2017). Briefly, intracellular metabolites were extracted with 0.25 ml-20° C. methanol, 0.1 ml 4° C. cold water, and 0.25 ml-20° C. chloroform. The extracts were vortexed for 10 minutes at 4° C. and centrifuged at 16,000×g for 5 minutes at 4° C. The upper aqueous phase was evaporated under vacuum at −4° C., the lower organic phase under airflow. To determine labeling on lactate in cell culture media, 10 μl of medium was extracted with 90 μl of extraction buffer consisting of 8 parts (v/v) methanol and 1 part (v/v) water, centrifuged at 16,000×g for 5 minutes at 4° C., and 60 μl was dried under vacuum. Derivatization for polar metabolites was performed using a Gerstel MPS with 15 μl of 2% (w/v) methoxyamine hydrochloride (Thermo Scientific) in pyridine (incubated for 60 minutes at 45° C.) and 15 μl N-tertbutyldimethylsilyl-N-methyltrifluoroacetamide (MTBSTFA) with 1% tert-butyldimethylchlorosilane (Regis Technologies) (incubated further for 30 minutes at 45° C.). Polar derivatives were analyzed by GC-MS using a DB-35MSUI column (30 m×0.25 i.d.×0.25 μm) installed in an Agilent 7890B gas chromatograph (GC) interfaced with an Agilent 5977B mass spectrometer (MS) operating under electron impact ionization at 70 eV. The MS source was held at 230° C., the quadrupole at 150° C., helium was used as carrier gas and the GC oven was held at 100° C. for 1 minute, increased to 300° C. at 10° C./minute, and held at 325° C. for 3 minutes. Intracellular labeling on metabolites (corrected for natural abundance using in-house software) is depicted as 1-MO. YSI (yellow springs instrument) was used to quantify the concentration of media lactate and GC/MS to determine lactate labeling in cell culture media at 0 hours and after 24 hours of culture.

### Measurement of NAD Levels

Quantification was carried out using the NAD/NADH quantification colorimetric kit (Cat. No. MAK037, Sigma) according to the manufacturer's protocol. At least three independent measurements were carried out.

### Luciferase Assay

Subconfluent cultures were transfected with Lipofectamine Plus (Invitrogen) with 100 ng of a SQSTM1-luciferase reporter gene plasmid and 2 ng of the Renilla control reporter pRL-CMV (Promega). After 48 hours, cells were incubated with conditioned medium for 48 hours. The level of promoter activity was evaluated by determining the firefly luciferase activity relative to Renilla luciferase activity using the Dual Luciferase Assay System (Promega) according to the manufacturer's instruction.

### Immunofluorescence Analysis

WPMY-1 cells were seeded in coverslides and cultured in DMEN containing 10% FBS (basal conditions) or 10% charcoal stripped FBS (ADT conditions), then the cells were treated with vehicle (DMSO), Olaparib 10 μM, Olaparib 10 μM+PEGPH20 2.5 μg/ml for 96 hours, refreshing media and stimuli at 48 hours. Cells were fixed in PFA 4% for 20 minutes at room temperature, permeabilize and blocked following abcam's immunofluorescence protocol. Then, cells were incubated overnight with Hyaluronic acid binding protein (HABP) at 4° C. Cells were incubated with Alexa conjugated streptavidin and mounted in slides with mowiol. Pictures were taken by Zeiss LSM 710 NLO Confocal Microscope.

### Histological Analysis

Tissues from indicated mice were isolated, fixed in zinc buffered formalin overnight at 4° C., dehydrated, and embedded in paraffin. Sections (5 μm) were stained with hematoxylin and eosin (H&E). For immunohistochemistry, sections were deparaffinized, rehydrated, and then treated for antigen retrieval. After blocking in Protein Block Serum-Free solutions (DAKO), tissues were incubated with primary antibody overnight at 4° C. followed by incubation with biotinylated secondary antibody. Endogenous peroxidase was quenched in 3% H₂O₂ in water for 10 minutes at room temperature. Antibodies were visualized with avidin-biotin complex (Vectastain Elite; Vector Laboratories) using diaminobenzidine as the chromogen. Stained sections were analyzed with a Zeiss light microscope supplemented with Zen 3.3 Bule edition software.

### Immunoblotting Analysis

Cells for protein analysis were lysed in RIPA buffer (20 mM Tris-HCl, 37 mM NaCl₂, 2 mM EDTA, 1% Triton-X, 10% glycerol, 0.1% SDS, and 0.5% sodium deoxycholate) with phosphatase and protease inhibitors. For immunoprecipitations, cells were lysed in IP lysis buffer (100 mM NaCl, 25 mM Tris, 1% Triton-X, 10% glycerol, with phosphatase and protease inhibitors) and immunoprecipitated with 25 μl of 50% slurry of protein Glutathione-Sepharose 4B beads (Bioworld). Immunoprecipitates were washed three times with lysis buffer, once with high salt (500 mM NaCl), and once more with lysis buffer. Protein concentration in lysates was determined by using Protein Assay Kit (Bio-Rad). Cell extracts and immunoprecipitated proteins were denatured, subjected to SDS-PAGE, transferred to PVDF membranes (GE Healthcare). After blocking with 5% nonfat dry milk in Tris-buffered saline and 0.1% Tween (TBS-T), the membranes were incubated with the specific antibodies (as listed in Key Resources Table) overnight at 4° C. After 2 hours incubation with the appropriate horseradish peroxidase-conjugated antibodies, the immune complexes were detected by chemiluminescence (Thermo Scientific) or Near-infrared fluorescence (LI-COR).

### RNA Extraction and Analysis

Total RNA from mouse tissues, cells and cultured organoids was extracted using the TRIZOL reagent (Invitrogen) and the RNeasy Mini Kit (QIAGEN), followed by DNase treatment. After quantification using a Nanodrop 1000 spectrophotometer (Thermo Scientific), RNA was either processed for RNA-seq or reverse-transcribed using random primers and MultiScribe Reverse Transcriptase (Applied Biosystems). Gene expression was analyzed by amplifying 20 ng of the complementary DNA using the CFX96 Real Time PCR Detection System with SYBR Green Master Mix (BioRad) and primers described in Table S1. The amplification parameters were set at 95° C. for 30 seconds, 58° C. for 30 seconds, and 72° C. for 30 seconds (40 cycles total). Gene expression values for each sample were normalized to the 18s RNA.

### Chromatin Immunoprecipitation Analysis

WPMY-1 cells were fixed by adding directly to the culture medium formaldehyde (HCHO; from a 37% HCHO-10% methanol stock, Calbiochem) to a final concentration of 1%. After, 20 minutes of incubation with 125 mM glycine, cells were washed with ice-cold phosphate-buffered saline (PBS) and lysed with in 50 mM Tris pH 8.0, 10 mM EDTA, 1% SDS and protease inhibitors and incubated 30 minutes at 4° C. Chromatin was sheared in a COVARIS S220 Focused-ultrasonicator to yield DNA fragment sizes of 200-1000 base pairs, and diluted 10 times in dilution buffer (20 mM Tris pH 8.0, 2 mM EDTA, 1% triton X-100, 150 mM NaCl and protease inhibitors). Immunoprecipitations were carried out overnight at 4° C. using the following protein A-antibodies complexes: cFOS, FOSB, cJUN, JUNB.

Immunocomplexes were washed three times with buffer TSEI (20 mM Tris pH 8.0, 2 mM EDTA, 1% triton X-100, 150 mM NaCl, 0.1% SDS and protease inhibitors), three washes with buffer TSEII (20 mM Tris pH 8.0, 2 mM EDTA, 1% triton X-100, 500 mM NaCl, 0.1% SDS and protease inhibitors), one wash with buffer TSEIII (10 mM Tris pH 8.0, 250 mM LiCl, 1 mM EDTA, 1% NP40, 1% Deoxycholate and protease inhibitors) and one wash with TE pH 8.0.

Immunocomplexes were extracted in TE containing 1% SDS, and protein-DNA cross-links were reverted by heating at 65° C. overnight. DNA was extracted by using a PCR purification kit (Qiagen). One-tenth of the immunoprecipitated DNA was used in each PCR, for which the promoter-specific primers were used.

### RNA-seq Preparation and Sequencing

Total RNA was extracted using Quick-RNA MiniPrep kit (Zymo Research). Libraries were prepared from 200 ng of total RNA using the QuantSeq 3′ mRNA-Seq Library Prep Kit FWD for Illumina from Lexogen, and optional UMIs (Vienna, Austria). Barcoded libraries were pooled, and single end sequenced (1×75) on the Illumina NextSeq 500 using the High output V2.5 kit (Illumina Inc., San Diego CA).

### ATAC-Seq Library Preparation and Sequencing

The cell pellet was resuspended in 50 μl lysis buffer and then spun down 500×g for 10 minutes at 4° C. The nuclei pellet was resuspended into 50 μl transposition reaction mixture containing Tn5 transposase from Nextera DNA Library Prep Kit (Illumina) and incubated at 37° C. for 30 minutes. Then the transposase-associated DNA was purified using MinElute PCR purification kit (QIAGEN). To amplify the library, the DNA was first amplified for 5 cycles using indexing primer from Nextera kit and NEBNext High-Fidelity 2×PCR master mix. To reduce the PCR amplification bias, 5 μl of amplified DNA after the first 5 cycles was used to do qPCR of 20 cycles to decide the number of cycles for the second round of PCR. Usually, the maximum cycle of the second round of PCR is 5 cycles. Then the total amplified DNA was size selected to fragments less than 800 bp using SPRI beads. Quantification of the ATAC-seq library was done with QuBit. The size of the pooled library was examined by TapeStation. Barcoded ATAC-seq libraries were pooled and paired end sequenced (sX75) on the Illumina NextSeq 500 using the High output V2.5 kit (Illumina Inc., San Diego, CA).

### Prostate Tissue Digestion and 10× Library Preparation and Sequencing

Prostate lobes dissected and washed with cold PBS. Prostate tissue was gently minced with scissors and digested in Advance DMEN/F12 media (Gibco, Thermo Fisher) with 5 mg/ml Collagenase type II (Sigma) supplemented with Y27632 (Tocris) for 1 hour at 37° C. Cells were passed through a 70 μm strainer and centrifuge 5 minutes, 1000 rpm at 4° C. After that, cells were incubated with TrypLE Express (Gibco, Thermo Fisher) supplemented with Y27632 for 20 minutes in agitation at 37° C. to achieve a single cell suspension. TrypLE Express was quenched and washed with Advance DMEN/F12 media and then cells were centrifuged for 5 minutes, 1000 rpm at 4° C. Dead cells were removed by Annexin V (STEMCELL technologies). scRNA-seq libraries were generated using the Chromium Single Cell 30 Reagent Kit v2 (10× Genomics). Cells were loaded onto the 10×Chromium Single Cell Platform (10× Genomics) at a concentration of 2,000 cells per μl (Single Cell 3′ library and Gel Bead Kit v.2) as described in the manufacturer's protocol (10× User Guide, Revision B). On average, approximately 8,000 cells were loaded. Generation of gel beads in emulsion (GEMs), barcoding, GEM-RT clean-up, complementary DNA amplification and library construction were all performed as per the manufacturer's protocol. Individual sample quality was checked using a Bioanalyzer Tapestation (Agilent). Qubit was used for library quantification before pooling. The final library pool was sequenced on an Illumina NovaSeq6000 instrument using a S1 flow cell.

### Bioinformatics Analysis

For RNA-Seq, sequencing Fastq files were uploaded to BaseSpace and processed with RNASeq Alignment App (Illumina) to obtain raw reads counts for each gene. For 3′RNA-Seq, read data was processed with the BlueBee Genomics Platform (BlueBee, San Mateo, CA). GenePattern (https://genepattern.broadinstitute.org/gp/pages/index.jsf) was used to collapse gene matrix files (CollapseDataset module) or to assess the statistical significance of differential gene expression (ComparativeMarkerSelection module for microarray data and DESeq2 module for RNA-seq data). Gene Set Enrichment Analysis (GSEA) was performed using GSEA v4.1.0 software (http://www.broadinstitute.org/gsea/index.jsp) using the default parameters, with customized signatures, using the default parameters with customized signatures. Preranked GSEA analysis was performed using default parameters with MSigDB c2.all.v7.0.symbols (C2) and c5.all.v7.0.symbols (C5) collections. Briefly, differential express genes between Olaparib treated cells and vehicle were obtain using DESeq2, genes were sorted by log 2FC≥+0.5 and padj≥0.05. Ranked genes were used as input for GSEA Preranked analysis. For ATACSeq, FASTQ files from ATAC-seq reads were aligned to UCSC mm10 with Bowtie2 (bowtie2-very-sensitive-x mm10-1 FILE_merged_R1.fastq-2 FILE_merged_R2.fastq-X 1000-p 12 | samtools view-u-| samtools sort->FILE.bam). Peak calling was performed with MACS2 with a threshold of q<0.05. Peaks were annotated with ChipSeeker R package. For motif enrichment analysis in the differential peaks between vehicle and Olaparib-treated cells, p-values were calculated using findMotifsGenome.pl with—size given,—len 6,8,10,12 and—mask program of HOMER v4.10.3. (Heinz et al., 2010) within 1-2 Kb from the TSS. Preranked GSEA analysis was performed using default parameters with MSigDB c2.all.v7.0.symbols (C2) and c5.all.v7.0.symbols (C5) collections. Briefly, differential express genes between Olaparib treated cells and vehicle were obtain using DESeq2, genes were sorted by log 2FC≥+0.5 and padj≥0.05. Ranked genes were used as input for GSEA Preranked analysis. For ATACSeq, FASTQ files from ATAC-seq reads were aligned to UCSC mm10 with Bowtie2 (bowtie2-very-sensitive-x mm10-1 FILE_merged_R1.fastq-2 FILE_merged_R2.fastq-X 1000-p 12 | samtools view-u-I samtools sort->FILE.bam). Peak calling was performed with MACS2 with a threshold of q<0.05. Peaks were annotated with ChIPSeeker R package. For motif enrichment analysis in the differential peaks between vehicle and Olaparib-treated cells, p-values were calculated using findMotifsGenome.pl with -size given, -len 6,8, 10,12 and-mask program of HOMER v4.10.3. (Heinz et al., 2010) within 1-2 Kb from the TSS. For scRNA-seq, raw sequence reads were quality-checked using FastQC software. The Cell Ranger version 2.1.1 software suite from 10× Genomics (https://support.10xgenomics.com/single-cell-gene-expression/software/downloads/latest) was used to process, align and summarize unique molecular identifier (UMI) counts against the mouse mm10 assembly reference genome analysis set, obtained from the University of California Santa Cruz (UCSC). Raw, unfiltered count matrices were imported into R for further processing. Raw UMI count matrices were filtered using the Seurat v 3.0 R package (Butler et al., 2018) to remove: barcodes with very low (empty wells) and very high (probably doublets) total UMI counts; matrices for which a high percentage of UMIs originated from mitochondrial features (more than 20%); and matrices for which fewer than 250 genes were expressed. Clustering was performed as follows: The percentage of mitochondrial features was a source of unwanted variation and was regressed out using the Seurat package. Dimensionality reduction was performed by principal component analysis and UMAP embedding with resolution 0.5. Cluster gene markers and differentially expressed genes between groups in each cluster were detected using the Wilcoxon Rank Sum test. CellPhoneDB analysis was performed using Cellphonedb method statistical analysis command.

### Quantification and Statistical Analysis

All the statistical tests were justified for every figure. All samples represent biological replicates. Data are presented as the mean±SEM. Statistical analysis was performed using GraphPad Prism 8 or R software environment (http://www.r-project.org/). Significant differences between groups were determined using a Student's t-test (two-tailed) when the data met the normal distribution tested by D'Agostino test. If the data did not meet this test, a Mann-Whitney U-test was used. Differences between more than 3 groups were determined using one-way ANOVA test (parametric) or Brown-Forsythe and Welch ANOVA tests (nonparametric) followed by Dunnett post hoc test. If the data did not meet this test, a Mann-Whitney test was used. Differences in Kaplan Meier plots were analyzed by Gehan-Breslow-Wilcoxon test. The chi-square test or Fisher's exact test was used to determine the significance of differences between covariates. Logistic regression analysis was employed to estimate univariate and multivariate odds ratio and 95% confidence interval (CI). Values of p<0.05 were considered as significantly different.

TABLE 1
List of primers used.

| Gene symbol | Forward | Reverse |
|---|---|---|
| 18s | 5′-GTAACCCGTTGAACCCCAT-3′ | 5′-CCATCCAATCGGTAGTAGCG-3′ |
| | (SEQ ID NO: 20) | (SEQ ID NO: 21) |
| SQSTM1 | 5′-AGCGTCTGCGAGGGAAAG-3′ | 5′-ACCCGAAGTGTCCGTGTTT-3′ |
| | (SEQ ID NO: 22) | (SEQ ID NO: 23) |
| PARP1 | 5′-TCTGCCTTGCTACCAATTCC-3′ | 5′-GATGGGTTCTCTGAGCTTCG-3′ |
| | (SEQ ID NO: 24) | (SEQ ID NO: 25) |
| PARP2 | 5′-CAACACGGCTCCAGAAGACT-3′ | 5′-GCCTTCACAGATTCATCTTGCT-3′ |
| | (SEQ ID NO: 26) | (SEQ ID NO: 27) |
| CD38 | 5′-TGCTGATGACCTCACATGGT-3′ | 5′-CCATTGAGCATCACATGGAC-3′ |
| | (SEQ ID NO: 28) | (SEQ ID NO: 29) |
| MCT1 | 5′-TGTTCCTCTGTACTCTGGCC-3′ | 5′-GCAGTTTAGTAGCAAGCCCC-3′ |
| | (SEQ ID NO: 30) | (SEQ ID NO: 31) |
| MCT4 | 5′-CTCGCTCATCATGCTGAACC-3′ | 5′-ACACAGGAAGACAGGGCTAC-3′ |
| | (SEQ ID NO: 32) | (SEQ ID NO: 33) |
| ACTA2 | 5′-CGATAGAACACGGCATCATC-3′ | 5′-CATCAGGCAGTTCGTAGCTC-3′ |
| | (SEQ ID NO: 34) | (SEQ ID NO: 35) |
| TGFB1 | 5′-CGTGGAGCTGTACCAGAAATAC-3′ | 5′-CACAACTCCGGTGACATCAA-3′ |
| | (SEQ ID NO: 36) | (SEQ ID NO: 37) |
| HAS1 | 5′-GACTCCTGGGTCAGCTTCCTAAG- | 5′-AAACTGCTGCAAGAGGTTATTCCT-3′ |
| | 3′ (SEQ ID NO: 38) | (SEQ ID NO: 39) |
| HAS3 | 5′-AGCACCTTCTCGTGCATCATGC-3′ | 5′-TCCTCCAGGACTCGAAGCATCT-3′ |
| | (SEQ ID NO: 40) | (SEQ ID NO: 41) |
| SFRP1 | 5′-TCAGGGGCTTCTTCTTCTTTG-3′ | 5′-TCTGAGGCCATCATTGAACA-3′ |
| | (SEQ ID NO: 42) | (SEQ ID NO: 43) |
| MMP9 | 5′-TCTTCCCTGGAGACCTGAGAAC-3′ | 5′-GACACCAAACTGGATGACGATG-3′ |
| | (SEQ ID NO: 44) | (SEQ ID NO: 45) |
| SIRT1 | 5′-TGCTGGCCTAATAGAGTGGCA-3′ | 5′-CTCAGCGCCATGGAAAATGT-3′ |
| | (SEQ ID NO: 46) | (SEQ ID NO: 47) |
| SIRT2 | 5′-CCATCTGTCACTACTTCATGC-3′ | 5′-AAGTCCTCCTGTTCCAGC-3′ |
| | (SEQ ID NO: 48) | (SEQ ID NO: 49) |
| SIRT3 | 5′-GCTGGACAGAAGAGATGC-3′ | 5′-GTGGATGTCTCCTATGTTACC-3′ |
| | (SEQ ID NO: 50) | (SEQ ID NO: 51) |
| SIRT4 | 5′-GCGTGTAAAAGAAGCCGACT-3′ | 5′-TTCTTCTCCCAGGCAGTCAG-3′ |
| | (SEQ ID NO: 52) | (SEQ ID NO: 53) |
| SIRT5 | 5′-CCCAGAACATCGATGAGC-3′ | 5′-GCCACAACTCCACAAGAGG-3′ |
| | (SEQ ID NO: 54) | (SEQ ID NO: 55) |
| SIRT6 | 5′-AGGGACAAACTGGCAGAGC-3′ | 5′-TTAGCCACGGTGCAGAGC-3′ |
| | (SEQ ID NO: 56) | (SEQ ID NO: 57) |
| SIRT7 | 5′-GCAGAGCAGACACCATCC-3′ | 5′-GTTCACGATGTAAAGCTTCG-3′ |
| | (SEQ ID NO: 58) | (SEQ ID NO: 59) |
| Chip-AP1-A | 5′-ATCCCCCTATTACGACAGCG-3′ | 5′-GGCCTCCCGGAGGTAAACA-3′ |
| | (SEQ ID NO: 60) | (SEQ ID NO: 61) |
| Mutagenesis | 5′CGGTCATGGGACGCTCTGTCACTG | 5′GGTCAGGTGGTCTGGCCGGCAGTG |
| AP1-A | CCGGCCAGACCACCTGACC-3′ | ACAGAGCGTCCCATGACCG-3′ |
| | SEQ ID NO: 62) | (SEQ ID NO: 63) |
| Mutagenesis | 5′CAGGCGCCTGGGCTGCTCTGTCAC | 5′GACAGGTGCTGGCCAAGCGTGACA |
| AP1-B | GCTTGGCCAGCACCTGTC-3′ | GAGCAGCCCAGGCGCCTG-3′ |
| | (SEQ ID NO: 64) | (SEQ ID NO: 65) |
| Mutagenesis | 5′CCCAACTGAGGATATTGCTCTGTC | 5′GGCTTGGGCCTGGCCATGACAGAG |
| AP1-C | ATGGCCAGGCCCAAGCC-3′ | CAATATCCTCAGTTGGG-3′ |
| | (SEQ ID NO: 66) | (SEQ ID NO: 67) |

TABLE 2
List of siRNA oligonucleotides and guides.

| Oligonucleotides | Source | Identifier |
|---|---|---|
| Human PARP1 siRNA | Thermo | 10038 |
| | Fisher Cat# | |
| Human MCT1 siRNA | Thermo | 104272 |
| | Fisher Cat# | |
| Human MCT4 siRNA | Thermo | 107503 |
| | Fisher Cat# | |
| Human FOS siRNA | Thermo | 115631 |
| | Fisher Cat# | |
| Human CD38 siRNA | Thermo | 119605 |
| | Fisher Cat# | |
| Human PARP2 siRNA | Thermo | 111561 |
| | Fisher Cat# | |
| Human SIRT1 siRNA | Thermo | 136457 |
| | Fisher Cat# | |
| Human SIRT2 siRNA | Thermo | 136455 |
| | Fisher Cat# | |
| Human SIRT3 siRNA | Thermo | 136460 |
| | Fisher Cat# | |
| Human SIRT4 siRNA | Thermo | 136462 |
| | Fisher Cat# | |
| Human SIRT5 siRNA | Thermo | 19661 |
| | Fisher Cat# | |
| Human SIRT6 siRNA | Thermo | 116148 |
| | Fisher Cat# | |
| Human SIRT7 siRNA | Thermo | 116146 |
| | Fisher Cat# | |
| gRNA targeting human PARP-1 5′- | Synthego | N/A |
| GGGACTTTTCCATCAAACAT-3′ (SEQ ID NO: 68) | | |
| gRNA targeting AP1-A in human SQSTM1 promoter 5′- | Synthego | N/A |
| ATGGGACGCTGACTCACTGC-3′ (SEQ ID NO: 69) | | |
| ssODN for human SQSTM1 promoter editing | IDT | N/A |
| 5′TCCCCAGCCCAGCCTCCAGGTAAGAGTCACTGAGATGGG | | |
| TGGCAGCAGGGGCCGGGGATCCCCCTATTACGACAGCGGT | | |
| CATGGGACGCCTGCCGGCCAGACCACCTGACCTCCGCGGC | | |
| GGGAGGAGAGGGCC-3′ (SEQ ID NO: 70) | | |

### Results

Prostate Cancer Cells Secrete a Soluble Factor that Reduces p62 Expression in Stromal Fibroblasts

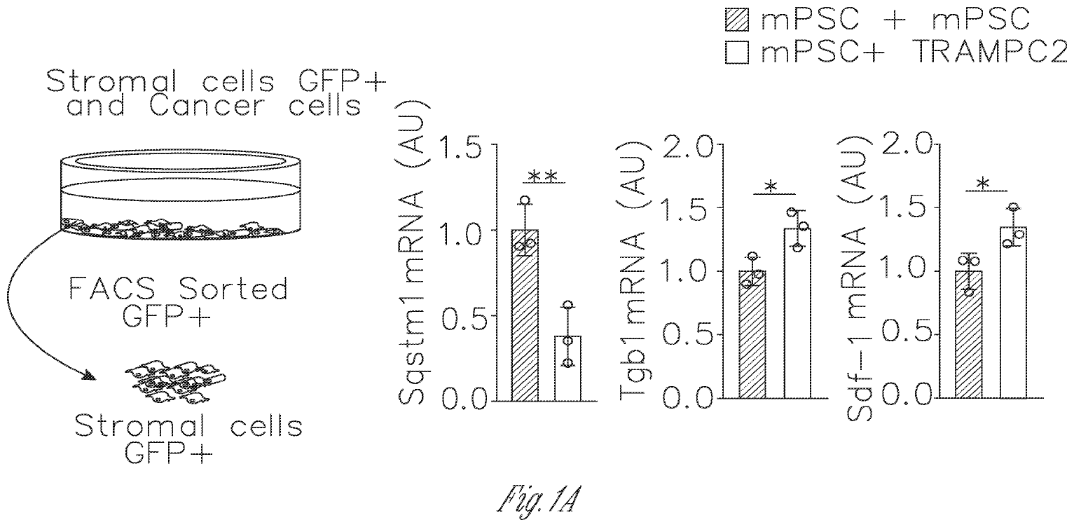

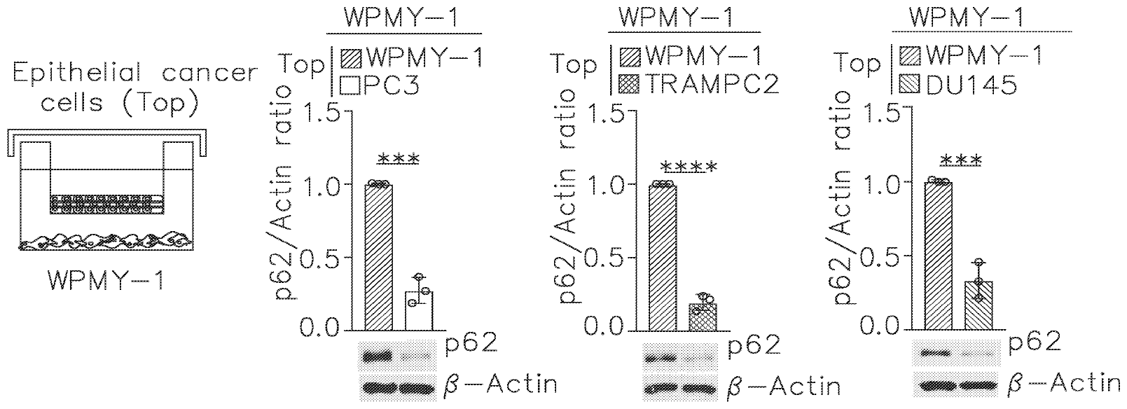

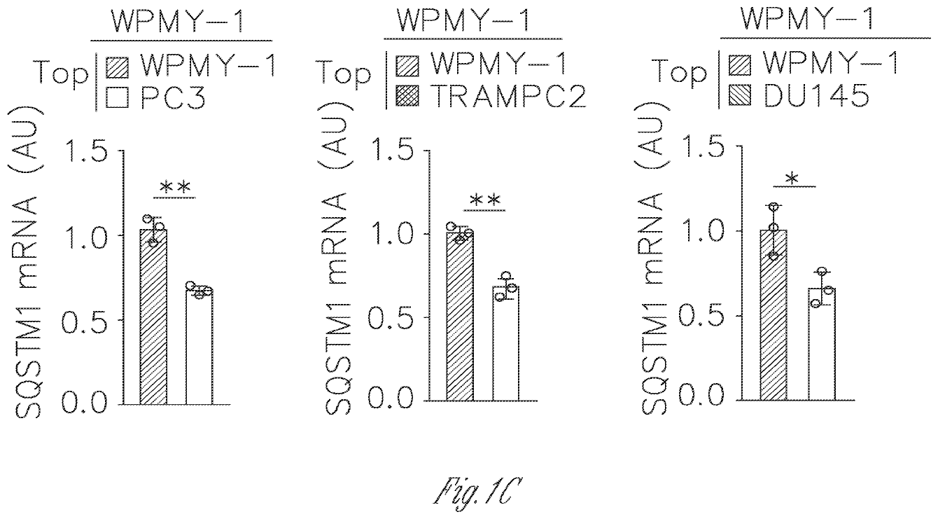

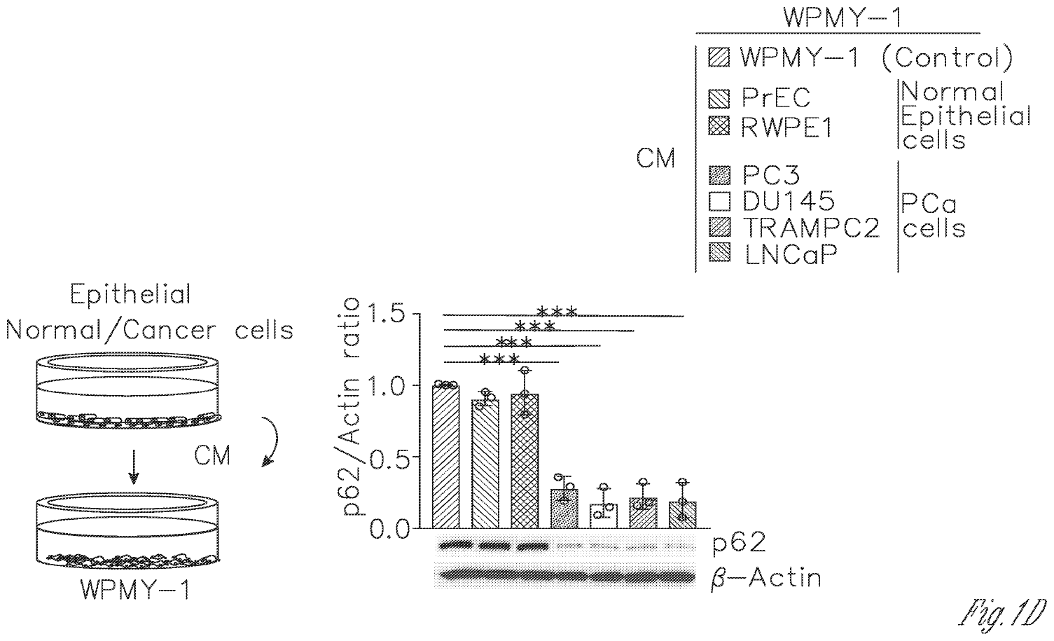

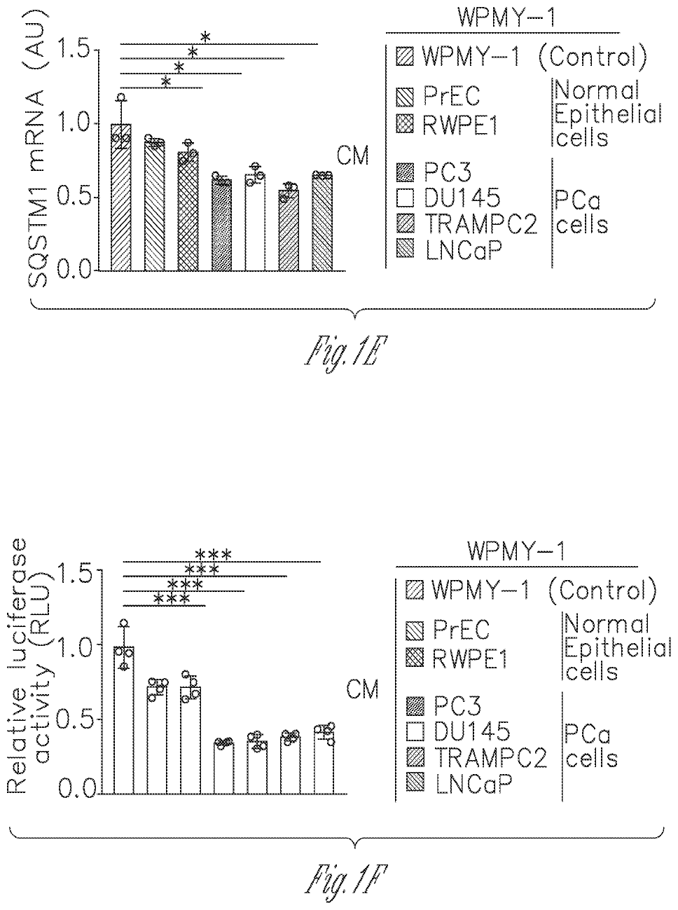

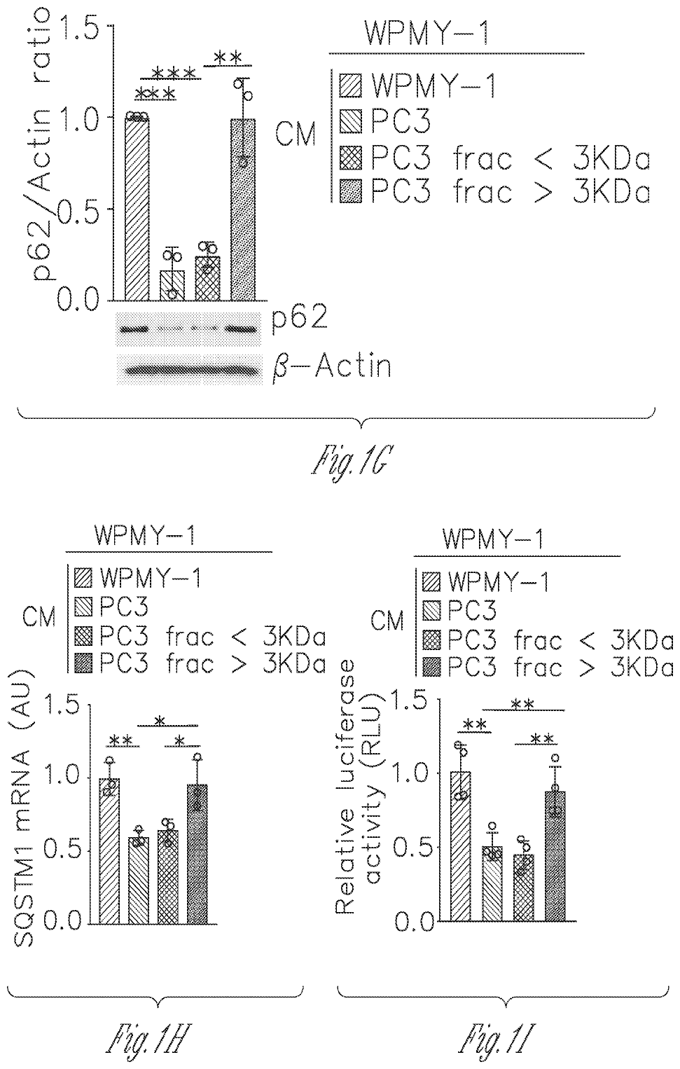

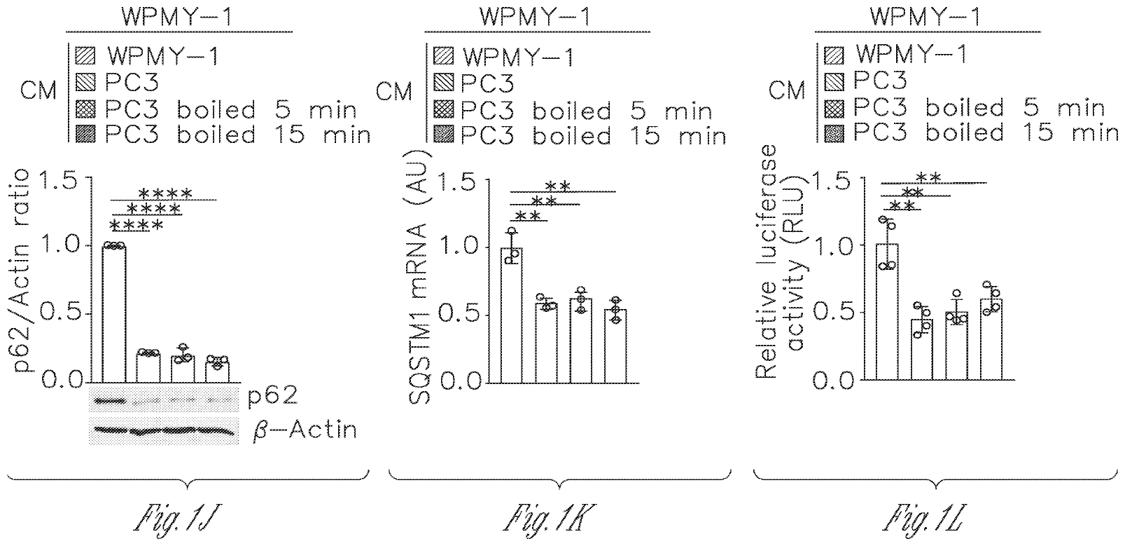

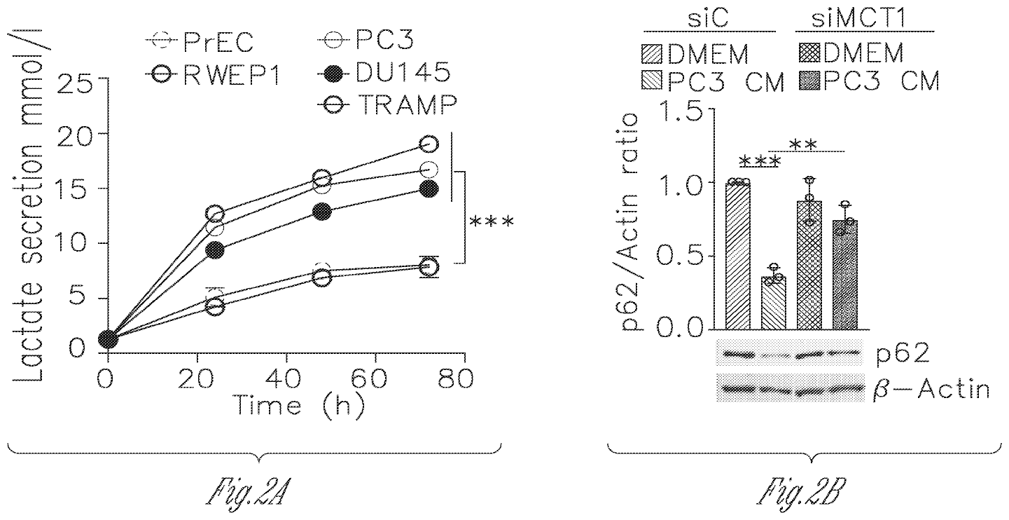

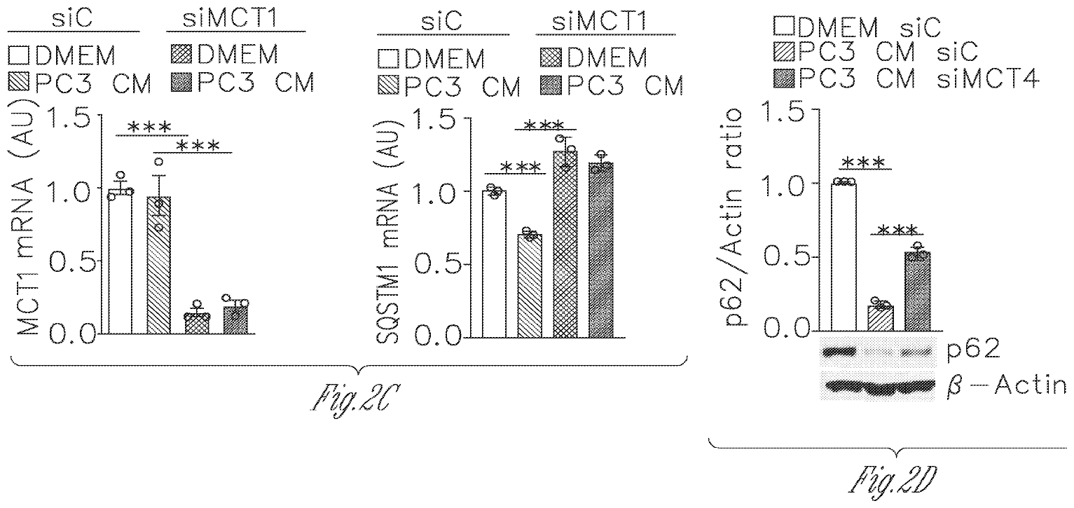

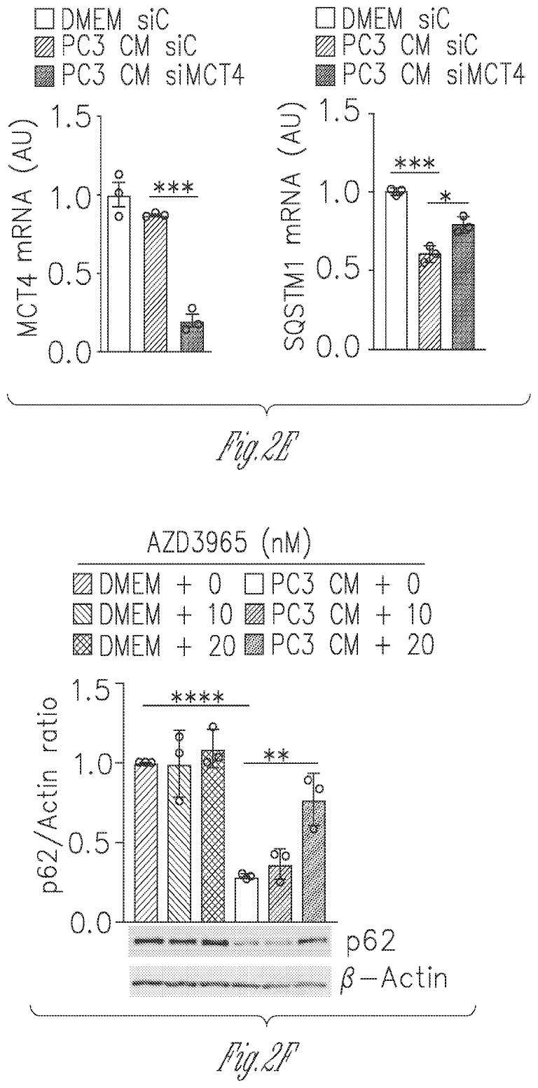

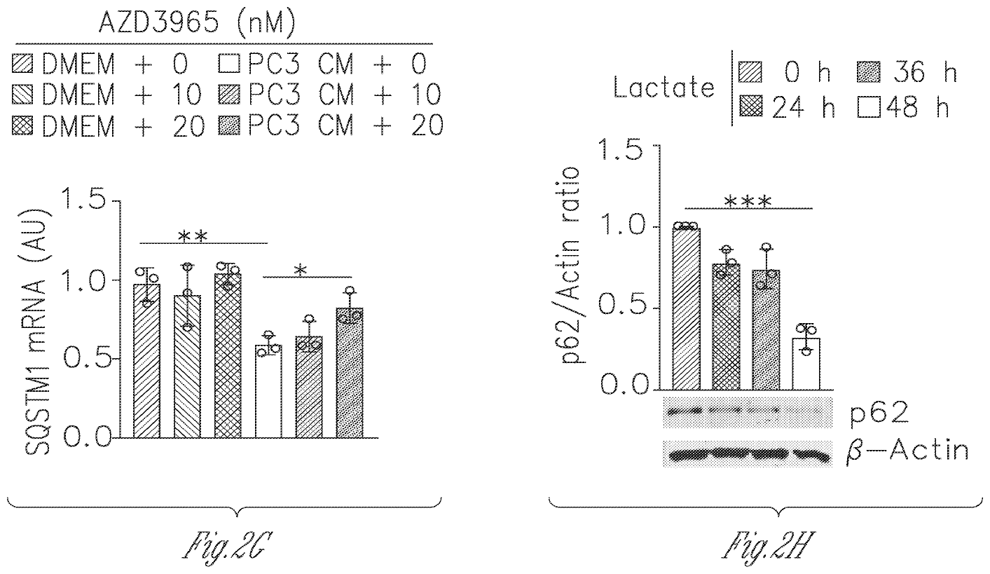

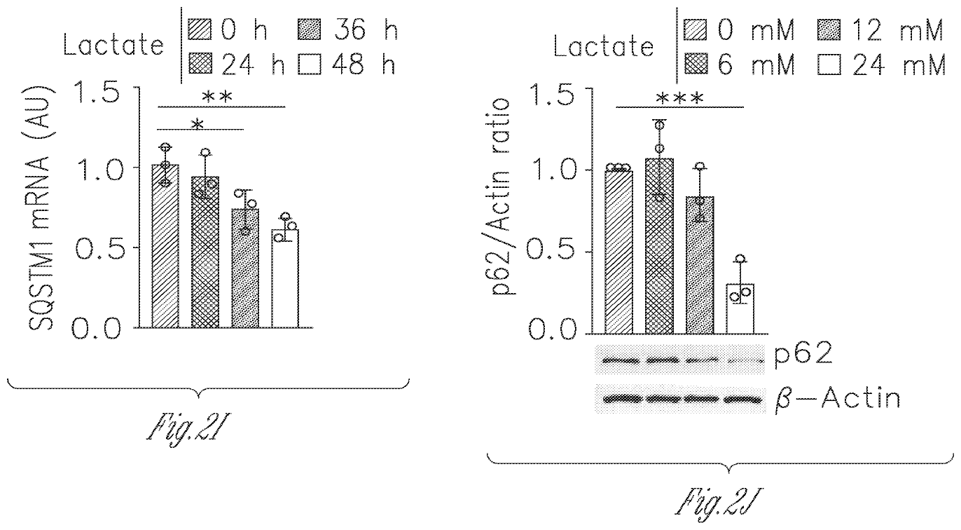

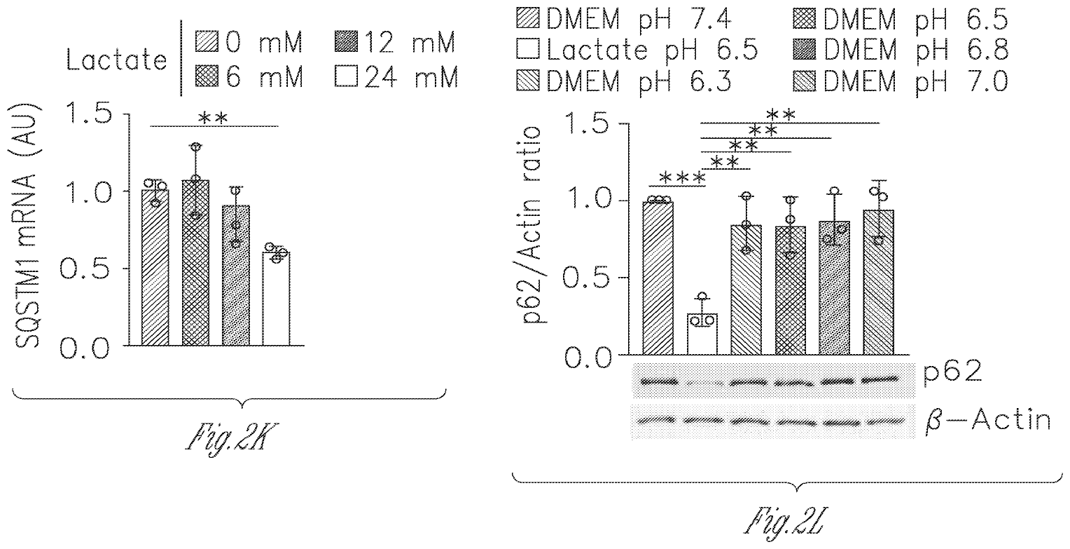

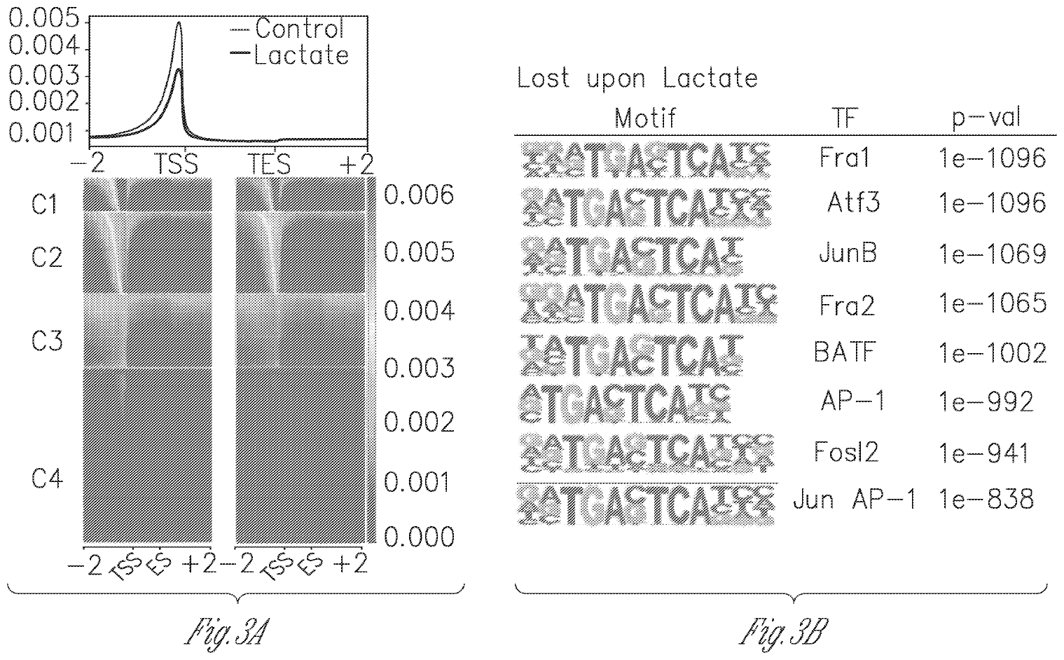

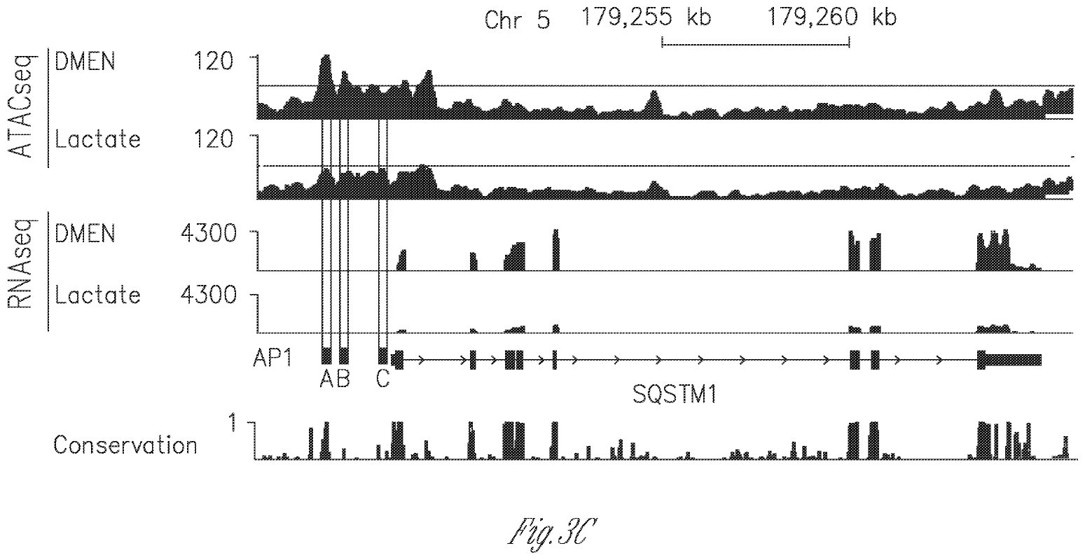

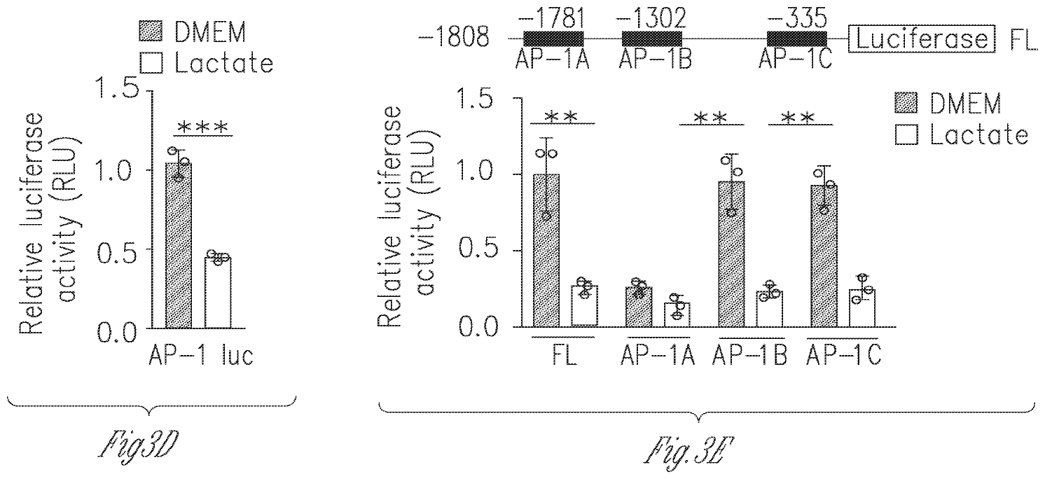

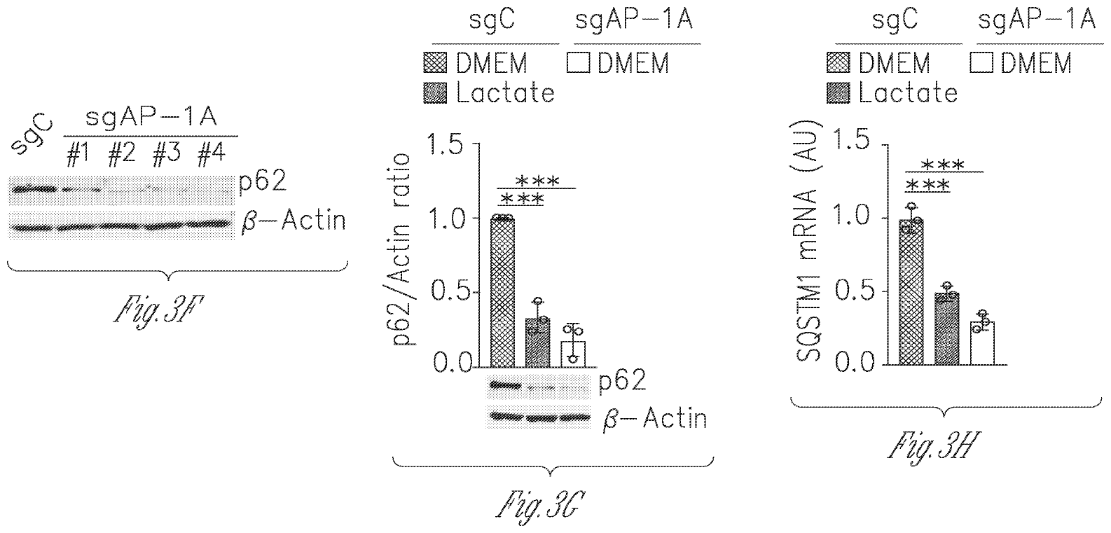

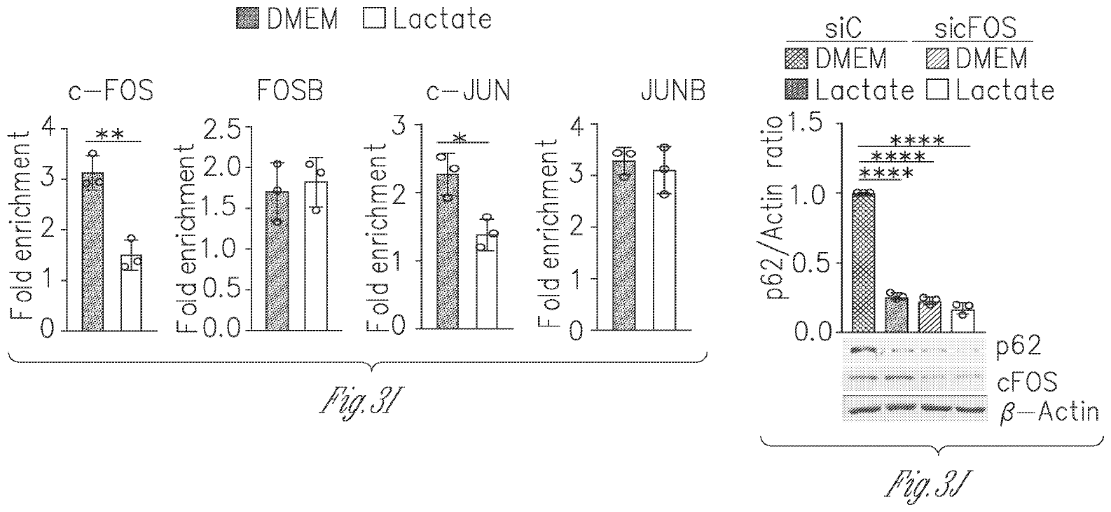

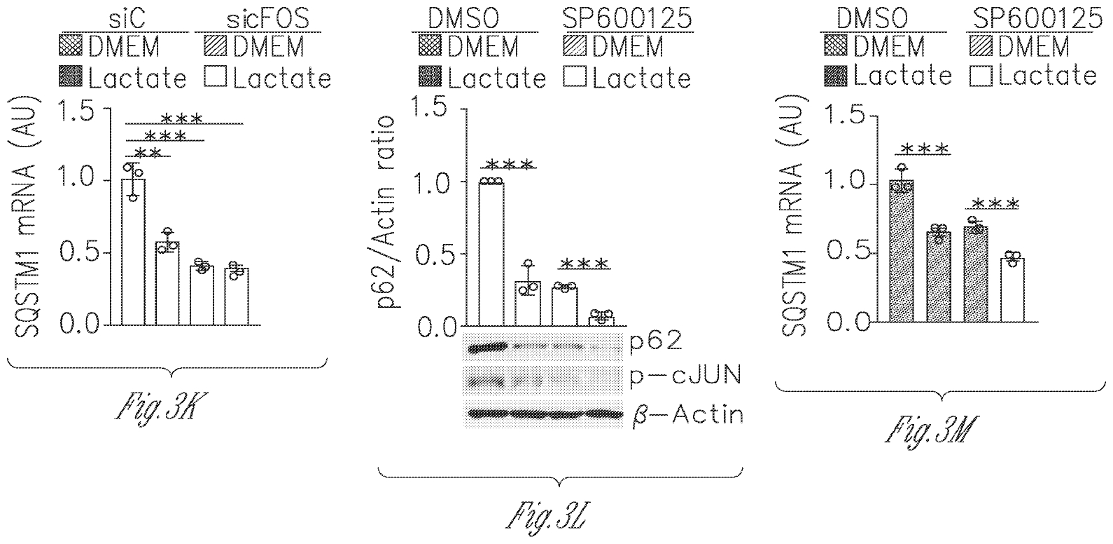

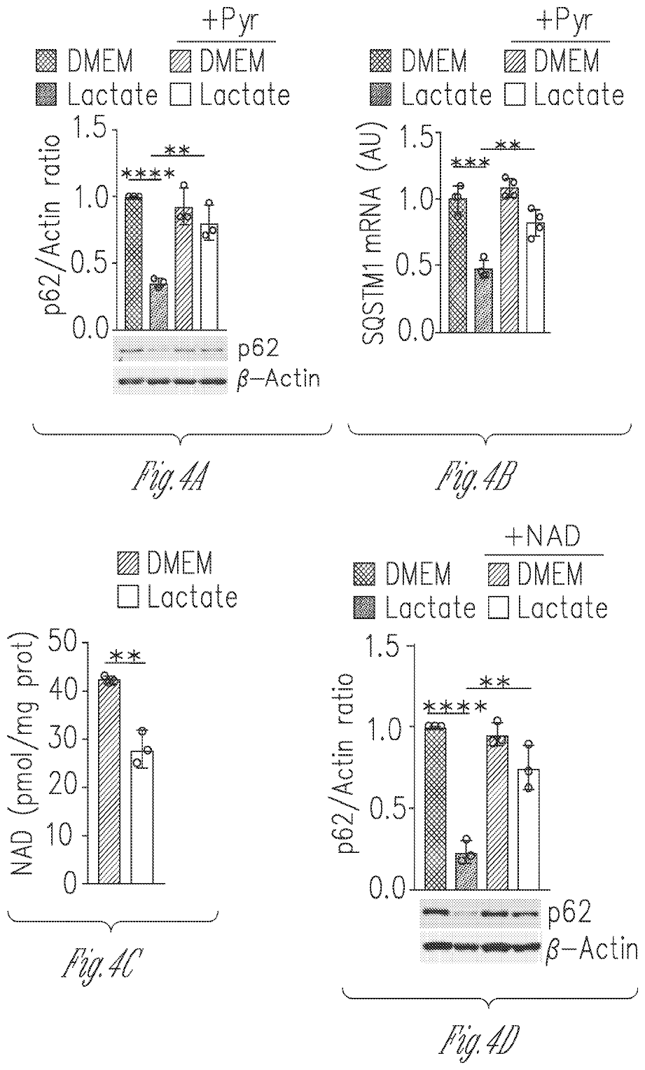

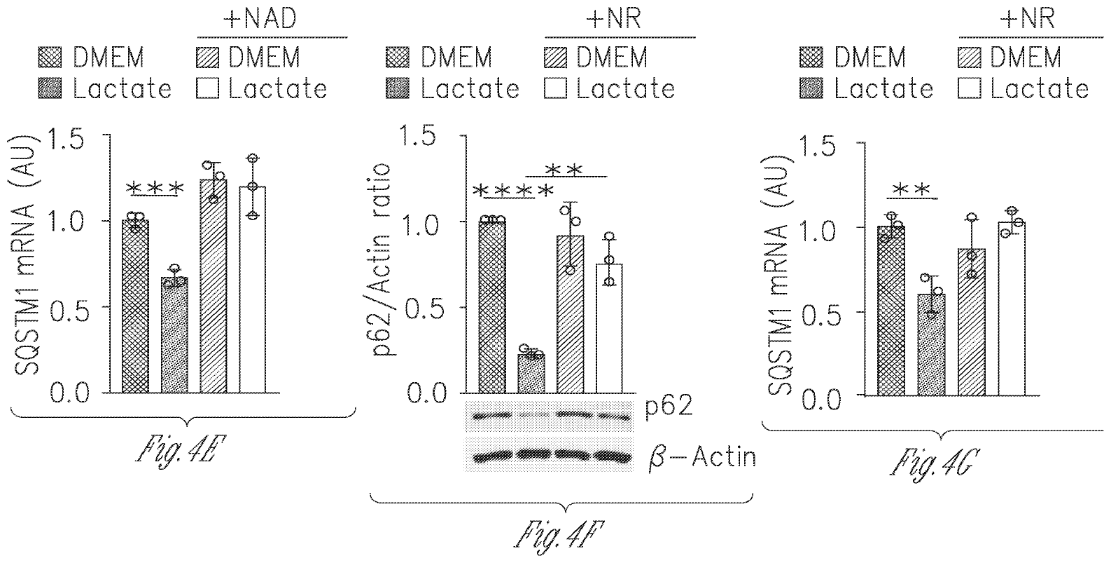

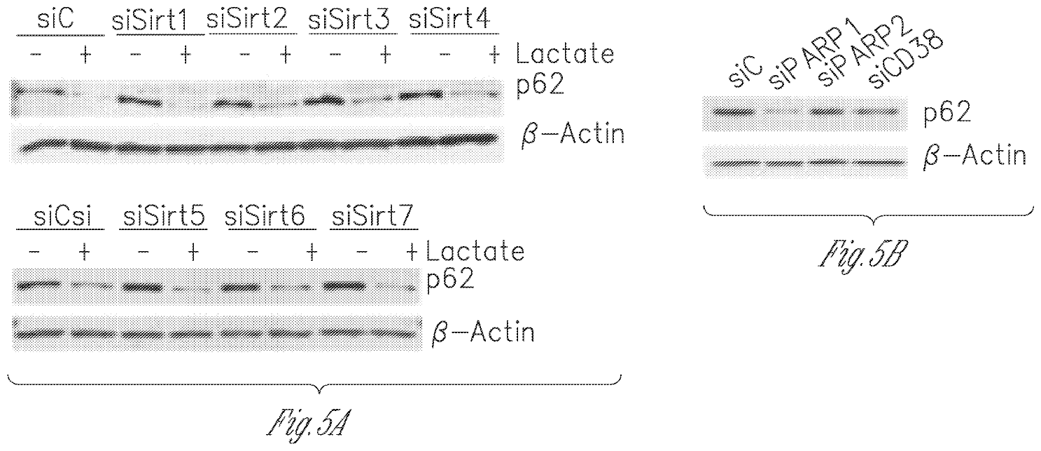

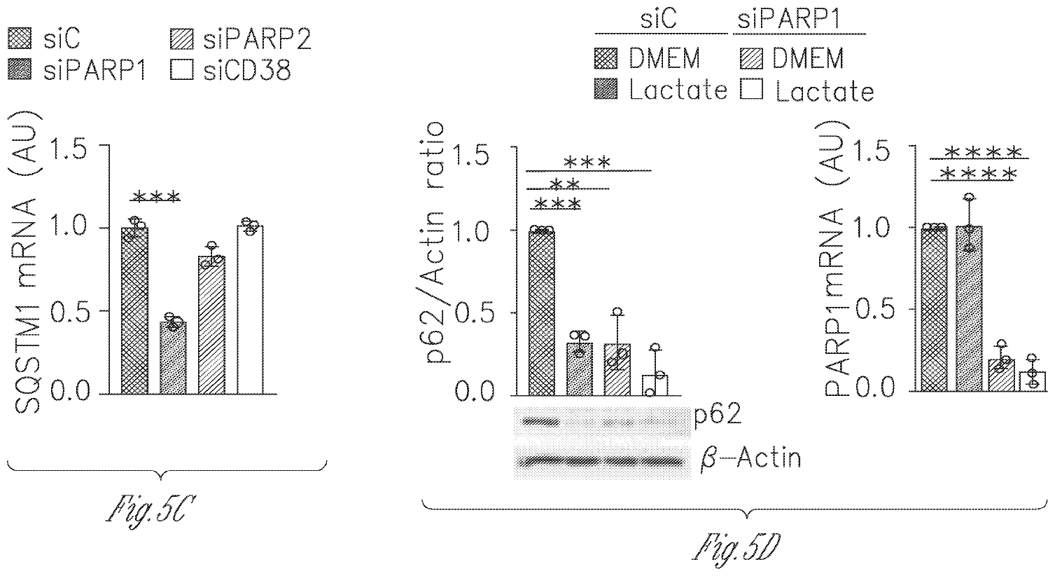

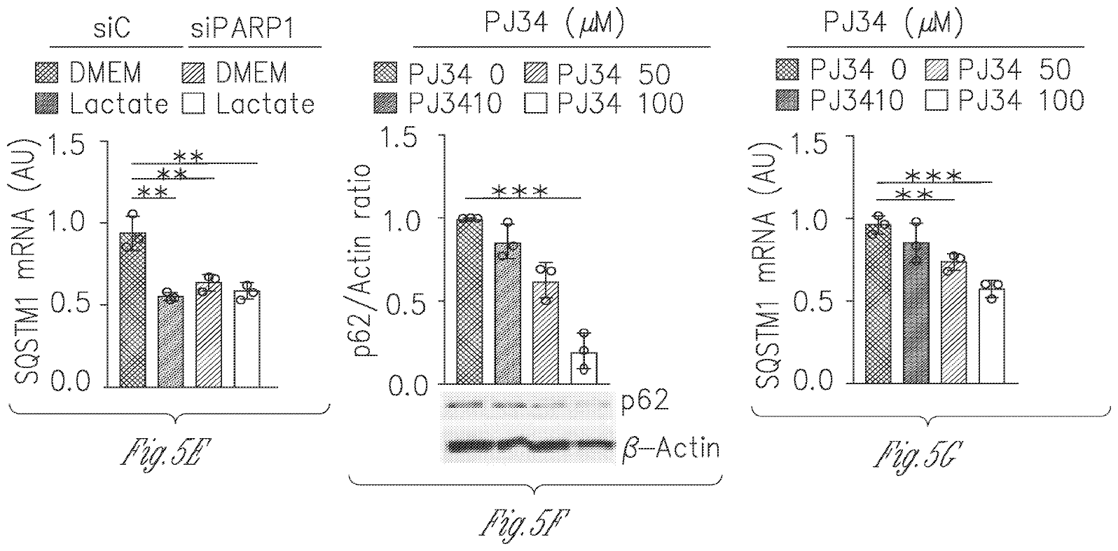

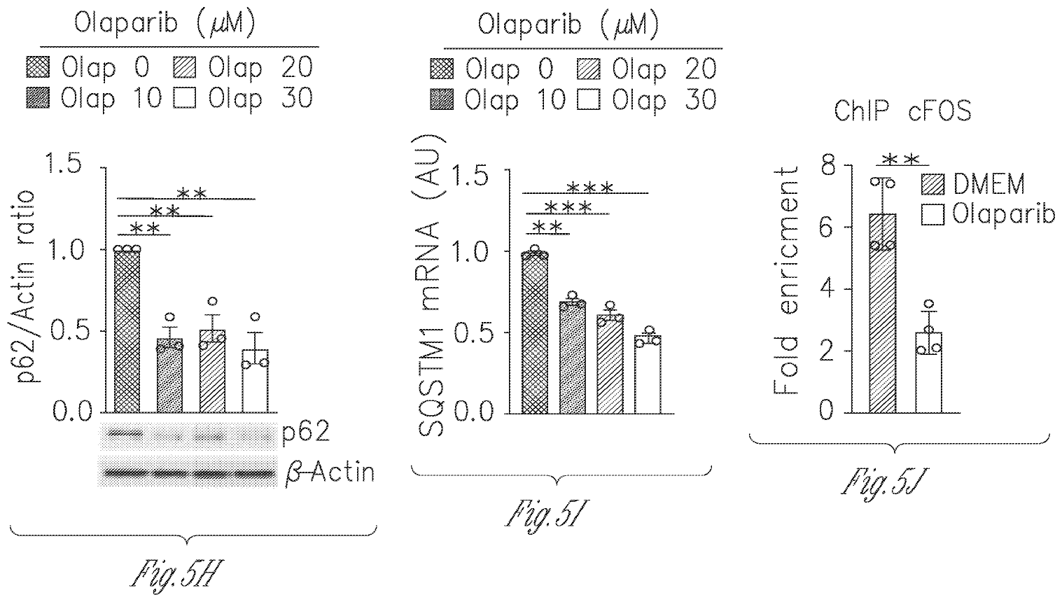

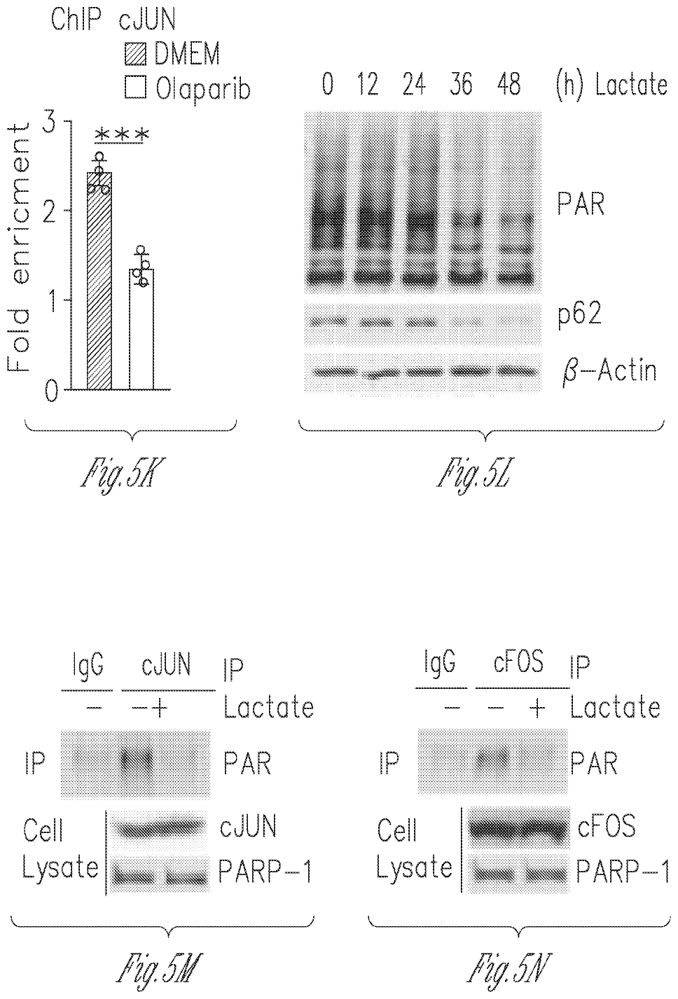

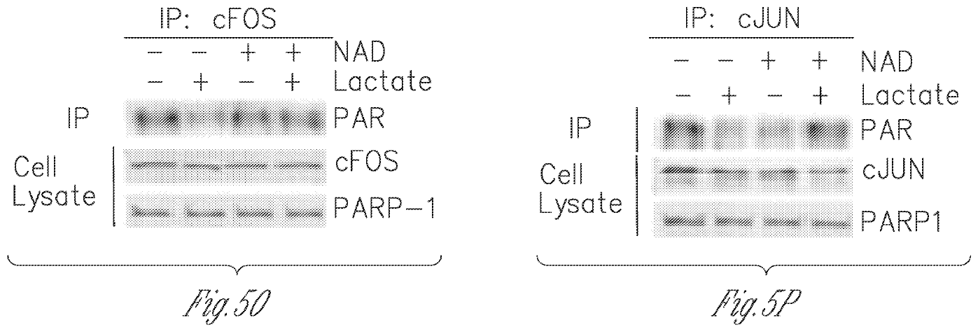

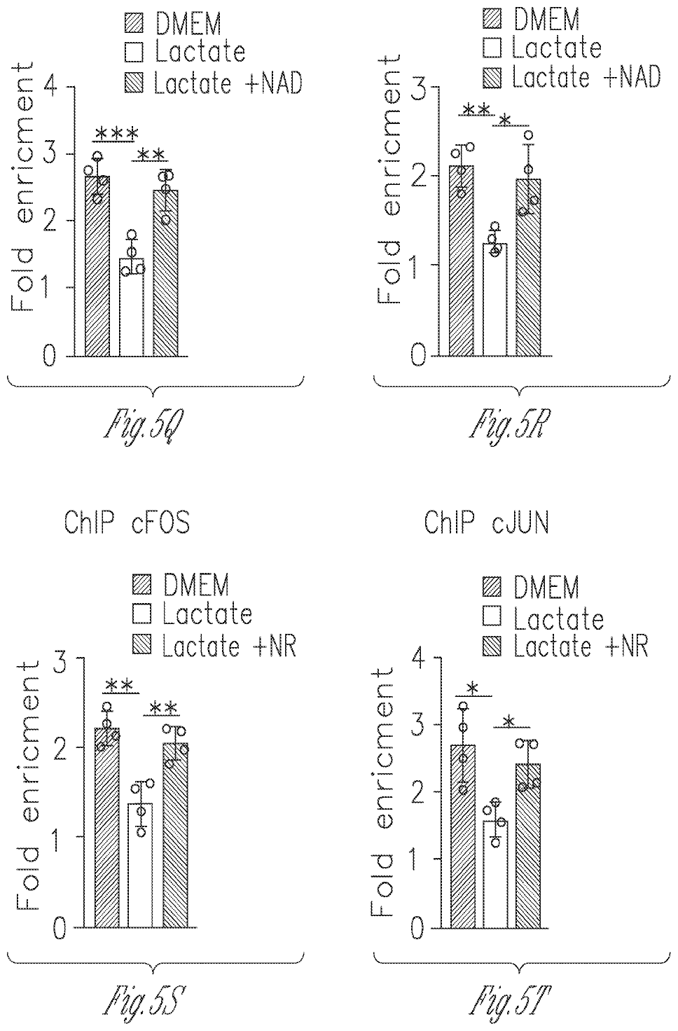

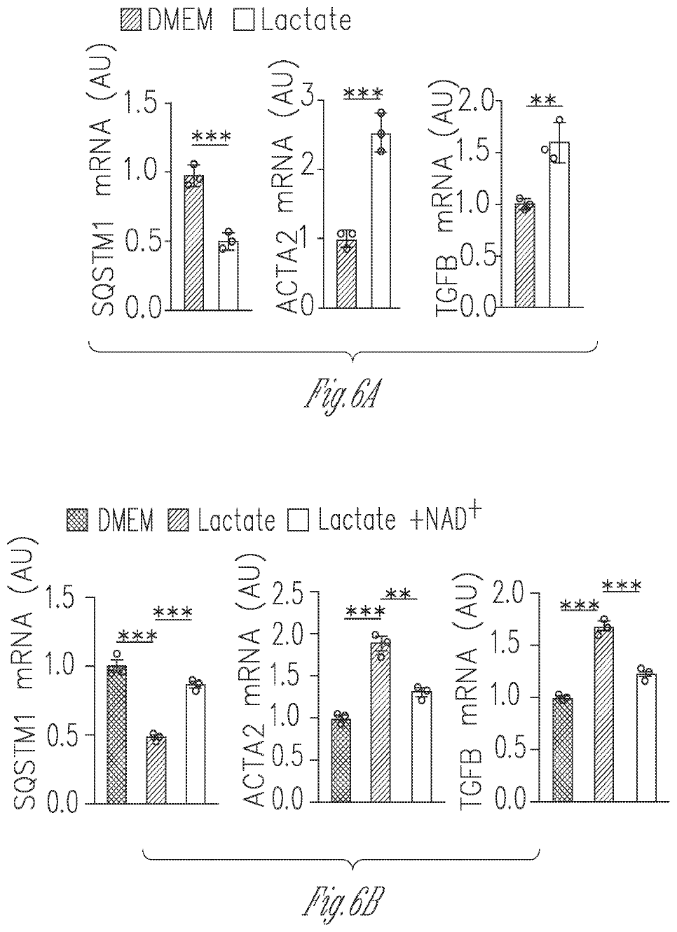

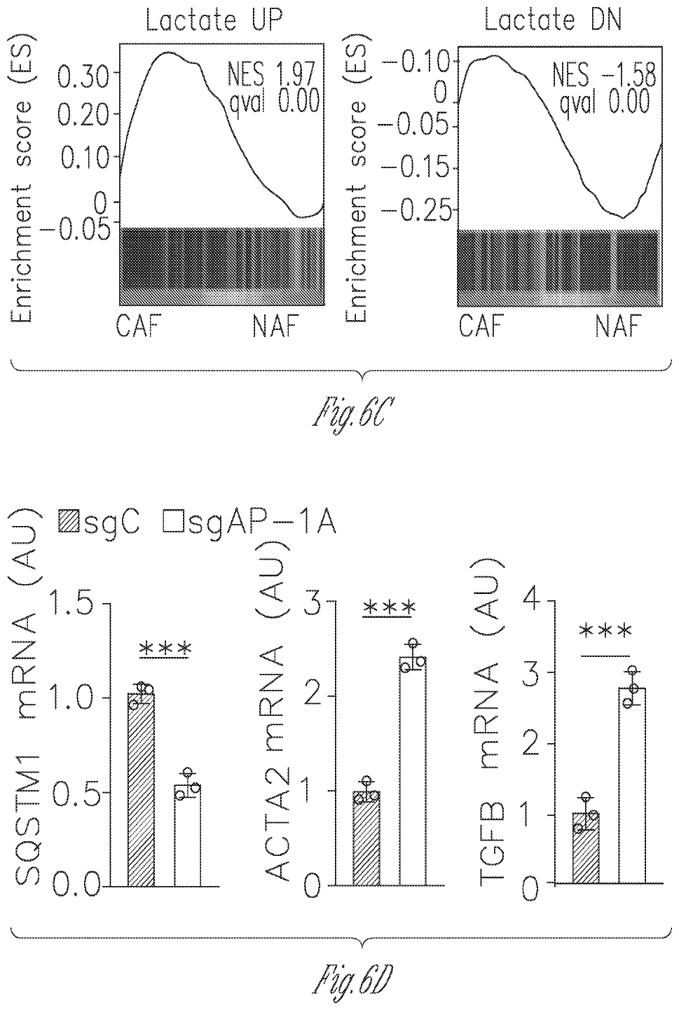

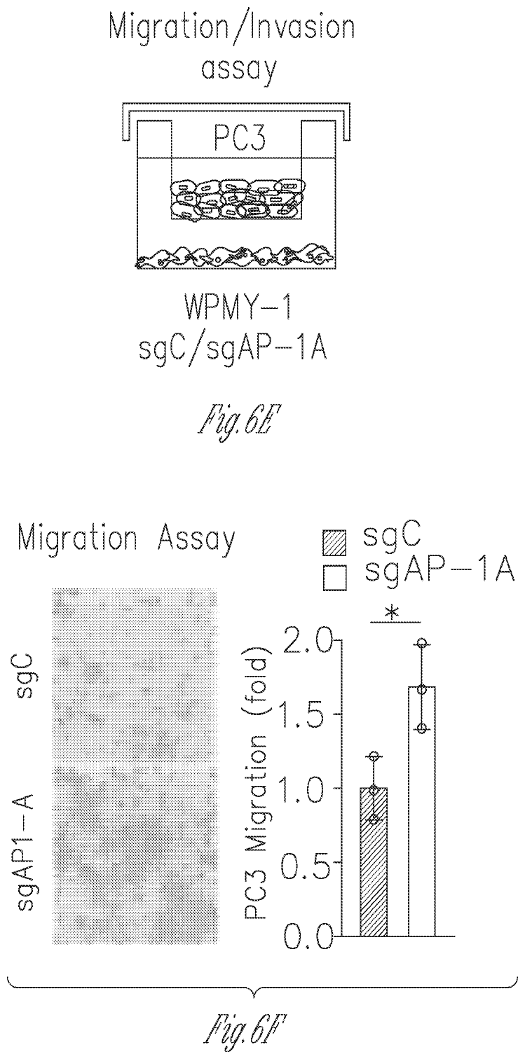

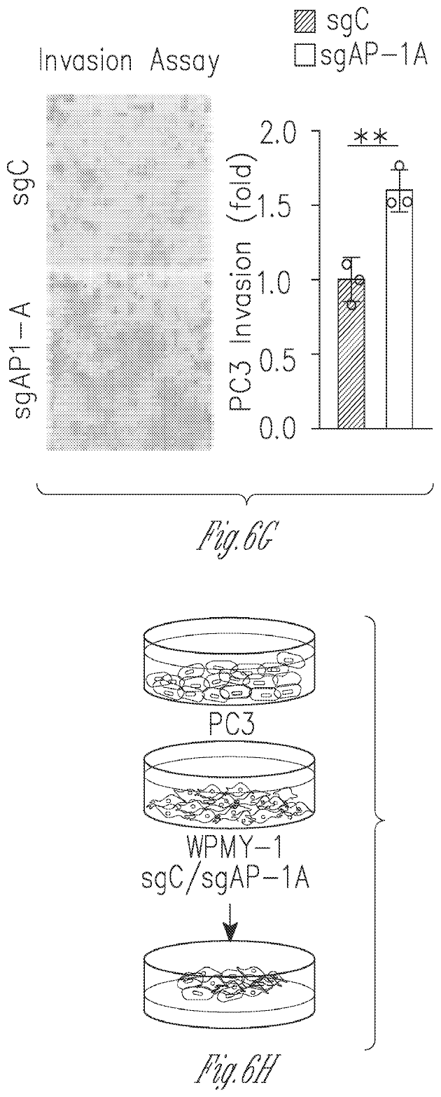

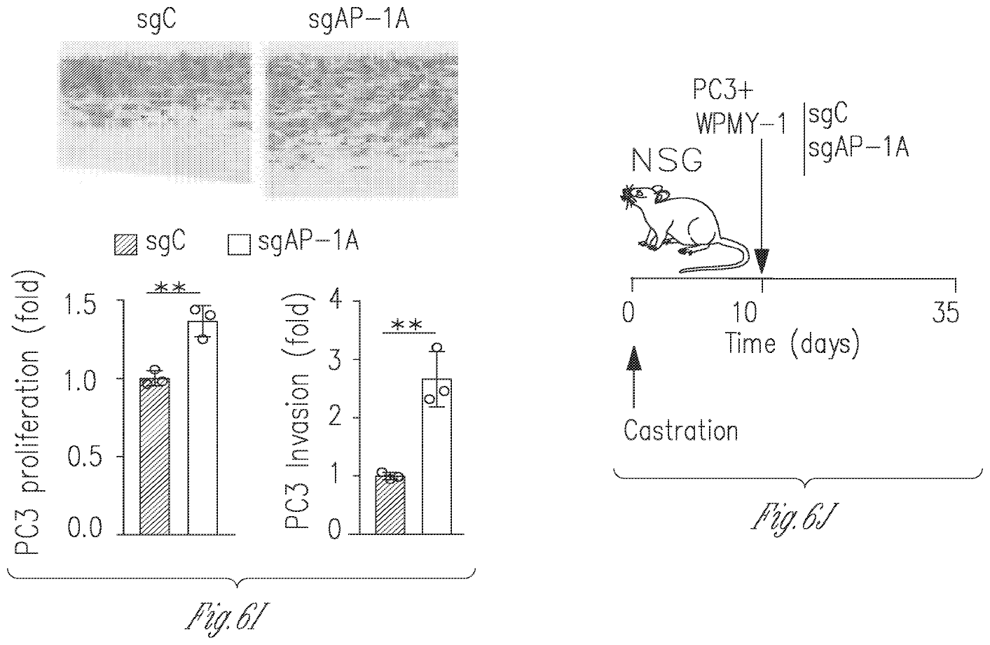

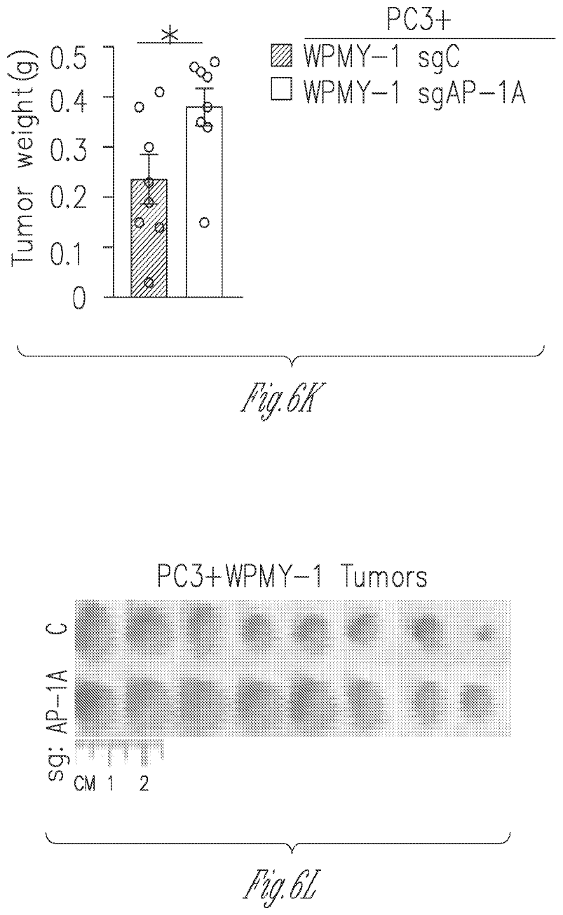

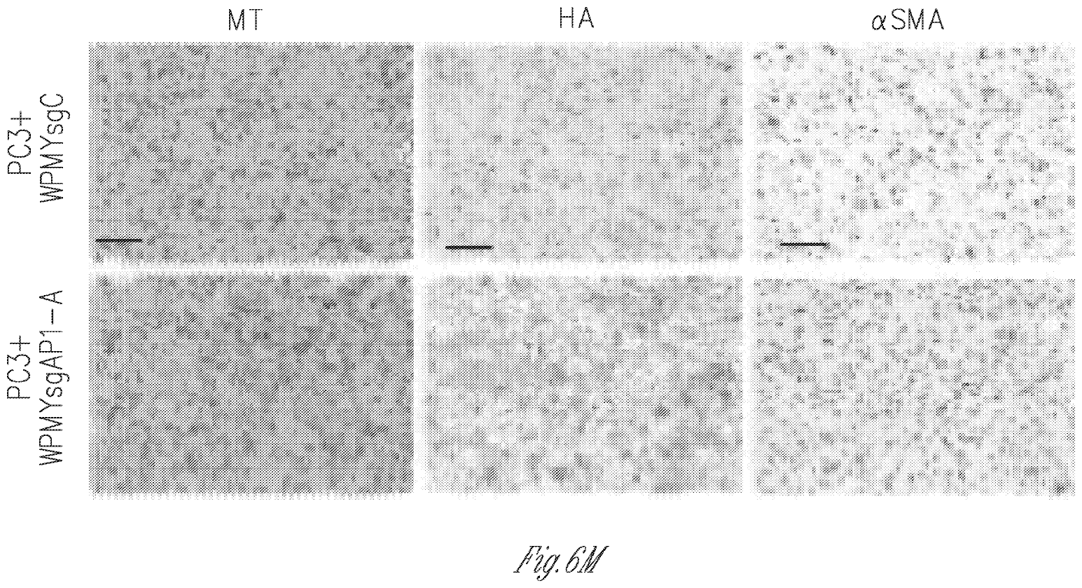

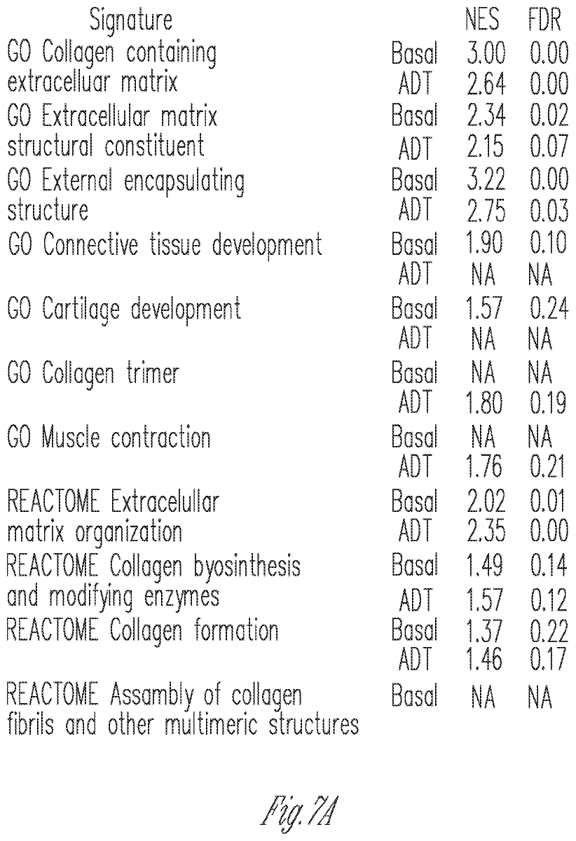

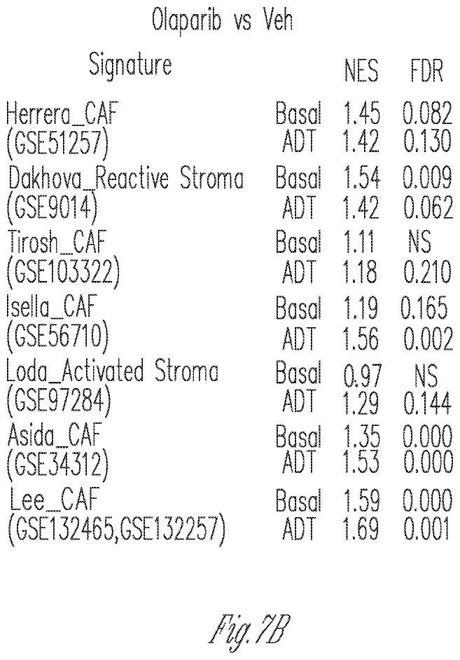

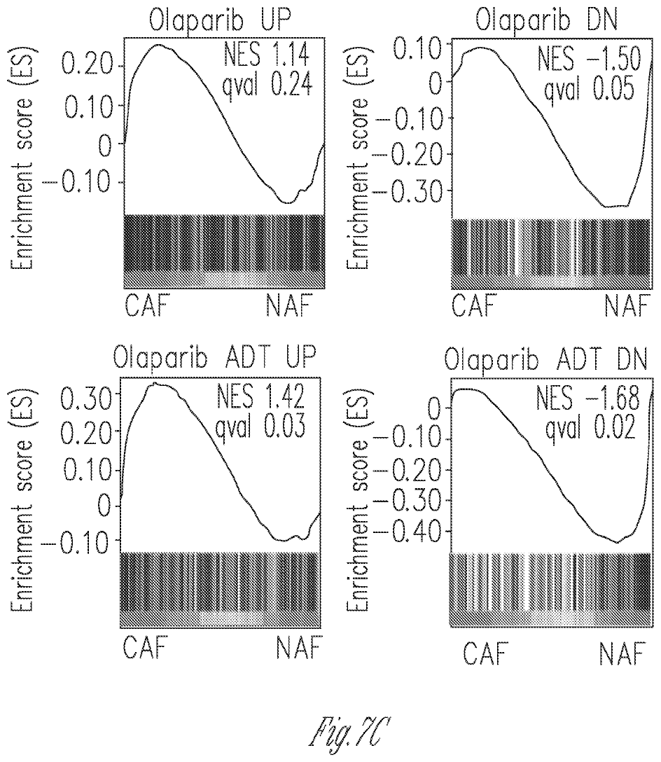

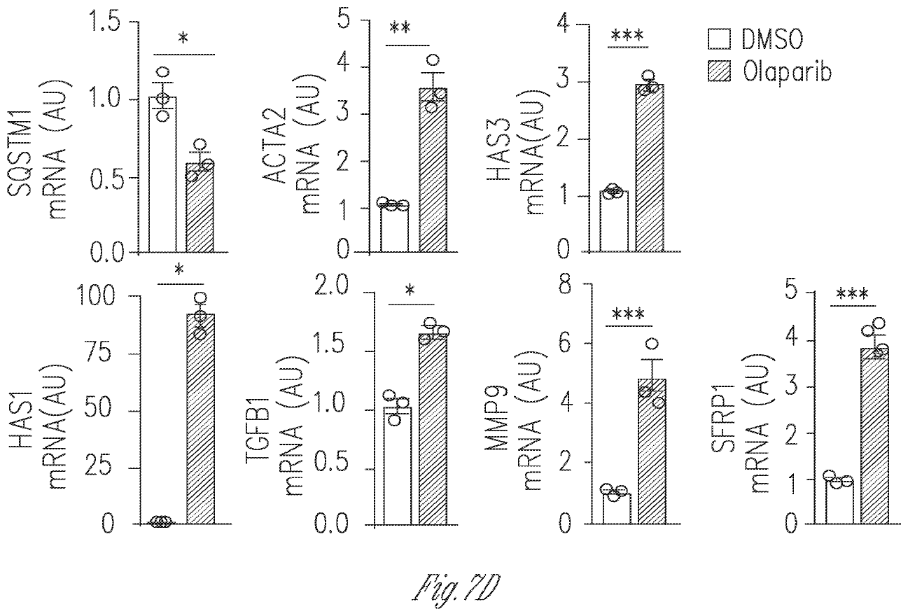

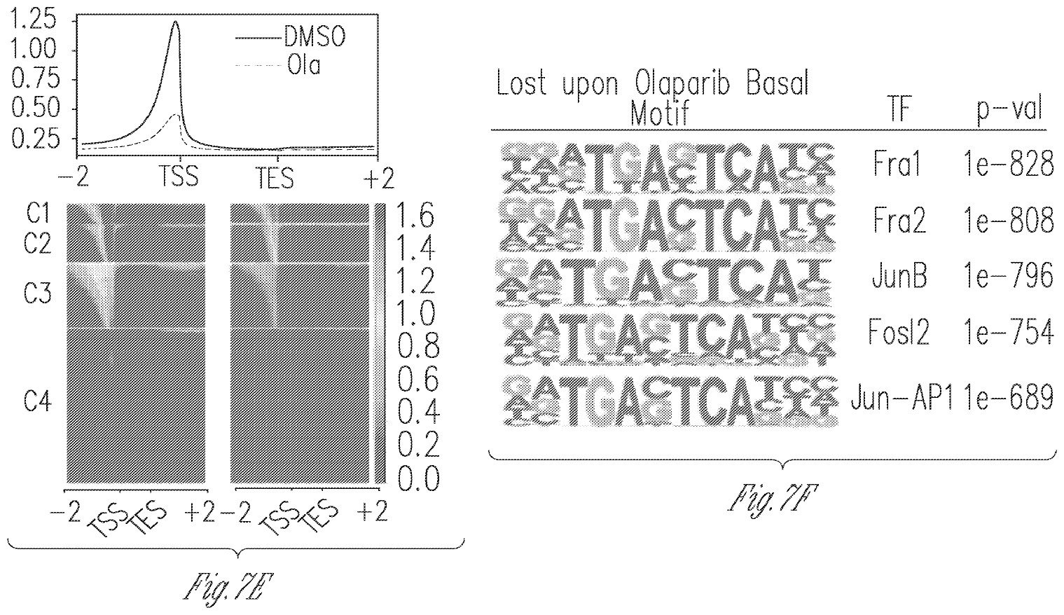

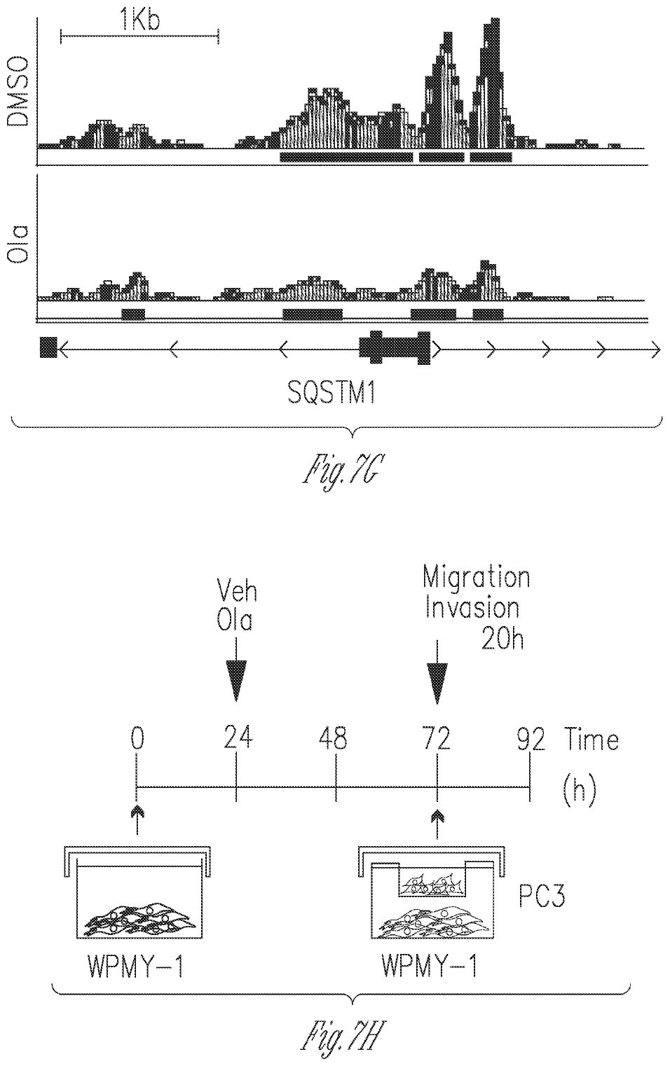

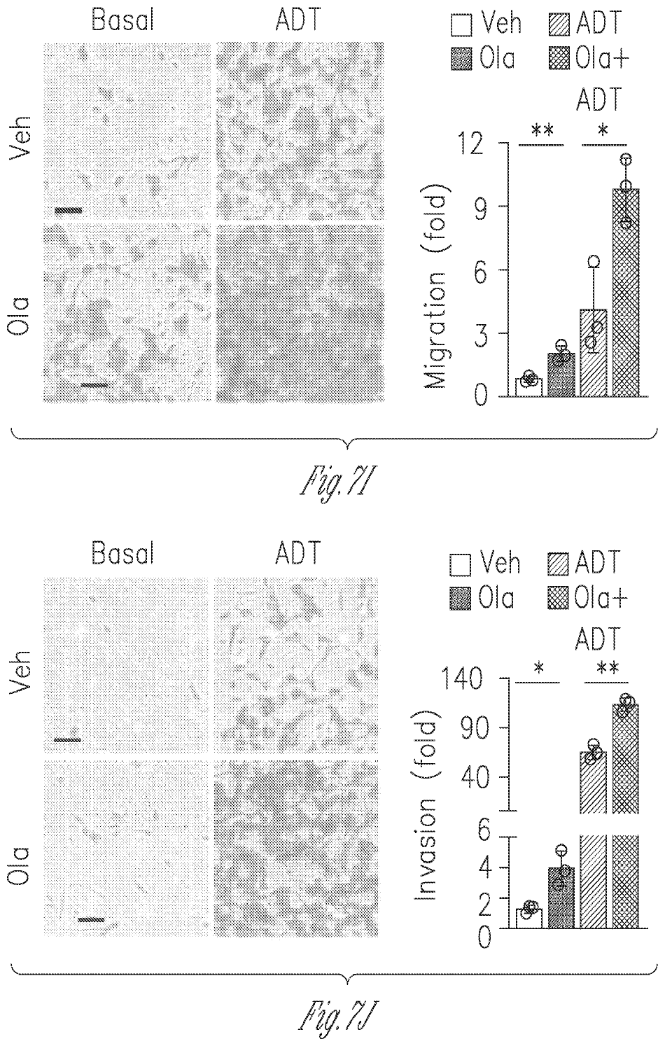

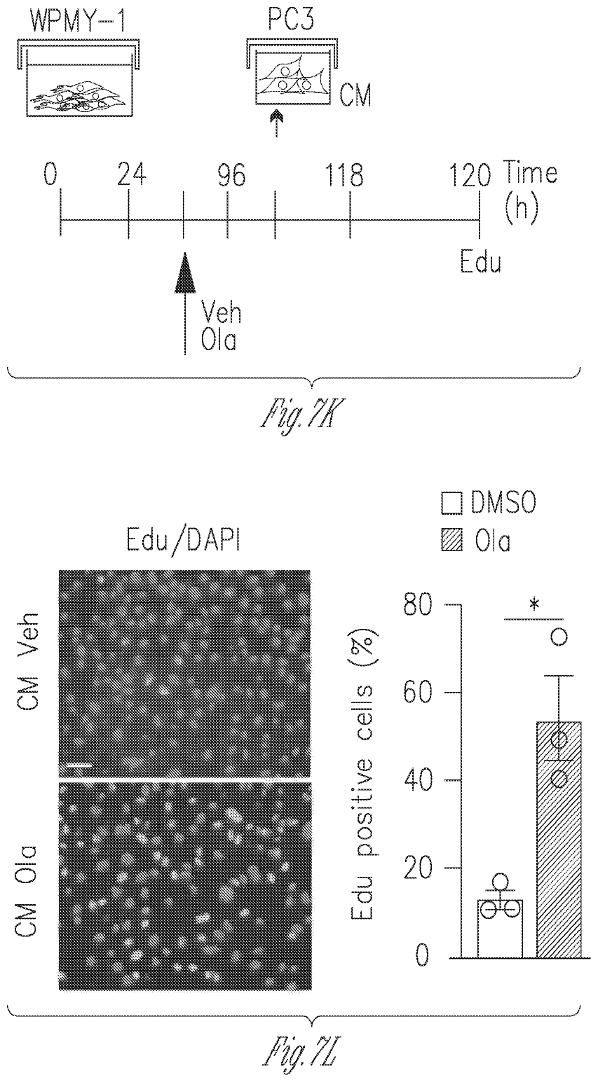

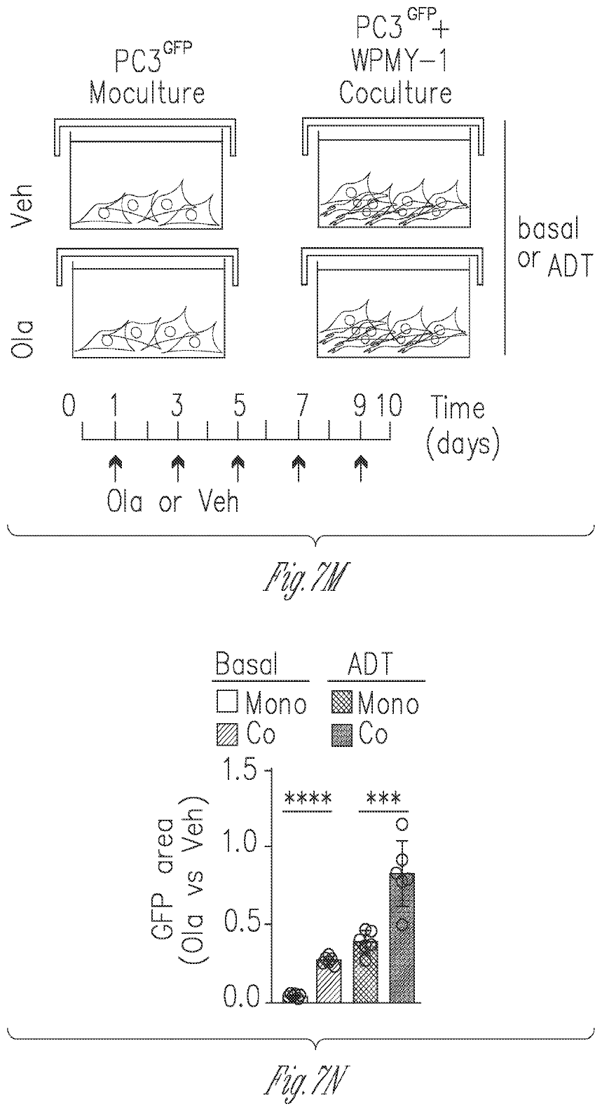

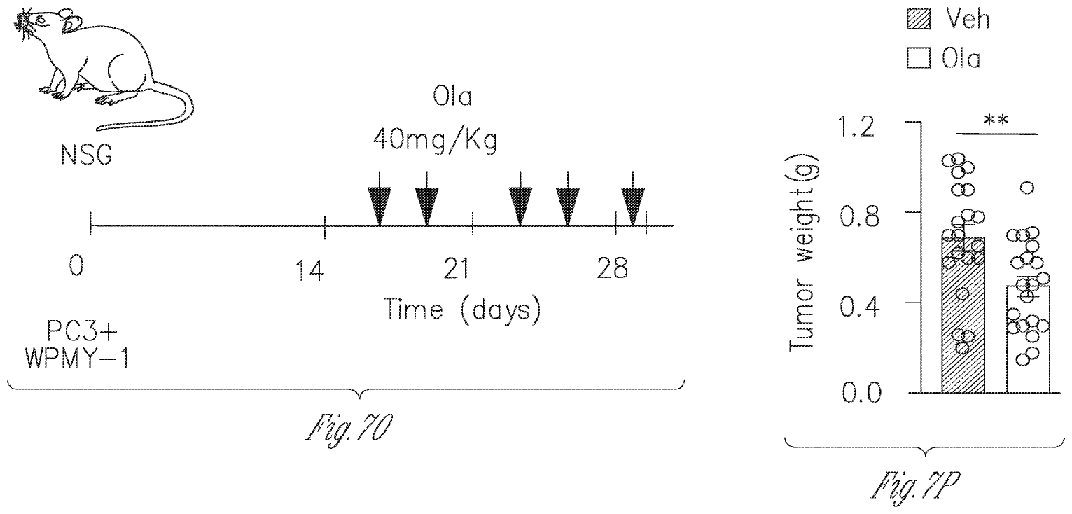

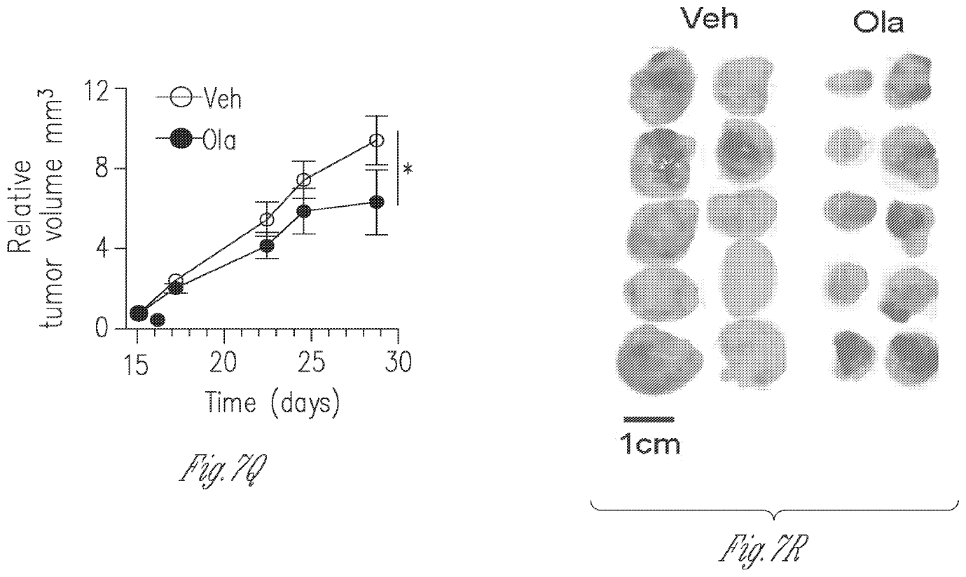

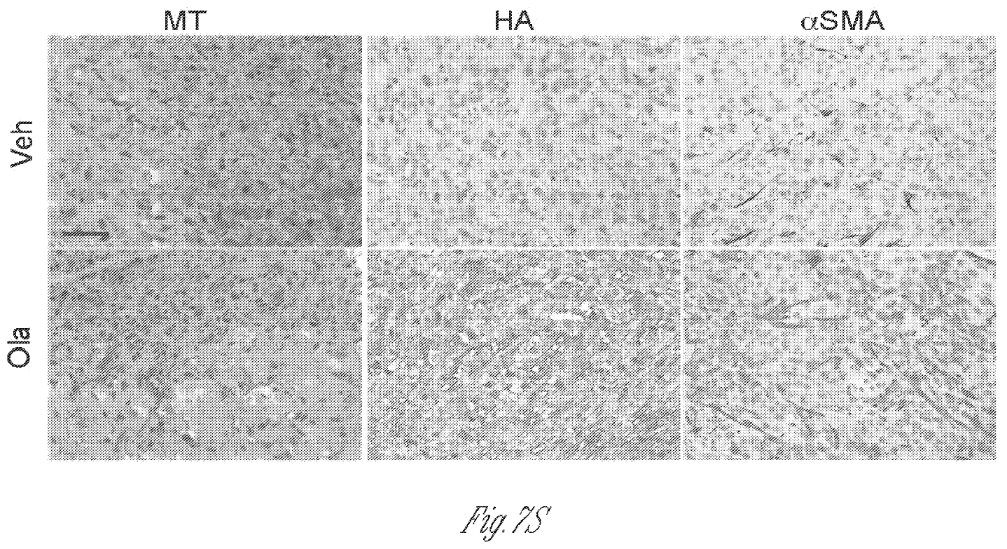

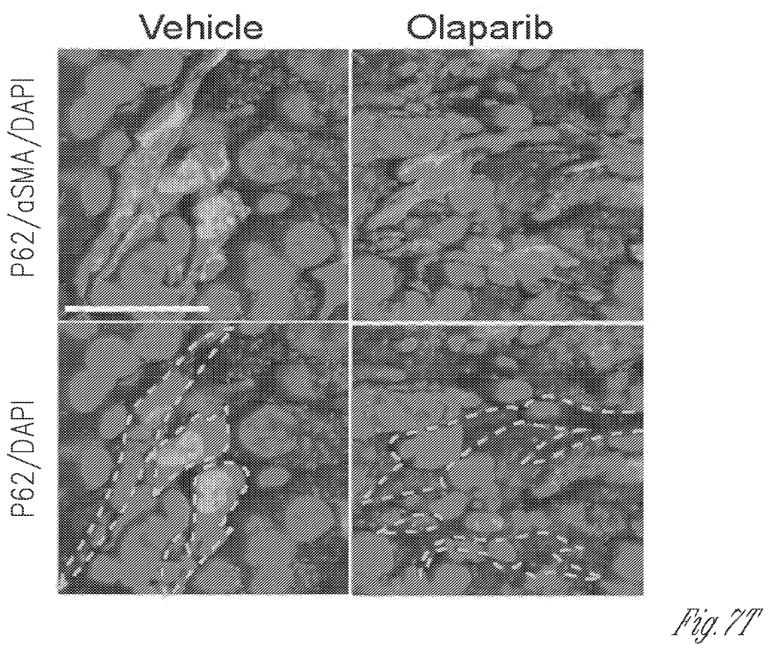

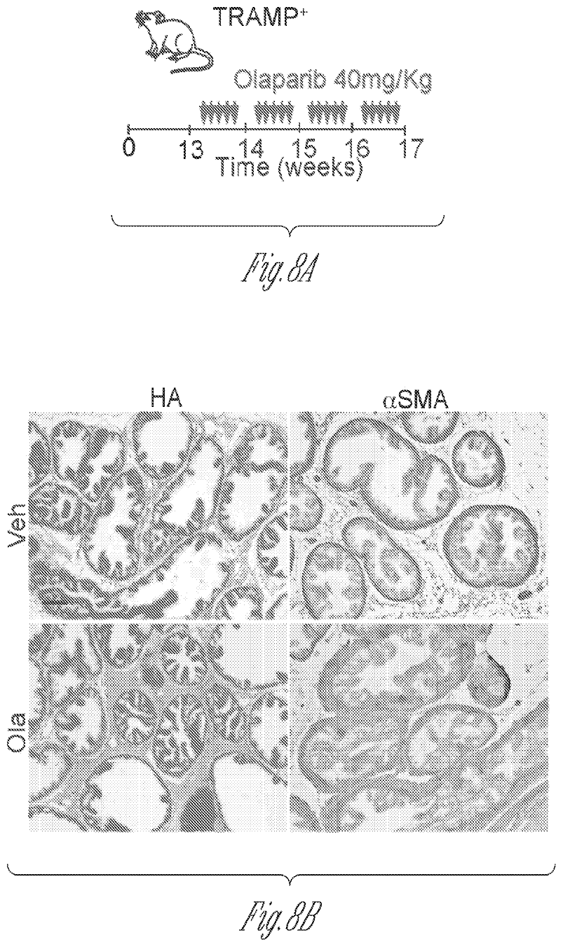

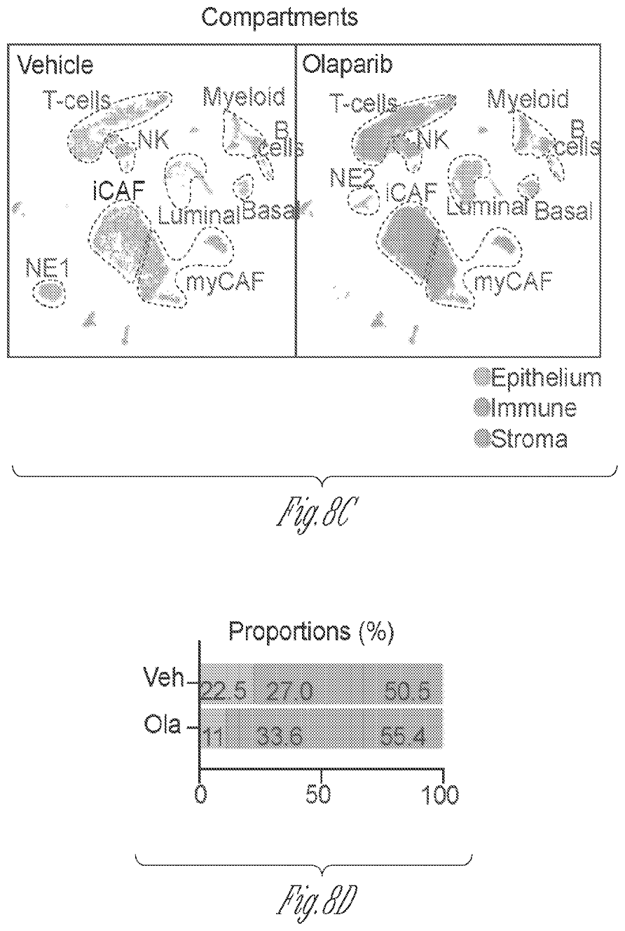

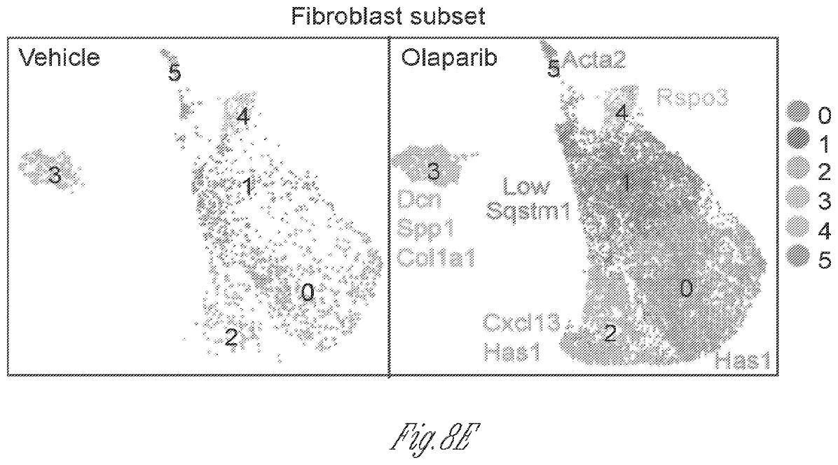

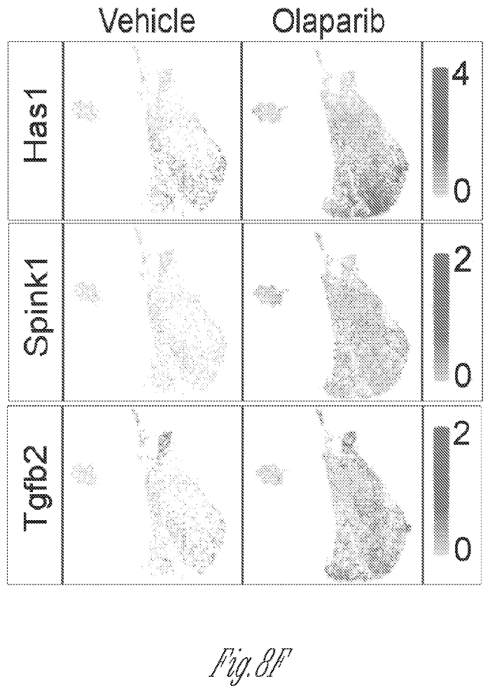

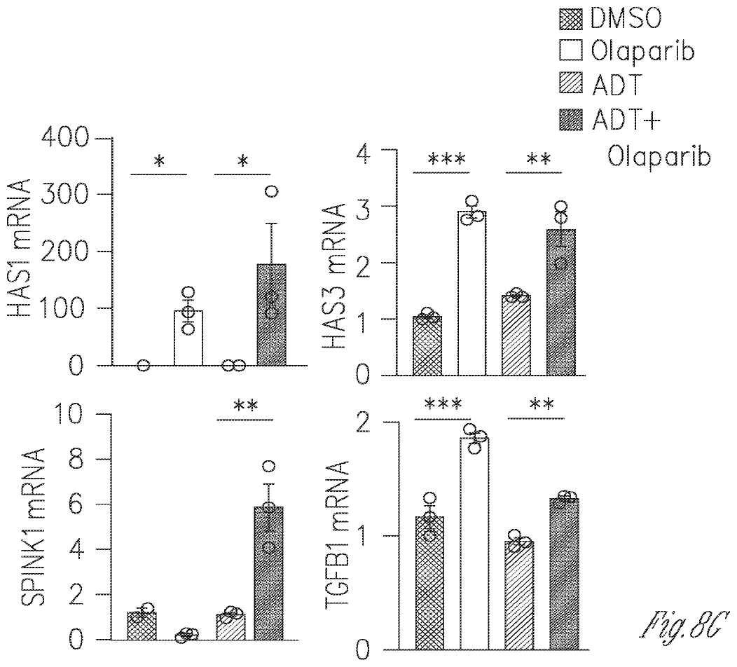

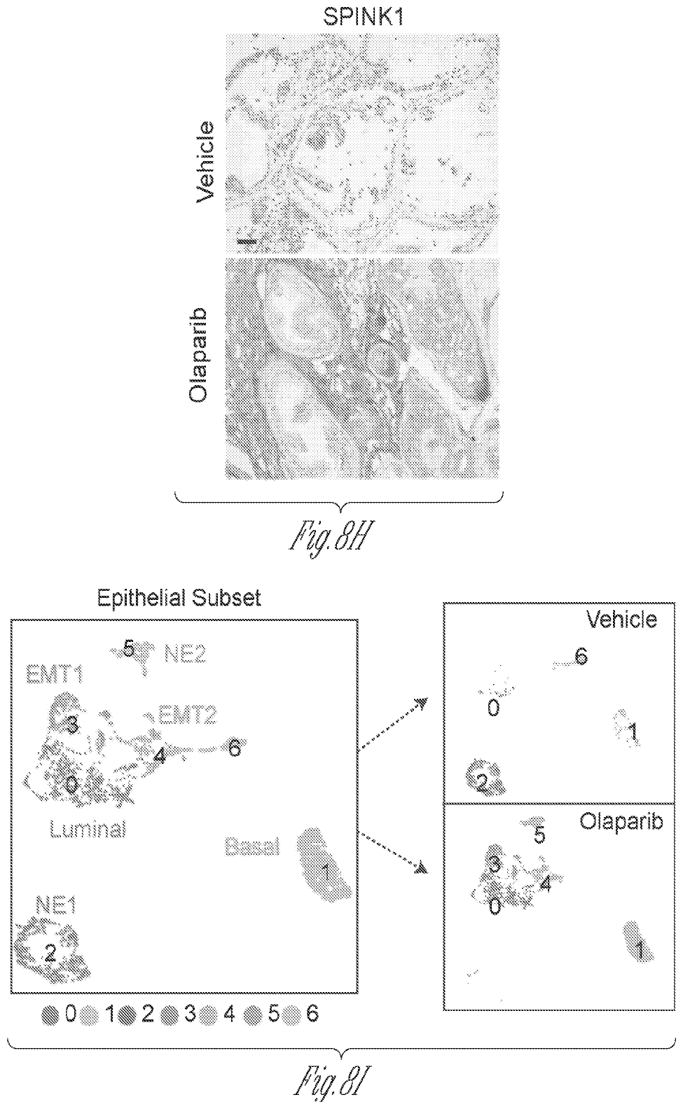

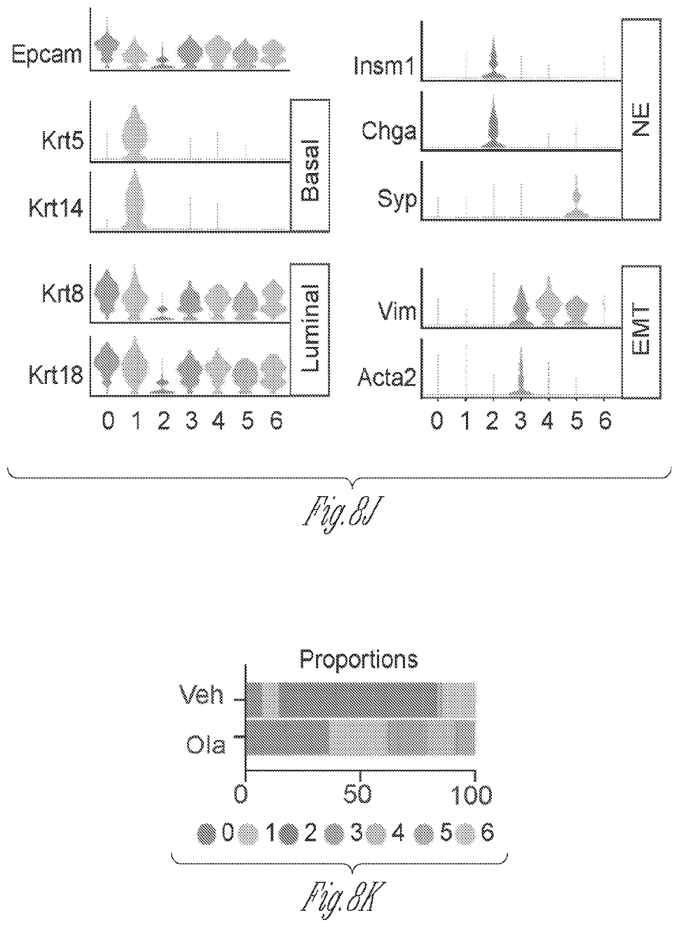

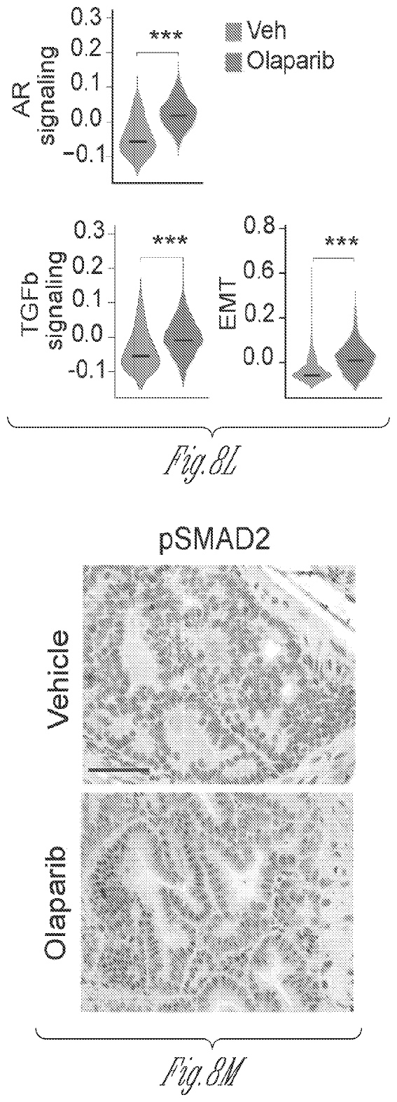

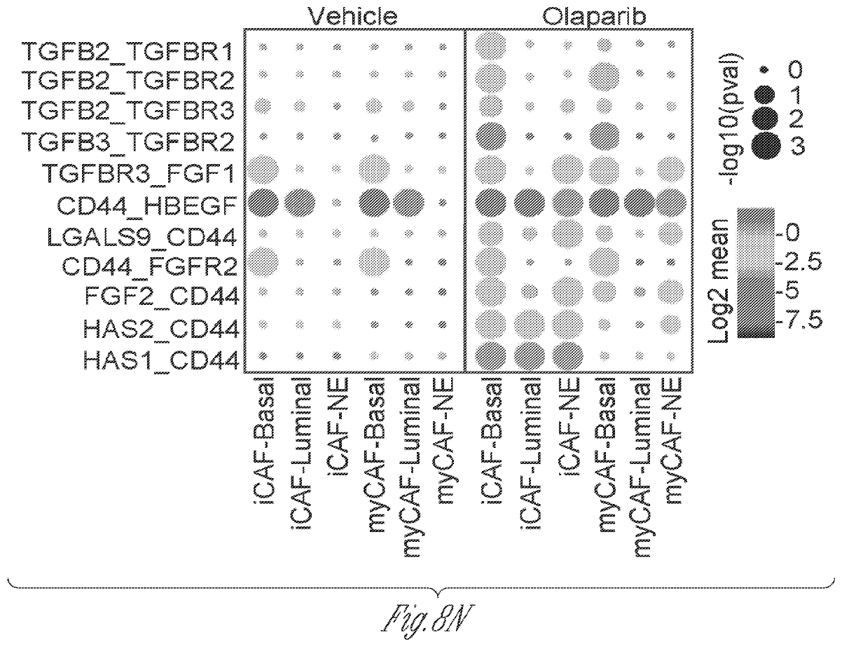

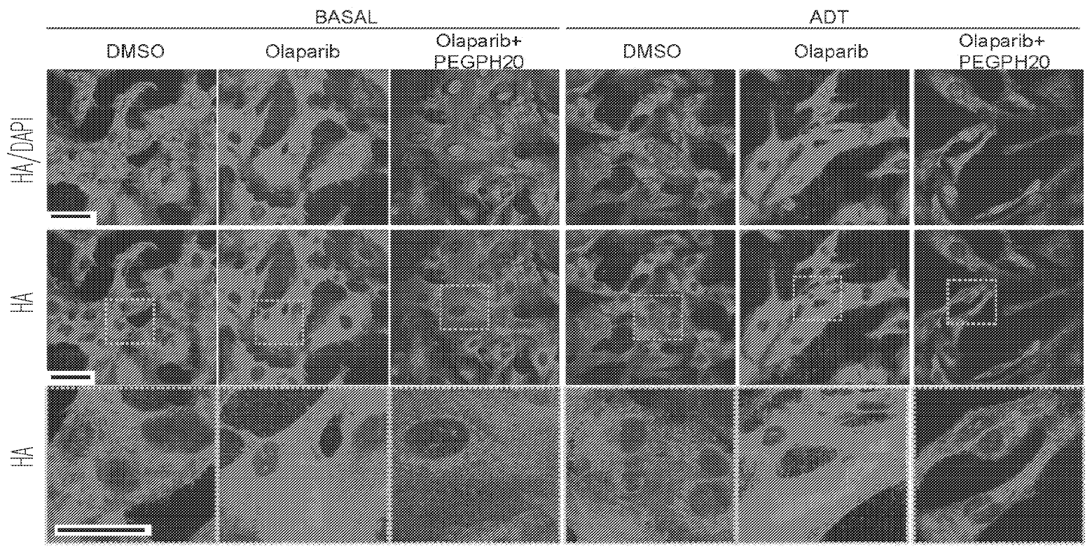

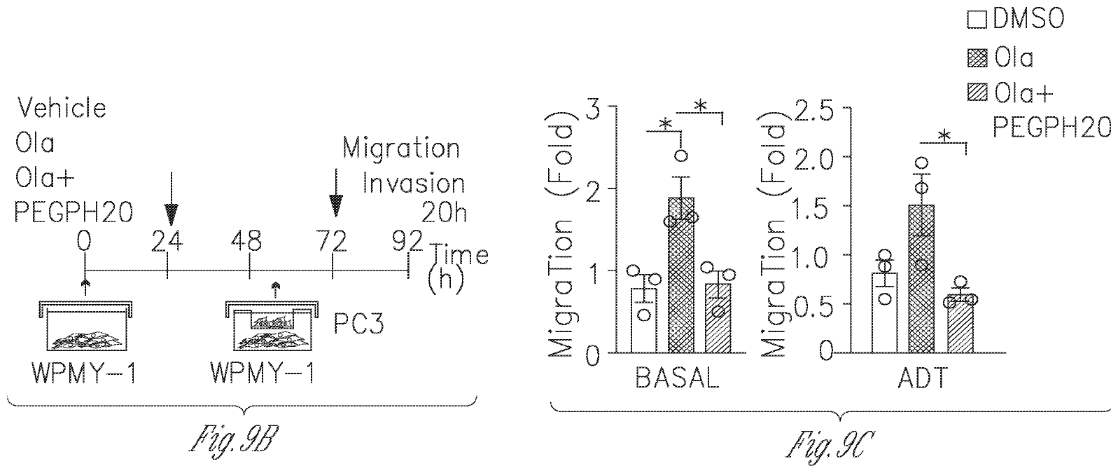

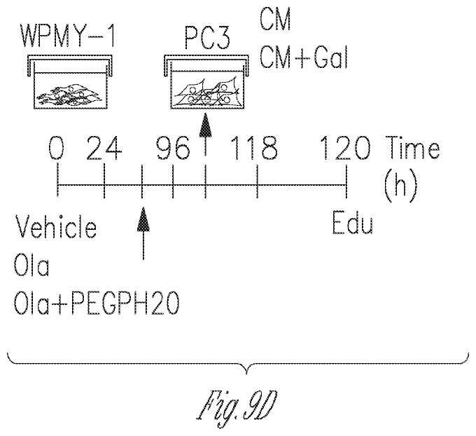

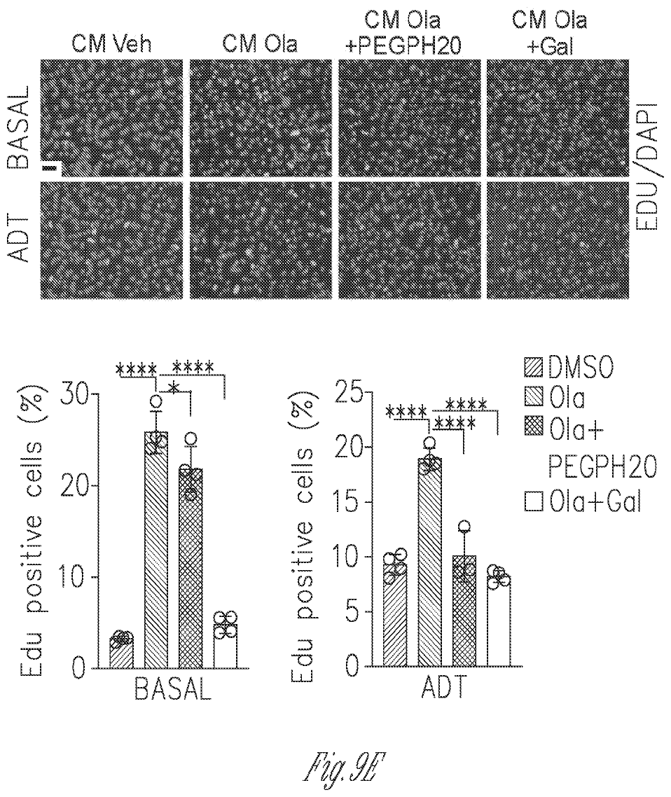

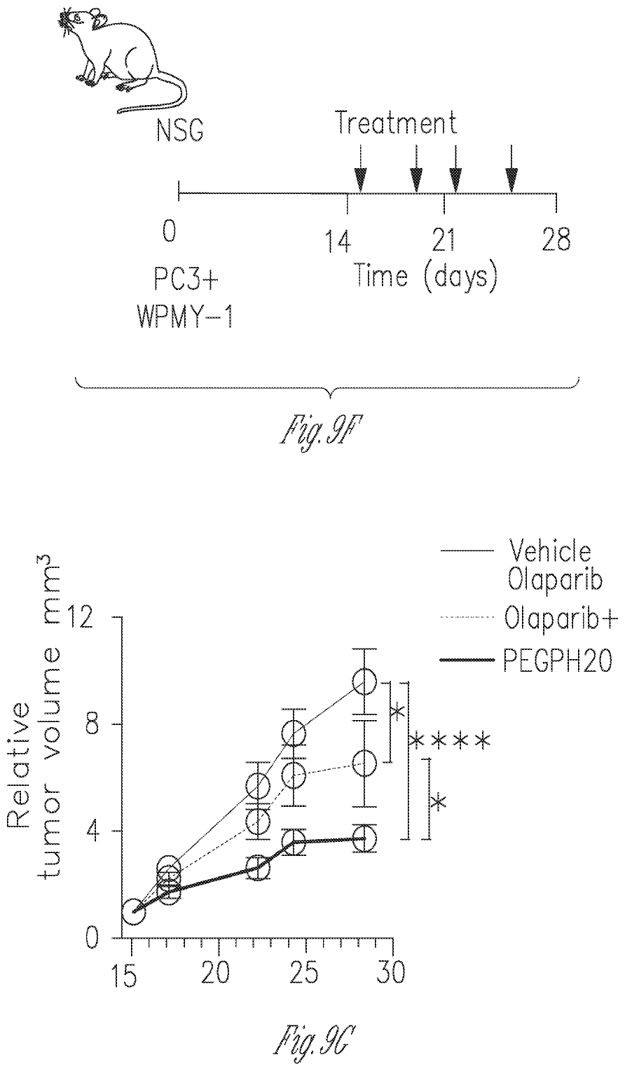

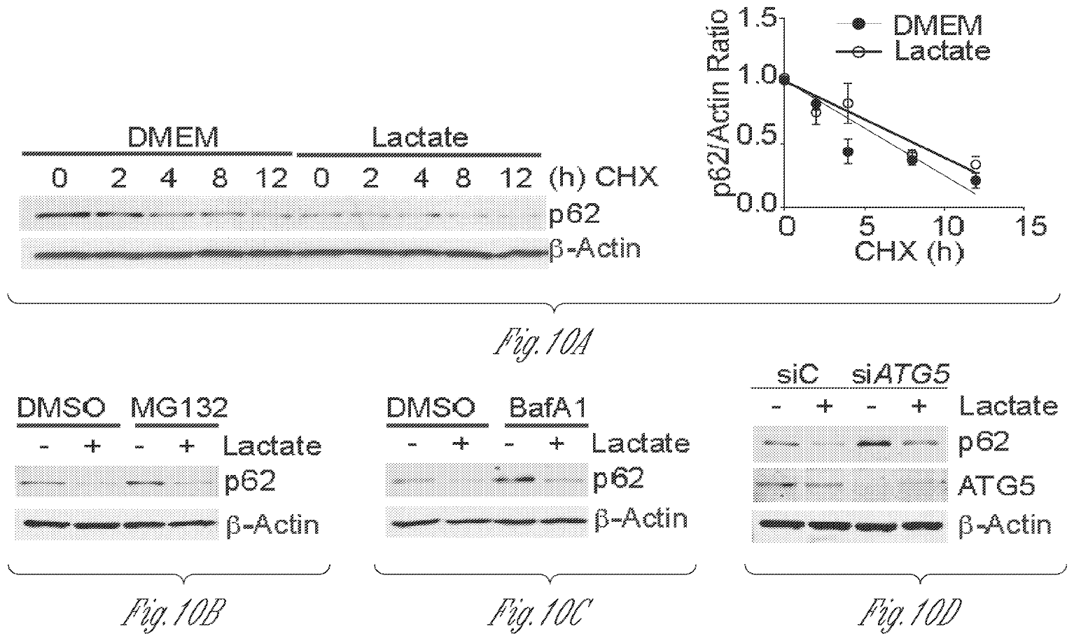

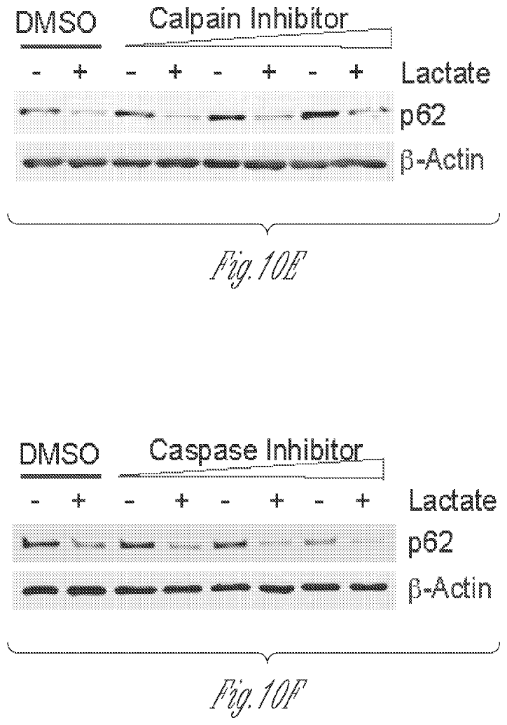

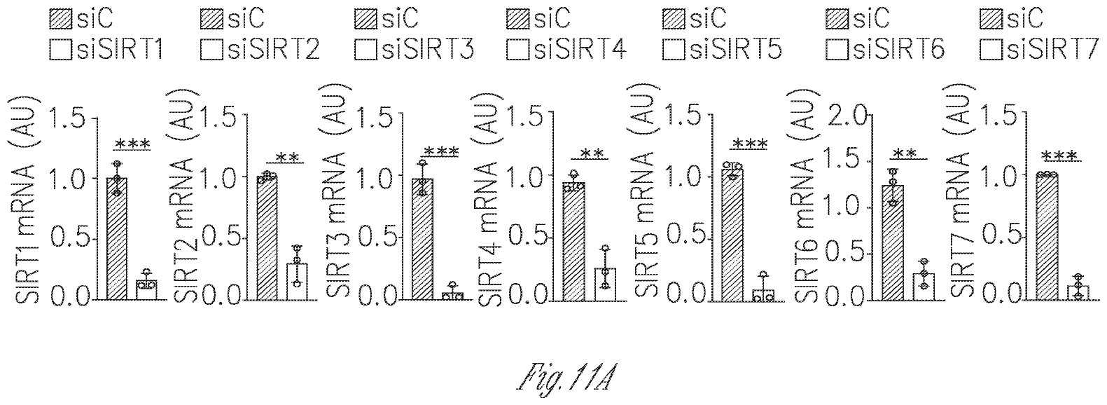

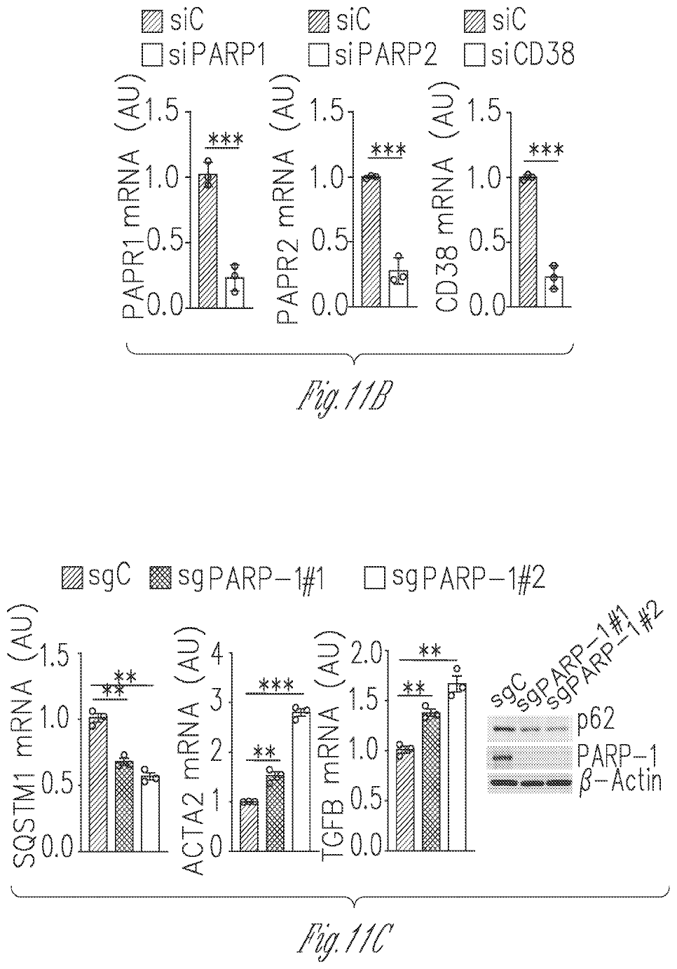

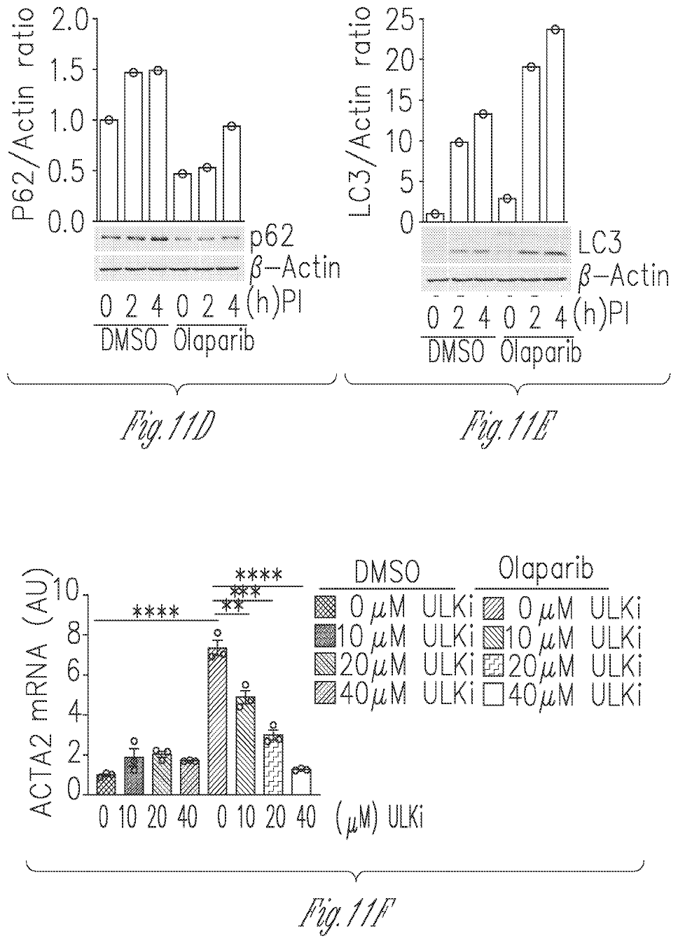

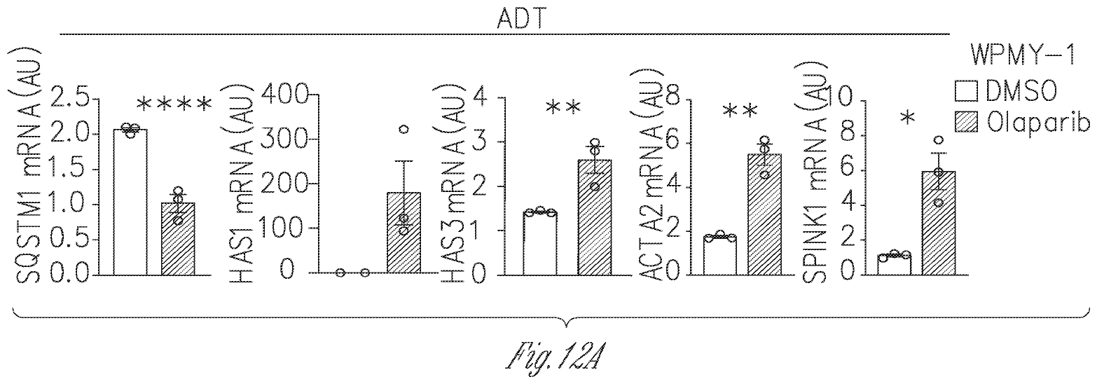

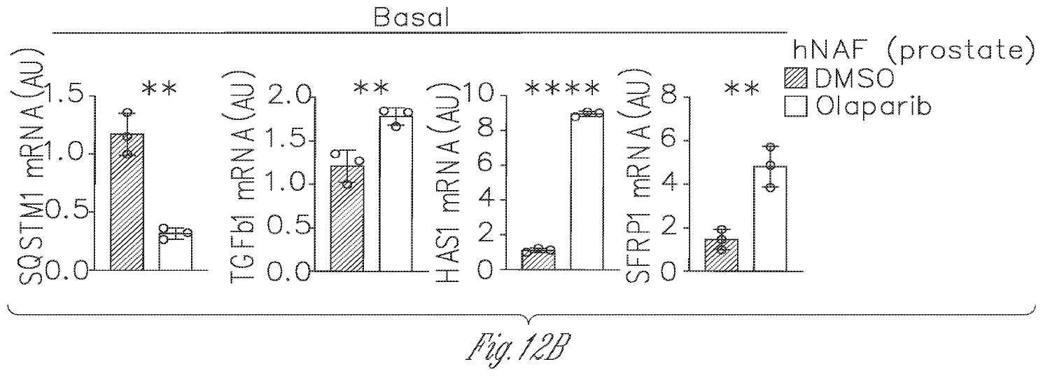

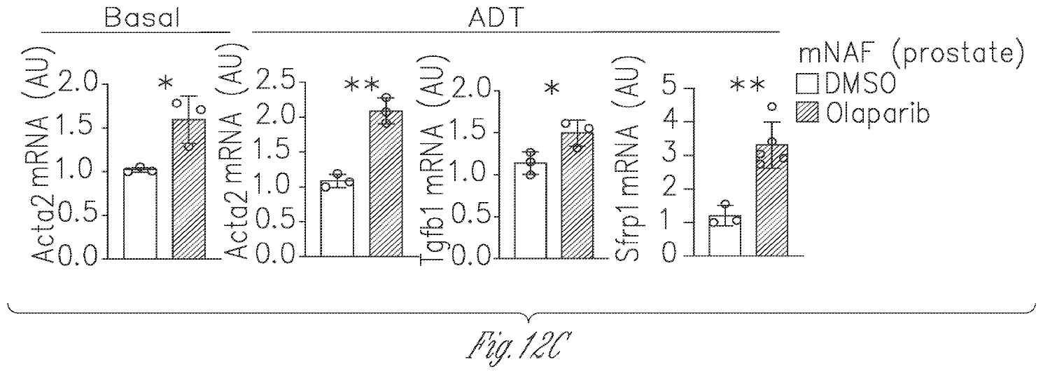

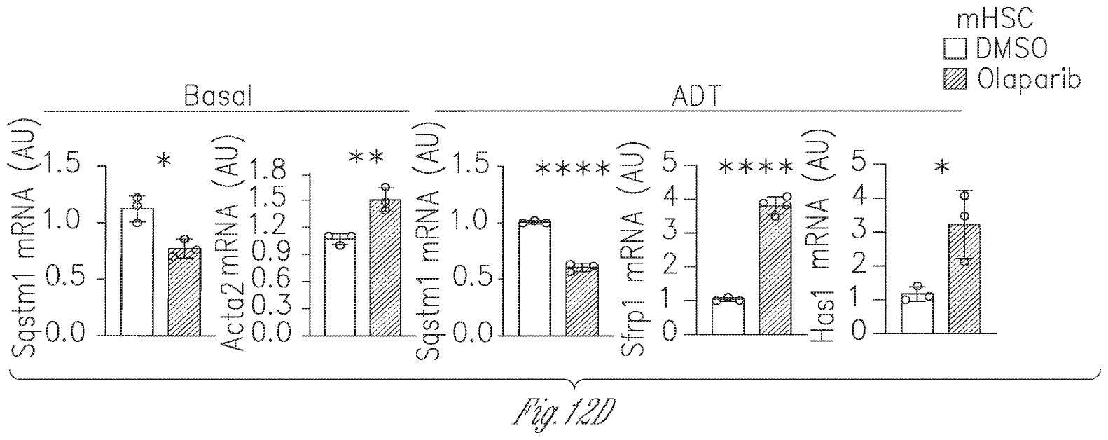

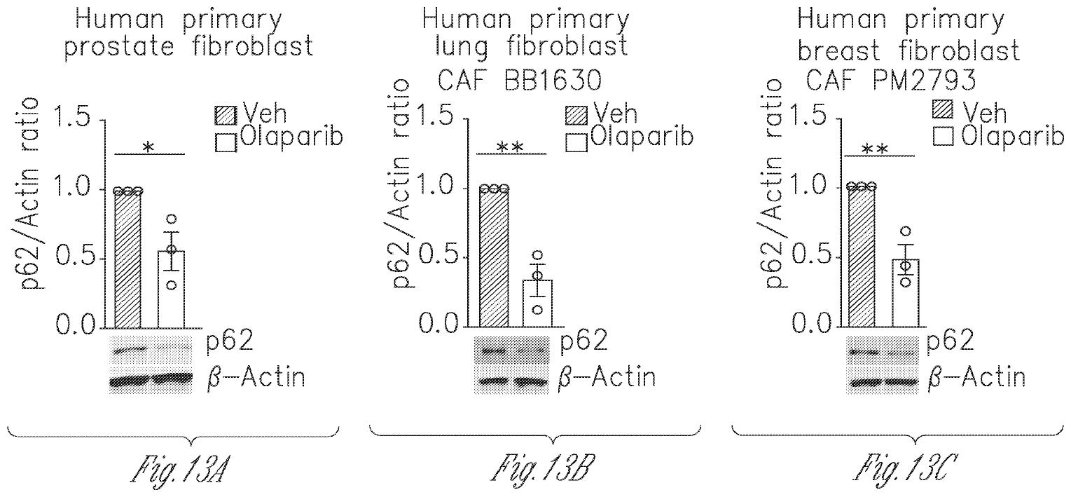

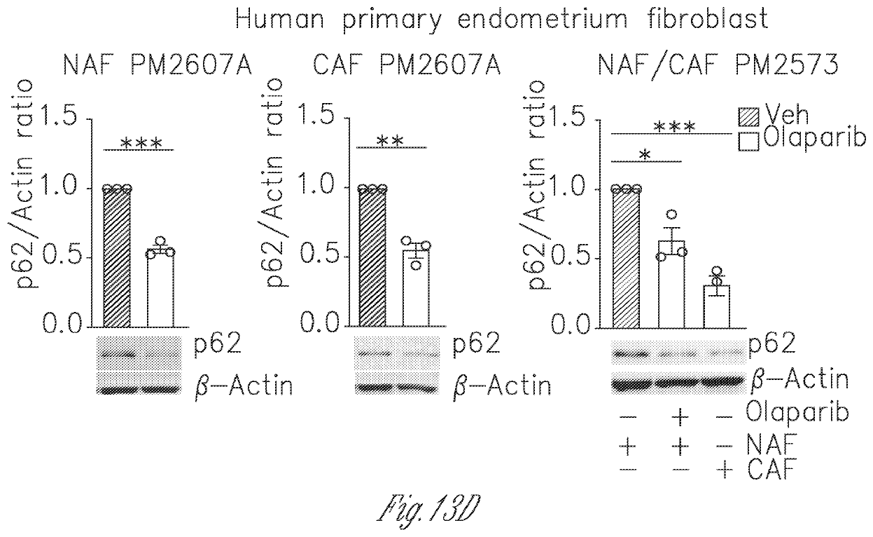

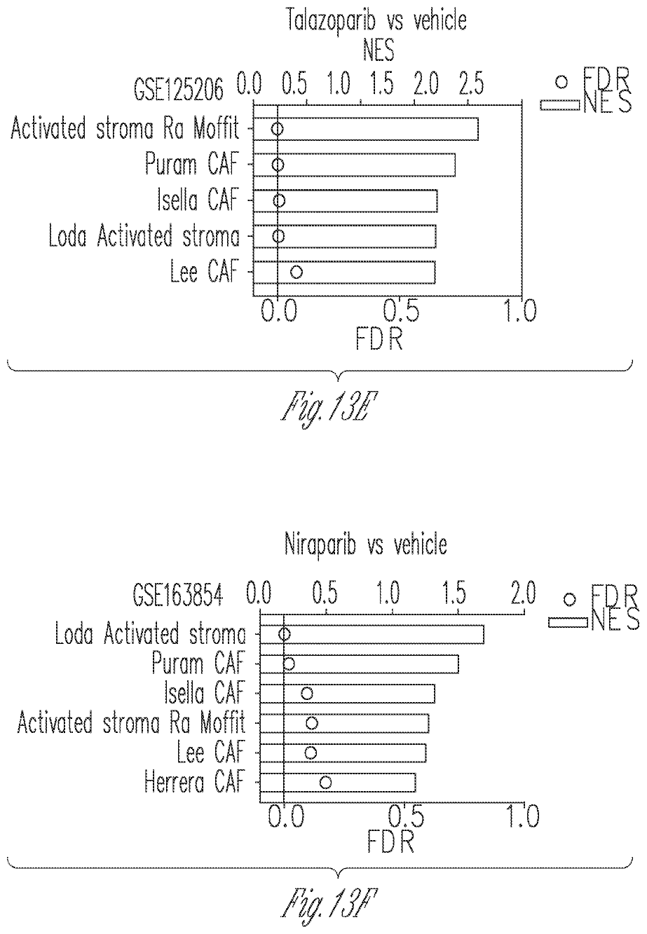

To investigate how p62 expression is downregulated in the tumor stroma, an in vitro cell system was generated in which mouse GFP-labeled prostate stromal cells (mPSC) mixed with TRAMPC2 PCa epithelial cells were incubated. Analysis of GFP-positive stromal cells isolated by FACS sorting showed reduced Sqstm1 (coding for p62) mRNA levels in stromal cells upon incubation with tumor cells, which also correlated with an increase in bona-fide CAF markers, such as Tgfb1 and 10 Sdf-1 (FIG. 1A). To test whether the downregulation of p62 required cell contact between tumor and stromal cells, human prostate stromal fibroblasts (WPMY-1) were co-cultured with different PCa cell lines (PC3, TRAMPC2, and DU145) in a double-chamber setting. p62 was also downregulated at the protein and mRNA levels in stromal cells under these conditions (FIGS. 1B and 1C), indicating that a soluble factor secreted by PCa epithelial cells was sufficient to downregulate p62 in stromal cells. In fact, incubation of stromal cells with conditioned media (CM) from PCa cells (PC3, TRAMPC2, DU145 or LNCAP) effectively reduced p62 protein and mRNA levels in stromal cells, without any effect by the CM from normal prostate epithelial cells (PrEC or RWEP1) (FIGS. 1D and 1E). To determine if the reduction in the mRNA amount of SQSTM1 was due to inhibited transcriptional activity, cells transfected with a luciferase reporter under the control of the SQSTM1 promoter (Duran et al., 2008) were used. PCa-conditioned media reduced the activity of the luciferase reporter (FIG. 1F), strongly suggesting that the downregulation of stromal p62 by PCa epithelial cells is mediated through the repression of the SQSTM1 promoter in the stroma by a soluble factor secreted by epithelial tumor cells.

To identify this putative tumor-derived soluble factor, conditioned medium from PC3 cells was size-fractionated to separate molecules that were either less or more than 3 kDa in size, and the ability of either fraction to decrease the amount of stromal p62 was tested. Both the unfractionated tumor-conditioned medium and the <3 kDa fraction comparably downregulated p62 at the protein and mRNA levels in stromal fibroblasts (FIGS. 1G and 1H), as well as the activity of the SQSTM1 promoter-driven luciferase reporter (FIG. 1I). However, the >3 kDa fraction was totally inactive in these assays (FIGS. 1G-1I). The active soluble factor was heat-stable (FIGS. 1J-L), which suggested that it could be a metabolite.

### Lactate Downregulates p62 in Stromal Fibroblasts

Since cancer cells secrete large amounts of lactate that accumulates in the tumor microenvironment as a consequence of the Warburg effect (Vander Heiden et al., 2009), it was speculated that lactate could be a good candidate to be the tumor-derived factor responsible for the downregulation of p62 in stroma cells. Consistent with this hypothesis, it was found that the amount of lactate in the media of different PCa cell lines was higher than in the media of normal prostate epithelial cells (FIG. 2A). The cellular uptake of lactate is mediated by different monocarboxylate transporters (MCT1, MCT2, MCT3, and MCT4) (Halestrap, 2013). A prediction would be that blocking these transporters could alleviate the lactate-induced downregulation of p62. Notably, knockdown of MCT1, which controls lactate cellular import in WPMY-1 cells resulted in the inhibition of p62 downregulation induced by CM from PC3 cells (FIGS. 2B and 2C). Likewise, p62 downregulation in WPMY-1 cells by the same CM was abrogated when MCT4, which controls lactate export, was knocked-down in PC3 cells (FIGS. 2D and 2E). Furthermore, treatment with the MCT1 inhibitor, AZD3965, impaired p62 downregulation in WPMY-1 cells treated with PC3 CM (FIGS. 2F and 2G).

Together these results demonstrate that secretion of lactate by PCa cells and its uptake by stromal cells is a step in the stromal downregulation of p62. To demonstrate that lactate is sufficient to downregulate p62 in stromal cells, WPMY-1 cells were incubated with different concentrations of lactate for 24 hours or 48 hours, which resulted in the downregulation of p62 in a time (FIGS. 2H and 21) and dose-dependent (FIGS. 2J and 2K) manner. The amount of lactate necessary to downregulate p62 in stromal cells (FIGS. 2J and 2K) was similar to the secreted by PCa cells (FIG. 2A), and consistent with reported data from tumors in vivo (Walenta et al., 2000). MCTs are proton-coupled symporters, which explains why lactate-mediated downregulation of p62 was dependent on an acidic pH (FIG. 2L). However, the simple acidification of the culture media was not sufficient to downregulate p62 in stromal cells (FIG. 2L). These results demonstrated that lactate secreted by epithelial PCa cells impaired p62 expression in stromal cells at the transcriptional level. To rule out the potential contribution of a hypothetical role of p62 protein stability in the response to lactate, cycloheximide-treated cells were incubated with lactate and p62 protein levels analyzed. Lactate does not affect p62 stability (FIG. 10A). In agreement with these results, inhibition of the proteasome (FIG. 10B), autophagy (FIGS. 10C and 10D), or cysteine proteases (FIGS. 10E and 10F) did not rescue the reduced p62 protein levels in lactate-treated WPMY-1 cells.

### API Controls p62 Downregulation by Lactate

To unravel the mechanisms whereby lactate downregulates stromal p62, the chromatin accessibility landscape of WPMY-1 cells treated or not with lactate by genome-wide ATAC-seq (assay for transposase-accessible chromatin with high-throughput sequencing) was investigated. This analysis revealed a widespread decrease in chromatin accessibility in lactate-treated WPMY-1 cells (FIG. 3A). HOMER (Hypergeometric Optimization of Motif EnRichment) analysis showed a significant enrichment of AP-1 transcription factors in the closed chromatin regions of lactate-treated cells (FIG. 3B). Analysis of the SQSTM1 promoter demonstrated the presence of three AP-1 regulatory sites that also showed reduced chromatin accessibility under lactate-treated conditions (FIG. 3C). Consistently lactate repressed an AP-1-driven luciferase reporter (FIG. 3D), which collectively indicated that the repression of AP-1 sites in the SQSTM1 promoter could account for lactate effects in stromal p62 expression.

To establish the contribution of the different AP-1 sites in the SQSTM1 promoter, AP-1A, AP-1B, and AP-1C) were individually mutated and the impact of these mutations in the SQSTM1 promoter activity was determined by luciferase assay. Mutation of AP-1A completely inactivates the SQSTM1 promoter to levels comparable to those produced by lactate in the intact SQSTM1 promoter, whereas mutation of AP-1B, or AP-1C had no effect (FIG. 3E). These results demonstrate that AP-1A is the important enhancer element in the SQSTM1 promoter and the most likely target of lactate-induced repressive effects. To test this hypothesis, in a more physiologically relevant system, the AP-1A element in the endogenous SQSTM1 promoter was selectively deleted using CRISPR/Cas9 editing to generate four independent WPMY-1 cell cultures (sgAP-1A), which resulted in the inhibition of mRNA and protein p62 expression (FIGS. 3F-H). These results establish the relevance of AP-1A for the regulation of p62 expression in stromal cells. In keeping with this notion, chromatin immunoprecipitation (ChIP) analysis demonstrated that the recruitment of c-FOS and c-JUN, but not of FOSB or JUNB, to the AP-1A regulatory site was significantly decreased in stromal fibroblasts stimulated with lactate (FIG. 3I). Consistently, the downregulation of c-FOS (FIGS. 3J and 3K) or the inhibition of c-JUN phosphorylation with a pharmacological inhibitor of JNK (FIGS. 3L and 3M), mimicked the effect of lactate on p62 expression both at the protein and mRNA levels. Overall, these data indicate that the impaired recruitment of a c-JUN/c-FOS AP-1 transcriptional complex plays a role in p62 downregulation by lactate in the stroma.

### Reduction in NAD⁺ Levels by Lactate Metabolism Mediates p62 Downregulation

Lactate can be reversibly converted into pyruvate with the subsequent depletion of NAD⁺ levels in favor of the generation of NADH (Covarrubias et al., 2021). Importantly, increasing the levels of pyruvate in the culture media, which drives this reaction in the opposite direction, clearly impairs the ability of lactate to downregulate p62 (FIGS. 4A and 4B). Therefore, changes in the NAD⁺/NADH ratio could be instrumental for the mechanisms of action of lactate on the regulation of p62 levels. Consistent with this hypothesis, NAD⁺ levels in stromal fibroblasts treated with lactate are lower than in untreated cells (FIG. 4C). Treatment with NAD⁺ or with the NAD⁺ precursor, nicotinamide riboside (NR), completely rescued the lactate-induced reduction in p62 (FIGS. 4D-4G).

### PARP-1 Inhibition by Lactate Downregulates p62 in Stromal Cells

NAD⁺ is a cofactor for three types of transcriptional and posttranslational modulators (Covarrubias et al., 2021). These include the sirtuins (SIRT1 through SIRT7), the cyclic ADP ribose synthases (CD38 and CD157), and the poly-ADP ribose transferases (PARP1/2). Interestingly, only the knockdown of PARP-1 fully mimicked the effect of lactate on p62 expression (FIGS. 5A-5E, FIGS. 11A and 11B). Moreover, treating WPMY-1 cells with the PARP-1 inhibitors PJ34 (FIGS. 5F and 5G) or Olaparib (FIGS. 5H and 51) also reduced the expression of p62 in a dose-dependent manner. Of note, Olaparib also induced autophagy activation as determined by LC3 lipidation (FIGS. 11D and 11E). Since p62 is a substrate of autophagy, Olaparib impaired p62 expression at the mRNA and protein levels. Interestingly, co-treatment with the autophagy inhibitors (ULKi, SBI-0202965) reverted the Olaparib-induced activation of stroma in a dose dependent manner (FIG. S2F), which suggest the use of autophagy inhibitors to overcome the stromal activation in response to PARP1 inhibitors.

PARP1, in addition to its well-known role in DNA damage repair, also controls many other biological processes including transcription (Feng et al., 2015). Thus, PARP-1 reportedly enhances the DNA binding and transactivation of many transcription factors (Gibson and Kraus, 2012). Of special relevance for this study, both c-FOS and c-JUN have been previously shown to be poly(ADP-ribosyl)ated by PARP-1, which increased their DNA binding activity (Huang et al., 2009). Consistently, Olaparib impaired the recruitment of c-FOS and c-JUN to the AP-1A binding site in the SQSTM1 promoter (FIGS. 5J and 5K). Taken together, these results suggest that the regulation of PARP-1 activity by changes in the levels of NAD+ triggered by lactate metabolism in stromal cells is instrumental for p62 downregulation. Therefore, it was posited that lactate could affect the activation of PARP-1 and, consequently, the poly(ADP-ribosyl) ation of c-JUN and c-FOS. Consistently, lactate-treated cells have reduced total poly(ADP-ribosyl) ation levels (FIG. 5L), and immunoprecipitation experiments demonstrated that lactate impaired the poly(ADP-ribosyl) ation of c-JUN and c-FOS (FIGS. 5M and 5N). Adding NAD⁺ to lactate-treated cells rescued the poly(ADP-ribosyl) ation of both transcription factors (FIGS. 50 and 5P) and, importantly, their recruitment to the AP-1 regulatory site in the SQSTM1 promoter (FIGS. 5Q and 5R). Similar results were obtained by treating these cells with NR (FIGS. 5S and 5T). Taken together, these data demonstrate that depletion of NAD⁺ by lactate metabolism and the subsequent impairment of the poly(ADP-ribosyl) ation of c-FOS and c-JUN, underlies the mechanism whereby lactate triggers the downregulation of p62 in stromal fibroblasts.

### AP-1 is Important for CAF Activation Driven by p62 Loss

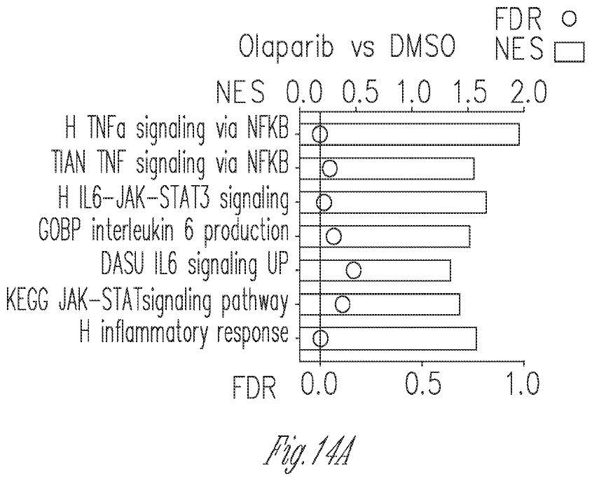

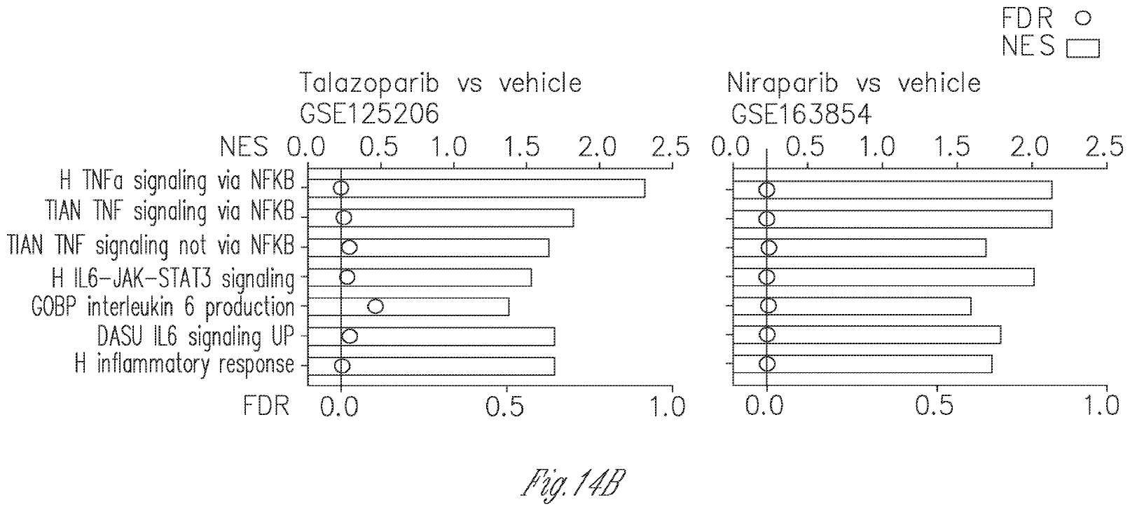

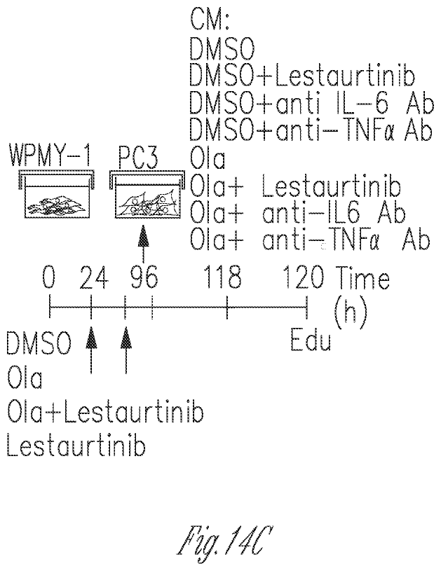

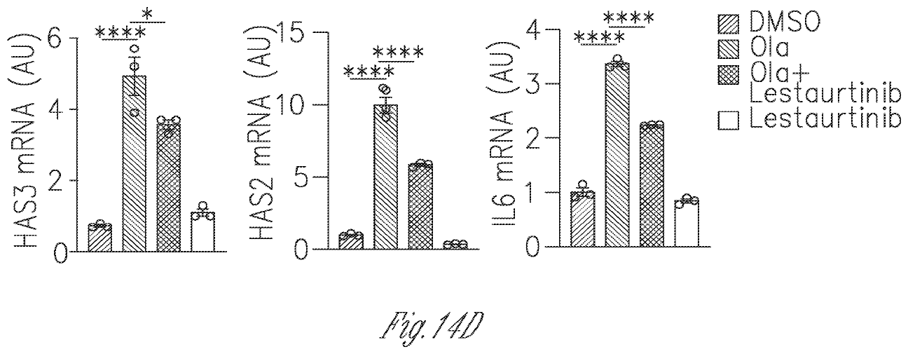

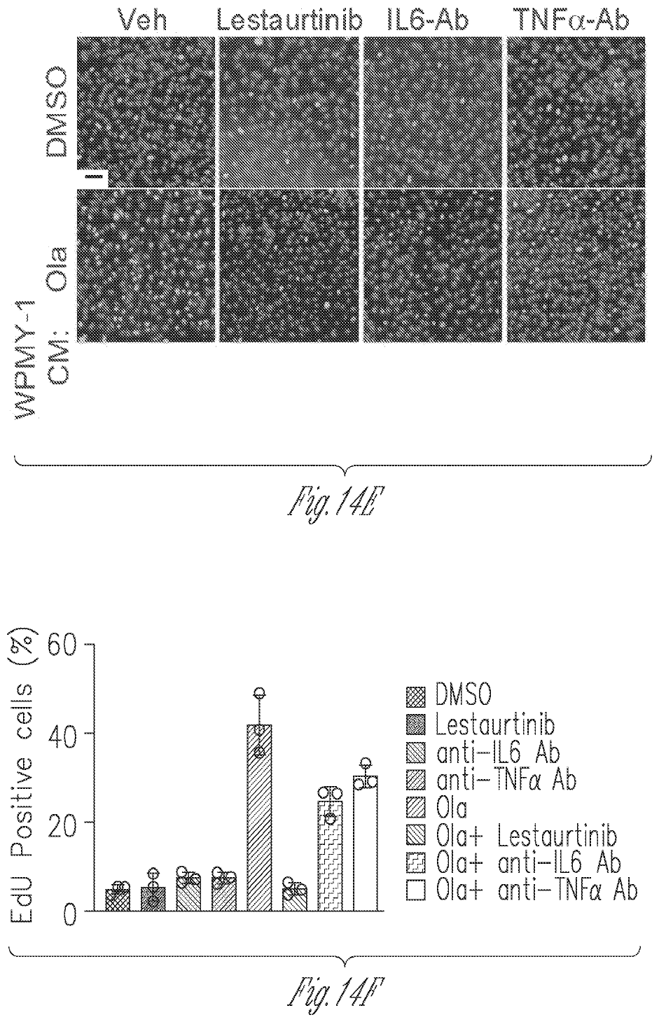

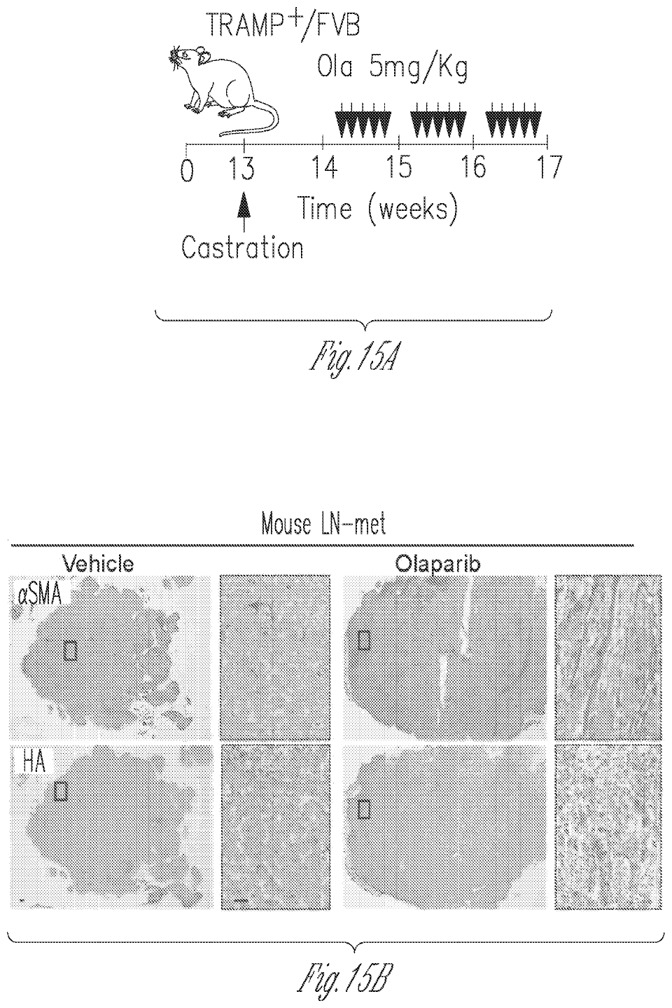

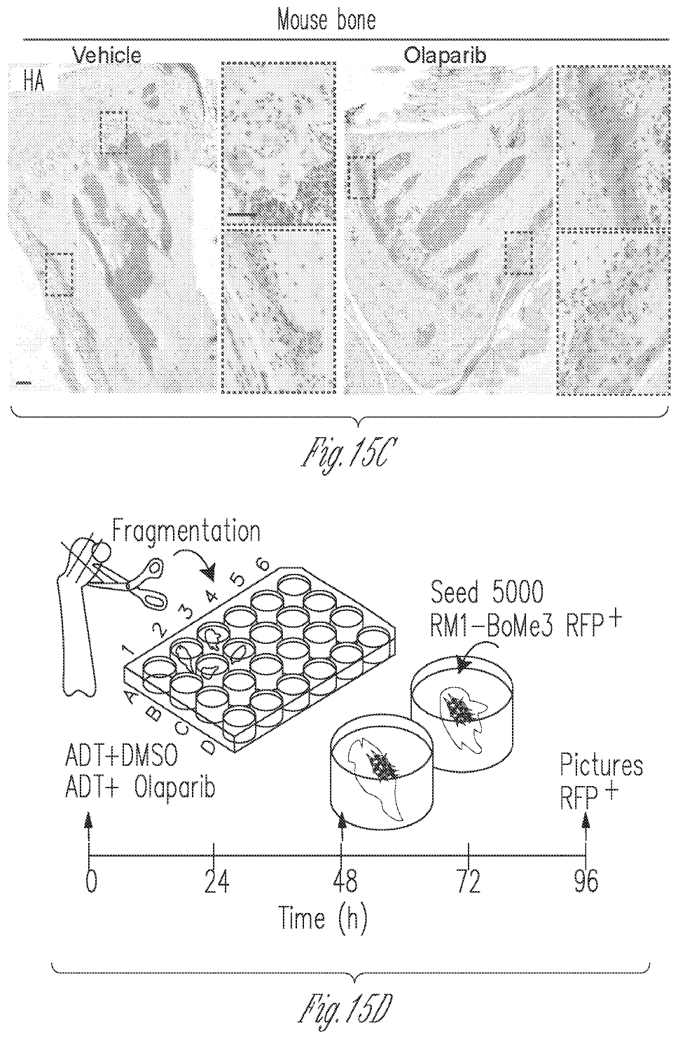

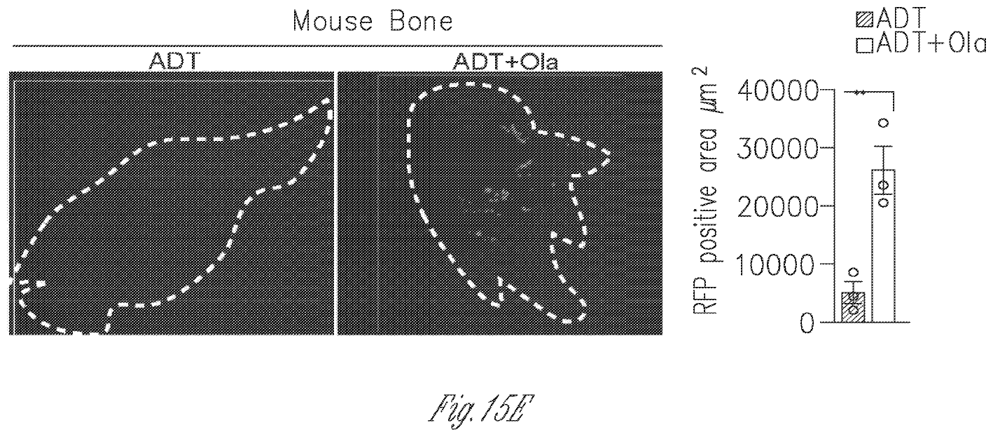

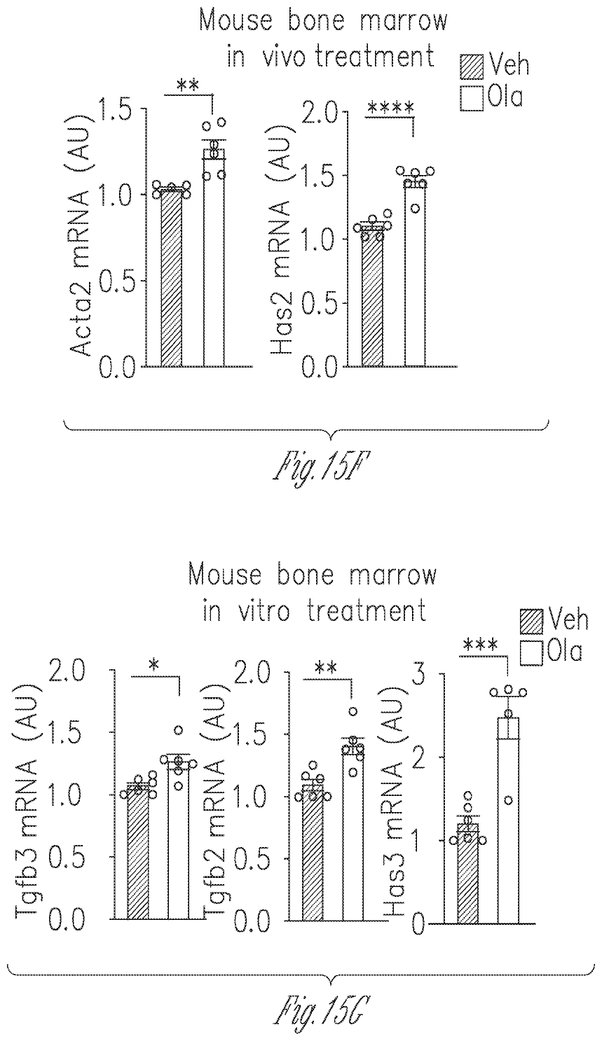

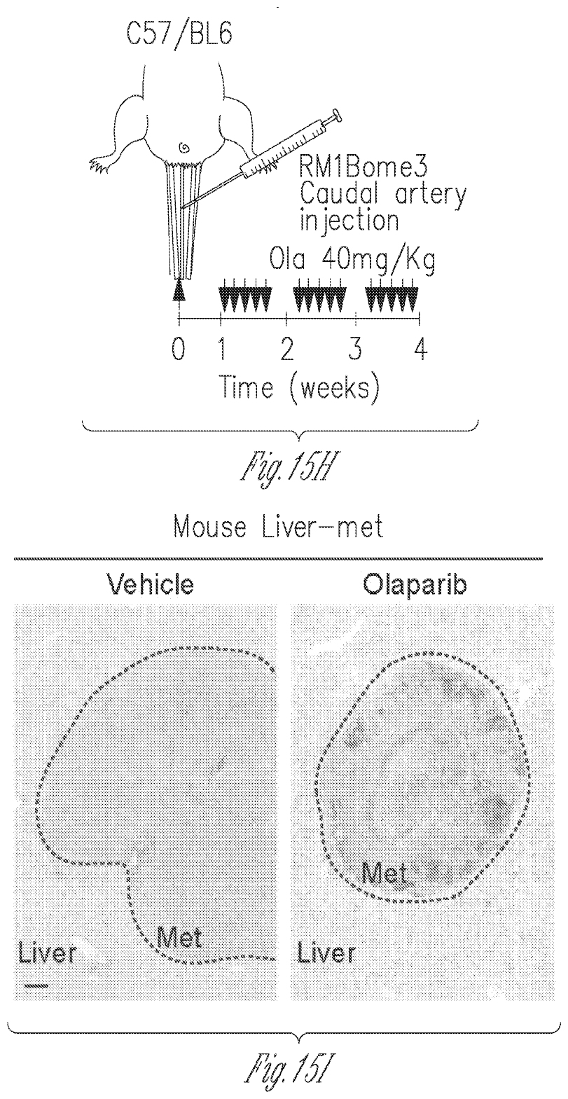

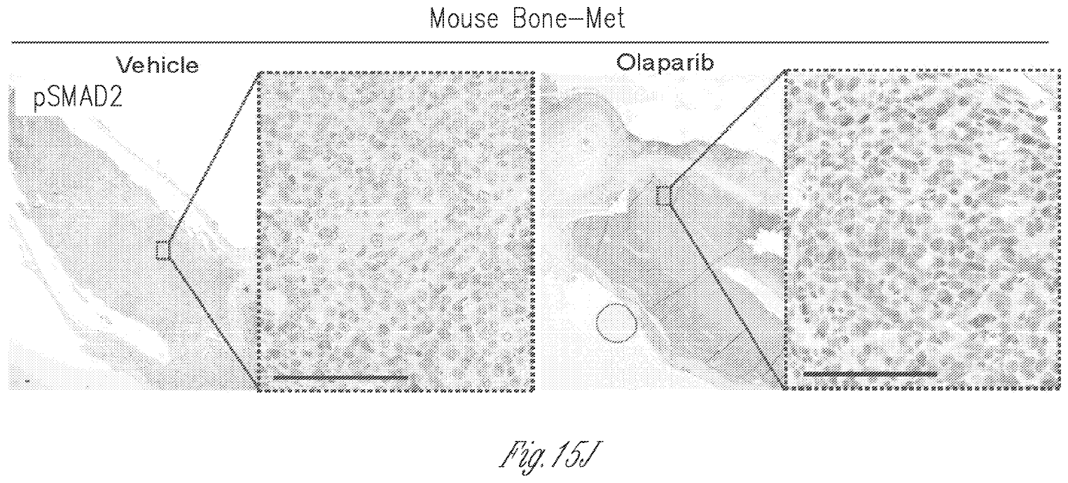

The loss of p62 drives a CAF phenotype in stromal cells, which promoted tumor progression (Linares et al., 2017; Valencia et al., 2014). Because lactate secretion by PCa cells is necessary and sufficient to downregulate p62 in stromal cells, we next determined whether treating WPMY-1 cells with lactate drives them to a CAF phenotype. Treatment of WPMY-1 cells with lactate increased the expression of bona fide markers of CAF activation such as ACTA2 and TGF-β (FIG. 6A), whose expression was reverted by NAD⁺ addition (FIG. 6B). Gene set enrichment analysis (GSEA) of genes differentially expressed in lactate-treated WPMY-1 cells showed enrichment in CAF signatures from a human stroma PCa data set (GSE34312) (FIG. 6C), validating the human relevance of these observations. Importantly, sgAP-1A fibroblasts in which the SQSTM1 promoter was endogenously inactivated (FIG. 3F) also showed increased expression of CAF markers (FIG. 6D). Consistently, experiments in a two-chamber co-culture system demonstrated that sgAP-1A but not sgC fibroblasts promoted the migration and invasion of PC3 cells (FIGS. 6E-6G). Similar results were obtained using a previously described 3D organotypic model that recapitulates key aspects of epithelial-stromal crosstalk in the tumor microenvironment (Valencia et al., 2014) (FIGS. 6H and 6I). In keeping with the in vitro data, subcutaneous xenograft co-implantation of PC3 cells with sgAP-1A in immunodeficient mice (NSG), resulted in increased tumor growth when compared with those co-implanted with sgC fibroblasts (FIGS. 6J-6L). Notably, tumors generated with sgAP-1A fibroblasts showed increased markers of stromal activation, such as the augmented deposition of collagen and hyaluronan (HA), and the enhanced expression of aSMA (FIG. 6M). Collectively, these results establish that the AP-1-regulatory site in the SQSTM1 promoter, which is the target of lactate-driven signals and has a pivotal role in the control of endogenous p62 levels in stromal cells, is important for the induction of the CAF phenotype.

### PARP-1 Inhibitors Promote a Desmoplastic Response in Stromal Fibroblasts Through p62

According to the model, lactate triggers the downregulation of p62 in stromal fibroblasts through PARP-1 inhibition. Therefore, it was hypothesized that PARP-1 inhibitors such as Olaparib would be sufficient to induce stromal activation. In agreement with this hypothesis, GSEA of RNAseq data from WPMY-1 fibroblasts, treated or not with Olaparib under basal or androgen deprivation (ADT) conditions, demonstrated enrichment of signatures corresponding to extracellular matrix remodeling, indicative of CAF activation (FIG. 7A), in Olaparib-treated cells, which correlated with well-established gene signatures for stromal activation in several types of cancers, including PCa (FIGS. 7B-7C). Consistently, Olaparib treatment reduced SQSTM1 mRNA levels and upregulated CAF markers such as ACTA2, SFRP1, TGF B, MMP9 and the HA synthases in basal and ADT conditions (FIGS. 7D and 12A). Similar results were obtained in different human and mouse prostate stromal cell lines and in hepatic stellate cells (FIGS. 12B-D), suggesting that the stromal activation by Olaparib treatment is a general phenotype not linked exclusively to the prostate stroma. Furthermore, ATAC-seq analysis of Olaparib-treated WPMY-1 cells revealed a strong decrease in chromatin accessibility across the genome, like what was observed with lactate treatment (FIGS. 3A and 7E). Also, consistent with previous findings in lactate-treated fibroblasts, motif enrichment analysis using HOMER showed a significant loss of chromatin accessibility in regions with consensus motifs for AP-1 transcription factors (FIGS. 3B and 7F). Close inspection of ATAC-seq data demonstrated reduce accessibility of the SQSTM1 promoter (FIG. 7G).

Interestingly, co-culture of PC3 cells with Olaparib-pretreated WPMY-1 cells resulted in increased migration and invasion of PCa cells under basal conditions that was even more pronounced in ADT conditions (FIGS. 7H-7J). Moreover, Olaparib-treated WPMY-1 CM led to increased PC3 proliferation (FIGS. 7K and 7L). Based on these observations, it was posited that stromal activation in response to Olaparib treatment could hamper its anti-tumor activity. To test this hypothesis, PC3 cells that were labelled with GFP (PC3^{GFP}) were cultured either with (co-culture) or without (monoculture) WPMY-1 cells in basal or ADT conditions and treated or not with Olaparib for 10 days (FIG. 7M). Interestingly, co-culture with WPMY-1 cells rendered PC3^{GFP} cells more resistant to Olaparib as compared to treatment under monoculture conditions (FIG. 7N). This effect was even more apparent under ADT conditions (FIG. 7N). To investigate the pro-CAF phenotype of Olaparib in vivo, PC3 and WPMY-1 cells were co-implanted in NSG mice and then treated either with vehicle or Olaparib (FIG. 7O). In vivo Olaparib treatment efficiently reduced tumor growth (FIGS. 7P-7R), consistent with previous reports (Li et al., 2017; Zhang et al., 2018), but also produced a strong desmoplastic response, characterized by enhanced collagen and HA deposition and increased expression of aSMA and reduced p62 levels (FIGS. 7S and 7T).

### Single Cell Analysis of Stromal Activation and Epithelial Remodeling in Response to Olaparib In Vivo

To investigate how the treatment with Olaparib triggered the creation of a more activated, protumorigenic microenvironment in PCa, scRNAseq was performed using 10× Genomics Chromium platform on TRAMP+ prostate tumors after treatment with Olaparib for 4 weeks as compared to vehicle controls (FIG. 8A). Consistent with the results in the xenograft model (FIGS. 7O-7T), in vivo treatment of TRAMP⁺ mice induced a strong desmoplastic response in the prostate (FIG. 8B). Furthermore, unsupervised clustering identified epithelial cells as well as stromal and immune cells with selective markers for each population (FIG. 8C). Consistent with the in vitro data of stromal activation, there was an expansion of the stromal and immune populations in response to Olaparib treatment (FIGS. 8C and 8D). Further re-clustering of the fibroblast compartment identified six clusters, all enriched in CAF signatures (FIG. 8E). Clusters 0 and 2 correspond to the inflammatory CAF subtype (iCAF), whereas clusters 3 and 5 express myofibroblast markers (myCAF). Cluster 3 displayed high levels of two CD44 ligands, Colla1 and Spp1, while cluster 5 expresses Acta2. Interestingly, cluster 1 has features of both iCAF and myCAF and is enriched in growth factors and inflammatory cytokines as well as EMT markers and constitutes the subpopulation with the lowest levels of p62/Sqstm1. Cluster 4 is enriched in Rspo3 and resembled the mesenchymal cell population termed Mes2, previously described in scRNAseq analysis of normal mouse and human prostates (Karthaus et al., 2020). Importantly, differential expressed gene analysis identified Has1, Spink1 and Tgfb2 among the most upregulated transcripts in the Olaparib-treated fibroblast population (FIG. 8F). Has1 is a hyaluronan (HA) synthase, one of the three enzymes that synthesize HA, which has been linked to tumor promotion, therapy resistance, and poor outcomes in various tumors, including PCa (Aaltomaa et al., 2002; Lipponen et al., 2001; Toole, 2004; Toole and Slomiany, 2008). Also, recent reports point to a role of HAS-expressing fibroblasts in the promotion of metastatic spreading (Affo et al., 2021). These data are in good agreement with the results in human prostate stromal cells and in in vivo mouse models, in which increased deposition of HA upon Olaparib treatment was found. Notably, Has1 and Has3 were upregulated in Olaparib-treated WPMY in both basal and ADT conditions (FIG. 8G). Of great relevance, SPINK1 (Serine Peptidase Inhibitor, Kazal type 1) expression in PCa has been associated with poor response to ADT, faster progression to the castrate-resistant stage and cancer-associated mortalities (Johnson et al., 2016; Leinonen et al., 2010; Tomlins et al., 2008). Moreover, recent reports showed increased tumor Spink 1 levels in response to AR-targeted therapies (Tiwari et al., 2020) and in prostate stroma after chemotherapy (Chen et al., 2018). The present results show that SPINK1 levels were highly upregulated in human prostate cells under ADT conditions (FIG. 8G) and, more importantly, in tumor stroma in vivo in Ola-treated mice (FIG. 8H). Tgfb2 is one of the ligands of the TGFB pathway, which has been long recognized to exert opposing roles in cancer progression but that it is an important component of reactive stromas (Lu et al., 2017; Massague, 2008). While TGFβ signaling blocks tumorigenesis in primary PCa (Bjerke et al., 2014), it can promote PCa bone metastasis (Fournier et al., 2015) and has been recently associated with resistance to enzalutamide in scRNAseq studies of metastatic CRPC patients (He et al., 2021). Furthermore, anti-TGFB therapies have been proposed to reduce the development and progression of bone metastasis and to enhance the efficacy of immune checkpoint therapy in metastatic CRPC (Jiao et al., 2019). Consistent with enhanced TGFβ markers in our scRNAseq data, increased levels of Tgfb1 were found in both basal and ADT conditions in WPMY cells (FIG. 8G). Altogether these results are of great significance for PCa therapy since they identified regulators in the stroma whose therapeutic targeting impairs Ola-induced stromal activation and increases Ola anti-tumor efficacy.

A question that is still to be determined is the precise impact that Olaparib-mediated stromal activation has in the tumor epithelium. To address this question, the scRNAseq data of the PCa tumor epithelial compartment (FIGS. 81-8K) was interrogated. Of potential relevance, Olaparib treatment completely abolished the neuroendocrine cluster 2 (NE1; FIGS. 81-8K), supporting the concept that neuroendocrine tumors display enhanced sensitivity to the treatment with Olaparib (Liu et al., 2019; Zhang et al., 2018). However, Olaparib also expanded several other epithelial clusters: Cluster 0 is a luminal population; cluster 1 is a basal population; clusters 3 and 4 that are luminal but express high levels of EMT markers such as Vimentin (Vim) and Acta 2; and cluster 5 is a new neuroendocrine population that, in contrast to cluster 2, highly expresses the neuronal marker Synaptophysin (Syp), in addition to luminal and EMT markers (FIGS. 8I and 8J). Importantly, the Olaparib-treated tumor epithelium showed increased AR, EMT and TGFβ signaling, which suggests potential mechanisms of resistance to Olaparib therapy (FIG. 8L). Consistent with enrichment in TGFβ signaling signature in Olaparib-treated epithelial cells, there was an increased pSMAD2 staining, an established marker of TGFB activation, in Olaparib-treated prostates (FIG. 8M). Moreover, identification of ligand-receptor interactions between the stromal and epithelial populations by CellPhoneDB analysis revealed changes in receptors for HAS1/2 in Olaparib-treated populations (FIG. 8N); these included gaining novel CD44-mediated pairs between the stromal populations and the different clusters of tumor cells. Of note, CD44 is a receptor of HA that integrates extracellular matrix cues with growth factor and cytokine signals (Misra et al., 2015; Orian-Rousseau and Sleeman, 2014; Ponta et al., 2003). The data showed that Olaparib increased the HAS1/2-CD44 pairs, but also promoted the interaction of CD44 with other growth-factor receptors, such as FGFR2 or HBEGF (FIG. 8N). There was also gain of multiple interactions of the TGFβ receptor family and their ligands (FIG. 8N) that could account for the increased TGFβ signaling and EMT phenotype observed in tumor cells in Olaparib conditions. In this regard, binding of HA induces clustering of CD44 and the complex formation with other receptors (including the receptors for TGFB), which serve to further reinforce the activation of these pathways.

### Co-Targeting the Stroma Increases Olaparib Anti-Tumor Activity

Based on the data that Olaparib induces stromal activation, it was hypothesized that strategies targeting the stroma could improve the efficacy of Olaparib as an anti-tumor therapy. Since treatment with Olaparib induces an HA-rich stroma, and HA can be degraded by PEGPH20, a clinical-grade recombinant hyaluronidase (provided by Halozyme Therapeutics), it was tested whether treatment with PEGPH20 could revert Olaparib-induced stroma activation and enhance its anti-tumor activity. As shown in FIG. 9A, PEGPH20 efficiently degraded HA deposition in response to Olaparib treatment in WPMY-1 cells, in basal and ADT conditions (FIG. 9A). More importantly, PEGPH20 completely blocked the enhanced migration of PC3 cells in response to conditioned media from Olaparib-treated WPMY-1 cells under basal and ADT conditions (FIGS. 9B and 9C). Moreover, PEGPH20 also blocked Olaparib-induced proliferation in stromal cells (FIGS. 9D and 9E). Likewise, treatment with Galunisertib, a TGFβR inhibitor, completely abolished the stromal proliferation induced by Olaparib (FIGS. 9D and 9E). These results indicate that strategies aimed at targeting the stroma could normalize the stromal activation promoted by Olaparib. Consistent with this notion, the in vivo data showed that co-treatment with PEGPH20 enhanced Olaparib anti-tumor activity (FIGS. 9F and 9G). Collectively, these results demonstrate that combination therapies co-targeting the activated stroma could increase the efficacy of PARP inhibitor therapies such as Olaparib.

### Discussion

Despite recent advances in the characterization of the heterogeneity of fibroblast populations in the tumor stroma and the mounting evidence on their role in tumor progression, the molecular mechanisms that control stromal activation by the tumor epithelium remain largely unknown. Here, tumor-secreted lactate was identified as the main signal from the epithelium that activates tumor fibroblasts by downregulating p62. Previous work has shown that p62 is a stromal tumor suppressor whose downregulation in fibroblasts is a key event for CAF activation (Duran et al., 2016; Goruppi and Dotto, 2013; Linares et al., 2017; Valencia et al., 2014). However, the mechanisms whereby p62 is downregulated by the tumor in the stroma were not understood. p62 regulation is subjected to a fine-tune balance between transcription and post-translational mechanisms that control its degradation by autophagy or the proteosome machinery (Moscat and Diaz-Meco, 2009; Moscat et al., 2016). Herein, it was shown that lactate modulates p62 at the transcriptional level. Thus, although lactate reportedly induces autophagy (Brisson et al., 2016), which can contribute to lactate effects in tumor cells and the stroma, we show here that blocking autophagy or the proteosome failed to prevent p62-downregulation by lactate. At the transcriptional level, p62 has been shown to be regulated by several transcription factors including AP1, NRF2, NF-kB and TFEB under different conditions (Duran et al., 2008; Jain et al., 2010; Ling et al., 2012; Park et al., 2019). The AP-1 binding site in the p62 promoter was identified as the element accounting for lactate-mediated p62 downregulation. Interestingly, these findings are reminiscent of previously published data showing that AP-1 was also the major transcription factor for Ras-induced p62 mRNA expression in the tumor epithelium (Duran et al., 2008), which suggests that AP-1 is a hot spot for the upregulation of p62 by oncogenic transformation in the epithelium and its downregulation in the stroma. Regarding the composition of the AP-1 transcription factors mediating this effect in the stroma, the data demonstrate that c-FOS and c-JUN complexes are important since their recruitment to the p62 promoter was impaired in lactate-treated cells, whereas no changes were observed in the binding of FOSB and JUNB. This is interesting because JUNB has recently been shown to be key in the repression of p62 in keratinocytes (Sukseree et al., 2021), which suggests that different AP-1 proteins play distinct roles in p62 regulation under different cellular contexts.

Lactate has been considered for years as just the end product of glycolysis and as such, a metabolic waste (Ippolito et al., 2019). However more recent evidence has recognized many new roles for lactate including its relevance as a carbon source for cellular metabolism, and potential new functions as a signaling molecule in the TME (Brooks, 2018; Ippolito et al., 2019). Lactate is produced from pyruvate and is exported to the TME, where it can reach high concentrations of up to 40 mM, especially in tumor tissues (Walenta et al., 2000). Most tumor cells can engage in secretion and utilization of lactate depending on the context and extracellular microenvironment (Brooks, 2018). In addition to the shuttling that can take place among different tumor cells, lactate accumulation in the TME has significant effects on the non-malignant compartment, including, as shown here, on the activation of the fibroblasts surrounding the tumor, which results in their acquisition of the CAF phenotype (Bhagat et al., 2019). Therefore, targeting lactate metabolism as a potential therapeutic approach needs to integrate its effects not only on the tumor cell but also in the TME.

Lactate dehydrogenase catalyzes the conversion of lactate into pyruvate. This results in the reduction of NAD⁺ cellular levels, which is an important metabolic and signaling cofactor that impacts multiple functions, often dysregulated in cancer (Chini et al., 2021; Fang et al., 2017). Herein it was shown that low NAD⁺ levels, as consequence of lactate conversion to pyruvate, is central to the ability of lactate to downregulate p62 in fibroblasts and their subsequent conversion into CAF. Our data demonstrating that supplementation with NAD⁺ precursors, such as NR, rescued p62 levels and reverted the CAF phenotype, established that altering the cell NAD⁺/NADH ratio could be a mechanism to modulate stromal activation. In keeping with this notion, downregulation of nicotinamide phosphoribosyl transferase (NAMPT), a rate-limiting enzyme in NAD⁺ synthesis, has been shown to promote renal and lung fibrosis with increased collagen deposition and ECM remodeling (Muraoka et al., 2019). Furthermore, recent evidence demonstrated that stromal nicotinamide N-methyltransferase (NNMT), an enzyme that impairs NAD+ biosynthesis by depleting its precursor N-nicotinamide, induces CAF differentiation and cancer progression (Eckert et al., 2019). All these evidences are of potential relevance because they strongly suggest that the stromal activation promoted by NAD⁺ inhibition could limit the efficacy of anti-cancer therapies targeting NAD⁺ biosynthesis, such as NAMPT inhibitors, and could explain their reported limited efficacy in vivo (Galli et al., 2020). Supplementation with NR has been studied extensively and its dietary administration is considered safe and effective to increase NAD⁺ levels (Martens et al., 2018). NR supplements are proposed to ameliorate inflammation and metabolic dysfunction in aging, and there is a renewed interest in the potential of NAD-boosting therapies to treat human diseases (Chini et al., 2021; Fang et al., 2017). Based on the present data, an additional benefit of increasing NAD⁺ levels might be to reprogram the stroma back to a less activated phenotype by restoring p62 levels. However, the role of NAD⁺ in cancer is complex and manipulating its levels could be a double-edged sword (Chini et al., 2021; Fang et al., 2017). Thus, increasing the NAD⁺/NADH ratio by administering nicotinamide mononucleotide (NMN), another NAD⁺ precursor, has been shown to have a protumorigenic effect in K-Ras-driven pancreatic cancer (Nacarelli et al., 2019), unveiling the complexity of these therapeutic approaches.

The work herein identified PARP-1 inhibition as the key target of the reduced NAD₊ levels in the transcriptional downregulation of p62 by lactate. PARP1 is the major isoform of the PARP enzyme family and, like the other PARPs, its PARylation activity impacts multiple biological processes with a main dual function in DNA damage repair (DDR) and transcriptional regulation (Feng et al., 2015; Gibson and Kraus, 2012). Most of the therapeutic strategies using PARP inhibitors rely on their blockade of DDR and the creation of synthetic lethality in combination with genetic defects in homologous recombination-mediated repair (Farmer et al., 2005; Lord and Ashworth, 2017; McCabe et al., 2006), which are the basis for the treatment of patients with BRCA1- and BRCA2-associated cancers (Mateo et al., 2019). The use of PARP inhibitors has been most successfully exploited in BRCA1/2-deficient breast and ovarian cancers (Sonnenblick et al., 2015), and recently Olaparib has been approved for the treatment of mCRPC in patients with DDR gene mutations (de Bono et al., 2020; Hussain et al., 2020). However, the fact that the percentage of mCRPC patients with these mutations that could benefit from that type of therapy is a minority has led to new efforts for combinatorial strategies by chemically inducing DDR (Li et al., 2017; Zhang et al., 2018). Most of these efforts have been focused on the direct effect of PARP inhibitors on the tumor cell, and much less is known on how the tumor stroma responds to this type of treatment. The present data, showing that lactate inhibits PARP activity, and that PARP-1 knockdown downregulates p62 through the impaired PARylation of AP-1 transcription factors, resulting in CAF activation, might have important therapeutic implications. The results also highlight the increasingly recognized role of PARP1 in transcriptional regulation as a key mechanism of action beyond its role in DDR (Feng et al., 2015; Liu et al., 2019), and identify an unanticipated function for these inhibitors in the activation of the pro-tumorigenic potential of the tumor stroma. These data are consistent with the previously reported protumorigenic phenotype of PARP1 KO mice in the context of the TRAMP⁺ model, indicating the dominant protumorigenic role of the TME in PCa progression under conditions of PARP1 deficiency (Pu et al., 2014). This is an important finding because CAF activation in the TME by PARP inhibitors might limit or even blunt their therapeutic efficacy. Therefore, a better understanding of the fundamental mechanisms controlling the activation of the stromal has the potential to identify vulnerabilities that can improve cancer therapies.

### Example 2

### Methods

### In Vivo Experiments

For Olaparib treatment in mice, 13 weeks-old male TRAMP⁺/FVB were surgical castrated, after 10 days n=16, were randomly distributed to receive vehicle, n=8, or Olaparib n=8 (5 mg/kg/, 5 days each, i.p.). Mice were euthanized 3 weeks after the initiation of the treatment. Prostate and other organs were collected and analyzed histologically.

For the metastasis model, caudal artery injection was performed with 500000 RM1-BoMe3-RFP-Luc prostate cancer epithelial cells in 150 μl of PBS n=10 in 8-week-old C57B16 male mice. After 1-week luciferase was visualized using IVIS to detect signal in the lower limbs and in less frequently in visceral organs, mice with signal were randomly distributed to receive vehicle, n=5, or Olaparib n=5 (40 mg/kg/, 5 days each, i.p.). After 3 weeks, mice were euthanized and ex-vivo IVIS was performed in lower limbs, vertebrae, and visceral organs to detect metastasis sites. Tissues were collected and analyzed histologically.

### Bone in Culture

8-week-old C57B16 male mice were euthanized, and lower limb bone were collected. Femur epiphyses were dissected and fragmented in 4 pieces by sterilized micro dissecting scissors. Bone pieces were aligned into low-attachment 96-well plates pre-loaded with 200 μL DMEM/F12 supplemented with 5% charcoal striped FBS for ADT conditions. Bone pieces were treated with DMSO or Olaparib 20 μM for 48 h and then 5000 RM1-BoMe3-RFP-Luc cells were seed on top of the bone pieces. After 48 hours, pictures were taken using EVOS M5000 Imaging System and RFP⁺ positive areas were measured using Fiji.

### Stromal-Induced Activation by PARP Inhibitors in Other Cancers Beyond Prostate Cancer

Olaparib (Ola) downregulated p62 expression, a master regulator of CAFs activation, in stromal cell lines from different tissue origins, including prostate, lung, breast or endometrium (FIGS. 13A-13D). These results demonstrate that the stromal activation induced by PARP inhibitors is a general mechanism taking place not only in the prostate stroma but also in stromal cells from other types of cancer. Consistent with these results, gene set enrichment analysis (GSEA) of RNAseq data of BRCA deficient breast tumors treated with the PARP inhibitor Talazoparib (gene dataset GEO: GSE125206) showed significant enrichment of signatures of stromal activation (FIG. 13E). Similar enrichment in signatures of stromal activation was observed in GSEA of RNAseq data from a PDX model of high grade serous ovarian cancer treated with niraparib (FIG. 13F). These results support the notion that co-targeting the stroma enhances the efficacy of PARP inhibitors in other types of cancer beyond prostate cancer, such as breast and ovarian cancer, where this therapy is approved especially for BRCA deficient tumors.

### Blocking Inflammatory Signals Reverts Stromal Activation by PARP Inhibitors

To further characterize the molecular mechanisms involved in the stromal activation by PARP inhibitors, RNAseq data of human prostate fibroblasts treated with Ola was analyzed by GSEA. Interestingly, a significant enrichment was found in inflammatory signaling pathways mediated by inflammatory cytokines such as TNFα and IL-6 and the activation of the JAK-STAT cascade (FIG. 11A). Importantly, similar results were found in the analysis of other datasets of two different in vivo models of breast and ovarian tumors, treated with two different PARP inhibitors, Talazoparib and Niraparib (FIG. 11B). These results indicate that the activation of these inflammatory pathways is a general signaling mechanism in response to PARP inhibitors, regardless the tumor type. Therefore, it was investigated whether blocking these inflammatory pathways could revert the stromal activation mediated by PARP inhibitors. To do that, fibroblasts were treated with Ola in the presence or absence of a JAK inhibitor, Lestaurtinib (FIG. 11C). Of note, incubation with Lestaurtinib and Ola reduces the expression of HAS3, HAS2 and IL6 induced by Ola (FIG. 11D). More importantly, conditioned media from fibroblasts treated with the Lestaurtinib and Ola combination completely blocks the proliferation of cancer cells induced by Ola-treated stromal cells, as determined by Edu (5-ethynyl-2′-deoxyuridine) incorporation (FIG. 11E). Similar results were obtained with conditioned media was incubated with anti-IL6 or anti-TNFα antibodies. These results demonstrate that the inflammatory response mediated by TNFα, IL-6 and JAK activation are important events for the stromal activation by PARP inhibitors and that strategies to therapeutically target these signaling cascades could enhance the efficacy of PARP inhibitors. Therefore, the data support the clinical use of combinations of PARP inhibitors and therapies blocking TNFα, IL6 or JAK to improve the clinical response to PARP inhibition in cancer.

### PARP Inhibitors Induce Stromal Activation in Metastatic Sites

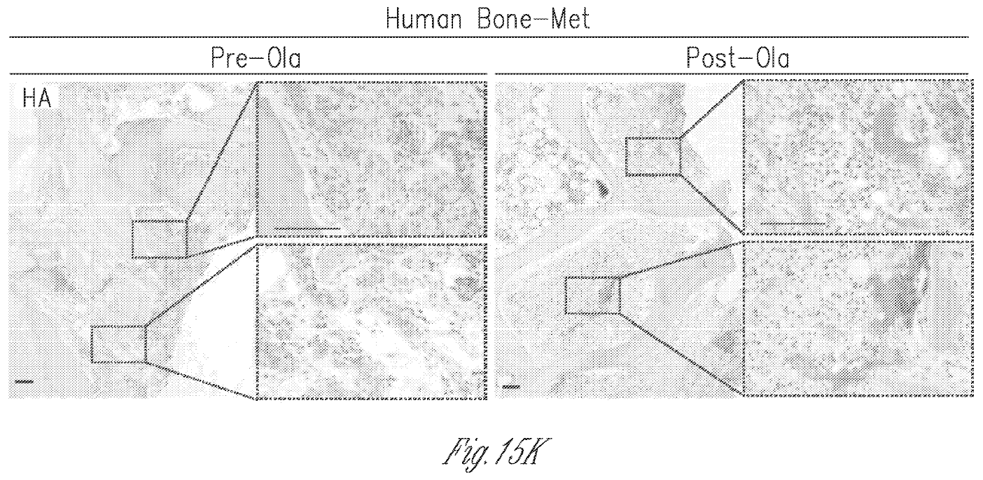

Since Ola is approved for the treatment of human metastatic castration-resistant prostate cancer (mCRPC), it was investigated whether Ola was able to induce stromal activation not only in the prostate but also in metastatic sites. Treatment with Ola of castrated TRAMP⁺/FVB F1 mice, which have a high incidence of spontaneous metastasis in lymph nodes, showed increased «SMA expression and higher hyaluronan (HA) deposition in metastatic lymph nodes from Ola-treated mice as compared to those from vehicle-treated mice (FIGS. 15A and 15B). Furthermore, analysis of bones from these mice demonstrated higher levels of HA in surrounding bone marrow stromal cells and in the growth plate of Ola-treated mice (FIG. 15C). These in vivo mouse data demonstrate that Ola treatment remodels the stromal niche not only in the prostate but also in metastatic sites such as lymph nodes or bone. This remodeling of the metastatic niche could support metastatic growth and predispose these sites to further metastasis seeding. To test this hypothesis, bone-in-culture-arrays (BICA) were used to model bone colonization of cancer cells in ex vivo cultures. Mouse fragmented epiphysis and metaphysis of hindlimb bones were treated with Ola in androgen-deprivation conditions (ADT) prior to the seed of luciferase-modified RM1 prostate cancer cells. Of note, Ola significantly increased the seeding of cancer cells onto the bone (FIGS. 15D and 15E). In addition, analysis of mouse bone marrow stromal cell cultures isolated from in vivo Ola-treated TRAMP⁺ mice showed enhanced expression of markers of stromal activation such as Acta2 and of the HA synthase Has2, which is important for HA production (FIG. 15F). Likewise, in vitro Ola treatment of bone marrow stromal cells also showed increased expression of markers of TGFβ family (Tgfb3 and Tgfb2) and Has3 (FIG. 15G). These results demonstrate that Ola treatment promotes stromal activation in the bone, which predisposes the bone niche to the seed of metastatic cancer cells. Moreover, deposition of HA was observed in other metastatic sites such as liver after treatment with Ola of mice in an in vivo metastasis model of injection via caudal artery of metastatic cancer cells (FIG. 15I). Also, analysis of bone metastasis treated with Ola demonstrated an increased activation of TGFβ signaling as shown by enhanced nuclear pSMAD2 in cancer cells (FIG. 15J). These observations support clinical studies of combinatorial therapy of PARP inhibitors with TGFβ inhibition. To further strengthen the human relevance of these observations, paired biopsies of bone metastasis from a mCRPC patient pre- and post-treatment with Ola were analyzed. Importantly, there was a robust increase in HA deposition in the Ola-treated bone metastasis consistent with our mouse data (FIG. 15K).

### REFERENCES

Aaltomaa et al., Urol. Int., 69:266 (2002).
Affo et al., Endocr. Relat. Cancer, 19: R187 (2021).
Bhagat et al., Elife, 8: (2019).
Bjerke et al., Oncogene, 33:3660 (2014).
Bluemn et al., Cancer Cell., 32:474 (2017).
Brisson et al., Cancer Cell., 30:418 (2016).
Brooks, Cell Metab., 27:757 (2018).
Chen et al., Nat. Commun., 9:4315 (2018).
Chini et al., Cell Metab., 33:1076 (2021).
Covarrubias et al., Nat. Rev. Mol. Cell. Biol., 22:119 (2021).
Cunha et al., Differentiation; Res. Biol. Div., 70:473 (2002).
de Bono et al., N. Engl. J. Med., 382:2091 (2020).
Duran et al., Mol. Cell., 44:134 (2011).
Duran et al., Cancer Cell., 30:595 (2016).
Duran et al., Cancer Cell., 13:343 (2008).
Eckert et al., Nature, 569:723 (2019).
Fang et al., Trends Mol. Med., 23:899 (2017).
Farmer et al., Nature, 434:917 (2005).
Feng et al., Mol. Cell., 58:925 (2017).
Fournier et al., Cancer Cell., 27:809 (2015).
Galli et al., Front Pharmacol., 11:656 (2020).
Gibson & Kraus, Nat. Rev. Mol. Cell Biol., 13:411 (2012).
Goruppi & Dotto, Trends Cell. Biol., 23:593 (2013).
Halestrap, Mol. Aspects Med., 34:337 (2013).
Hanahan & Weinberg, Cell, 144:646 (2011).
He et al., Nat. Med., 27:426 (2021).
Heinz et al., Mol. Cell., 38:576 (2010).
Huang et al., J. Mol. Cell. Cardiol., 46:25 (2009).
Huang et al., Cancer Cell., 33:770 (2018).
Hussain et al., N. Engl. J. Med., 383:2345 (2020).
Ippolito et al., Trends Biochem. Sci., 44:153 (2019).
Jain et al., J. Biol. Chem., 285:22576 (2010).
Jiao et al., Cell, 179:1177 (2019).
Johnson et al., J. Urol., 196:1436 (2016).
Karthaus et al., Science, 368:497 (2020).
Kasashima et al., Dev. Cell. _:_ (2020).
Klemm & Joyce, Trends Cell Biol., 25:198 (2015).
Leinonen et al., Clin. Cancer Res., 16:2845 (2010).
Li et al., Sci. Signal, 10: (2017).
Linares et al., Cell. Metab., 26:817 (2017).
Ling et al., Cancer Cell., 21:105 (2012).
Lipponen et al., Eur. J. Cancer, 37:849 (2001).
Liu et al., Clin. Cancer Res., 25:6839 (2019).
Lord & Ashworth, Science, 355:1152 (2017).
Lu et al., Genes Dev., 31:2337 (2017).
Martens et al., Nat. Commun., 9:1286 (2018).
Massague, Cell, 134:215 (2008).
Mateo et al., Ann. Oncol., 30:1437 (2019).
McCabe et al., Cancer Res., 66:8109 (2006).
Misra et al., Front Immunol., 6:201 (2015).
Moscat & Diaz-Meco, Cell, 137:1001 (2009).
Moscat et al., Cell, 167:606 (2016).
Muraoka et al., Cell Rep., 27:199 (2019).
Nacarelli et al., Nat. Cell Biol., 21:397 (2019).
Nakanishi et al., Immunity, 49, 1132 (2018).
Orian-Rousseau & Sleeman, Adv. Cancer Res., 123:231 (2014).
Park et al., Sci. Rep., 9:14354 (2019).
Ponta et al., Nat. Rev. Mol. Cell Biol., 4:33 (2003).
Pu et al., Carcinogenesis, 35:2592 (2014).
Reina-Campos et al., Biochim. Biophys. Acta, _:_ (2018).
Rowley, Cancer Metast. Rev., 17:411 (1998).
Sahai et al., Nat. Rev. Cancer, 20:174 (2020).
Schindelin et al., Nat. Methods, 9:676 (2012).
Sonnenblick et al., Nat. Rev. Clin. Oncol., 12:27 (2015).
Sukseree et al., J. Allergy Clin. Immunol., 147:2386 (2021).
Tiwari et al., Nat. Commun., 11:384 (2020).
Tomlins et al., Cancer Cell., 13:519 (2008).
Toole, Nat. Rev. Cancer, 4:528 (2004).
Toole & Slomiany, Semin. Cancer Biol., 18:244 (2008).
Tuxhorn et al., J. Urol., 166:2472 (2001).
Umemura et al., Cancer Cell., _:_ (2016)
Valencia et al., Cancer Cell., 26:121 (2014).
Vander Heiden et al., Science, 324:1029 (2009).
Walenta et al., Cancer Res., 60:916 (2000).
Zhang et al., Clin. Cancer Res., 24:696 (2018).
Zhang et al., Cell, 154:1060 (2013).
Zhang et al., Cancer Cell., 38:279 (2020).

All patents and publications referenced or mentioned herein are indicative of the levels of skill of those skilled in the art to which the invention pertains, and each such referenced patent or publication is hereby specifically incorporated by reference to the same extent as if it had been incorporated by reference in its entirety individually or set forth herein in its entirety. Applicants reserve the right to physically incorporate into this specification any and all materials and information from any such cited patents or publications.

The following statements are intended to describe and summarize various embodiments of the invention according to the foregoing description in the specification.

### Statements

1. A method comprising administering to a subject in need thereof a composition comprising one or more PARP1 inhibitors and one or more inhibitors of activated stromal/activated cancer-associated fibroblasts or one or more inhibitors of TNFalpha, IL6 or JAK.
2. The method of statement 1, wherein the subject has cancer or is suspected of a having cancer.
3. The method of statement 1 or 2, wherein the subject has primary cancer, metastatic cancer, or recurrent cancer.
4. The method of statement 1, 2 or 3, wherein the subject has cancer of the breast, cancer of the lung, cancer of the adrenal cortex, cancer of the cervix, cancer of the endometrium, cancer of the esophagus, cancer of the head, cancer of the neck, cancer of the liver, cancer of the pancreas, cancer of the prostate, cancer of the thymus, carcinoid tumors, chronic lymphocytic leukemia, Ewing's sarcoma, gestational trophoblastic tumors, hepatoblastoma, multiple myeloma, non-small cell lung cancer, retinoblastoma, or tumor in the ovaries.
5. The method of any of statements 1-4, wherein the one or more PARP1 inhibitors are olaparib (Lynparza), niraparib (Zejula), rucaparib (Rubraca), talazoparib (Talzenna), veliparib (ABT-888), or a combination thereof.
6. The method of any of statements 1-5, wherein the one or more inhibitors of activated stromal/activated cancer-associated fibroblasts include hyaluronan (HA) synthase inhibitors, hyaluronan build-up inhibitors (e.g., PEGylated Recombinant Human Hyaluronidase, PEGPH20), autophagy inhibitors (e.g., ULK inhibitors), fibroblast activation protein alpha (FAPa) inhibitors, GW4064 (farnesoid X receptor (FXR) agonists, Pirfenidone (PDF) combined with doxorubicin, SMO-inhibitors (e.g., vismodegib, sonidegib), amphiregulin inhibitors, CXCL12 antagonists (e.g., an E5 antagonistic peptide), DDR2 inhibitors (e.g., WRG-28, which is a small molecule inhibitor of DDR2), or a combination thereof.
7. The method of any of statements 1-6, wherein the composition comprises Olaparib and PEGPH20.

The specific methods and compositions described herein are representative of preferred embodiments and are exemplary and not intended as limitations on the scope of the invention. Other objects, aspects, and embodiments will occur to those skilled in the art upon consideration of this specification and are encompassed within the spirit of the invention as defined by the scope of the claims. It will be readily apparent to one skilled in the art that varying substitutions and modifications may be made to the invention disclosed herein without departing from the scope and spirit of the invention.

The invention illustratively described herein suitably may be practiced in the absence of any element or elements, or limitation or limitations, which is not specifically disclosed herein as essential. The methods and processes illustratively described herein suitably may be practiced in differing orders of steps, and the methods and processes are not necessarily restricted to the orders of steps indicated herein or in the claims.

As used herein and in the appended claims, the singular forms “a,” “an,” and “the” include plural reference unless the context clearly dictates otherwise. Thus, for example, a reference to “a nucleic acid” or “a protein” or “a cell” includes a plurality of such nucleic acids, proteins, or cells (for example, a solution or dried preparation of nucleic acids or expression cassettes, a solution of proteins, or a population of cells), and so forth. In this document, the term “or” is used to refer to a nonexclusive or, such that “A or B” includes “A but not B,” “B but not A,” and “A and B,” unless otherwise indicated.

Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims and statements of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein. In addition, where features or aspects of the invention are described in terms of Markush groups, those skilled in the art will recognize that the invention is also thereby described in terms of any individual member or subgroup of members of the Markush group.

1. A method comprising administering to a subject in need thereof one or more PARP1 inhibitors and one or more inhibitors of activated stromal/activated cancer-associated fibroblasts or one or more modulators of TNFalpha, IL6 or JAK.

2. (canceled)

3. The method of claim 1, wherein the subject has cancer or is suspected of a having cancer.

4. The method of claim 1, wherein the subject has primary cancer, metastatic cancer, or recurrent cancer.

5. (canceled)

6. The method of claim 1, wherein the one or more PARP1 inhibitors are olaparib (Lynparza), niraparib (Zejula), rucaparib (Rubraca), talazoparib (Talzenna), veliparib (ABT-888), or a combination thereof.

7. The method of claim 1, wherein the one or more inhibitors of activated stromal/activated cancer-associated fibroblasts include hyaluronan (HA) synthase inhibitors, hyaluronan build-up inhibitors, autophagy inhibitors, fibroblast activation protein alpha (FAPα) inhibitors, GW4064 (farnesoid X receptor (FXR) agonists, Pirfenidone (PDF) combined with doxorubicin, SMO-inhibitors, amphiregulin inhibitors, CXCL12 antagonists, DDR2 inhibitors, or a combination thereof.

8. The method of claim 7, wherein the DDR2 inhibitor comprises WRG-28.

9. The method of claim 7, wherein the hyaluronan build-up inhibitor comprises PEGylated Recombinant Human Hyaluronidase, PEGPH20.

10. The method of claim 7, wherein the autophagy inhibitor comprises a ULK inhibitor.

11. The method of claim 7, wherein the SMO inhibitor comprises vismodegib or sonidegib.

12. The method of claim 7, wherein the CXCL12 antagonist comprises an E5 antagonistic peptide.

13. The method of claim 1, wherein the Olaparib and PEGPH20 are administered.

14. The method of claim 1, wherein the one or more PARP1 inhibitors are administered before the one or more inhibitors of activated stromal/activated cancer-associated fibroblasts or one or more modulators of TNFalpha, IL6 or JAK.

15. The method of claim 1, wherein the one or more PARP1 inhibitors are administered after the one or more inhibitors of activated stromal/activated cancer-associated fibroblasts or one or more modulators of TNFalpha, IL6 or JAK.

16. The method of claim 1, wherein the one or more PARP1 inhibitors are administered concurrently with the one or more inhibitors of activated stromal/activated cancer-associated fibroblasts or one or more modulators of TNFalpha, IL6 or JAK.

17.-20. (canceled)

21. The method of claim 1, wherein the one or more inhibitors of activated stromal/activated cancer-associated fibroblasts or one or more modulators of TNFalpha, IL6 or JAK are systemically administered.

22. The method of claim 1, wherein the JAK inhibitor comprises lesataurtinib, baricitinib, tofacitinib, upadacitinib, filgonitib, delgocitinib, deucravacitinib, LS104, ON044580,NVP-BBT594, or NVP-CHZ868.

23. The method of claim 1, wherein the IL6 inhibitor comprises an antibody or an antigen binding portion thereof.

24. (canceled)

25. The method of claim 1, wherein the IL6 inhibitor comprises ARGX-109, FE301 or FM101.

26. The method of claim 1, wherein the TNFalpha inhibitor comprises adalimumab, certolizumab pegol, etanercept, golimumab, or infliximab.

27. A composition comprising one or more PARP1 inhibitors and one or more inhibitors of activated stromal/activated cancer-associated fibroblasts or one or more modulators of TNFalpha, IL6 or JAK.
